SEQUENCE LISTING (164 sequences)

SEQ ID NO: 1 - protein, 1368 aa, Streptococcus pyogenes [sequence omitted]

SEQ ID NO: 2 - protein, 1269 aa, Mycoplasma gallisepticum
MNNSIKSKPEVTIGLDLGVGSVGWAIVDNETNIIHHLGSRLFSQAKTAEDRRSFRGVRRLIRRRKYKLKRFVNLIWKYNSYFGFKNKEDILNNYQEQQKLHNTVLNLKSEALNAKIDPKALSWILHDYLKNRGHFYEDNRDFNVYPTKELAKYFDKYGYYKGIIDSKEDNDNKLEEELTKYKFSNKHWLEEVKKVLSNQTGLPEKFKEEYESLFSYVRNYSEGPGSINSVSPYGIYHLDEKEGKVVQKYNNIWDKTIGKCNIFPDEYRAPKNSPIAMIFNEINELSTIRSYSIYLTGWFINQEFKKAYLNKLLDLLIKTNGEKPIDARQFKKLREETIAESIGKETLKDVENEEKLEKEDHKWKLKGLKLNTNGKIQYNDLSSLAKFVHKLKQHLKLDFLLEDQYATLDKINFLQSLFVYLGKHLRYSNRVDSANLKEFSDSNKLFERILQKQKDGLFKLFEQTDKDDEKILAQTHSLSTKAMLLAITRMTNLDNDEDNQKNNDKGWNFEAIKNFDQKFIDITKKNNNLSLKQNKRYLDDRFINDAILSPGVKRILREATKVFNAILKQFSEEYDVTKVVIELARELSEEKELENTKNYKKLIKKNGDKISEGLKALGISEDEIKDILKSPTKSYKFLLWLQQDHIDPYSLKEIAFDDIFTKTEKFEIDHIIPYSISFDDSSSNKLLVLAESNQAKSNQTPYEFISSGNAGIKWEDYEAYCRKFKDGDSSLLDSTQRSKKFAKMMKTDTSSKYDIGFLARNLNDTRYATIVFRDALEDYANNHLVEDKPMFKVVCINGSVTSFLRKNFDDSSYAKKDRDKNIHHAVDASIISIFSNETKTLFNQLTQFADYKLFKNTDGSWKKIDPKTGVVTEVTDENWKQIRVRNQVSEIAKVIEKYIQDSNIERKARYSRKIENKTNISLFNDTVYSAKKVGYEDQIKRKNLKTLDIHESAKENKNSKVKRQFVYRKLVNVSLLNNDKLADLFAEKEDILMYRANPWVINLAEQIFNEYTENKKIKSQNVFEKYMLDLTKEFPEKFSEFLVKSMLRNKTAIIYDDKKNIVHRIKRLKMLSSELKENKLSNVIIRSKNQSGTKLSYQDTINSLALMIMRSIDPTAKKQYIRVPLNTLNLHLGDHDFDLHNMDAYLKKPKFVKYLKANEIGDEYKPWRVLTSGTLLIHKKDKKLMYISSFQNLNDVIEIKNLIETEYKENDDSDSKKKKKANRFLMTLSTILNDYILLDAKDNFDILGLSKNRIDEILNSKLGLDKIVK

SEQ ID NO: 3 - protein, 1334 aa, Staphylococcus pseudintermedius
MGRKPYILSLDIGTGSVGYACMDKGFNVLKYHDKDALGVYLFDGALTAQERRQFRTSRRRKNRRIKRLGLLQELLAPLVQNPNFYQFQRQFAWKNDNMDFKNKSLSEVLSFLGYESKKYPTIYHLQEALLLKDEKFDPELIYMALYHLVKYRGHFLFDHLKIENLTNNDNMHDFVELIETYENLNNIKLNLDYEKTKVIYEILKDNEMTKNDRAKRVKNMEKKLEQFSIMLLGLKFNEGKLFNHADNAEELKGANQSHTFADNYEENLTPFLTVEQSEFIERANKIYLSLTLQDILKGKKSMAMSKVAAYDKFRNELKQVKDIVYKADSTRTQFKKIFVSSKKSLKQYDATPNDQTFSSLCLFDQYLIRPKKQYSLLIKELKKIIPQDSELYFEAENDTLLKVLNTTDNASIPMQINLYEAETILRNQQKYHAEITDEMIEKVLSLIQFRIPYYVGPLVNDHTASKFGWMERKSNESIKPWNFDEVVDRSKSATQFIRRMTNKCSYLINEDVLPKNSLLYQEMEVLNELNATQIRLQTDPKNRKYRMMPQIKLFAVEHIFKKYKTVSHSKFLEIMLNSNHRENFMNHGEKLSIFGTQDDKKFASKLSSYQDMTKIFGDIEGKRAQIEEIIQWITIFEDKKILVQKLKECYPELTSKQINQLKKLNYSGWGRLSEKLLTHAYQGHSIIELLRHSDENFMEILTNDVYGFQNFIKEENQVQSNKIQHQDIANLTTSPALKKGIWSTIKLVRELTSIFGEPEKIIMEFATEDQQKGKKQKSRKQLWDDNIKKNKLKSVDEYKYIIDVANKLNNEQLQQEKLWLYLSQNGKCMYSGQSIDLDALLSPNATKHYEVDHIFPRSFIKDDSIDNKVLVIKKMNQTKGDQVPLQFIQQPYERIAYWKSLNKAGLISDSKLHKLMKPEFTAMDKEGFIQRQLVETRQISVHVRDFLKEEYPNTKVIPMKAKMVSEFRKKFDIPKIRQMNDAHHAIDAYLNGVVYHGAQLAYPNVDLFDFNFKWEKVREKWKALGEFNTKQKSRELFFFKKLEKMEVSQGERLISKIKLDMNHFKINYSRKLANIPQQFYNQTAVSPKTAELKYESNKSNEVVYKGLTPYQTYVVAIKSVNKKGKEKMEYQMIDHYVFDFYKFQNGNEKELALYLAQRENKDEVLDAQIVYSLNKGDLLYINNHPCYFVSRKEVINAKQFELTVEQQLSLYNVMNNKETNVEKLLIEYDFIAEKVINEYHHYLNSKLKEKRVRTFFSESNQTHEDFIKALDELFKVVTASATRSDKIGSRKNSMTHRAFLGKGKDVKIAYTSISGLKTTKPKSLFKLAESRNEL

SEQ ID NO: 4 - protein, 1084 aa, Corynebacterium diphtheriae
MKYHVGIDVGTFSVGLAAIEVDDAGMPIKTLSLVSHIHDSGLDPDKIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDEPSDAFKAIREEIKRASGQPVPETATVGQMVTLCELGTLKLRGEGGVLSARLQQSDHAREIQEICRMQEIGQELYRKIIDVVFAAESPKGSASSRVGKDPLQPGKNRALKASDAFQRYRIAALIGNLRVRVDGEKRILSVEEKNLVFDHLVNLAPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLADGVDLYTARLQEFGIEPSWTPPAPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLEFLDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHEETIGKLSKVKLGSQLSVSDIDKASSEALWCALTREPDFDPKDGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFGDTRLRLRPLQMSKEGIKKESAPELSKIIDRPGWLPAVNKLFSEGNVTVVRRDSLGRVRLESTAHLPVTWKVQ

SEQ ID NO: 5 - protein, 967 aa, Unknown
  Description of Unknown Candidatus Parvarchaeum acidiphilum ARMAN-4 sequence
MLGSSRYLRYNLTSFEGKEPFLIMGYYKEYNKELSSKAQKEFNDQISEFNSYYKLGIDLGDKTGIAIVKGNKIILAKTLIDLHSQKLDKRREARRNRRTRLSRKKRLARLRSWVMRQKVGNQRLPDPYKIMHDNKYWSIYNKSNSANKKNWIDLLIHSNSLSADDFVRGLTIIFRKRGYLAFKYLSRLSDKEFEKYIDNLKPPISKYEYDEDLEELSSRVENGEIEEKKFEGLKNKLDKIDKESKDFQVKQREEVKKELEDLVDLFAKSVDNKIDKARWKRELNNLLDKKVRKIRFDNRFILKCKIKGCNKNTPKKEKVRDFELKMVLNNARSDYQISDEDLNSFRNEVINIFQKKENLKKGELKGVTIEDLRKQLNKTFNKAKIKKGIREQIRSIVFEKISGRSKFCKEHLKEFSEKPAPSDRINYGVNSAREQHDFRVLNFIDKKIFKDKLIDPSKLRYITIESPEPETEKLEKGQISEKSFETLKEKLAKETGGIDIYTGEKLKKDFEIEHIFPRARMGPSIRENEVASNLETNKEKADRTPWEWFGQDEKRWSEFEKRVNSLYSKKKISERKREILLNKSNEYPGLNPTELSRIPSTLSDFVESIRKMFVKYGYEEPQTLVQKGKPIIQVVRGRDTQALRWRWHALDSNIIPEKDRKSSFNHAEDAVIAACMPPYYLRQKIFREEAKIKRKVSNKEKEVTRPDMPTKKIAPNWSEFMKTRNEPVIEVIGKVKPSWKNSIMDQTFYKYLLKPFKDNLIKIPNVKNTYKWIGVNGQTDSLSLPSKVLSISNKKVDSSTVLLVHDKKGGKRNWVPKSIGGLLVYITPKDGPKRIVQVKPATQGLLIYRNEDGRVDAVREFINPVIEMYNNGKLAFVEKENEEELLKYFNLLEKGQKFERIRRYDMITYNSKFYYVTKINKNHRVTIQEESKIKAESDKVKSSSGKEYTRKETEELSLQKLAELISI

SEQ ID NO: 6 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 7 - protein, 1338 aa, Coprococcus catus
MKQEYFLGLDMGTGSLGWAVTDSTYQVMRKHGKALWGTRLFESASTAEERRMFRTARRRLDRRNWRIQVLQEIFSEEISKVDPGFFLRMKESKYYPEDKRDAEGNCPELPYALFVDDNYTDKNYHKDYPTIYHLRKMLMETTEIPDIRLVYLVLHHMMKHRGHFLLSGDISQIKEFKSTFEQLIQNIQDEELEWHISLDDAAIQFVEHVLKDRNLTRSTKKSRLIKQLNAKSACEKAILNLLSGGTVKLSDIFNNKELDESERPKVSFADSGYDDYIGIVEAELAEQYYIIASAKAVYDWSVLVEILGNSVSISEAKIKVYQKHQADLKTLKKIVRQYMTKEDYKRVFVDTEEKLNNYSAYIGMTKKNGKKVDLKSKQCTQADFYDFLKKNVIKVIDHKEITQEIESEIEKENFLPKQVTKDNGVIPYQVHDYELKKILDNLGTRMPFIKENAEKIQQLFEFRIPYYVGPLNRVDDGKDGKFTWSVRKSDARIYPWNFTEVIDVEASAEKFIRRMTNKCTYLVGEDVLPKDSLVYSKFMVLNELNNLRLNGEKISVELKQRIYEELFCKYRKVTRKKLERYLVIEGIAKKGVEITGIDGDFKASLTAYHDFKERLTDVQLSQRAKEAIVLNVVLFGDDKKLLKQRLSKMYPNLTTGQLKGICSLSYQGWGRLSKTFLEEITVPAPGTGEVWNIMTALWQTNDNLMQLLSRNYGFTNEVEEFNTLKKETDLSYKTVDELYVSPAVKRQIWQTLKVVKEIQKVMGNAPKRVFVEMAREKQEGKRSDSRKKQLVELYRACKNEERDWITELNAQSDQQLRSDKLFLYYIQKGRCMYSGETIQLDELWDNTKYDIDHIYPQSKTMDDSLNNRVLVKKNYNAIKSDTYPLSLDIQKKMMSFWKMLQQQGFITKEKYVRLVRSDELSADELAGFIERQIVETRQSTKAVATILKEALPDTEIVYVKAGNVSNFRQTYELLKVREMNDLHHAKDAYLNIVVGNAYFVKFTKNAAWFIRNNPGRSYNLKRMFEFDIERSGEIAWKAGNKGSIVTVKKVMQKNNILVTRKAYEVKGGLFDQQIMKKGKGQVPIKGNDERLADIEKYGGYNKAAGTYFMLVKSLDKKGKEIRTIEFVPLYLKNQIEINHESAIQYLAQERGLNSPEILLSKIKIDTLFKVDGFKMWLSGRTGNQLIFKGANQLILSHQEAAILKGVVKYVNRKNENKDAKLSERDGMTEEKLLQLYDTFLDKLSNTVYSIRLSAQIKTLTEKRAKFIGLSNEDQCIVLNEILHMFQCQSGSANLKLIGGPGSAGILVMNNNITACKQISVINQSPTGIYEKEIDLIKL

SEQ ID NO: 8 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 9 - protein, 1150 aa, Enterococcus faecalis
MYSIGLDLGISSVGWSVIDERTGNVIDLGVRLFSAKNSEKNLERRTNRGGRRLIRRKTNRLKDAKKILAAVGFYEDKSLKNSCPYQLRVKGLTEPLSRGEIYKVTLHILKKRGISYLDEVDTEAAKESQDYKEQVRKNAQLLTKYTPGQIQLQRLKENNRVKTGINAQGNYQLNVFKVSAYANELATILKTQQAFYPNELTDDWIALFVQPGIAEEAGLIYRKRPYYHGPGNEANNSPYGRWSDFQKTGEPATNIFDKLIGKDFQGELRASGLSLSAQQYNLLNDLTNLKIDGEVPLSSEQKEYILTELMTKEFTRFGVNDVVKLLGVKKERLSGWRLDKKGKPEIHTLKGYRNWRKIFAEAGIDLATLPTETIDCLAKVLTLNTEREGIENTLAFELPELSESVKLLVLDRYKELSQSISTQSWHRFSLKTLHLLIPELMNATSEQNTLLEQFQLKSDVRKRYSEYKKLPTKDVLAEIYNPTVNKTVSQAFKVIDALLVKYGKEQIRYITIEMPRDDNEEDEKKRIKELHAKNSQRKNDSQSYFMQKSGWSQEKFQTTIQKNRRFLAKLLYYYEQDGICAYTGLPISPELLVSDSTEIDHIIPISISLDDSINNKVLVLSKANQVKGQQTPYDAWMDGSFKKINGKFSNWDDYQKWVESRHFSHKKENNLLETRNIFDSEQVEKFLARNLNDTRYASRLVLNTLQSFFTNQETKVRVVNGSFTHTLRKKWGADLDKTRETHHHHAVDATLCAVTSFVKVSRYHYAVKEETGEKVMREIDFETGEIVNEMSYWEFKKSKKYERKTYQVKWPNFREQLKPVNLHPRIKFSHQVDRKANRKLSDATIYSVREKTEVKTLKSGKQKITTDEYTIGKIKDIYTLDGWEAFKKKQDKLLMKDLDEKTYERLLSIAETTPDFQEVEEKNGKVKRVKRSPFAVYCEENDIPAIQKYAKKNNGPLIRSLKYYDGKLNKHINITKDSQGRPVEKTKNGRKVTLQSLKPYRYDIYQDLETKAYYTVQLYYSDLRFVEGKYGITEKEYMKKVAEQTKGQVVRFCFSLQKNDGLEIEWKDSQRYDVRFYNFQSANSINFKGLEQEMMPAENQFKQKPYNNGAINLNIAKYGKEGKKLRKFNTDILGKKHYLFYEKEPKNIIK

SEQ ID NO: 10 - protein, 1233 aa, Unknown
  Description of Unknown Mycoplasmopsis canis PG 14 sequence
MEKKRKVTLGFDLGIASVGWAIVDSETNQVYKLGSRLFDAPDTNLERRTQRGTRRLLRRRKYRNQKFYNLVKRTEVFGLSSREAIENRFRELSIKYPNIIELKTKALSQEVCPDEIAWILHDYLKNRGYFYDEKETKEDFDQQTVESMPSYKLNEFYKKYGYFKGALSQPTESEMKDNKDLKEAFFFDFSNKEWLKEINYFFNVQKNILSETFIEEFKKIFSFTRDISKGPGSDNMPSPYGIFGEFGDNGQGGRYEHIWDKNIGKCSIFTNEQRAPKYLPSALIFNFLNELANIRLYSTDKKNIQPLWKLSSVDKLNILLNLFNLPISEKKKKLTSTNINDIVKKESIKSIMISVEDIDMIKDEWAGKEPNVYGVGLSGLNIEESAKENKFKFQDLKILNVLINLLDNVGIKFEFKDRNDIIKNLELLDNLYLFLIYQKESNNKDSSIDLFIAKNESLNIENLKLKLKEFLLGAGNEFENHNSKTHSLSKKAIDEILPKLLDNNEGWNLEAIKNYDEEIKSQIEDNSSLMAKQDKKYLNDNFLKDAILPPNVKVTFQQAILIFNKIIQKFSKDFEIDKVVIELAREMTQDQENDALKGIAKAQKSKKSLVEERLEANNIDKSVFNDKYEKLIYKIFLWISQDFKDPYTGAQISVNEIVNNKVEIDHIIPYSLCFDDSSANKVLVHKQSNQEKSNSLPYEYIKQGHSGWNWDEFTKYVKRVFVNNVDSILSKKERLKKSENLLTASYDGYDKLGFLARNLNDTRYATILFRDQLNNYAEHHLIDNKKMFKVIAMNGAVTSFIRKNMSYDNKLRLKDRSDFSHHAYDAAIIALFSNKTKTLYNLIDPSLNGIISKRSEGYWVIEDRYTGEIKELKKEDWTSIKNNVQARKIAKEIEEYLIDLDDEVFFSRKTKRKTNRQLYNETIYGIATKTDEDGITNYYKKEKFSILDDKDIYLRLLREREKFVINQSNPEVIDQIIEIIESYGKENNIPSRDEAINIKYTKNKINYNLYLKQYMRSLTKSLDQFSEEFINQMIANKTFVLYNPTKNTTRKIKFLRLVNDVKINDIRKNQVINKFNGKNNEPKAFYENINSLGAIVFKNSANNFKTLSINTQIAIFGDKNWDIEDFKTYNMEKIEKYKEIYGIDKTYNFHSFIFPGTILLDKQNKEFYYISSIQTVRDIIEIKFLNKIEFKDENKNQDTSKTPKRLMFGIKSIMNNYEQVDISPFGINKKIFE

SEQ ID NO: 11 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYIAIWASQRPVASKDDIEKKVGFCAFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKEKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 12 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 13 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 14 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 15 - protein, 1377 aa, Streptococcus agalactiae
MNKPYSIGLDIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATMQEEKYYHEKFPTIYHLRKELADKKEKADLRLVYLALAHIIKFRGHFLIEDDRFDVRNTDIQKQYQAFLEIFDTTFENNHLLSQNVDVEAILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHHEDLKHLKQFVKASLPENYREVFADSSKDGYAGYIEGKTNQEAFYKYLLKLLTKQEGSEYFLEKIKNEDFLRKQRTFDNGSIPHQVHLTELRAIIRRQSEYYPFLKENQDRIEKILTFRIPYYVGPLAREKSDFAWMTRKTDDSIRPWNFEDLVDKEKSAEAFIHRMTNNDLYLPEEKVLPKHSLIYEKFTVYNELTKVRFLAEGFKDFQFLNRKQKETIFNSLFKEKRKVTEKDIISFLNKVDGYEGIAIKGIEKQFNASLSTYHDLKKILGKDFLDNTDNELILEDIVQTLTLFEDREMIKKCLDIYKDFFTESQLKKLYRRHYTGWGRLSAKLINGIRNKENQKTILDYLIDDGSANRNFMQLINDDDLSFKPIIDKARTGSHSDNLKEVVGELAGSPAIKKGILQSLKIVDELVKVMGYEPEQIVVEMARENQTTAKGLSRSRQRLTTLRESLANLKSNILEEKKPKYVKDQVENHHLSDDRLFLYYLQNGRDMYTKKALDIDNLSQYDIDHIIPQAFIKDDSIDNRVLVSSAKNRGKSDDVPSIEIVKARKMFWKNLLDAKLMSQRKYDNLTKAERGGLTSDDKARFIQRQLVETRQITKHVARILDERFNNEVDNGKKICKVKIVTLKSNLVSNFRKEFGFYKIREVNDYHHAHDAYLNAVVAKAILTKYPQLEPEFVYGMYRQKKLSKIVHEDKEEKYSEATRKMFFYSNLMNMFKRVVRLADGSIVVRPVIETGRYMRKTAWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNESKGKPEEIEKKQEFVNQHVSYFDDILQLINDFSKRVILADANLEKINKLYQDNKENIPVDELANNIINLFTFTSLGAPAAFKFFDKIVDRKRYTSTKEVLNSTLIHQSITGLYETRIDLGKLGED

SEQ ID NO: 16 - protein, 1130 aa, Streptococcus pasteurianus
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 17 - protein, 1082 aa, Neisseria lactamica
MAAFKPNPMNYILGLDIGIASVGWAMVEVDEEENPIRLIDLGVRVFERAEVPKTGDSLAMARRLARSVRRLTRRRAHRLLRARRLLKREGVLQDADFDENGLVKSLPNTPWQLRAAALDRKLTCLEWSAVLLHLVKHRGYLSQRKNEGETADKELGALLKGVADNAHALQTGDFRTPAELALNKFEKESGHIRNQRGDYSHTFSRKDLQAELNLLFEKQKEFGNPHVSDGLKEDIETLLMAQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSTELQDEIGTAFSLFKTDKDITGRLKDRVQPEILEALLKHISFDKFVQISLKALRRIVPLMEQGKRYDEACAEIYGDHYCKKNAEEKIYLPPIPADEIRNPVVLRALSQARKVINCVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHILLTGKGKRRVFASNGQITNLLRGFWGLRKVRTENDRHHALDAVVVACSTVAMQQKITRFVRYKEMNAFDGKTIDKETGEVLHQKAHFPQPWEFFAQEVMIRVFGKPDGKPEFEEADTPEKLRTLLAEKLSSRPEAVHEYVTPLFVSRAPNRKMSGQGHMETVKSAKRLDEGISVLRVPLTQLKLKGLEKMVNREREPKLYDALKAQLETHKDDPAKAFAEPFYKYDKAGSRTQQVKAVRIEQVQKTGVWVRNHNGIADNATMVRVDVFEKGGKYYLVPIYSWQVAKGILPDRAVVAFKDEEDWTVMDDSFEFRFVLYANDLIKLTAKKNEFLGYFVSLNRATGAIDIRTHDTDSTKGKNGIFQSVGVKTALSFQKNQIDELGKEIRPCRLKKRPPVR

SEQ ID NO: 18 - protein, 1125 aa, Francisella tularensi
MNVKILPIAIDLDVKNTGVFSAFYQKGTSLEKLDNKNGKVYELSKDSYTLLMNNRTARRHQRRGIDRKQLVKRLFKLVWTEQLNLEWDKDTQQAISFLFNRRGFSFITDGYSTEYLNIVPEQVKAILMDIFDDYNGEDDLDSYLKLATEQESKISEIYNKLMQKILEFKLRKLCTDIKDDKVSTKTLKEITSYEFELLADYLANYSESLKIQKFSYTDKQGNLKELSYYHHDKYNIQEFLKRHATINDEILDTLLTDDFDIWNFNFEKFDFDKNEEKLQNQEDKDHTQAHLHHFVFAVNKIKSEMASGGRHRSQYFQEITNVLDENNHQEGYLKNFCENLHNKKYSNLSVKNLVNLVGNLSNLELKPLRKYFNDKIHAKADHWDEQKFTETYCHWILGEWRVGVKDQDKKDGAKYSYKDLCNELKQKVTKAGLIDFLLELDPCRTIPPYLDNNNRKPPKCQSLILNPKFLDNQYPNWQQYLQELKKLQSIQDYLDSFETDLKDLKSSKDQPYFVEYKSSNQQMASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLDKYNNTGRFDDNNQLLTYCNHKPRQKRYQLLNDLAGVLQVSRNQLLSSVEEWFQQAQRVGEISKSQDEQIFEWLKSFKIASYCKAAVEMQKQYRGTLKNAIQTAIFRQSENINKNKNKNTGNQQQALSENSKDVKSLTADEKKLLKLIENIAKASQKIGESLGLNDKQIKKFNSIYSFAQIQQIAFAERKGNANTCAVCSADNAHRMQQIKITELVEDNKDNIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRDDNKNIFAIDTSKWFEIETPSDLRDIGVATIQYKIDNNSRPKVRVKLDYVIDDDSKINYFMNHSLLKSRYPDKVLEILKQSTIIEFESSGFNKTIKEMLGMTLAGIYNETSNN

SEQ ID NO: 19 - protein, 1064 aa, Bradyrhizobium sp.
MKRTSLRAYRLGVDLGANSLGWFVVWLDDHGQPEGLGPGGVRIFPDGRNPQSKQSNAAGRRLARSARRRRDRYLQRRGKLMGLLVKHGLMPADEPARKRLECLDPYGLRAKALDEVLPLHHVGRALFHLNQRRGLFANRAIEQGDKDASAIKAAAGRLQTSMQACGARTLGEFLNRRHQLRATVRARSPVGGDVQARYEFYPTRAMVDAEFEAIWAAQAPHHPTMTAEAHDTIREAIFSQRAMKRPSIGKCSLDPATSQDDVDGFRCAWSHPLAQRFRIWQDVRNLAVVETGPTSSRLGKEDQDKVARALLQTDQLSFDEIRGLLGLPSDARFNLESDRRDHLKGDATGAILSARRHFGPAWHDRSLDRQIDIVALLESALDEAAIIASLGTTHSLDEAAAQRALSALLPDGYCRLGLRAIKRVLPLMEAGRTYAEAASAAGYDHALLPGGKLSPTGYLPYYGQWLQNDVVGSDDERDTNERRWGRLPNPTVHIGIGQLRRVVNELIRWHGPPAEITVELTRDLKLSPRRLAELEREQAENQRKNDKRTSLLRKLGLPASTHNLLKLRLWDEQGDVASECPYTGEAIGLERLVSDDVDIDHLIPFSISWDDSAANKVVCMRYANREKGNRTPFEAFGHRQGRPYDWADIAERAARLPRGKRWRFGPGARAQFEELGDFQARLLNETSWLARVAKQYLAAVTHPHRIHVLPGRLTALLRATWELNDLLPGSDDRAAKSRKDHRHHAIDALVAALTDQALLRRMANAHDDTRRKIEVLLPWPTFRIDLETRLKAMLVSHKPDHGLQARLHEDTAYGTVEHPETEDGANLVYRKTFVDISEKEIDRIRDRRLRDLVRAHVAGERQQGKTLKAAVLSFAQRRDIAGHPNGIRHVRLTKSIKPDYLVPIRDKAGRIYKSYNAGENAFVDILQAESGRWIARATTVFQANQANESHDAPAAQPIMRVFKGDMLRIDHAGAEKFVKIVRLSPSNNLLYLVEHHQAGVFQTRHDDPEDSFRWLFASFDKLREWNAELVRIDTLGQPWRRKRGLETGSEDATRIGWTRPKKWP

SEQ ID NO: 20 - protein, 1136 aa, Streptococcus gordonii
MNGLVLGLDIGIASVGVGILEKDTGKIIHASSRLFPAATADNNVERRSNRQGRRLNRRKKHRSVRLQDLFEGYGLLTDFSKVSMNLNPYQLRVQGMENQLTNEELFVALKNIVKRRGISYLDDASEDGGTVSSDYGKAVEENRKLLAEKTPGQIQLERFEKYGQLRGDFTVEENGEKHRLINVFSTSAYRKEAERILRKQQEFNSKITDEFIEDYLIILTGKRKYYHGPGNEKSRTDYGRFRTDGTTLDNIFGILIGKCTFYTEEYRASKASYTAQEFNLLNDLNNLTVPTETKKLSEEQKKLIIEYAKSAKTLGASTLLKYIAKMIDASVDQIRGYRVDVNNKPEMHTFEVYRKMQSLETIKVEELPRKVLDELAHILTLNTEREGIEEAINSKLKDIFNRDQVLELVQFRKNNSSLFSKGWHNFSIKLMMELIPELYETSEEQMTILTRLGKQRSKETSKRTKYIDEKELTEEIYNPVVAKSVRQAIKIINEATKKYGIFDNIVIEMARENNEEDAKKDYIKRQKANQDEKNAAMEKAAFQYNGKKELPDNIFHGHKELTTKIRLWHQQGEKCLYTGKNIPISDLIHNQYKYEIDHILPLSLSFDDSLSNKVLVLATANQEKGQRTPFQALDSMDDAWSYREFKSYVKDSKLLSNKKKDYLLTEEDISKIEVKQKFIERNLVDTRYSSRVVLNALQDFYKSHQLDTTISVVRGQFTSQLRRKWGIEKSRETYHHHAVDALIIAASSQLRLWKKHSNPLIAYKEGQFVDSETGEIVSLSDEEYKELVFKAPYDHFVDTLRSKKFEDSILFSYQVDSKYNRKISDATIYATRKAKLDKEKKEYTYTLGKIKDIYALGTKTPSKTGFYKFLDLYKTDKSQFLMYQKDRKTWDEVIEKIIEQYRPFKEYDKNGKEVDFNPFEKYRIGNGPIRKYSKKGNGPEIKSLKYYDILLGKHKNITPDGSRNTVALLSLNPWRTDVYYNSETKKYEFLGLKYADLCFEEGGAYGISEVKYKKIREKEGIGKNSEFKFTLYKNDLILIKDTETNCQQFFRFWSRTGKDNPKSFEKHKIELKPYEKAKFEKGEELKVLGKVPPSSNQFQKNMQIENLSIYKVKTDILGNKHFIKKEGDEPKLKFKK

SEQ ID NO: 21 - protein, 984 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNNGDEEKSEILKAIKQNEEKLVNYQSVGEYLYKEYFQKFKENSKEFINVRNKKESYERCITQSFLKDELKLIFQKQREFGFSFSKKFEEEVLSVAFYKRALKDFSHLVGNCSFFTDEKRAPKNSPLAFMFVALTRIINILNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKILNALLKKYGKVHKINIELAREVGKNHSQRAKIEKEQNENYKAKKDAEIECEKLGLKINSKNILKLRLFKEQKEFCAYSGEKIKLSDLQDEKMLEIDHIYPYSRSFDDSYMNKVLVFTKQNQEKLNKTPFEAFGNDSTKWQKIEVLAKNLPEKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAAIIAYANNSIVKAFSDFKKEQESNSVELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPELVYFNAFTSSTVSLIVSKHDNKFETLSKNQKILFKNANEKEVIAKSIGIQNLKVFEKYIVSALGEVTKAEFRQREDFKK

SEQ ID NO: 22 - protein, 1079 aa, Dinoroseobacter shibae
MRLGLDIGTSSIGWWLYETDGAGSDARITGVVDGGVRIFSDGRDPKSGASLAVDRRAARAMRRRRDRYLRRRATLMKVLAETGLMPADPAEAKALEALDPFALRAAGLDEPLPLPHLGRALFHLNQRRGFKSNRKTDRGDNESGKIKDATARLDMEMMANGARTYGEFLHKRRQKATDPRHVPSVRTRLSIANRGGPDGKEEAGYDFYPDRRHLEEEFHKLWAAQGAHHPELTETLRDLLFEKIFFQRPLKEPEVGLCLFSGHHGVPPKDPRLPKAHPLTQRRVLYETVNQLRVTADGREARPLTREERDQVIHALDNKKPTKSLSSMVLKLPALAKVLKLRDGERFTLETGVRDAIACDPLRASPAHPDRFGPRWSILDADAQWEVISRIRRVQSDAEHAALVDWLTEAHGLDRAHAEATAHAPLPDGYGRLGLTATTRILYQLTADVVTYADAVKACGWHHSDGRTGECFDRLPYYGEVLERHVIPGSYHPDDDDITRFGRITNPTVHIGLNQLRRLVNRIIETHGKPHQIVVELARDLKKSEEQKRADIKRIRDTTEAAKKRSEKLEELEIEDNGRNRMLLRLWEDLNPDDAMRRFCPYTGTRISAAMIFDGSCDVDHILPYSRTLDDSFPNRTLCLREANRQKRNQTPWQAWGDTPHWHAIAANLKNLPENKRWRFAPDAMTRFEGENGFLDRALKDTQYLARISRSYLDTLFTKGGHVWVVPGRFTEMLRRHWGLNSLLSDAGRGAVKAKNRTDHRHHAIDAAVIAATDPGLLNRISRAAGQGEAAGQSAELIARDTPPPWEGFRDDLRVRLDRIIVSHRADHGRIDHAARKQGRDSTAGQLHQETAYSIVDDIHVASRTDLLSLKPAQLLDEPGRSGQVRDPQLRKALRVATGGKTGKDFENALRYFASKPGPYQAIRRVRIIKPLQAQARVPVPAQDPIKAYQGGSNHLFEIWRLPDGEIEAQVITSFEAHTLEGEKRPHPAAKRLLRVHKGDMVALERDGRRVVGHVQKMDIANGLFIVPHNEANADTRNNDKSDPFKWIQIGARPAIASGIRRVSVDEIGRLRDGGTRPI

SEQ ID NO: 23 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELNLLFEKQKEFGNPHISDGLKEGIETLLMTQRPALSGDAVQKMLGHCTFEPAEPKAAKNTYTAERFIWLTKLNNLRILEQGSERPLTDTERTTLIDEPYRKSKLTYAQARKLLELDNTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGKRYDEACAEIYGDHYGKKNTEEKIDLPPIPADEIRNPVVLRALSQARKVINGVVRRYGSPARIHIETAREVGKSFKDRKEIEKRQEENRKDREKAATKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHMLLTGKGKRRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 24 - protein, 1195 aa, Elusimicrobium minutum
MQKNINTKQNHIYIKQAQKIKEKLGDKPYRIGLDLGVGSIGFAIVSMEENDGNVLLPKEIIMVGSRIFKASAGAADRKLSRGQRNNHRHTRERMRYLWKVLAEQKLALPVPADLDRKENSSEGETSAKRFLGDVLQKDIYELRVKSLDERLSLQELGYVLYHIAGHRGSSAIRTFENDSEEAQKENTENKKIAGNIKRLMAKKNYRTYGEYLYKEFFENKEKHKREKISNAANNHKFSPTRDLVIKEAEAILKKQAGKDGFHKELTEEYIEKLTKAIGYESEKLIPESGFCPYLKDEKRLPASHKLNEERRLWETLNNARYSDPIVDIVTGEITGYYEKQFTKEQKQKLFDYLLTGSELTPAQTKKLLGLKNTNFEDIILQGRDKKAQKIKGYKLIKLESMPFWARLSEAQQDSFLYDWNSCPDEKLLTEKLSNEYHLTEEEIDNAFNEIVLSSSYAPLGKSAMLIILEKIKNDLSYTEAVEEALKEGKLTKEKQAIKDRLPYYGAVLQESTQKIIAKGFSPQFKDKGYKTPHTNKYELEYGRIANPVVHQTLNELRKLVNEIIDILGKKPCEIGLETARELKKSAEDRSKLSREQNDNESNRNRIYEIYIRPQQQVIITRRENPRNYILKFELLEEQKSQCPFCGGQISPNDIINNQADIEHLFPIAESEDNGRNNLVISHSACNADKAKRSPWAAFASAAKDSKYDYNRILSNVKENIPHKAWRFNQGAFEKFIENKPMAARFKTDNSYISKVAHKYLACLFEKPNIICVKGSLTAQLRMAWGLQGLMIPFAKQLITEKESESFNKDVNSNKKIRLDNRHHALDAIVIAYASRGYGNLLNKMAGKDYKINYSERNWLSKILLPPNNIVWENIDADLESFESSVKTALKNAFISVKHDHSDNGELVKGTMYKIFYSERGYTLTTYKKLSALKLTDPQKKKTPKDFLETALLKFKGRESEMKNEKIKSAIENNKRLFDVIQDNLEKAKKLLEEENEKSKAEGKKEKNINDASIYQKAISLSGDKYVQLSKKEPGKFFAISKPTPTTTGYGYDTGDSLCVDLYYDNKGKLCGEIIRKIDAQQKNPLKYKEQGFTLFERIYGGDILEVDFDIHSDKNSFRNNTGSAPENRVFIKVGTFTEITNNNIQIWFGNIIKSTGGQDDSFTINSMQQYNPRKLILSSCGFIKYRSPILKNKEG

SEQ ID NO: 25 - protein, 1101 aa, Akkermansia muciniphila
MSRSLTFSFDIGYASIGWAVIASASHDDADPSVCGCGTVLFPKDDCQAFKRREYRRLRRNIRSRRVRIERIGRLLVQAQIITPEMKETSGHPAPFYLASEALKGHRTLAPIELWHVLRWYAHNRGYDNNASWSNSLSEDGGNGEDTERVKHAQDLMDKHGTATMAETICRELKLEEGKADAPMEVSTPAYKNLNTAFPRLIVEKEVRRILELSAPLIPGLTAEIIELIAQHHPLTTEQRGVLLQHGIKLARRYRGSLLFGQLIPRFDNRIISRCPVTWAQVYEAELKKGNSEQSARERAEKLSKVPTANCPEFYEYRMARILCNIRADGEPLSAEIRRELMNQARQEGKLTKASLEKAISSRLGKETETNVSNYFTLHPDSEEALYLNPAVEVLQRSGIGQILSPSVYRIAANRLRRGKSVTPNYLLNLLKSRGESGEALEKKIEKESKKKEADYADTPLKPKYATGRAPYARTVLKKVVEEILDGEDPTRPARGEAHPDGELKAHDGCLYCLLDTDSSVNQHQKERRLDTMTNNHLVRHRMLILDRLLKDLIQDFADGQKDRISRVCVEVGKELTTFSAMDSKKIQRELTLRQKSHTDAVNRLKRKLPGKALSANLIRKCRIAMDMNWTCPFTGATYGDHELENLELEHIVPHSFRQSNALSSLVLTWPGVNRMKGQRTGYDFVEQEQENPVPDKPNLHICSLNNYRELVEKLDDKKGHEDDRRRKKKRKALLMVRGLSHKHQSQNHEAMKEIGMTEGMMTQSSHLMKLACKSIKTSLPDAHIDMIPGAVTAEVRKAWDVFGVFKELCPEAADPDSGKILKENLRSLTHLHHALDACVLGLIPYIIPAHHNGLLRRVLAMRRIPEKLIPQVRPVANQRHYVLNDDGRMMLRDLSASLKENIREQLMEQRVIQHVPADMGGALLKETMQRVLSVDGSGEDAMVSLSKKKDGKKEKNQVKASKLVGVFPEGPSKLKALKAAIEIDGNYGVALDPKPVVIRHIKVFKRIMALKEQNGGKPVRILKKGMLIHLTSSKDPKHAGVWRIESIQDSKGGVKLDLQRAHCAVPKNKTHECNWREVDLISLLKKYQMKRYPTSYTGTPR

SEQ ID NO: 26 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEIAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDLQKAQVSGQGDSLHEHIANLAGSPAIKKGILQTVKVVDELVKVMGRHKPENIVIEMARENQTTQKGQKNSRERMKRIEEGIKELGSDILKEYPVETTQLQNEKLYLYYLQNGRDMYVDQELDINRLSDYDVDHIVPQSFLKDDSIDNKVLTRSDKNRGKSDNVPSEEVVKKMKNYWKQLLNAKLITQRKFDNLTKAERGGLSELDKAGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDIRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELVGITIMERSSFEKDPVDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKCFDTTIGRNRYKSIKEVLDATLIHQSITGLYETRIDLSQLGGD

SEQ ID NO: 27 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 28 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 29 - protein, 1131 aa, Acidovorax sp.
MAQHVFGLDIGIASVGWAILGEQRIIDLGVRCFDKAETAKEGDPLNLTRRQARLLRRRLYRRAWRLTQLSRLLKRKGLIADAKLFAKAPSYGDSAWELRRQGLDRLLTPLEWARVIYHQCKHRGFHWTSKAEEAKADSDAEGGRVKQGLAHTKALMQAKNYRSAAEMVLAEFPDAQRNKRGQYDKALSRVLLGEELALLFATQRRLGNPHASDFFEKLILGDGDRKSGLFWQQKPALSGADLLKMLGKCTFEKGEYRAPKASFSVERHVWLTRLNNLRIVVDGRSRPLNEAERQAALLLPYQTETSKYKTLKNAFIKAGLWGDGVRFGGLAYPSQAQIDAEKTKDPEDQFLVKLPAWHELRKAFKAAGHEALWQQISTPALDGDPTLLDQIATVLSVYKDGAEVVQQLRQLALPEPAASIAVLEKISFDKFSSLSLKALRRIVPLMQSGLRYDEAVAQIPEYGHHSQRIEPGAAKHLYLPPFYEAQRKYAGKGDHIGSMQFRDDADIPRNPVVLRALNQARKVVNALIREYGSPIAVNIEMARDLSRPLDERNKVKRAQEEFRDRNDRARSEFERDFGYKPKAAAFEKWMLYREQLGQCAYSQQPLDIQRVLDDHNYAQVDHALPYSRSYDDSKNNKVLVLTHENQNKGNRTAFEYLTSFPDGEDGERWRTFVAWVQGNKAYRMAKRNRLLRKNYGVDESKGFIDRNLNDTRYICKFFKNYVEEHLQLAARADGDTARRCVVVNGQLTAFLRARWGLTKVRGDSDRHHALDAAVVAACTHGMVKALADYSRRKEISFLQEGFPDPETGEILNPAAFDRARQHFPEPWTHFAHELKARLFTDDLAALREDMQRLGSYTTEDLGRLRTLFVSRAPQRRSGGAVHKETIYAQPESLKQQGGVIEKILLTSLKLQDFDKLLNPESNDHFVEPHRNERLYAAIRQRLEQFGGRADKAFGPDNLFHKPDKNNQPTGPVVRSIKLVRGKQTGIPIRGGLAKNDSMLRVDIFTKAGKFHLVPVYVHHRVTGLPNRAIVAFKDEDEWTLIDESFAFLFSVYPNDYVKVTLKKEQQSGYYSGADRSTGAMNLWAHDRAASVGKDGLIRGIGVKTALSVEKFNVDVLGRIYLAPPETRSGLA

SEQ ID NO: 30 - protein, 1082 aa, Neisseria meningitidis
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 31 - protein, 1363 aa, Unknown
  Description of Unknown Lacticaseibacillus rhamnosus GG sequence
MTKLNQPYGIGLDIGSNSIGFAVVDANSHLLRLKGETAIGARLFREGQSAADRRGSRTTRRRLSRTRWRLSFLRDFFAPHITKIDPDFFLRQKYSEISPKDKDRFKYEKRLFNDRTDAEFYEDYPSMYHLRLHLMTHTHKADPREIFLAIHHILKSRGHFLTPGAAKDFNTDKVDLEDIFPALTEAYAQVYPDLELTFDLAKADDFKAKLLDEQATPSDTQKALVNLLLSSDGEKEIVKKRKQVLTEFAKAITGLKTKFNLALGTEVDEADASNWQFSMGQLDDKWSNIETSMTDQGTEIFEQIQELYRARLLNGIVPAGMSLSQAKVADYGQHKEDLELFKTYLKKLNDHELAKTIRGLYDRYINGDDAKPFLREDFVKALTKEVTAHPNEVSEQLLNRMGQANFMLKQRTKANGAIPIQLQQRELDQIIANQSKYYDWLAAPNPVEAHRWKMPYQLDELLNFHIPYYVGPLITPKQQAESGENVFAWMVRKDPSGNITPYNFDEKVDREASANTFIQRMKTTDTYLIGEDVLPKQSLLYQKYEVLNELNNVRINNECLGTDQKQRLIREVFERHSSVTIKQVADNLVAHGDFARRPEIRGLADEKRFLSSLSTYHQLKEILHEAIDDPTKLLDIENIITWSTVFEDHTIFETKLAEIEWLDPKKINELSGIRYRGWGQFSRKLLDGLKLGNGHTVIQELMLSNHNLMQILADETLKETMTELNQDKLKTDDIEDVINDAYTSPSNKKALRQVLRVVEDIKHAANGQDPSWLFIETADGTGTAGKRTQSRQKQIQTVYANAAQELIDSAVRGELEDKIADKASFTDRLVLYFMQGGRDIYTGAPLNIDQLSHYDIDHILPQSLIKDDSLDNRVLVNATINREKNNVFASTLFAGKMKATWRKWHEAGLISGRKLRNLMLRPDEIDKFAKGFVARQLVETRQIIKLTEQIAAAQYPNTKIIAVKAGLSHQLREELDFPKNRDVNHYHHAFDAFLAARIGTYLLKRYPKLAPFFTYGEFAKVDVKKFREFNFIGALTHAKKNIIAKDTGEIVWDKERDIRELDRIYNFKRMLITHEVYFETADLFKQTIYAAKDSKERGGSKQLIPKKQGYPTQVYGGYTQESGSYNALVRVAEADTTAYQVIKISAQNASKIASANLKSREKGKQLLNEIVVKQLAKRRKNWKPSANSFKIVIPRFGMGTLFQNAKYGLFMVNSDTYYRNYQELWLSRENQKLLKKLFSIKYEKTQMNHDALQVYKAIIDQVEKFFKLYDINQFRAKLSDAIERFEKLPINTDGNKIGKTETLRQILIGLQANGTRSNVKNLGIKTDLGLLQVGSGIKLDKDTQIVYQSPSGLFKRRIPLADL

SEQ ID NO: 32 - protein, 1259 aa, Streptobacillus moniliformis
MKYILGLDLGVASIGWAVTEINEKGEPVGLIDANSVIFKPLDNDKGKLYNVERRDKRGSRRILRRKKHRVERTKMLLVNSSFLTNEEIDNLYVGKLDNIYEVRLKGLKEQLSKNEIARLMIYYCKNRGFKSNRKTEDLEILKSLGEKISEKDSDEKKLKPIIAKNTELIESKGLTPIEVIYMIRENDNSILGFKNKEGNYKFGFKRNQVIDEVRKILGTQQILSKEIVDEYIDILSSQRDFSDGPGGDSKYKIDYTKLAGKCKYTGEVRAVKSAPSYEIFTMLQKLNDIRYVKFTSEDKIEKKKLSKEVIHKLYDLVVEKNKTLTYDLIEKSIDEENIKLLNIPKLSKSKYVELRKSYFKERNDNENLTEEEINAFNEKLNNERMKQELIRNNLKAYKELKSKFKKNNIDENRIKELGGIYFLDLVAELLTYSKTDEKLEYFVENVERYSLFKEQRDIVEIIKTFPNYTKNGNLSLSLVRELNKLLMEGHDYESSLSSLNYYIDTDVDWEKFPTISEIEDSLSTKITNPNVKHILVILRKLYNTLLFKYGRPEKVHLELSRDFSNDFSTRNKIQKEQLENKVRREVAAFEMYGANKDIVAGKDRLSNDDFVRIKLWEEQNKVCMYSGRTIEKYQLTSAEVQIDHILPYSKSFDNSYSNKVLVFSNENQDKKERTPYQWLKGTEKWNEFKQRVRLNLNISNKKKENLLFEDEVVNNEFLERELHATSYSSRLALNIFQRLIPVSDEDKYDEHGNEKVKYLYNRNVIAFQGKMTSMLRNLYSLNKYTHSFESDTLDIDNKAFILKEFEINKDNLKMTAYNVNTGLEITSETKVEKNKSGEFKTIKDELLKKELDKKENLIEISDYFKDKVLFDLKIVDFEEIDTVEPEIISILYKMITALKEEVYSKNRENHLHHSLDAFLLTIMNRSMQMKLIKYNQLISVLKNKEIEIFNEEKGEYIDSKEFAEELQKQKIIDIDGNERGIKFNVNGKTHRLNLVKPYENFLDDIKNKIFDKRENNKLPYHVVKSKVSGALHAETILGESKGEITKRISVFDINYKNLGKIFDKDGSQKEIYETLVKWLEAKSKDYPKLKNGNIIKKVKIVDGNKDKLIKLGNKRYVEMGRTTVKILVLKKEAEEGLKFASIGRYKYNLLKSEKDVNISIWKDAIHFCKVRYNKLKENGYRLIHELIPGETIELELNKGNISKCLVVGFSGGKIEISSVIGDALDLINDKISTRIKERYYITVSTIKSIKKINKNILGD

SEQ ID NO: 33 - protein, 1024 aa, Helicobacter mustelae
MIRTLGIDIGIASIGWAVIEGEYTDKGLENKEIVASGVRVFTKAENPKNKESLALPRTLARSARRRNARKKGRIQQVKHYLSKALGLDLECFVQGEKLATLFQTSKDFLSPWELRERALYRVLDKEELARVILHIAKRRGYDDITYGVEDNDSGKIKKAIAENSKRIKEEQCKTIGEMMYKLYFQKSLNVRNKKESYNRCVGRSELREELKTIFQIQQELKSPWVNEELIYKLLGNPDAQSKQEREGLIFYQRPLKGFGDKIGKCSHIKKGENSPYRACKHAPSAEEFVALTKSINFLKNLTNRHGLCFSQEDMCVYLGKILQEAQKNEKGLTYSKLKLLLDLPSDFEFLGLDYSGKNPEKAVFLSLPSTFKLNKITQDRKTQDKIANILGANKDWEAILKELESLQLSKEQIQTIKDAKLNFSKHINLSLEALYHLLPLMREGKRYDEGVEILQERGIFSKPQPKNRQLLPPLSELAKEESYFDIPNPVLRRALSEFRKVVNALLEKYGGFHYFHIELTRDVCKAKSARMQLEKINKKNKSENDAASQLLEVLGLPNTYNNRLKCKLWKQQEEYCLYSGEKITIDHLKDQRALQIDHAFPLSRSLDDSQSNKVLCLTSSNQEKSNKTPYEWLGSDEKKWDMYVGRVYSSNFSPSKKRKLTQKNFKERNEEDFLARNLVDTGYIGRVTKEYIKHSLSFLPLPDGKKEHIRIISGSMTSTMRSFWGVQEKNRDHHLHHAQDAIIIACIEPSMIQKYTTYLKDKETHRLKSHQKAQILREGDHKLSLRWPMSNFKDKIQESIQNIIPSHHVSHKVTGELHQETVRTKEFYYQAFGGEEGVKKALKFGKIREINQGIVDNGAMVRVDIFKSKDKGKFYAVPIYTYDFAIGKLPNKAIVQGKKNGIIKDWLEMDENYEFCFSLFKNDCIKIQTKEMQEAVLAIYKSTNSAKATIELEHLSKYALKNEDEEKMFTDTDKEKNKTMTRESCGIQGLKVFQKVKLSVLGEVLEHKPRNRQNIALKTTPKHV

SEQ ID NO: 34 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 35 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSSDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 36 - protein, 987 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNNGDEEKSEILKAIKQNEEKLVNYQSVGEYLYKEYFQKFKENSKEFINVRNKKESYERCIAQSFLKDELKLIFQKQREFGFSFSKKFEEEVLSVAFYKRALKDFSHLVGNCSFFTDEKRAPKNSPLAFMFVALTRIINLLNNLKNTEGILYTKDDLNTLLNEVLKNGTLTYKQTKKLLGLSDDYEFKGEKGTYFIEFKKYKEFIKALGEHNLSQDNLNEIAKDITLIKDEIKLKKALAKYDLNQNQIDSLSKLEFKDHLNISFKALKLITPLMLEGKKYDEAYNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYPVPIYTMDFALKVLPNKAVVQGKDKKSGLIKDWILMDENYEFCFSLYKDSLILIQTKDMQEPELVYFNAFTSSTVSLIVSKHDNKFETLSKNQKILFKNANEKEVIAKSIGIQNLKVFEKYIVSALGEVTKAEFRQREDFKK

SEQ ID NO: 37 - protein, 984 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGDHSLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNYSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREGFKK

SEQ ID NO: 38 - protein, 984 aa, Campylobacter sp.
MVRILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNKTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSTKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 39 - protein, 984 aa, Campylobacter sp.
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGDHSLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKILNALLKKYGKVHKINIELAREVGKNHSQRAKIEKEQNENYKAKKDAEIECEKLGLKINSKNILKLRLFKEQKEFCAYSGEKIKLSDLQDEKMLEIDHIYPYSRSFDDSYMNKVLVFTKQNQEKLNKTPFEAFGNDSTKWQKIEVLAKNLPEKKQKRILDKNYKDKEQKDFKDRNLNDTRYIARLVLNYTKDYLDFLPLSDDENTKLNDTQKGSKVHVEAKSGMLTSALRHTWGFSAKDRNNHLHHAIDAAIIAYANNSIVKAFSDFKKEQESNSVELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPELVYFNAFTSSTVSLIVSKHDNKFETLSKNQKILFKNANEKEVIAKSIGIQNLKVFEKYIVSALGEVTKAEFRQREDFKK

SEQ ID NO: 40 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 41 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 42 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 43 - protein, 1253 aa, Prevotella albensis
MNIKNFTGLYPLSKTLRFELKPIGKTKENIEKNGILTKDEQRAKDYLIVKGFIDEYHKQFIKDRLWDFKLPLESEGEKNSLEEYQELYELTKRNDAQEADFTEIKDNLRSSITEQLTKSGSAYDRIFKKEFIREDLVNFLEDEKDKNIVKQFEDFTTYFTGFYENRKNMYSSEEKSTAIAYRLIHQNLPKFMDNMRSFAKIANSSVSEHFSDIYESWKEYLNVNSIEEIFQLDYFSETLTQPHIEVYNYIIGKKVLEDGTEIKGINEYVNLYNQQQKDKSKRLPFLVPLYKQILSDREKLSWIAEEFDSDKKMLSAITESYNHLHNVLMGNENESLRNLLLNIKDYNLEKINITNDLSLTEISQNLFGRYDVFTNGIKNKLRVLTPRKKKETDENFEDRINKIFKTQKSFSIAFLNKLPQPEMEDGKPRNIEDYFITQGAINTKSIQKEDIFAQIENAYEDAQVFLQIKDTDNKLSQNKTAVEKIKTLLDALKELQHFIKPLLGSGEENEKDELFYGSFLAIWDELDTITPLYNKVRNWLTRKPYSTEKIKLNFDNAQLLGGWDVNKEHDCAGILLRKNDSYYLGIINKKTNHIFDTDITPSDGECYDKIDYKLLPGANKMLPKVFFSKSRIKEFEPSEAIINCYKKGTHKKGKNFNLTDCHRLINFFKTSIEKHEDWSKFGFKFSDTETYEDISGFYREVEQQGYRLTSHPVSASYIHSLVKEGKLYLFQIWNKDFSQFSKGTPNLHTLYWKMLFDKRNLSDVVYKLNGQAEVFYRKSSIEHQNRIIHPAQHPITNKNELNKKHTSTFKYDIIKDRRYTVDKFQFHVPITINFKATGQNNINPIVQEVIRQNGITHIIGIDRGERHLLYLSLIDLKGNIIKQMTLNEIINEYKGVTYKTNYHNLLEKREKERTEARHSWSSIESIKELKDGYMSQVIHKITDMMVKYNAIVVLEDLNGGFMRGRQKVEKQVYQKFEKKLIDKLNYLVDKKLDANEVGGVLNAYQLTNKFESFKKIGKQSGFLFYIPAWNTSKIDPITGFVNLFNTRYESIKETKVFWSKFDIIRYNKEKNWFEFVFDYNTFTTKAEGTRTKWTLCTHGTRIQTFRNPEKNAQWDNKEINLTESFKALFEKYKIDITSNLKESIMQETEKKFFQELHNLLHLTLQMRNSVTGTDIDYLISPVADEDGNFYDSRINGKNFPENADANGAYNIARKGLMLIRQIKQADPQKKFKFETITNKDWLKFAQDKPYLKD

SEQ ID NO: 44 - DNA, 600 bp, Artificial Sequence
  Description of Artificial Sequence Synthetic polynucleotide
cagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgc

SEQ ID NO: 45 - DNA, 724 bp, Artificial Sequence
  Description of Artificial Sequence Synthetic polynucleotide
cttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgc

SEQ ID NO: 46 - DNA, 524 bp, Artificial Sequence
  Description of Artificial Sequence Synthetic polynucleotide
gagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagagagcgccggggaaggagaggcccgcgccctctcctggagccagattctgcaggtgcactgggtggggatgatcggcgggctaggttgcaagtaagtgccttttcttttgctgctttctttatttctttgtttccatccaggcctcttatgtgaggagctgaagaggaattaaaatatacaggatgaaaaggcggccgccatggtgagcaagggcgaggaggataa

SEQ ID NO: 47 - DNA, 1000 bp, Artificial Sequence
  Description of Artificial Sequence Synthetic polynucleotide
tcgaattaacacaagacagaagtcatataaatcttaagaaggtagaaaatgttacttctagaaaactccaccaaaagttggttggataatttcaggtcttctttctggaaaacgaagagtcacaaagtctatttttctttctactcccaagttccactcattctccaattaaatgtgactcataggtgactgaatgtcatgttttggccactctaagcaaagggattccttggacttgggtacacatattttaagaatattgtatatataagaatattgtatatattcaactgtccatcttgctgcttctctcttacaccaacttcattttgtagcatcagctgcataagctcttgcttttccccctatgcagacatctattcatcaggctctgtgtacactggcaacataggaataaactggataaggttgctgttcccagaagaatctcagaatattgtatttatttaatggcatcaggaaaaaccagtccaccaattttagaaagaaaaaagtcttaaagctgcacatttttatgaacttagacaattactatattttgactattattgactctaatctattccaagaattatacagacagaaagagctttggtaacagcctacatatcaatatttactgaaccaagaactatcacaaaacgtctgttgaaaatttccatgttttaatgatgatgatgctaatggcagctatgatttattaaacactttctatgtgcaggatgtaagctaagtaatttgtacacattatcttatttaaacttattacaagtcttattttcttgaggtagatgttattatcctattttcacagacagacttatgccttgaagaatttggtaaagtcactaagtagtcagtagccaaatcatgtattcttaatccttattcaatattgtccccctatagaagaaaccttgagtttgtaacttttcagtaaggctatttagcttgtgtcctgaagacactctcacctata

SEQ ID NO: 48 - DNA, 1000 bp, Artificial Sequence; identical to SEQ ID NO: 47
  Description of Artificial Sequence Synthetic polynucleotide

SEQ ID NO: 49 - DNA, 1000 bp, Artificial Sequence
  Description of Artificial Sequence Synthetic polynucleotide
tcaattataacagacatgtcttagtctaccttttattgctgtaactgaatacctgagactggttaaagtattcaataaagaaatttatttcttaaagttctggaagctgggaagtctaaggtgaagggtccacatctggtgggaactttcttgctggtgggcacaccagagagtcccaaggcagctcatggcatcacatggcgaggggtgttcactgagagccaaactgggttttataataaacccactcttgtgatatctaactcactcccatgataacccattaagcccttactctatattccattcatgaaggcagaaccttcatgacctcatcaccttttaaaggctctgcctcttaatactgttacattgggaattaagttccaacatgagtttcaaaagggacaaacattcaaaccacagcaaaacattaaaaaaaaaattttctgattaaagagggagcatacatattttcaagtgttcttaagtggaaaataatggatgtgaatgactgggcaaatttaagagtggcatgaggagtaatttattctgctaaatacgtagctttaggaatttaaaaaaaaaagccatgttacatttagtgagacatctggaaatactacatgaggatgagcactttaaaacacttcccgcttcatcagtcagtttacctgtctctgatggtaacagcaatgaactttttgtgaacaatgactttttctacgcctgaaatagataaatatataaagttggctctcatctgttcctattcttccctcgaaaaattgaatctctcagtcactaacttattcgtaatgaagaactgcattccagacatttttcttagacatcgtctcctgctacacaggatgtgaggcaaatgaatattttccatggaactaattcagagagtaaacatatttatagatccagatctaaggtcatggaggagctcattgctttgtaactaaaatgtgtattgattgtattgctgttc

SEQ ID NO: 50 - DNA, 1440 bp, Artificial Sequence
  Description of Artificial Sequence Synthetic polynucleotide
cagtctcagtcactgaagtttccttccttcaatagcacatcttacaagtcaagaatgtaaggtccaaagccccaggacaaatctcagaagcaaagaaagcagcagggaaaagtagaccctgggatttgatttccttccggttatctctaagcatcatttccatgatagaaggtgtggaagcaaataataaaagtgcccgtcactagtgtttatcctgcaaagtggtctgcccttttgagaggcaccctgccctatggccatcctttgatttcttcccttggtggaaatttcctgtttctctttgagaaagataattcaaccctgtttccattgttcttcctcccaggctgctgtaggaaagcgcacgcacacactccacacaaaatgaatttttaaaaaatttattttcacagtcgctcctaccagctctgaaattcagaacccatatgactgatggcatattcagataatcgggtcccaggtctggaaaagcagccttttccccacgtttctttccccacctaggacctcctctgattcttcactgcatcttcgaaagaaaatgtattatttgcttgcctggaagacgctgcaattcaattgattttatatatacatatatataaagaaaacagaaaacatagcctagataccggtcttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagaga

SEQ ID NO: 51 - DNA, 449 bp, Artificial Sequence
  Description of Artificial Sequence Synthetic polynucleotide
cagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 52 - DNA, 22 bp, Artificial Sequence
  Description of Artificial Sequence Synthetic oligonucleotide
ggctgctagcggcaggcgtccc

SEQ ID NO: 53 - DNA, 22 bp, Artificial Sequence
  Description of Artificial Sequence Synthetic oligonucleotide
gcagaatctggctccaggagag

SEQ ID NO: 54 - DNA, 22 bp, Artificial Sequence
  Description of Artificial Sequence Synthetic oligonucleotide
ggtggggatgatcggcgggcta

SEQ ID NO: 55 - DNA, 22 bp, Artificial Sequence
  Description of Artificial Sequence Synthetic oligonucleotide
taattcctcttcagctcctcac

SEQ ID NO: 56 - DNA, 22 bp, Artificial Sequence
  Description of Artificial Sequence Synthetic oligonucleotide
agggctcttctggctgctggac

SEQ ID NO: 57 - DNA, 22 bp, Artificial Sequence
  Description of Artificial Sequence Synthetic oligonucleotide
gggacgcctgccgctagcagcc

SEQ ID NO: 58 - DNA, 22 bp, Artificial Sequence
  Description of Artificial Sequence Synthetic oligonucleotide
tccccacagaagcagcaaaaga

SEQ ID NO: 59 - DNA, 22 bp, Artificial Sequence
  Description of Artificial Sequence Synthetic oligonucleotide
tttcgcccgtgctcgcctcaac

SEQ ID NO: 60 - DNA, 22 bp, Artificial Sequence
  Description of Artificial Sequence Synthetic oligonucleotide
gtttttctccagctccccacca

SEQ ID NO: 61 - DNA, 22 bp, Artificial Sequence
  Description of Artificial Sequence Synthetic oligonucleotide
aagggaagggctcttctggctg

SEQ ID NO: 62 - DNA, 22 bp, Artificial Sequence
  Description of Artificial Sequence Synthetic oligonucleotide
gttccagaaccgagatgtgaaa

SEQ ID NO: 63 - DNA, 20 bp, Artificial Sequence
  Description of Artificial Sequence Synthetic oligonucleotide
ttttctccagctccccacca

SEQ ID NO: 64 - DNA, 20 bp, Artificial Sequence
  Description of Artificial Sequence Synthetic oligonucleotide
cctctcttctttccaggtgg

SEQ ID NO: 65 - DNA, 22 bp, Artificial Sequence
  Description of Artificial Sequence Synthetic oligonucleotide
ggggtccaagtcctccaggggc

SEQ ID NO: 66 - DNA, 20 bp, Artificial Sequence
  Description of Artificial Sequence Synthetic oligonucleotide
cccagcgcgcccgggtcccc

SEQ ID NO: 67 - DNA, 20 bp, Artificial Sequence
  Description of Artificial Sequence Synthetic oligonucleotide
gcgcgctgggaggagtggag

SEQ ID NO: 68 - DNA, 20 bp, Artificial Sequence
  Description of Artificial Sequence Synthetic oligonucleotide
gctagcccagcctcagccga

SEQ ID NO: 69 - DNA, 20 bp, Artificial Sequence
  Description of Artificial Sequence Synthetic oligonucleotide
tgcacctgcagaatctggct

SEQ ID NO: 70 - DNA, 20 bp, Artificial Sequence
  Description of Artificial Sequence Synthetic oligonucleotide
acccagtgcacctgcagaat

SEQ ID NO: 71 - DNA, 20 bp, Artificial Sequence
  Description of Artificial Sequence Synthetic oligonucleotide
caggtgcactgggtggggat

SEQ ID NO: 72 - DNA, 20 bp, Artificial Sequence
  Description of Artificial Sequence Synthetic oligonucleotide
gcctcttatgtgaggagctg

SEQ ID NO: 73 - DNA, 20 bp, Artificial Sequence
  Description of Artificial Sequence Synthetic oligonucleotide
cttcagctcctcacataaga

SEQ ID NO: 74 - DNA, 20 bp, Artificial Sequence
  Description of Artificial Sequence Synthetic oligonucleotide
tatattttaattcctcttca

SEQ ID NO: 75 - DNA, 20 bp, Artificial Sequence
  Description of Artificial Sequence Synthetic oligonucleotide
cccggcatggtgtcagagcc

SEQ ID NO: 76 - DNA, 20 bp, Artificial Sequence
  Description of Artificial Sequence Synthetic oligonucleotide
ggcggtcgccagcgctccag

SEQ ID NO: 77 - DNA, 20 bp, Artificial Sequence
  Description of Artificial Sequence Synthetic oligonucleotide
gccacctggaaagaagagag

SEQ ID NO: 78 - DNA, 20 bp, Artificial Sequence
  Description of Artificial Sequence Synthetic oligonucleotide
ggtcgccagcgctccagcgg

SEQ ID NO: 79 - DNA, 20 bp, Artificial Sequence
  Description of Artificial Sequence Synthetic oligonucleotide
gccagcaatgggaggaagaa

SEQ ID NO: 80 - DNA, 20 bp, Artificial Sequence
  Description of Artificial Sequence Synthetic oligonucleotide
gttccaggtggcgtaataca

SEQ ID NO: 81 - DNA, 20 bp, Artificial Sequence
  Description of Artificial Sequence Synthetic oligonucleotide
ggcggggctgctacctccac

SEQ ID NO: 82 - DNA, 20 bp, Artificial Sequence
  Description of Artificial Sequence Synthetic oligonucleotide
gggcgcagtctgcttgcagg

SEQ ID NO: 83 - DNA, 20 bp, Artificial Sequence
  Description of Artificial Sequence Synthetic oligonucleotide
ggcgctccagcggcggctgt

SEQ ID NO: 84 - DNA, 20 bp, Artificial Sequence
  Description of Artificial Sequence Synthetic oligonucleotide
gaccgggtggttccagcaat

SEQ ID NO: 85 - DNA, 20 bp, Artificial Sequence
  Description of Artificial Sequence Synthetic oligonucleotide
ggggtggttccagcaatggg

SEQ ID NO: 86 - DNA, 20 bp, Artificial Sequence
  Description of Artificial Sequence Synthetic oligonucleotide
gtttactccggagtcactgg

SEQ ID NO: 87 - DNA, 20 bp, Artificial Sequence
  Description of Artificial Sequence Synthetic oligonucleotide
gctatctccaccagtgactc

SEQ ID NO: 88 - DNA, 20 bp, Artificial Sequence
  Description of Artificial Sequence Synthetic oligonucleotide
gacatcacccagggccaagg

SEQ ID NO: 89 - DNA, 20 bp, Artificial Sequence
  Description of Artificial Sequence Synthetic oligonucleotide
gtagtttcgagggatccaat

SEQ ID NO: 90 - DNA, 20 bp, Artificial Sequence
  Description of Artificial Sequence Synthetic oligonucleotide
gctcccagcagaactgatcg

SEQ ID NO: 91 - DNA, 20 bp, Artificial Sequence
  Description of Artificial Sequence Synthetic oligonucleotide
gtgactccggagtaaagcga

SEQ ID NO: 92 - DNA, 20 bp, Artificial Sequence
  Description of Artificial Sequence Synthetic oligonucleotide
gggagctcaccatagaactt

SEQ ID NO: 93 - DNA, 20 bp, Artificial Sequence
  Description of Artificial Sequence Synthetic oligonucleotide
gacggatctagatcctccag

SEQ ID NO: 94 - DNA, 20 bp, Artificial Sequence
  Description of Artificial Sequence Synthetic oligonucleotide
gccgggtaagagctactagt

SEQ ID NO: 95 - DNA, 20 bp, Artificial Sequence
  Description of Artificial Sequence Synthetic oligonucleotide
gcccggtgtgtgctgtagaa

SEQ ID NO: 96 - protein, 30 aa, Artificial Sequence
  Description of Artificial Sequence Synthetic polypeptide
YCQKWMWTCDSERKCCEGMVCRLWCKKKLW

SEQ ID NO: 97 - protein, 32 aa, Artificial Sequence
  Description of Artificial Sequence Synthetic polypeptide
GPYCQKWMQTCDSERKCCEGMVCRLWCKKKLL

SEQ ID NO: 98 - protein, 35 aa, Artificial Sequence
  Description of Artificial Sequence Synthetic polypeptide
DCRYMFGDCEKDEDCCKHLGCKRKMKYCAWDFTFT

SEQ ID NO: 99 - protein, 29 aa, Artificial Sequence
  Description of Artificial Sequence Synthetic peptide
YCQKWMWTCDSKRACCEGLRCKLWCRKII

SEQ ID NO: 100 - protein, 29 aa, Artificial Sequence
  Description of Artificial Sequence Synthetic peptide
YCQKWMWTCDSKRACCEGLRCKLWCRKEI

SEQ ID NO: 101 - protein, 29 aa, Artificial Sequence
  Description of Artificial Sequence Synthetic peptide
YCQKWLWTCDSERKCCEDMVCRLWCKKRL

SEQ ID NO: 102 - protein, 34 aa, Artificial Sequence
  Description of Artificial Sequence Synthetic polypeptide
ACLGQWDSCDPKASKCCPNYACEWKYPWCRYKLF

SEQ ID NO: 103 - protein, 34 aa, Artificial Sequence
  Description of Artificial Sequence Synthetic polypeptide
ACLGQWASCDPKASKCCPNYACEWKYPWCRYKLF

SEQ ID NO: 104 - protein, 33 aa, Artificial Sequence
  Description of Artificial Sequence Synthetic polypeptide
DCLKFGWKCNPRNDKCCSGLKCGSNHNWCKLHL

SEQ ID NO: 105 - protein, 34 aa, Artificial Sequence
  Description of Artificial Sequence Synthetic polypeptide
DCLGFMRKCIPDNDKCCRPNLVCSRTHKWCKYVF

SEQ ID NO: 106 - protein, 34 aa, Artificial Sequence
  Description of Artificial Sequence Synthetic polypeptide
DCLGFFRKCIPDNDKCCRPNLVCSRLHRWCKYVF

SEQ ID NO: 107 - protein, 34 aa, Artificial Sequence
  Description of Artificial Sequence Synthetic polypeptide
DCLGAMRKCIPDNDKCCRPNLVCSRTHKWCKYVF

SEQ ID NO: 108 - protein, 35 aa, Artificial Sequence
  Description of Artificial Sequence Synthetic polypeptide
ECLEIFKACNPSNDQCCKSSKLVCSRKTRWCKYQI

SEQ ID NO: 109 - protein, 35 aa, Artificial Sequence
  Description of Artificial Sequence Synthetic polypeptide
GCLGIFKACNPSNDQCCKSSKLVCSRKTRWCKWQI

SEQ ID NO: 110 - protein, 33 aa, Artificial Sequence
  Description of Artificial Sequence Synthetic polypeptide
DCLGMFKSCDPENDKCCKRLVCSRSHRWCKWKL

SEQ ID NO: 111 - protein, 33 aa, Artificial Sequence
  Description of Artificial Sequence Synthetic polypeptide
ICLGMFKSCDPENDKCCKRLVCSRSHRWCKWKL

SEQ ID NO: 112 - protein, 33 aa, Artificial Sequence
  Description of Artificial Sequence Synthetic polypeptide
DCGTIWHYCGTDQSECCEGWKCSRQLCKYVIDW

SEQ ID NO: 113 - protein, 35 aa, Artificial Sequence
  Description of Artificial Sequence Synthetic polypeptide
ECRYLFGGCSSTSDCCKHLSCRSDWKYCAWDGTFS

SEQ ID NO: 114 - protein, 29 aa, Artificial Sequence
  Description of Artificial Sequence Synthetic peptide
  MOD_RES (6)..(6): Norleucine
  MOD_RES (17)..(17): Propargylglycine
YCQKWLWTCDSKRACCGGLRCKLWCRKEI

SEQ ID NO: 115 - protein, 65 aa, Artificial Sequence
  Description of Artificial Sequence Synthetic polypeptide
GVRDAYIADDKNCVYTCASNGYCNTECTKNGAESGYCQWIGRYGNACWCIKLPDEVPIRIPGKCR

SEQ ID NO: 116 - protein, 18 aa, Artificial Sequence
  Description of Artificial Sequence Synthetic peptide
LLIILRRRIRKQAHAHSK

SEQ ID NO: 117 - protein, 21 aa, Artificial Sequence
  Description of Artificial Sequence Synthetic peptide
KETWWETWWTEWSQPKKKRKV

SEQ ID NO: 118 - protein, 18 aa, Artificial Sequence
  Description of Artificial Sequence Synthetic peptide
KLALKLALKALKAALKLA

SEQ ID NO: 119 - protein, 28 aa, Artificial Sequence
  Description of Artificial Sequence Synthetic peptide
LSTAADMQGVVTDGMASGLDKDYLKPDD

SEQ ID NO: 120 - protein, 32 aa, Artificial Sequence
  Description of Artificial Sequence Synthetic polypeptide
LLKKKCWLRCVMGECCKRESDCTQMWKQCYPG

SEQ ID NO: 121 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSTSGHLSRHIRTHTGEKPFACDICGRKFADRSHLARHTKIHTGSQKPFQCRICMRNFSQSGDLTRHIRTHTGEKPFACDICGRKFAYRWLRNNHTKIHTGSQKPFQCRICMRNFSRSDTLSEHIRTHTGEKPFACDICGRKFARAQHLQQHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 122 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSDRSHLSRHIRTHTGEKPFACDICGRKFADRSALARHTKIHTGSQKPFQCRICMRNFSQSSDLSRHIRTHTGEKPFACDICGRKFARSDDLTRHTKIHTGSQKPFQCRICMRNFSRSDDLTRHIRTHTGEKPFACDICGRKFAQRSTLSSHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 123 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSQSSDLSRHIRTHTGEKPFACDICGRKFARSDALARHTKIHTGSQKPFQCRICMRNFSQSGDLTRHIRTHTGEKPFACDICGRKFARSWGLQVHTKIHTGSQKPFQCRICMRNFSRSDHLSQHIRTHTGEKPFACDICGRKFADSSTRKKHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 124 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSDRSHLTRHIRTHTGEKPFACDICGRKFAQSGDLTRHTKIHTGSQKPFQCRICMRNFSRSDDLTRHIRTHTGEKPFACDICGRKFAQRSTLSSHTKIHTGSQKPFQCRICMRNFSRSDVLSEHIRTHTGEKPFACDICGRKFARNQHRKTHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 125 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSDNLSEHIRTHTGEKPFACDICGRKFAERANRNSHTKIHTGSQKPFQCRICMRNFSRSDNLARHIRTHTGEKPFACDICGRKFAWRGDRVKHTKIHTGSQKPFQCRICMRNFSDRSDLSRHIRTHTGEKPFACDICGRKFARRTDLRRHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 126 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSDRSDLSRHIRTHTGEKPFACDICGRKFARRTDLRRHTKIHTGSQKPFQCRICMRNFSDRSDLSRHIRTHTGEKPFACDICGRKFARSHHLKAHTKIHTGSQKPFQCRICMRNFSRSANLARHIRTHTGEKPFACDICGRKFARSDNLREHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 127 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSAHLSRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 128 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSQSGDLTRHIRTHTGEKPFACDICGRKFAQSGARNIHTKIHTGSQKPFQCRICMRNFSRSANLARHIRTHTGEKPFACDICGRKFARSDALARHTKIHTGSQKPFQCRICMRNFSDRSDLSRHIRTHTGEKPFACDICGRKFARRTDLRRHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 129 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSDVLSRHIRTHTGEKPFACDICGRKFADSRDRKNHTKIHTGSQKPFQCRICMRNFSRSADLTRHIRTHTGEKPFACDICGRKFADRSHLARHTKIHTGSQKPFQCRICMRNFSRSDNLSEHIRTHTGEKPFACDICGRKFASKQYLIKHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 130 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSRSADLTRHIRTHTGEKPFACDICGRKFAQSGHLSRHTKIHTGSQKPFQCRICMRNFSQSSDLSRHIRTHTGEKPFACDICGRKFAHRKSLSRHTKIHTGSQKPFQCRICMRNFSDRSDLSRHIRTHTGEKPFACDICGRKFAQSSTRARHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 131 - protein, 295 aa, Artificial Sequence
  Description of Artificial Sequence Synthetic polypeptide
MDYKDHDGDYKDHDIDYKDDDDKMAPKKKRKVGIHGVPAAMAERPFQCRICMRNFSDRSHLTRHIRTHTGEKPFACDICGRKFARSDDLTRHTKIHTGSQKPFQCRICMRNFSDRSHLTRHIRTHTGEKPFACDICGRKFARSDNLTRHTKIHTGSQKPFQCRICMRNFSQSGHLARHIRTHTGEKPFACDICGRKFAQKGTLGEHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 132 - protein, 62 aa, Artificial Sequence
  Description of Artificial Sequence Synthetic polypeptide
VTFEDVTVNFTQGEWQRLNPEQRNLYRDVMLENYSNLVSVGQGETTKPDVILRLEQGKEPWL

SEQ ID NO: 133 - protein, 62 aa, Artificial Sequence
  Description of Artificial Sequence Synthetic polypeptide
VTFKDVFVDFTREEWKLLDTAQQIVYRNVMLENYKNLVSLGYQLTKPDVILRLEKGEEPWLV

SEQ ID NO: 134 - protein, 62 aa, Artificial Sequence
  Description of Artificial Sequence Synthetic polypeptide
VTFEDVSMDFSQEEWELLEPAQKNLYREVMLENYRNVVSLEALKNQCTDVGIKEGPLSPAQT

SEQ ID NO: 135 - protein, 61 aa, Artificial Sequence
  Description of Artificial Sequence Synthetic polypeptide
VTFDDVAVTFTKEEWGQLDLAQRTLYQEVMLENCGLLVSLGCPVPKAELICHLEHGQEPWT

SEQ ID NO: 136 - protein, 61 aa, Artificial Sequence
  Description of Artificial Sequence Synthetic polypeptide
MAFEDVAVYFSQEEWGLLDTAQRALYRRVMLDNFALVASLGLSTSRPRVVIQLERGEEPWV

SEQ ID NO: 137 - protein, 286 aa, Artificial Sequence
  Description of Artificial Sequence Synthetic polypeptide
VQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 138 - protein, 220 aa, Artificial Sequence
  Description of Artificial Sequence Synthetic polypeptide
ARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKILEDALMADILSRAADTEEMDIEMDSGDEAEF

SEQ ID NO: 139 - protein, 29 aa, Artificial Sequence
  Description of Artificial Sequence Synthetic peptide
MNIQMLLEAADYLERREREAEHGYASMLP

SEQ ID NO: 140 - protein, 121 aa, Artificial Sequence
  Description of Artificial Sequence Synthetic polypeptide
MKEGENNKPREKSEGNKRKSSFSNSADDIKSKKKREQSNDIARGFERGLEPEKIIGATDSCGDLMFLMKWKDTDEADLVLAKEANVKCPQIVIAFYEERLTWHAYPEDAENKEKESAKSEF

SEQ ID NO: 141 - protein, 267 aa, Artificial Sequence
  Description of Artificial Sequence Synthetic polypeptide
SSSMADFRKFFAKAKHIVIISGAGVSAESGVPTFRGAGGYWRKWQAQDLATPLAFAHNPSRVWEFYHYRREVGSKEPNAGHRAIAECETRLGKQGRRVWITQNIDELHRKAGTKNLLEIHGSLFKTRCTSCGWAENYKSPICPALSGKGAPEPGTQDASIPVEKLPRCEEAGCGGLLRPHWWFGENLDPAILEEVDRELAHCDLCLWGTSSWYPAAFAPQVAARGVPVAEFNTETTPATNRFRFHFQGPCGTTLPEALACHENETVS

SEQ ID NO: 142 - protein, 209 aa, Artificial Sequence
  Description of Artificial Sequence Synthetic polypeptide
SCDSTNAAIAKQALKKPIKGKQAPRKKAQGKTQQNRKLTDFYPVRRSSRKSKAELQSEERKRIDELIESGKEEGKIDLIDGKGRGVIATKQFSRGDFVVEYHGDLIEITDAKKREALYAQDPSTGCYYYFQYLSKTYCVDATRETNRLGRLINHSKCGNCQTKLHDIDGVPHLILIASRDIAAGEELLYDYGDRSKASIEAFPWLKHEF

SEQ ID NO: 143 - protein, 213 aa, Artificial Sequence
  Description of Artificial Sequence Synthetic polypeptide
MEFWGIEVKSGKPVTVTPEEGILIHVSQASLGECKNKKGEFVPLHVKVGNQNLVLGTLSTENIPQLFCDLVFDKEFELSHTWGKGSVYFVGYKTPNIEPQGYSEEEEEEEEEVPAGNAAKAVAKPKAKPAEVKPAVDDEEDESDSDGMDEDDSDGEDSEEEEPTPKKPASSKKRANETTPKAPVSAKKAKVAVTPQKTDEKKKGGKAANQSEF

SEQ ID NO: 144 - protein, 6 aa, Artificial Sequence
  Description of Artificial Sequence Synthetic peptide
DLELRL

SEQ ID NO: 145 - DNA, 28 bp, Unknown
  Description of Unknown ZFP target sequence
gcagtctgcttgcaggcggtcgccagcg

SEQ ID NO: 146 - DNA, 28 bp, Unknown
  Description of Unknown ZFP target sequence
  modified_base (26)..(28): a, c, t, g, unknown or other
ctccagcggcggctgtcggctttccnnn

SEQ ID NO: 147 - DNA, 28 bp, Unknown
  Description of Unknown ZFP target sequence
tagcctgggtgccagtggctgctagcgg

SEQ ID NO: 148 - DNA, 28 bp, Unknown
  Description of Unknown ZFP target sequence
agtggctgctagcggcaggcgtcccctg

SEQ ID NO: 149 - DNA, 28 bp, Unknown
  Description of Unknown ZFP target sequence
aggcgtcccctgagcaacaggagcccag

SEQ ID NO: 150 - DNA, 28 bp, Unknown
  Description of Unknown ZFP target sequence
ggaaggagaggcccgcgccctctcctgg

SEQ ID NO: 151 - DNA, 28 bp, Unknown
  Description of Unknown ZFP target sequence
ctgcaggtgcactgggtggggatgatcg

SEQ ID NO: 152 - DNA, 28 bp, Unknown
  Description of Unknown ZFP target sequence
gcgcgcccgtggaggtagcagccccgcc

SEQ ID NO: 153 - DNA, 28 bp, Unknown
  Description of Unknown ZFP target sequence
cctgccagggcgcgcccgtggaggtagc

SEQ ID NO: 154 - DNA, 28 bp, Unknown
  Description of Unknown ZFP target sequence
cgacagccgccgctggagcgctggcgac

SEQ ID NO: 155 - DNA, 28 bp, Unknown
  Description of Unknown ZFP target sequence
ctccaggagagggcgcgggcctctcctt

SEQ ID NO: 156 - protein, 30 aa, Artificial Sequence
  Description of Artificial Sequence Synthetic polypeptide
  SITE (4)..(30): This region may encompass 1-9 “Gly Gly Ser” repeating units
  MOD_RES (4)..(4): Gly or absent
  MOD_RES (5)..(5): Gly or absent
  MOD_RES (6)..(6): Ser or absent
  MOD_RES (7)..(7): Gly or absent
  MOD_RES (8)..(8): Gly or absent
  MOD_RES (9)..(9): Ser or absent
  MOD_RES (10)..(10): Gly or absent
  MOD_RES (11)..(11): Gly or absent
  MOD_RES (12)..(12): Ser or absent
  MOD_RES (13)..(13): Gly or absent
  MOD_RES (14)..(14): Gly or absent
  MOD_RES (15)..(15): Ser or absent
  MOD_RES (16)..(16): Gly or absent
  MOD_RES (17)..(17): Gly or absent
  MOD_RES (18)..(18): Ser or absent
  MOD_RES (19)..(19): Gly or absent
  MOD_RES (20)..(20): Gly or absent
  MOD_RES (21)..(21): Ser or absent
  MOD_RES (22)..(22): Gly or absent
  MOD_RES (23)..(23): Gly or absent
  MOD_RES (24)..(24): Ser or absent
  MOD_RES (25)..(25): Gly or absent
  MOD_RES (26)..(26): Gly or absent
  MOD_RES (27)..(27): Ser or absent
  MOD_RES (28)..(28): Gly or absent
  MOD_RES (29)..(29): Gly or absent
  MOD_RES (30)..(30): Ser or absent
GGSXXXXXXXXXXXXXXXXXXXXXXXXXXX

SEQ ID NO: 157 - protein, 40 aa, Artificial Sequence
  Description of Artificial Sequence Synthetic polypeptide
  SITE (5)..(40): This region may encompass 1-9 “Gly Gly Gly Ser” repeating units
  MOD_RES (5)..(5): Gly or absent
  MOD_RES (6)..(6): Gly or absent
  MOD_RES (7)..(7): Gly or absent
  MOD_RES (8)..(8): Ser or absent
  MOD_RES (9)..(9): Gly or absent
  MOD_RES (10)..(10): Gly or absent
  MOD_RES (11)..(11): Gly or absent
  MOD_RES (12)..(12): Ser or absent
  MOD_RES (13)..(13): Gly or absent
  MOD_RES (14)..(14): Gly or absent
  MOD_RES (15)..(15): Gly or absent
  MOD_RES (16)..(16): Ser or absent
  MOD_RES (17)..(17): Gly or absent
  MOD_RES (18)..(18): Gly or absent
  MOD_RES (19)..(19): Gly or absent
  MOD_RES (20)..(20): Ser or absent
  MOD_RES (21)..(21): Gly or absent
  MOD_RES (22)..(22): Gly or absent
  MOD_RES (23)..(23): Gly or absent
  MOD_RES (24)..(24): Ser or absent
  MOD_RES (25)..(25): Gly or absent
  MOD_RES (26)..(26): Gly or absent
  MOD_RES (27)..(27): Gly or absent
  MOD_RES (28)..(28): Ser or absent
  MOD_RES (29)..(29): Gly or absent
  MOD_RES (30)..(30): Gly or absent
  MOD_RES (31)..(31): Gly or absent
  MOD_RES (32)..(32): Ser or absent
  MOD_RES (33)..(33): Gly or absent
  MOD_RES (34)..(34): Gly or absent
  MOD_RES (35)..(35): Gly or absent
  MOD_RES (36)..(36): Ser or absent
  MOD_RES (37)..(37): Gly or absent
  MOD_RES (38)..(38): Gly or absent
  MOD_RES (39)..(39): Gly or absent
  MOD_RES (40)..(40): Ser or absent
GGGSXXXXXXXXXXXXXXXXXXXXXXXXXXXXXXXXXXXX

SEQ ID NO: 158 - protein, 50 aa, Artificial Sequence
  Description of Artificial Sequence Synthetic polypeptide
  SITE (6)..(50): This region may encompass 1-9 “Gly Gly Gly Gly Ser” repeating units
  MOD_RES (6)..(6): Gly or absent
  MOD_RES (7)..(7): Gly or absent
  MOD_RES (8)..(8): Gly or absent
  MOD_RES (9)..(9): Gly or absent
  MOD_RES (10)..(10): Ser or absent
  MOD_RES (11)..(11): Gly or absent
  MOD_RES (12)..(12): Gly or absent
  MOD_RES (13)..(13): Gly or absent
  MOD_RES (14)..(14): Gly or absent
  MOD_RES (15)..(15): Ser or absent
  MOD_RES (16)..(16): Gly or absent
  MOD_RES (17)..(17): Gly or absent
  MOD_RES (18)..(18): Gly or absent
  MOD_RES (19)..(19): Gly or absent
  MOD_RES (20)..(20): Ser or absent
  MOD_RES (21)..(21): Gly or absent
  MOD_RES (22)..(22): Gly or absent
  MOD_RES (23)..(23): Gly or absent
  MOD_RES (24)..(24): Gly or absent
  MOD_RES (25)..(25): Ser or absent
  MOD_RES (26)..(26): Gly or absent
  MOD_RES (27)..(27): Gly or absent
  MOD_RES (28)..(28): Gly or absent
  MOD_RES (29)..(29): Gly or absent
  MOD_RES (30)..(30): Ser or absent
  MOD_RES (31)..(31): Gly or absent
  MOD_RES (32)..(32): Gly or absent
  MOD_RES (33)..(33): Gly or absent
  MOD_RES (34)..(34): Gly or absent
  MOD_RES (35)..(35): Ser or absent
  MOD_RES (36)..(36): Gly or absent
  MOD_RES (37)..(37): Gly or absent
  MOD_RES (38)..(38): Gly or absent
  MOD_RES (39)..(39): Gly or absent
  MOD_RES (40)..(40): Ser or absent
  MOD_RES (41)..(41): Gly or absent
  MOD_RES (42)..(42): Gly or absent
  MOD_RES (43)..(43): Gly or absent
  MOD_RES (44)..(44): Gly or absent
  MOD_RES (45)..(45): Ser or absent
  MOD_RES (46)..(46): Gly or absent
  MOD_RES (47)..(47): Gly or absent
  MOD_RES (48)..(48): Gly or absent
  MOD_RES (49)..(49): Gly or absent
  MOD_RES (50)..(50): Ser or absent
GGGGSXXXXXXXXXXXXXXXXXXXXXXXXXXXXXXXXXXXXXXXXXXXXX

SEQ ID NO: 159 - protein, 10 aa, Artificial Sequence
  Description of Artificial Sequence Synthetic peptide
  SITE (2)..(10): This region may encompass 1-9 residues
  MOD_RES (2)..(2): Gly or absent
  MOD_RES (3)..(3): Gly or absent
  MOD_RES (4)..(4): Gly or absent
  MOD_RES (5)..(5): Gly or absent
  MOD_RES (6)..(6): Gly or absent
  MOD_RES (7)..(7): Gly or absent
  MOD_RES (8)..(8): Gly or absent
  MOD_RES (9)..(9): Gly or absent
  MOD_RES (10)..(10): Gly or absent
GXXXXXXXXX

SEQ ID NO: 160 - protein, 50 aa, Artificial Sequence
  Description of Artificial Sequence Synthetic polypeptide
  SITE (6)..(50): This region may encompass 1-9 “Glu Ala Ala Ala Lys” repeating units
  MOD_RES (6)..(6): Glu or absent
  MOD_RES (7)..(7): Ala or absent
  MOD_RES (8)..(8): Ala or absent
  MOD_RES (9)..(9): Ala or absent
  MOD_RES (10)..(10): Lys or absent
  MOD_RES (11)..(11): Glu or absent
  MOD_RES (12)..(12): Ala or absent
  MOD_RES (13)..(13): Ala or absent
  MOD_RES (14)..(14): Ala or absent
  MOD_RES (15)..(15): Lys or absent
  MOD_RES (16)..(16): Glu or absent
  MOD_RES (17)..(17): Ala or absent
  MOD_RES (18)..(18): Ala or absent
  MOD_RES (19)..(19): Ala or absent
  MOD_RES (20)..(20): Lys or absent
  MOD_RES (21)..(21): Glu or absent
  MOD_RES (22)..(22): Ala or absent
  MOD_RES (23)..(23): Ala or absent
  MOD_RES (24)..(24): Ala or absent
  MOD_RES (25)..(25): Lys or absent
  MOD_RES (26)..(26): Glu or absent
  MOD_RES (27)..(27): Ala or absent
  MOD_RES (28)..(28): Ala or absent
  MOD_RES (29)..(29): Ala or absent
  MOD_RES (30)..(30): Lys or absent
  MOD_RES (31)..(31): Glu or absent
  MOD_RES (32)..(32): Ala or absent
  MOD_RES (33)..(33): Ala or absent
  MOD_RES (34)..(34): Ala or absent
  MOD_RES (35)..(35): Lys or absent
  MOD_RES (36)..(36): Glu or absent
  MOD_RES (37)..(37): Ala or absent
  MOD_RES (38)..(38): Ala or absent
  MOD_RES (39)..(39): Ala or absent
  MOD_RES (40)..(40): Lys or absent
  MOD_RES (41)..(41): Glu or absent
  MOD_RES (42)..(42): Ala or absent
  MOD_RES (43)..(43): Ala or absent
  MOD_RES (44)..(44): Ala or absent
  MOD_RES (45)..(45): Lys or absent
  MOD_RES (46)..(46): Glu or absent
  MOD_RES (47)..(47): Ala or absent
  MOD_RES (48)..(48): Ala or absent
  MOD_RES (49)..(49): Ala or absent
  MOD_RES (50)..(50): Lys or absent
EAAAKXXXXXXXXXXXXXXXXXXXXXXXXXXXXXXXXXXXXXXXXXXXXX

SEQ ID NO: 161 - protein, 106 aa, Artificial Sequence
  Description of Artificial Sequence Synthetic polypeptide
  SITE (7)..(51): This region may encompass 1-9 “Glu Ala Ala Ala Lys” repeating units
  MOD_RES (7)..(7): Glu or absent
  MOD_RES (8)..(8): Ala or absent
  MOD_RES (9)..(9): Ala or absent
  MOD_RES (10)..(10): Ala or absent
  MOD_RES (11)..(11): Lys or absent
  MOD_RES (12)..(12): Glu or absent
  MOD_RES (13)..(13): Ala or absent
  MOD_RES (14)..(14): Ala or absent
  MOD_RES (15)..(15): Ala or absent
  MOD_RES (16)..(16): Lys or absent
  MOD_RES (17)..(17): Glu or absent
  MOD_RES (18)..(18): Ala or absent
  MOD_RES (19)..(19): Ala or absent
  MOD_RES (20)..(20): Ala or absent
  MOD_RES (21)..(21): Lys or absent
  MOD_RES (22)..(22): Glu or absent
  MOD_RES (23)..(23): Ala or absent
  MOD_RES (24)..(24): Ala or absent
  MOD_RES (25)..(25): Ala or absent
  MOD_RES (26)..(26): Lys or absent
  MOD_RES (27)..(27): Glu or absent
  MOD_RES (28)..(28): Ala or absent
  MOD_RES (29)..(29): Ala or absent
  MOD_RES (30)..(30): Ala or absent
  MOD_RES (31)..(31): Lys or absent
  MOD_RES (32)..(32): Glu or absent
  MOD_RES (33)..(33): Ala or absent
  MOD_RES (34)..(34): Ala or absent
  MOD_RES (35)..(35): Ala or absent
  MOD_RES (36)..(36): Lys or absent
  MOD_RES (37)..(37): Glu or absent
  MOD_RES (38)..(38): Ala or absent
  MOD_RES (39)..(39): Ala or absent
  MOD_RES (40)..(40): Ala or absent
  MOD_RES (41)..(41): Lys or absent
  MOD_RES (42)..(42): Glu or absent
  MOD_RES (43)..(43): Ala or absent
  MOD_RES (44)..(44): Ala or absent
  MOD_RES (45)..(45): Ala or absent
  MOD_RES (46)..(46): Lys or absent
  MOD_RES (47)..(47): Glu or absent
  MOD_RES (48)..(48): Ala or absent
  MOD_RES (49)..(49): Ala or absent
  MOD_RES (50)..(50): Ala or absent
  MOD_RES (51)..(51): Lys or absent
  SITE (61)..(105): This region may encompass 1-9 “Glu Ala Ala Ala Lys” repeating units
  MOD_RES (61)..(61): Glu or absent
  MOD_RES (62)..(62): Ala or absent
  MOD_RES (63)..(63): Ala or absent
  MOD_RES (64)..(64): Ala or absent
  MOD_RES (65)..(65): Lys or absent
  MOD_RES (66)..(66): Glu or absent
  MOD_RES (67)..(67): Ala or absent
  MOD_RES (68)..(68): Ala or absent
  MOD_RES (69)..(69): Ala or absent
  MOD_RES (70)..(70): Lys or absent
  MOD_RES (71)..(71): Glu or absent
  MOD_RES (72)..(72): Ala or absent
  MOD_RES (73)..(73): Ala or absent
  MOD_RES (74)..(74): Ala or absent
  MOD_RES (75)..(75): Lys or absent
  MOD_RES (76)..(76): Glu or absent
  MOD_RES (77)..(77): Ala or absent
  MOD_RES (78)..(78): Ala or absent
  MOD_RES (79)..(79): Ala or absent
  MOD_RES (80)..(80): Lys or absent
  MOD_RES (81)..(81): Glu or absent
  MOD_RES (82)..(82): Ala or absent
  MOD_RES (83)..(83): Ala or absent
  MOD_RES (84)..(84): Ala or absent
  MOD_RES (85)..(85): Lys or absent
  MOD_RES (86)..(86): Glu or absent
  MOD_RES (87)..(87): Ala or absent
  MOD_RES (88)..(88): Ala or absent
  MOD_RES (89)..(89): Ala or absent
  MOD_RES (90)..(90): Lys or absent
  MOD_RES (91)..(91): Glu or absent
  MOD_RES (92)..(92): Ala or absent
  MOD_RES (93)..(93): Ala or absent
  MOD_RES (94)..(94): Ala or absent
  MOD_RES (95)..(95): Lys or absent
  MOD_RES (96)..(96): Glu or absent
  MOD_RES (97)..(97): Ala or absent
  MOD_RES (98)..(98): Ala or absent
  MOD_RES (99)..(99): Ala or absent
  MOD_RES (100)..(100): Lys or absent
  MOD_RES (101)..(101): Glu or absent
  MOD_RES (102)..(102): Ala or absent
  MOD_RES (103)..(103): Ala or absent
  MOD_RES (104)..(104): Ala or absent
  MOD_RES (105)..(105): Lys or absent
AEAAAKXXXXXXXXXXXXXXXXXXXXXXXXXXXXXXXXXXXXXXXXXXXXXALEAEAAAKXXXXXXXXXXXXXXXXXXXXXXXXXXXXXXXXXXXXXXXXXXXXXA

SEQ ID NO: 162 - protein, 5 aa, Artificial Sequence
  Description of Artificial Sequence Synthetic peptide
PAPAP

SEQ ID NO: 163 - protein, 12 aa, Artificial Sequence
  Description of Artificial Sequence Synthetic peptide
AEAAAKEAAAKA

SEQ ID NO: 164 - protein, 68 aa, Artificial Sequence
  Description of Artificial Sequence Synthetic polypeptide
  SITE (21)..(68): This region may encompass 1-24 “Ala Pro” repeating units
  MOD_RES (21)..(21): Ala or absent
  MOD_RES (22)..(22): Pro or absent
  MOD_RES (23)..(23): Ala or absent
  MOD_RES (24)..(24): Pro or absent
  MOD_RES (25)..(25): Ala or absent
  MOD_RES (26)..(26): Pro or absent
  MOD_RES (27)..(27): Ala or absent
  MOD_RES (28)..(28): Pro or absent
  MOD_RES (29)..(29): Ala or absent
  MOD_RES (30)..(30): Pro or absent
  MOD_RES (31)..(31): Ala or absent
  MOD_RES (32)..(32): Pro or absent
  MOD_RES (33)..(33): Ala or absent
  MOD_RES (34)..(34): Pro or absent
  MOD_RES (35)..(35): Ala or absent
  MOD_RES (36)..(36): Pro or absent
  MOD_RES (37)..(37): Ala or absent
  MOD_RES (38)..(38): Pro or absent
  MOD_RES (39)..(39): Ala or absent
  MOD_RES (40)..(40): Pro or absent
  MOD_RES (41)..(41): Ala or absent
  MOD_RES (42)..(42): Pro or absent
  MOD_RES (43)..(43): Ala or absent
  MOD_RES (44)..(44): Pro or absent
  MOD_RES (45)..(45): Ala or absent
  MOD_RES (46)..(46): Pro or absent
  MOD_RES (47)..(47): Ala or absent
  MOD_RES (48)..(48): Pro or absent
  MOD_RES (49)..(49): Ala or absent
  MOD_RES (50)..(50): Pro or absent
  MOD_RES (51)..(51): Ala or absent
  MOD_RES (52)..(52): Pro or absent
  MOD_RES (53)..(53): Ala or absent
  MOD_RES (54)..(54): Pro or absent
  MOD_RES (55)..(55): Ala or absent
  MOD_RES (56)..(56): Pro or absent
  MOD_RES (57)..(57): Ala or absent
  MOD_RES (58)..(58): Pro or absent
  MOD_RES (59)..(59): Ala or absent
  MOD_RES (60)..(60): Pro or absent
  MOD_RES (61)..(61): Ala or absent
  MOD_RES (62)..(62): Pro or absent
  MOD_RES (63)..(63): Ala or absent
  MOD_RES (64)..(64): Pro or absent
  MOD_RES (65)..(65): Ala or absent
  MOD_RES (66)..(66): Pro or absent
  MOD_RES (67)..(67): Ala or absent
  MOD_RES (68)..(68): Pro or absent
APAPAPAPAPAPAPAPAPAPXXXXXXXXXXXXXXXXXXXXXXXXXXXXXXXXXXXXXXXXXXXXXXXX

(19) United States
(12) Patent Application Publication
(10) Pub. No.: US 2024/0366795 A1
(43) Pub. Date: Nov. 7, 2024

(54) EPIGENETIC GENE REGULATION TO TREAT NEUROLOGICAL DISEASES AND PAIN

(71) Applicant: Navega Therapeutics, Inc., San Diego, CA (US)

(72) Inventors: Fernando ALEMAN GUILLEN, San Diego, CA (US); Ana Maria MORENO COLLADO, San Diego, CA (US)

(21) Appl. No.: 18/274,978

(22) PCT Filed: Jan. 31, 2022
(86) PCT No.: PCT/US2022/014601

§ 371 Date: Jul. 28, 2023

Related U.S. Application Data
(60) Provisional application No. 63/144,408, filed on Feb. 1, 2021.

(51) Int. Cl. A61K 48/00; A61K 38/46; C12N 9/22

(52) U.S. Cl. CPC A61K 48/0058; A61K 38/465; A61K 48/0033; C12N 9/22

(57) ABSTRACT

Described herein are compositions and methods to modulate gene expression without permanently editing the genome. The compositions and methods may be useful to treat neurological diseases and pain.

### CROSS REFERENCE TO RELATED APPLICATION

This application claims the benefit of U.S. provisional application 63/144,408, filed Feb. 1, 2021, the entirety of which is incorporated by reference herein.

### GOVERNMENT SUPPORT

This invention was made with government support under 1R43CA239940 awarded by The Department of Health and Human Services. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 15, 2024, is named 423157-701041_SL.txt and is 529,434 bytes in size.

### BACKGROUND

Chronic pain affects between 19% to 50% of the world population, with more than 100 million people affected in the U.S. alone (Institute of Medicine (US) Committee on Advancing Pain Research, Care, 2011). Despite their side-effects and limited efficacy, opioids have been a preferred treatment for chronic pain among both private and VA prescribers in recent years. Opioids, however, are highly addictive, and over 130 Americans die each day due to an overdose. Thus, opioid overdose represents a threat that significantly impacts public health. Even though chronic pain is more prevalent than cancer, diabetes and cardiovascular disease combined, drug development for chronic pain has not undergone the remarkable progress seen in these other therapeutic areas. Despite decades of research, broad-acting, long-lasting, non-addictive and effective therapeutics for chronic pain remain elusive.

### SUMMARY

Epigenetic modulation of gene(s) associated with pain can provide a non-permanent, long-lasting therapy for pain. In one aspect, epigenetic modulation comprises delivering a nuclease-null Cas protein with one or more guide-RNAs and a repression and/or activation domain to repress and/or activate the target gene(s). Similarly, Zinc Finger Proteins (ZFP) can be delivered with the repressor and/or activation domains to repress and/or activate the target gene(s).

In one aspect, provided herein is a nucleic acid comprising a sequence encoding a nucleic acid binding domain, a sequence encoding an epigenetic modulator that regulates transcription of one or more target molecules, and a promoter comprising a Nav1.7 promoter, a promoter having a sequence at least about 90% identical to a sequence of Table 5, or a promoter of a gene selected from Tables 1-3. In some embodiments, the one or more target molecules comprises a nucleic acid encoding an ion channel and/or is associated with an ion channel. In some embodiments, the one or more target molecules comprises SCN9A, SCN10A, or SCN11A, or a combination thereof. In some embodiments, the one or more target molecules is associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof. In some embodiments, the one or more target molecules comprises one or more genes of Table 1. In some embodiments, the one or more target molecules is associated with pain. In some embodiments, the one or more target molecules comprises one or more genes of Table 2 or Table 3. In some embodiments, the nucleic acid binding domain comprises a zinc finger protein. In some embodiments, the nucleic acid binding domain comprises a nuclease dead Clustered Regularly Interspaced Short Palindromic Repeats associated protein (dCas). In some embodiments, the epigenetic modulator comprises a domain having transcription repression activity (repressor domain). In some embodiments, the repressor domain comprises a ZIM3 repressor domain or a Krueppel-associated box (KRAB) domain (recruitment of histone methyltransferases and deacetylases). In some embodiments, the epigenetic modulator comprises a domain having transcription activation activity (activator domain). In some embodiments, the epigenetic modulator comprises ZIM3, KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, ZNF257, VP64, Rta, P16, P65, p300, TET1 catalytic domain, TDG, Ldb 1 self-association domain, SAM activator (VP64, p65, HSF1), VPR (VP64, p65, Rta), CD, or SunTag, or a variant or combination thereof. In some embodiments, the promoter comprises the Nav1.7 promoter. In some embodiments, the promoter comprises the sequence at least about 90% identical to a sequence of Table 5, optionally promoter 1 or promoter 2. In some embodiments, the promoter is the promoter of a gene selected from Tables 1-3. In some embodiments, the nucleic acid is delivered to the subject via a delivery vehicle. In some embodiments, the delivery vehicle is a viral delivery vehicle (e.g., retroviral vector, lentiviral vector, or adenoviral vector), liposome, nanoparticle, or exosome. In some embodiments, the viral delivery vehicle is a recombinant adeno-associated virus (AAV). In some embodiments, the AAV is AAV1, AAV2, AAV3, AAV4, AAV5, AAV6, AAV7, AAV8, AAV9, AAV10, AAVhu68, AAVrh. 10, AAVrh74 or AAVDJ, or a combination thereof. In some embodiments, the AAV is AAV9. In some embodiments, the AAV has a recombinant capsid. In some embodiments, the recombinant capsid comprises or comprises a sequence encoding a targeting moiety. In some embodiments, the delivery vehicle comprises or is connected to a targeting moiety. In some embodiments, the targeting moiety targets a cell comprising the target molecule in the subject. In some embodiments, the targeting moiety binds to a peptide product of the target molecule. In some embodiments, the target molecule is present on a target cell. In some embodiments, the target cell is associated with the disease or condition in the subject. In some embodiments, the targeting moiety comprises a peptide. In some embodiments, the peptide comprises JNJ63955, m3-Huwentoxin-IV, Phlotoxin 1 (PhITx1), Protoxin-II (ProTx-II), or Ceratotoxin-1 (CcoTx1), or a variant thereof. In some embodiments, the peptide comprises a sequence at least about 90% identical to a peptide of Table 7.

In one aspect, provided herein is a composition comprising a) a nucleic acid comprising a sequence encoding a nucleic acid binding domain and a sequence encoding an epigenetic modulator that regulates transcription of one or more target molecules, and b) a delivery vehicle for delivering the nucleic acid, wherein the delivery vehicle comprises a targeting peptide comprising JNJ63955, m3-Huwentoxin-IV, Phlotoxin 1 (PhITx1), Protoxin-II (ProTx-II), or Ceratotoxin-1 (CcoTx1), or a variant thereof; or a sequence at least about 90% identical to a peptide of Table 7. In some embodiments, the delivery vehicle is a viral delivery vehicle (e.g., retroviral vector, lentiviral vector, or adenoviral vector), liposome, nanoparticle, or exosome. In some embodiments, the viral delivery vehicle is a recombinant adeno-associated virus (AAV). In some embodiments, the AAVis AAV1, AAV2, AAV3, AAV4, AAV5, AAV6, AAV7, AAV8, AAV9, AAV10, AAVhu68, AAVrh. 10, AAVrh74 or AAVDJ, or a combination thereof. In some embodiments, the AAV is AAV9. In some embodiments, the AAV has a recombinant capsid. In some embodiments, the recombinant capsid comprises or comprises a sequence encoding a targeting moiety. In some embodiments, the delivery vehicle comprises or is connected to a targeting moiety. In some embodiments, the targeting moiety targets a cell comprising the target molecule in the subject. In some embodiments, the targeting moiety binds to a peptide product of the target molecule. In some embodiments, the target molecule is present on a target cell. In some embodiments, the target cell is associated with the disease or condition in the subject. In some embodiments, the targeting moiety comprises a peptide. In some embodiments, the peptide comprises JNJ63955, m3-Huwentoxin-IV, Phlotoxin 1 (Ph1Tx1), Protoxin-II (ProTx-II), or Ceratotoxin-1 (CcoTx1), or a variant thereof. In some embodiments, the peptide comprises a sequence at least about 90% identical to a peptide of Table 7.

In one aspect, provided herein is a method for modulating expression of one or more target molecules associated with a disease or condition in a subject, the method comprising administering to the subject a nucleic acid sequence encoding a nucleic acid binding domain and an epigenetic modulator that regulates transcription of the one or more target molecules. In some embodiments, regulation of the transcription of the one or more target molecules is transient. In some embodiments, the genome of the subject is not edited. In some embodiments, the disease or condition comprises pain. In some embodiments, the pain comprises neuropathic pain, inflammatory pain (e.g., rheumatoid arthritis), visceral pain, migraine pain, erythromelalgia pain, fibromyalgia pain, idiopathic pain, or somatic pain, or a combination thereof. In some embodiments, the disease or condition comprises a channelopathy. In some embodiments, the channelopathy comprises Dravet syndrome, Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof. In some embodiments, the disease or condition comprises a neurological disease. In some embodiments, the neurological disease is dementia. In some embodiments, the disease or condition comprises Alzheimer's disease. In some embodiments, the disease or condition comprises Parkinson's disease. In some embodiments, the disease or condition comprises Huntington's disease. In some embodiments, the disease or condition comprises schizophrenia. In some embodiments, the disease or condition comprises Amyotrophic lateral sclerosis (ALS). In some embodiments, the disease or condition comprises Multiple Sclerosis. In some embodiments, the disease or condition comprises a central nervous system ailment. In some embodiments, the disease or condition comprises an infection. In some embodiments, the disease or condition comprises Beta-thalassemia, Fragile X, centronuclear myopathy, Prion disease, Angelman Syndrome, Lafora disease, or Alexander disease.

In some embodiments, the one or more target molecules comprise DNA. In some embodiments, the one or more target molecules comprise RNA. In some embodiments, the one or more target molecules comprise a coding region of a gene. In some embodiments, the one or more target molecules comprise DNA complementary to non-coding RNA. In some embodiments, the non-coding RNA is associated with neuropathic pain. In some embodiments, the neuropathic pain comprises spinal nerve ligation, spared nerve injury, chronic constriction injury, or diabetic neuropathy, or a combination thereof. In some embodiments, the non-coding RNA comprises a SCN9A natural antisense transcript (NAT), a Kcna2 antisense RNA, H19, Gm21781, MRAK009713, uc.48+, NONRATT021972, BC168687, Speer7-ps, Uc007pbc.1, XLOC_041439, Mlxipl, Rn50_X_0739.1, CCATI, mno circ 0004058, rno_circRNA_007512, or Egr2 antisense RNA, or a combination thereof. In some embodiments, the one or more target molecules comprises a nucleic acid encoding a channel. In some embodiments, the one or more target molecules comprises a nucleic acid associated with a channel. In some embodiments, the channel is an ion channel. In some embodiments, the ion channel is a sodium channel. In some embodiments, the ion channel is a potassium channel. In some embodiments, the ion channel is a calcium channel. In some embodiments, the ion channel is a chloride channel. In some embodiments, the one or more target molecules is associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof. In some embodiments, the one or more target molecules comprises one or more genes of Table 1. In some embodiments, the one or more target molecules comprises SCN9A, SCN10A, or SCN11A, or a combination thereof. In some embodiments, the one or more target molecules comprise a natural antisense transcript for SCN9A. In some embodiments, the one or more target molecules is associated with pain. In some embodiments, the pain comprises neuropathic pain, inflammatory pain (e.g., rheumatoid arthritis), visceral pain, migraine pain, erythromelalgia pain, fibromyalgia pain, idiopathic pain, or somatic pain, or a combination thereof. In some embodiments, the one or more target molecules comprises one or more genes of Table 2. In some embodiments, the one or more target molecules is associated with a neurological disease. In some embodiments, the neurological disease comprises dementia. In some embodiments, the one or more target molecules is associated with Alzheimer's disease. In some embodiments, the one or more target molecules comprises one or more genes of Table 3. In some embodiments, the one or more target molecules is associated with Parkinson's disease. In some embodiments, the one or more target molecules comprises one or more genes selected from the group comprising SNCA, GBA, and LRRK2. In some embodiments, the one or more target molecules is associated with Huntington's disease. In some embodiments, the one or more target molecules is associated with schizophrenia. In some embodiments, the one or more target molecules comprises GPR52. In some embodiments, the one or more target molecules is associated with Amyotrophic lateral sclerosis (ALS). In some embodiments, the one or more target molecule comprises one or more genes selected from the group comprising SOD1, ataxin-2, TDP43, FUS, (9ORF72 and SCA2. In some embodiments, the one or more target molecules is associated with Multiple Sclerosis. In some embodiments, the one or more target molecules is associated with a central nervous system ailment. In some embodiments, the one or more target molecules comprise one or more genes selected from the group comprising BFD1, FUS, C9orf72, Brain-derived neurotrophic factor, Nerve growth factor, a Neurotrophin, BCL11A, FMR1, DNM2, PrP, UBE3A, GYS1, STING, and GFAP.

In some embodiments, the nucleic acid binding domain binds to at least one of the one or more target molecules. In some embodiments, the nucleic acid binding domain comprises a nuclease dead Clustered Regularly Interspaced Short Palindromic Repeats associated protein (dCas). In some embodiments, the dCas comprises a mutated Cas protein. In some embodiments, the dCas is a truncated Cas protein. In some embodiments, the dCas is a mutated or truncated Cas protein of Table 4. In some embodiments, the dCas comprises a sequence at least 90% identical to a dCas of Table 4. In some embodiments, the dCas comprises dCas9. In some embodiments, the dCas9 is a truncated or mutated Cas9 protein. In some embodiments, the dCas comprises dCas12. In some embodiments, the dCas 12 is a truncated or mutated Cas 12 protein. In some embodiments, the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteurianus, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9. In some embodiments, the dCas has a REC2 domain deletion. In some embodiments, the dCas has a REC3 domain deletion. In some embodiments, the dCas has a HNH deletion. In some embodiments, the dCas has a nuclease (NUC) lobe deletion. In some embodiments, the nucleic acid binding domain comprises a zinc finger protein. In some embodiments, the nucleic acid binding domain comprises a meganuclease. In some embodiments, the nucleic acid binding domain comprises a transcription activator-like effector nucleases (TALENs).

In some embodiments, the epigenetic modulator comprises a transcription regulatory domain. In some embodiments, the epigenetic modulator comprises a domain having transcription repression activity (repressor domain). In some embodiments, the repressor domain comprises a Krueppel-associated box (KRAB) domain (recruitment of histone methyltransferases and deacetylases). In some embodiments, the epigenetic modulator comprises a domain having transcription activation activity (activator domain). In some embodiments, the epigenetic modulator comprises KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, or G9a (EHMT2), or a variant or combination thereof. In some embodiments, the epigenetic modulator comprises ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, or ZNF257, or a variant or combination thereof. In some embodiments, the epigenetic modulator comprises VP64, Rta, P16, P65, p300, TET1 catalytic domain, TDG, Ldb 1 self-association domain, SAM activator (VP64, p65, HSF1), VPR (VP64, p65, Rta), CD, or SunTag, or a variant or combination thereof. In some embodiments, the epigenetic modulator comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof. In some embodiments, the epigenetic modulator comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb 1 self-association domain (recruits enhancer-associated endogenous Ldb1), SAM activator (VP64, p65, HSF1) (recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2) (histone methyltransferase), DNMT3a (DNA methyltransferase), or DNMT3a-DNMT3L (DNA methyltransferase), or a combination thereof. In some embodiments, the epigenetic modulator is linked to the nucleic acid binding domain at the N-terminus or C-terminus of the nucleic acid binding domain via a linker. In some embodiments, the linker is a flexible linker. In some embodiments, the linker is (GGS)n (SEQ ID NO: 156), (GGGS)n (SEQ ID NO: 157), (GGGGS)n (SEQ ID NO: 158), (G)n (SEQ ID NO: 159), (EAAAK)n (SEQ ID NO: 160), A (EAAAK)nALEA(EAAAK)nA (SEQ ID NO: 161), PAPAP (SEQ ID NO: 162), AEAAAKEAAAKA (SEQ ID NO: 163), (Ala-Pro)x (SEQ ID NO: 164), LE, GlySer-polyPro (Glyc)-polyPro (Glyc)-polyPro (Glyc), GlySer-polyPro-polyPro (Glyc)-polyPro, GlySer-polyPro-GlySer (Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-β2m-GlySer-β2m-polyPro, GlySer-polyPro-Ub-GlySer, GlySer-polyPro-ZAG-polyPro, GlySer-GlySer-ZAG-GlySer-ZAG-polyPro, GlySer (Glyc)-GlySer (Glyc)-polyPro, (G4S) 3-cTPR3-(G4S) 3, (G4S) 3-cTPR6-(G4S) 3, (G4S) 3-cTPR9-(G4S) 3, (G4S) 3-cTPR12-(G4S) 3, or (G4S)n (SEQ ID NO: 158); wherein n is independently selected from 1 to 10, x is 10-34, polyPro is proline-rich hinge sequence from IgA1, polyPro (Glyc) is proline-rich hinge sequence from IgA1 with an embedded potential N-linked glycosylation site (Asn-Ser-Ser), β2m is β2-microglobulin, Ub is ubiquitin, ZAG is Zn-α2-glycoprotein, and cTPRX is consensus tetratricopeptide repeat sequence with X number of repeats. In some embodiments, the nucleic acid binding domain and the epigenetic modulator are connected by a disulfide bond.

In some embodiments, the nucleic acid sequence comprises a nuclear localization sequence (NLS). In some embodiments, the nucleic acid sequence comprises a promoter. In some embodiments, the promoter comprises a sequence at least about 90% identical to sequence of Table 5. In some embodiments, the promoter comprises a nuclear localization sequence. In some embodiments, the NLS drives nuclear import of the nucleic acid. In some embodiments, the promoter drives expression of the nucleic acid binding domain. In some embodiments, the promoter drives expression of the epigenetic modulator. In some embodiments, the promoter is a promoter naturally associated with the target molecule. In some embodiments, the promoter is a promoter of a gene from Tables 1-3. In some embodiments, the promoter is a SCN9A promoter or SCN10A promoter. In some embodiments, the promoter is a pan-neuronal gene promoter. In some embodiments, the promoter is a promoter naturally associated with a gene related to pain. In some embodiments, the promoter is a promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, promoter of the NeuN gene (Fox-3, Rbfox3, or Hexaribonucleotide Binding Protein-3), promoter of the α-calcium/calmodulin-dependent protein kinase II [CaMKIIα]), promoter of the Rheb gene (ras homolog enriched in brain), TRKA promoter (Tyrosine Kinase A), or jET. In some embodiments, the promoter is a promoter naturally associated with a gene associated with a channel. In some embodiments, the promoter is a promoter naturally associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof. In some embodiments, the promoter is a promoter naturally associated with inflammatory pain, visceral pain, migraine pain, erythromelalgia pain, fibromyalgia pain, idiopathic pain, or somatic pain, or a combination thereof. In some embodiments, the promoter is a promoter naturally associated with a neurological disease. In some embodiments, the promoter is a promoter naturally associated with dementia. In some embodiments, the promoter is a promoter naturally associated with Alzheimer's disease. In some embodiments, the promoter is a promoter naturally associated with Parkinson's disease. In some embodiments, the promoter is a promoter naturally associated with ALS. In some embodiments, the promoter is a promoter naturally associated with Multiple Sclerosis. In some embodiments, the promoter is a promoter naturally associated with a gene associated with a central nervous system ailment. In some embodiments, the promoter is a promoter of a gene selected from SNCA, GBA, and LRRK2, SOD1, ataxin-2, SCA2, BFD1, FUS, C9orf72, Brain-derived neurotrophic factor, Nerve growth factor, a Neurotrophin, BCL11A, FMR1, DNM2, PrP, UBE3A, GYS1, and GFAP. In some embodiments, the promoter is a pol II promoter (e.g., Thy1 and H1xb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter. In some embodiments, the promoter is controlled by a small molecule. In some embodiments, the promoter is a tetracycline responsive promoter, glucocorticoid responsive promoter, RU-486 responsive promoter, peroxide inducible promoter or tamoxifen induced promoter. In some embodiments, expression of the epigenetic modulator and/or transcription regulatory domain occurs upon a natural or physiological induction of the promoter. In some embodiments, the promoter is induced when a pathology arises in the subject. In some embodiments, the pathology is injury and/or inflammation. In some embodiments, the promoter is a galanin promoter or NF-κB promoter. In some embodiments, the nucleic acid sequence comprises two or more promoters. In some embodiments, the nucleic acid sequence comprises tandem promoters.

In some embodiments, the nucleic acid sequence comprises an enhancer. In some embodiments, the nucleic acid sequence comprises an intron. In some embodiments, the nucleic acid comprises an inverted terminal repeat (ITR). In some embodiments, the nucleic acid comprises a terminator sequence.

In some embodiments, the method comprises administering to the subject one or more guide RNA sequences. In some embodiments, the one or more guide RNA sequences is selected from a sequence in Table 6. In some embodiments, the one or more guide RNA sequences binds to one or more of the target molecules. In some embodiments, the one or more target molecules is 2, 3, 4, or 5 target molecules. In some embodiments, the one or more guide RNA sequences is 2, 3, 4, or 5 guide RNA sequences. In some embodiments, the method comprises administering at least two guide RNA sequences, wherein at least two of the guide RNA sequences are different. In some embodiments, at least one or more guide RNA sequences target a promoter described herein, creating a regulatory feedback loop to control expression levels.

In some embodiments, the nucleic acid is delivered as a naked (or unmodified) nucleic acid. In some embodiments, the nucleic acid is delivered complexed with cationic molecules. In some embodiments, the nucleic acid is delivered to the subject via a vehicle. In some embodiments, the vehicle is a viral delivery vehicle (e.g., retroviral vector, lentiviral vector, or adenoviral vector), liposome, nanoparticle, or exosome. In some embodiments, the vehicle is a viral delivery vehicle. In some embodiments, the viral delivery vehicle is a retroviral vector, lentiviral vector, or adenoviral vector. In some embodiments, the viral delivery vehicle is a recombinant adeno-associated virus (AAV). In some embodiments, the AAV is AAV1, AAV2, AAV3, AAV4, AAV5, AAV6, AAV7, AAV8, AAV9, AAV10, AAVhu68, AAVrh. 10, AAVrh74 or AAVDJ, or a combination thereof. In some embodiments, the AAV is AAV9. In some embodiments, the AAV has a recombinant capsid. In some embodiments, the recombinant capsid encodes a targeting moiety. In some embodiments, the nucleic acid comprises a sequence encoding a targeting moiety. In some embodiments, the vehicle is a liposome, lipid nanoparticle, nanocapsule, or exosome. In some embodiments, the vehicle comprises or is connected to a targeting moiety. In some embodiments, the targeting moiety targets a cell comprising the target molecule in the subject. In some embodiments, the targeting moiety binds to a peptide product of the target molecule. In some embodiments, the target molecule is present on a target cell. In some embodiments, the target cell is associated with the disease or condition in the subject. In some embodiments, the targeting moiety comprises a peptide. In some embodiments, the peptide comprises JNJ63955, m3-Huwentoxin-IV, Phlotoxin 1, Protoxin-II (ProTx-II), Huwentoxin IV (HwTx-IV), or Ceratotoxin-1 (CcoTx1), or a variant thereof. In some embodiments, the peptide comprises a sequence at least about 90% identical to a peptide of Table 7.

In some embodiments, the method further comprises administering to the subject an additional therapeutic agent.

In some embodiments, the nucleic acid is administered via lumbar intrathecal puncture, epidural, intravenous, transdermal, intranasal, oral, mucosal, intracisterna magna administration, or intraganglionic administration.

In another aspect, further provided is a nucleic acid sequence encoding a nucleic acid binding domain and an epigenetic modulator that regulates transcription of the one or more target molecules. In some embodiments, regulation of the transcription of the one or more target molecules is transient. In some embodiments, the genome of the subject is not edited. In some embodiments, the one or more target molecules comprise DNA. In some embodiments, the one or more target molecules comprise RNA. In some embodiments, the one or more target molecules comprise a coding region of a gene. In some embodiments, the one or more target molecules comprise DNA complementary to non-coding RNA. In some embodiments, the non-coding RNA is associated with neuropathic pain. In some embodiments, the neuropathic pain comprises spinal nerve ligation, spared nerve injury, chronic constriction injury, or diabetic neuropathy, or a combination thereof. In some embodiments, the non-coding RNA comprises a SCN9A natural antisense transcript (NAT), a Kcna2 antisense RNA, H19, Gm21781, MRAK009713, uc.48+, NONRATT021972, BC168687, Speer7-ps, Uc007pbc. 1, XLOC_041439, Mlxipl, Rn50_X_0739.1, CCATI, mno circ 0004058, rno_circRNA_007512, or Egr2 antisense RNA, or a combination thereof. In some embodiments, the one or more target molecules comprises a nucleic acid encoding a channel. In some embodiments, the one or more target molecules comprises a nucleic acid associated with a channel. In some embodiments, the channel is an ion channel. In some embodiments, the ion channel is a sodium channel. In some embodiments, the ion channel is a potassium channel. In some embodiments, the ion channel is a calcium channel. In some embodiments, the ion channel is a chloride channel. In some embodiments, the one or more target molecules is associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof. In some embodiments, the one or more target molecules comprises one or more genes of Table 1. In some embodiments, the one or more target molecules comprises SCN9A, SCN10A, or SCN11A, or a combination thereof. In some embodiments, the one or more target molecules comprise a natural antisense transcript for SCN9A. In some embodiments, the one or more target molecules is associated with pain. In some embodiments, the pain comprises neuropathic pain, inflammatory pain, visceral pain, migraine pain, erythromelalgia pain, fibromyalgia pain, idiopathic pain, or somatic pain, or a combination thereof. In some embodiments, the one or more target molecules comprises one or more genes of Table 2. In some embodiments, the one or more target molecules is associated with a neurological disease. In some embodiments, the neurological disease comprises dementia. In some embodiments, the one or more target molecules is associated with Alzheimer's disease. In some embodiments, the one or more target molecules comprises one or more genes of Table 3. In some embodiments, the one or more target molecules is associated with Parkinson's disease. In some embodiments, the one or more target molecules comprises one or more genes selected from the group comprising SNCA, GBA, and LRRK2. In some embodiments, the one or more target molecules is associated with Huntington's disease. In some embodiments, the one or more target molecules is associated with schizophrenia. In some embodiments, the one or more target molecules comprises GPR52. In some embodiments, the one or more target molecules is associated with Amyotrophic lateral sclerosis (ALS). In some embodiments, the one or more target molecule comprises one or more genes selected from the group comprising SOD1, ataxin-2, TDP43, FUS, C9ORF72 and SCA2. In some embodiments, the one or more target molecules is associated with Multiple Sclerosis. In some embodiments, the one or more target molecules is associated with a central nervous system ailment. In some embodiments, the one or more target molecules comprise one or more genes selected from the group comprising BFD1, FUS, C9orf72, Brain-derived neurotrophic factor, Nerve growth factor, a Neurotrophin, BCL11A, FMR1, DNM2, PrP, UBE3A, GYS1, STING, and GFAP.

In some embodiments, the nucleic acid binding domain binds to at least one of the one or more target molecules. In some embodiments, the nucleic acid binding domain comprises a nuclease dead Clustered Regularly Interspaced Short Palindromic Repeats associated protein (dCas). In some embodiments, the dCas comprises a mutated Cas protein. In some embodiments, the dCas is a truncated Cas protein. In some embodiments, the dCas is a mutated or truncated Cas protein of Table 4. In some embodiments, the dCas comprises a sequence at least 90% identical to a dCas of Table 4. In some embodiments, the dCas comprises dCas9. In some embodiments, the dCas9 is a truncated or mutated Cas9 protein. In some embodiments, the dCas comprises dCas12. In some embodiments, the dCas 12 is a truncated or mutated Cas12 protein. In some embodiments, the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteurianus, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9. In some embodiments, the dCas has a REC2 domain deletion. In some embodiments, the dCas has a REC3 domain deletion. In some embodiments, the dCas has a HNH deletion. In some embodiments, the dCas has a nuclease (NUC) lobe deletion. In some embodiments, the nucleic acid binding domain comprises a zinc finger protein. In some embodiments, the nucleic acid binding domain comprises a meganuclease. In some embodiments, the nucleic acid binding domain comprises a transcription activator-like effector nucleases (TALENs).

In some embodiments, the epigenetic modulator comprises a transcription regulatory domain. In some embodiments, the epigenetic modulator comprises a domain having transcription repression activity (repressor domain). In some embodiments, the repressor domain comprises a Krueppel-associated box (KRAB) domain (recruitment of histone methyltransferases and deacetylases). In some embodiments, the epigenetic modulator comprises a domain having transcription activation activity (activator domain). In some embodiments, the epigenetic modulator comprises KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, or G9a (EHMT2), or a variant or combination thereof. In some embodiments, the epigenetic modulator comprises ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, or ZNF257, or a variant or combination thereof. In some embodiments, the epigenetic modulator comprises VP64, Rta, P16, P65, p300, TET1 catalytic domain, TDG, Ldb 1 self-association domain, SAM activator (VP64, p65, HSF1), VPR (VP64, p65, Rta), CD, or SunTag, or a variant or combination thereof. In some embodiments, the epigenetic modulator comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof. In some embodiments, the epigenetic modulator comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb1 self-association domain (recruits enhancer-associated endogenous Ldb1), SAM activator (VP64, p65, HSF1) (recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2) (histone methyltransferase), DNMT3a (DNA methyltransferase), or DNMT3α-DNMT3L (DNA methyltransferase), or a combination thereof. In some embodiments, the epigenetic modulator is linked to the nucleic acid binding domain at the N-terminus or C-terminus of the nucleic acid binding domain via a linker. In some embodiments, the linker is a flexible linker. In some embodiments, the linker is (GGS)n (SEQ ID NO: 156), (GGGS)n (SEQ ID NO: 157), (GGGGS)n (SEQ ID NO: 158), (G)n (SEQ ID NO: 159), (EAAAK)n (SEQ ID NO: 160), A (EAAAK)nALEA(EAAAK)nA (SEQ ID NO: 161), PAPAP (SEQ ID NO: 162), AEAAAKEAAAKA (SEQ ID NO: 163), (Ala-Pro)x (SEQ ID NO: 164), LE, GlySer-polyPro (Glyc)-polyPro (Glyc)-polyPro (Glyc), GlySer-polyPro-polyPro (Glyc)-polyPro, GlySer-polyPro-GlySer (Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-β2m-GlySer-β2m-polyPro, GlySer-polyPro-Ub-GlySer, GlySer-polyPro-ZAG-polyPro, GlySer-GlySer-ZAG-GlySer-ZAG-polyPro, Gly Ser (Glyc)-GlySer (Glyc)-polyPro, (G4S) 3-cTPR3-(G4S) 3, (G4S) 3-cTPR6-(G4S) 3, (G4S) 3-cTPR9-(G4S) 3, (G4S) 3-cTPR12-(G4S) 3, or (G4S)n (SEQ ID NO: 158); wherein n is independently selected from 1 to 10, x is 10-34, polyPro is proline-rich hinge sequence from IgA1, polyPro (Glyc) is proline-rich hinge sequence from IgA1 with an embedded potential N-linked glycosylation site (Asn-Ser-Ser), B2m is β2-microglobulin, Ub is ubiquitin, ZAG is Zn-α2-glycoprotein, and CTPRX is consensus tetratricopeptide repeat sequence with X number of repeats. In some embodiments, the nucleic acid binding domain and the epigenetic modulator are connected by a disulfide bond.

In some embodiments, the nucleic acid sequence comprises a nuclear localization sequence (NLS). In some embodiments, the nucleic acid sequence comprises a promoter. In some embodiments, the promoter comprises a sequence at least about 90% identical to sequence of Table 5. In some embodiments, the promoter comprises a nuclear localization sequence. In some embodiments, the NLS drives nuclear import of the nucleic acid. In some embodiments, the promoter drives expression of the nucleic acid binding domain. In some embodiments, the promoter drives expression of the epigenetic modulator. In some embodiments, the promoter is a promoter naturally associated with the target molecule. In some embodiments, the promoter is a promoter of a gene from Tables 1-3. In some embodiments, the promoter is a SCN9A promoter or SCN10A promoter. In some embodiments, the promoter is a pan-neuronal gene promoter. In some embodiments, the promoter is a promoter naturally associated with a gene related to pain. In some embodiments, the promoter is a promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, promoter of the NeuN gene (Fox-3, Rbfox3, or Hexaribonucleotide Binding Protein-3), promoter of the α-calcium/calmodulin-dependent protein kinase II [CaMKIIα]), promoter of the Rheb gene (ras homolog enriched in brain), TRKA promoter (Tyrosine Kinase A), or jET. In some embodiments, the promoter is a promoter naturally associated with a gene associated with a channel. In some embodiments, the promoter is a promoter naturally associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof. In some embodiments, the promoter is a promoter naturally associated with inflammatory pain, visceral pain, migraine pain, erythromelalgia pain, fibromyalgia pain, idiopathic pain, or somatic pain, or a combination thereof. In some embodiments, the promoter is a promoter naturally associated with a neurological disease. In some embodiments, the promoter is a promoter naturally associated with dementia. In some embodiments, the promoter is a promoter naturally associated with Alzheimer's disease. In some embodiments, the promoter is a promoter naturally associated with Parkinson's disease. In some embodiments, the promoter is a promoter naturally associated with ALS. In some embodiments, the promoter is a promoter naturally associated with Multiple Sclerosis. In some embodiments, the promoter is a promoter naturally associated with a gene associated with a central nervous system ailment. In some embodiments, the promoter is a promoter of a gene selected from SNCA, GBA, and LRRK2, SOD1, ataxin-2, SCA2, BFD1, FUS, C9orf72, Brain-derived neurotrophic factor, Nerve growth factor, a Neurotrophin, BCL11A, FMR1, DNM2, PrP, UBE3A, GYS1, and GFAP. In some embodiments, the promoter is a pol II promoter (e.g., Thy1 and H1xb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter. In some embodiments, the promoter is controlled by a small molecule. In some embodiments, the promoter is a tetracycline responsive promoter, glucocorticoid responsive promoter, RU-486 responsive promoter, peroxide inducible promoter or tamoxifen induced promoter. In some embodiments, expression of the epigenetic modulator and/or transcription regulatory domain occurs upon a natural or physiological induction of the promoter. In some embodiments, the promoter is induced when a pathology arises in the subject. In some embodiments, the pathology is injury and/or inflammation. In some embodiments, the promoter is a galanin promoter or NF-κB promoter. In some embodiments, the nucleic acid sequence comprises two or more promoters. In some embodiments, the nucleic acid sequence comprises tandem promoters.

In some embodiments, the nucleic acid sequence comprises an enhancer. In some embodiments, the nucleic acid sequence comprises an intron. In some embodiments, the nucleic acid comprises an inverted terminal repeat (ITR). In some embodiments, the nucleic acid comprises a terminator sequence.

In some embodiments, the nucleic acid is administered with one or more guide RNA sequences. In some embodiments, the one or more guide RNA sequences is selected from a sequence in Table 6. In some embodiments, the one or more guide RNA sequences binds to one or more of the target molecules. In some embodiments, the one or more target molecules is 2, 3, 4, or 5 target molecules. In some embodiments, the one or more guide RNA sequences is 2, 3, 4, or 5 guide RNA sequences. In some embodiments, at least two guide RNA sequences are administered, wherein at least two of the guide RNA sequences are different. In some embodiments, at least one or more guide RNA sequences target the promoter of claims 221-252, creating a regulatory feedback loop to control expression levels.

In some embodiments, the nucleic acid is delivered as a naked (or unmodified) nucleic acid. In some embodiments, the nucleic acid is delivered complexed with cationic molecules. In some embodiments, the nucleic acid is delivered to the subject via a vehicle. In some embodiments, the vehicle is a viral delivery vehicle (e.g., retroviral vector, lentiviral vector, or adenoviral vector), liposome, nanoparticle, or exosome. In some embodiments, the vehicle is a viral delivery vehicle. In some embodiments, the viral delivery vehicle is a retroviral vector, lentiviral vector, or adenoviral vector. In some embodiments, the viral delivery vehicle is a recombinant adeno-associated virus (AAV). In some embodiments, the AAV is AAV1, AAV2, AAV3, AAV4, AAV5, AAV6, AAV7, AAV8, AAV9, AAV10, AAVhu68, AAVrh. 10, AAVrh74 or AAVDJ, or a combination thereof. In some embodiments, the AAV is AAV9. In some embodiments, the AAV has a recombinant capsid. In some embodiments, the recombinant capsid encodes a targeting moiety. In some embodiments, the nucleic acid comprises a sequence encoding a targeting moiety. In some embodiments, the vehicle is a liposome, lipid nanoparticle, nanocapsule, or exosome. In some embodiments, the vehicle comprises or is connected to a targeting moiety. In some embodiments, the targeting moiety targets a cell comprising the target molecule in the subject. In some embodiments, the targeting moiety binds to a peptide product of the target molecule. In some embodiments, the target molecule is present on a target cell. In some embodiments, the target cell is associated with the disease or condition in the subject. In some embodiments, the targeting moiety comprises a peptide. In some embodiments, the peptide comprises JNJ63955, m3-Huwentoxin-IV, Phlotoxin 1, Protoxin-II (ProTx-II), or Ceratotoxin-1 (CcoTx1), Huwentoxin IV, or a variant thereof. In some embodiments, the peptide comprises a sequence at least about 90% identical to a peptide of Table 7.

Further provided is a combination comprising the nucleic acid sequence and an additional therapeutic agent.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a schematic representation of an example nucleic acid composition described herein.

FIG. 2 is a schematic representation of another example nucleic acid composition described herein.

FIG. 3 shows expression of mCherry driven by different Nav 1.7 promoters in a high Nav1.7-expressing human cell line, HuH7.

FIG. 4 shows repression of Nav1.7 gene expression after transfection of HuH7 cells with a nucleic acid composition encoding ZFP and KRAB targeting Nav1.7.

FIG. 5 is a schematic representation of an example nucleic acid composition described herein for repression of a single target molecule.

FIG. 6 is a schematic representation of an example nucleic acid composition described herein for repression of two target molecules.

FIGS. 7A-7B show improvements in reversing allodynia in male mice with single- and dual-target gene repression.

FIGS. 8A-8B show improvements in reversing allodynia in female mice with single- and dual-target gene repression.

FIGS. 9A-9C show expression of mCherry driven by different chemical conjugation of Nav1.7-binding peptides bound to the surface of AAV9 on different cell lines.

FIG. 10 shows expression of mCherry driven by the expression of Nav1.7-binding peptide JNJ63955918-Indel and a short 6 bp linker on the AAV9 capsid.

FIGS. 11A-11D show expression of mCherry from different Nav1.7-specific promoters on different cell lines.

FIG. 12 shows expression of mCherry from a Nav1.7-specific promoter in Nav1.7-expressing neurons of mouse dorsal root ganglia (DRG).

### DESCRIPTION OF THE INVENTION

Described herein are compositions and methods to modulate gene expression via nucleic acid binding proteins that do not permanently edit the genome. The compositions and methods may be used to treat a disease or condition such as a neurological disease and/or pain. The compositions include nucleic acid sequences encoding nuclease-inactivated CRISPR-Cas systems (dead Cas for CRISPRi or CRISPRa) or Zinc Finger Proteins, along with epigenetic modulators, for gene repression, activation and epigenetic editing. The methods include introducing the composition into a target cell and modulating expression of a gene or non-coding RNA in the cell, thereby treating a subject for a disease or condition associated with the gene or non-coding RNA. In exemplary embodiments, the methods provide an avenue for targeted nucleic acid delivery using specific promoters and adeno-associated virus (AAV) modifications to enhance viral tropism.

### Nucleic Acid Compositions

In one aspect, provided herein are nucleic acid compositions comprising a nucleic acid sequence encoding a DNA binding domain and an epigenetic modulator. Non-limiting examples of such DNA binding domains and epigenetic modulators are described herein. In example embodiments, the nucleic acid binding domain and epigenetic modulator are connected via a linker to form an epigenetic repressor or activator of gene expression. In some cases, the compositions further comprise one or more of the following components: guide RNA, promoter, enhancer, intron, nuclear localization signal, ITR, terminator sequence, and/or delivery vehicle.

Also provided herein are nucleic acid compositions comprising an adeno-associated virus modified with a sequence encoding a peptide specific for a protein product of a target molecule. Such compositions may be useful for targeting the nucleic acid to a cell expressing the target molecule for a targeted therapeutic approach. For example, the peptide specifically binds to the protein product of the target molecule. Non-limiting examples of specific binding include peptides that bind to the protein product of the target molecule with a high affinity, e.g., an affinity in the nanomolar range.

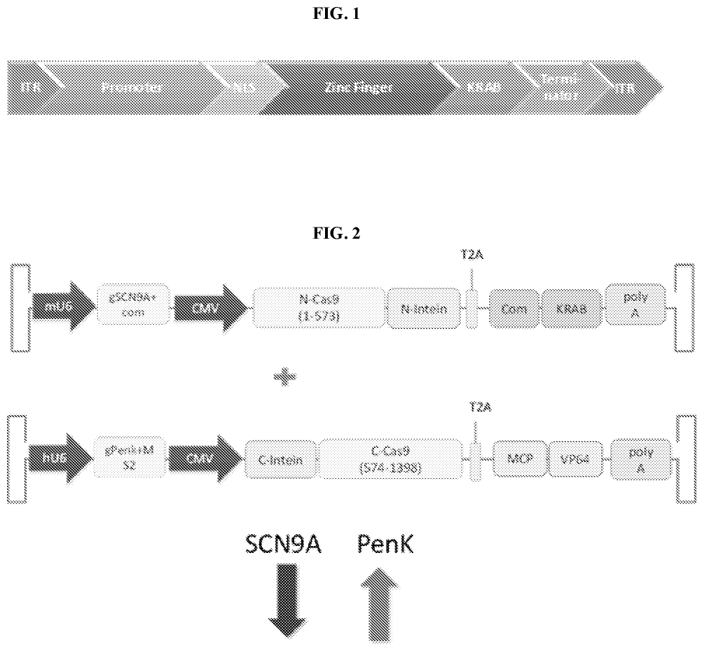

FIG. 1 provides a schematic of an example nucleic acid composition described herein. The composition of FIG. 1 comprises inverted terminal repeats (ITR), a promoter, a nuclear localization sequence, a sequence encoding a nucleic acid binding protein (exemplified as a Zinc Finger Protein), a sequence encoding an epigenetic modulator (exemplified as a KRAB domain), and a terminator sequence. The promoter may be a cell-specific promoter (e.g., specific for cells expressing Nav1.7, Nav1.8, TRPV1, TRPA1 or neuronal specific). The DNA sequence may be packaged in a single-stranded adeno-associated virus or self complementary adeno-associated virus (e.g., AAV9).

### Nucleic Acid Binding Proteins

Provided herein are nucleic acid binding proteins used to modulate gene expression without permanently editing the genome. In certain embodiments, the nucleic acid binding domain binds to a target molecule. In certain embodiments, the nucleic acid binding domain binds to DNA. In certain embodiments, the nucleic acid binding domain binds to RNA or DNA complementary to the RNA.

### CRISPR/Cas

An example nucleic acid binding domain is a nuclease dead Clustered Regularly Interspaced Short Palindromic Repeats associated protein (known as dCas or CRISPRi). The Cas molecules described herein do not have nuclease activity and therefore do not edit the genome. Non-limiting examples of dCas are provided in Table 4. In some cases, the dCas has a sequence at least about 85%, 86%, 87%, 88%, 89%, 90%, 91%, 92%, 93%, 94%, 95%, 96%, 97%, 98%, or 99% identical to a sequence of Table 4.

In some cases, the dCas comprises a mutated Cas protein. In some cases, the dCas is a truncated Cas protein. In some cases, dCas truncations are utilized to repress or activate genes. These truncations include but are not limited to, REC2 domain deletion, REC3 domain deletion, HNH deletion, and deletions of the domains of the nuclease (NUC) lobe, or any combination of the aforementioned domains.

In some cases, the dCas comprises dCas9. In some cases, the dCas9 is a truncated or mutated Cas9 protein. In some cases, the dCas comprises dCas12. In some cases, the dCas 12 is a truncated or mutated Cas 12 protein.

In some cases, the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteurianus, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9.

### Zinc Finger Proteins

Zinc finger proteins (ZFP) comprise a DNA-binding domain made up of Cys2His2 zinc fingers. ZFPs constitute the largest individual family of transcriptional modulators encoded by the genomes of higher organisms.

In some embodiments, the nucleic acid compositions comprise a sequence encoding a ZFP. The ZFP may comprise a native or modified sequence.

Non-limiting examples of ZFP include the molecules of Table 8.

### Additional Nucleic Acid Binding Domains

Further non-limiting examples of nucleic acid binding domains include meganuclease and transcription activator-like effector nucleases (TALENs).

### Guide RNA

For compositions and methods described herein using a CRISPR/Cas system, the nucleic acid comprises and/or is administered with one or more guide RNA that binds to one or more target molecules. In some cases, the one or more guide RNA is 2, 3, 4, or 5 guide RNA sequences.

Non-limiting example guide RNA are provided in Table 6. In some cases, the guide RNA comprises a sequence at least about 90%, 91%, 92%, 93%, 94%, 95%, 96%, 97%, 98%, or 99% identical to a sequence of Table 6.

In some embodiments, a guide RNA sequence may target the promoter used to drive the expression of the final construct, creating a regulatory feedback loop to control the expression levels of the therapeutic.

### Epigenetic Modulators

Provided herein are epigenetic modulators that modulate expression of a target molecule. In certain embodiments, the modulator activates expression. In certain embodiments, the modulator represses expression. The epigenetic modulator may be a transcription regulatory domain that has transcription repression activity (repressor domain) and/or transcription activation activity (activator domain). In some cases, the repressor domain comprises ZIM3. In some cases, the repressor domain comprises a Krueppel-associated box (KRAB) domain (recruitment of histone methyltransferases and deacetylases). Non-limiting examples of repressor domains are provided in Table 9 (e.g., ZIM3). Therefore, provided herein are compositions comprising or encoding a repressor domain of Table 9, or a sequence at least about 85%, 86%, 87%, 88%, 89%, 90%, 91%, 92%, 93%, 94%, 95%, 96%, 97%, 98%, or 99% identical to a sequence of Table 9. The repressor domain may be a variant or combination of repressor domains of Table 9. In some embodiments, the repressor domain comprises a sequence at least about 85%, 86%, 87%, 88%, 89%, 90%, 91%, 92%, 93%, 94%, 95%, 96%, 97%, 98%, or 99% identical to ZIM3, e.g., as shown in Table 9.

In certain embodiments, the epigenetic modulator comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb1 self-association domain (recruits enhancer-associated endogenous Ldb1), SAM activator (VP64, p65, HSF1) (recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2) (histone methyltransferase), DNMT3a (DNA methyltransferase), or DNMT3α-DNMT3L (DNA methyltransferase), p16, p300, CD, SunTag, or a variant or combination thereof.

In certain embodiments, the epigenetic modulator comprises KRAB (also referred to as KOX), SID, MBD2, MBD3, HPla, DNMT family (including DNMT1, DNMT3A, DNMT3B, DNMT3L, DNMT2A), Sin3a, Rb, MeCP2 (methyl-CpG binding protein 2), ROM2, AtHD2A, LSD1, SUV39H1, or G9a (EHMT2), or a variant or combination thereof. Variants of KRAB domains include ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, or ZNF257, or a variant or combination thereof.

In certain embodiments, the epigenetic modulator comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof.

### Simultaneous Activation and Repression

In some embodiments, two or more genes are activated, repressed, and/or one gene is activated and another gene is repressed. A non-limiting example of a system for simultaneous activation and repression of two genes is shown in the schematic of FIG. 2. In this figure, the system comprises a first sequence comprising the amino-terminus of dCas9 (1-573), guide RNA for SON9A, and a KRAB repressor domain; and second sequence comprising the carboxy-terminus of dCas9 (574-1398), guide RNA for PenK, and a VP64 activation domain. This system may be utilized to simultaneously repress SON9A and activate PenK. The simultaneous activation and repression is via gRNA-M2M recruiting MCP-VP64 and gRNA-Com recruiting Com-KRAB. This system may be utilized with other activation and/or repressor domains, and gRNA for simultaneous targeting of different target molecules. Although certain example components are shown, the figure should not be construed as limiting. For instance, although a CMV promoter is shown, it is understood that other promoters, e.g., a Nav1.7 promoter, may be used instead.

### Linkers

In certain embodiments, the epigenetic modulator is linked to the nucleic acid binding domain. The epigenetic modulator may be positioned at the N- or C-terminus of the nucleic acid binding domain. The domains may be linked via a peptide linker. The domains may be linked via a disulfide bond.

In some embodiments, the epigenetic modulator is linked to a nucleic acid binding domain via a peptide linker. Non-limiting examples of peptide linkers include (GGS)n (SEQ ID NO: 156), (GGGS)n (SEQ ID NO: 157), (GGGGS)n (SEQ ID NO: 158), (G)n (SEQ ID NO: 159), (EAAAK)n (SEQ ID NO: 160), A (EAAAK)nALEA(EAAAK)nA (SEQ ID NO: 161), PAPAP (SEQ ID NO: 162), AEAAAKEAAAKA (SEQ ID NO: 163), (Ala-Pro)x (SEQ ID NO: 164), LE, GlySer-polyPro (Glyc)-polyPro (Glyc)-polyPro (Glyc), GlySer-polyPro-polyPro (Glyc)-polyPro, GlySer-polyPro-GlySer (Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-β2m-GlySer-β2m-polyPro, GlySer-polyPro-Ub-GlySer, GlySer-polyPro-ZAG-polyPro, GlySer-GlySer-ZAG-GlySer-ZAG-polyPro, Gly Ser (Glyc)-Gly Ser (Glyc)-polyPro, (G4S) 3-cTPR3-(G4S) 3, (G4S) 3-cTPR6-(G4S) 3, (G4S) 3-cTPR9-(G4S) 3, (G4S) 3-cTPR12-(G4S) 3, and (G4S)n (SEQ ID NO: 158); wherein n is independently selected from 1 to 10, x is 10-34, polyPro is proline-rich hinge sequence from IgA1, polyPro (Glyc) is proline-rich hinge sequence from IgA1 with an embedded potential N-linked glycosylation site (Asn-Ser-Ser), B2m is β2-microglobulin, Ub is ubiquitin, ZAG is Zn-α2-glycoprotein, and cTPRX is consensus tetratricopeptide repeat sequence with X number of repeats.

### Promoters

Further provided herein are promoters that drive expression of a nucleic acid binding domain and/or epigenetic modulator herein. In some embodiments, the nucleic acid composition comprises one or more promoters. As an example, the nucleic acid composition comprises tandem promoters.

In some embodiments, the promoter is specific to a target molecule so that the nucleic acid composition is specific for, or only expressed in, those cells expressing the target for increased therapeutic selectivity. As a non-limiting example, the target molecule is SON9A.

In some embodiments, the promoter further increases the specificity of the AAV tropism for cells that express a target molecule. As a non-limiting example, the target molecule is SCN9A. Downregulating or upregulating a target molecule only in target molecule-expressing cells may reduce off-target effects and general toxicity. This is important to prevent the expression of the effectors (e.g., dCas or ZFP) in immune cells such as glial cells, microglia, macrophages, astrocytes, etcetera, that can mediate an immune reaction against the gene therapy proposed herein.

In some embodiments, the promoter is specific to SON9A (Nav1.7) and is utilized to drive the repression of Nav1.7 or other gene products. In some embodiments, the promoter is specific to any one of the genes in Tables 1-3.

In some embodiments, some promoters can be induced with small molecules or other means. These inducible expression promoters include tetracycline responsive promoter, a glucocorticoid responsive promoter, an RU-486 responsive promoter, a peroxide inducible promoter and tamoxifen induced promoter.

Furthermore, there are promoters that can be induced when a pathology arises, such as injury or inflammation. Injury induced promoters include the galanin promoter specific to nociceptive afferent neurons. The inflammation-inducible promoter NF-κB could also be used for pain associated with inflammation.

Tandem promoters and combinations of promoters can also be used to prevent immune responses and create more cell-type specific expression.

In some embodiments, expression of the epigenetic modulator and/or transcription regulatory domain occurs upon a natural or physiological induction of the promoter.

In some embodiments, pan-neuronal gene promoters are used for modulation of gene expression in neurological diseases and for repression of pain-related genes. Non-limiting example promoters include the promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, the promoter of the NeuN gene (Fox-3, Rbfox3, or Hexaribonucleotide Binding Protein-3), the promoter of the α-calcium/calmodulin-dependent protein kinase II [CaMKIIα]), the promoter of the Rheb gene (ras homolog enriched in brain), TRKA promoter (Tyrosine Kinase A). In some embodiments, the promoter is neuronal specific, such as pol II promoters, including Thy1 and H1xb9. Small latency-associated promoters from the herpesvirus pseudorabies virus can also be used for pan-neuronal expression of the effectors (dCas9 or ZFP) fused to a repressor domain.

Additional promoters include cytomegalovirus (CMV), SV40, elongation factor 1-alpha (EF1a) promoter, cytomegalovirus enhancer/chicken β-actin (CAG) promoter, jET promoter and herpes simplex virus (HSV).

In some embodiments, the promoter comprises a sequence at least about 90%, 91%, 92%, 93%, 94%, 95%, 96%, 97%, 98%, 99% or 100% identical to sequence of Table 5.

In some embodiments, the promoter is a SON9A promoter or SON10A promoter.

In some embodiments, the promoter is naturally associated with a gene associated with a channelopathy, Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), pain (e.g., inflammatory pain, visceral pain, migraine pain, erythromelalgia pain, fibromyalgia pain, idiopathic pain, somatic pain), a neurological disease, dementia, Alzheimer's disease, Parkinson's disease, ALS, Multiple Sclerosis, a central nervous system ailment, or a combination thereof.

In some embodiments, the promoter is a promoter of a gene selected from SNCA, GBA, LRRK2, SOD1, ataxin-2, SCA2, BED1, FUS, C9orf72, Brain-derived neurotrophic factor, Nerve growth factor, Neurotrophin, BCL11A, FMR1, DNM2, PrP, UBE3A, GYS1, and GFAP.

In some embodiments, the promoter is a pol II promoter (e.g., Thy1 and H1xb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter.

### Nucleic Acid Delivery

In some embodiments, a nucleic acid composition herein is delivered as a naked or unmodified nucleic acid. In other embodiments, the nucleic acid composition is delivered complexed with cationic molecules.

In some embodiments, the nucleic acid is delivered to the subject via a vehicle. The vehicle may be a liposome, lipid nanoparticle, nanocapsule, or exosome.

In some embodiments, the nucleic acid is delivered via a viral vehicle. Non-limiting viral vehicles include, but are not limited to, retroviral vectors, lentiviral vectors, adenoviruses vectors, adeno-associated viral vectors (e.g., AAV1, AAV2, AAV3, AAV4, AAV5, AAV6, AAV7, AAV8, AAV9, AAV10, AAVhu68, AAVrh. 10, AAVrh74, AAVDJ), and the like. In some cases, the vehicle is a recombinant adeno-associated virus (AAV). In some cases, the AAV is AAV9. AAV9 may be selected because it has the highest tropism for dorsal root ganglia (DRG) neurons where pain associated proteins such as Nav1.7 are highly expressed. Further, AAV9 has been shown to be safe.

All delivery vehicles (viral vectors or non-viral) can have an improved tropism towards cells that express the protein product of a target molecule. For example, the vehicles can comprise a peptide that binds to the protein product of a target molecule. As a non-limiting example, the peptide binds to Nav1.7 to target the nucleic acid to Nav1.7 expressing cells.

### Targeting Moieties

In some embodiments, a nucleic acid composition herein is delivered via a viral vehicle, e.g., an AAV capsid described herein, with a nucleic acid sequence encoding a peptide targeting moiety.

In some embodiments, a nucleic acid composition herein is delivered via a non-viral delivery vehicle, such as, without limitation, a liposome, lipid nanoparticle, nanocapsule, or exosome. For some such embodiments, the vehicle may comprise and/or be connected to a targeting moiety, such as a small molecule or peptide targeting moiety.

In some embodiments, the targeting moiety comprises a peptide targeting moiety that binds to protein product of the target molecule in order to target the nucleic acid to a specific cell. In some cases, the target molecule is present on a target cell. In some cases, the target cell is associated with the disease or condition in the subject.

Non-limiting examples of peptide targeting moieties for use with viral and non-viral delivery methods include JNJ63955, m3-Huwentoxin-IV, Phlotoxin 1 (PhITx1), Protoxin-II (ProTx-II), Ceratotoxin-1 (CcoTx1), Huwentoxin-IV (HwTx-IV), μ-TRTX-Pn3a, Jz-Tx-V, GsAFI, Tp1a (Protoxin-III), GpTx-1, HpTx1, Hm1a, and variants and combinations thereof. The peptide may originate from one or more of the following organisms: Thrixopelma prurient, Pamphobeteus nigricolor, Chilobrachys jingzhao, Grammostola spatulata, Phlogiellus genus, Thrixopelma pruriens, Grammostola porteri, Selenocosmia huwena, Ceratogyrus cornuatus, Heteropoda venatoria, and or Heteroscodra maculate.

In some cases, the peptide comprises a sequence at least about 90%, 91%, 92%, 93%, 94%, 95%, 96%, 97%, 98%, 99% or 100% identical to a peptide of Table 7. The peptides of Table 7 bind to Nav1.7 and thus may be useful to provide AAV tropism to Nav 1.7 expressing cells. In some cases, the peptide comprises ProTx-II, ProTx-III, the ProTx-II variant JNJ63955, HwTx-IV variant m3-HwTx-IV, CcoTx1 variant 2670, PhlTx1 variant D7A-PhITx1, or JxTx-V variant AM-0422.

### AAV Vectors

Recombinant adeno-associated viruses (AAVs) are among the most commonly used vehicles for in vivo gene delivery. To enter a host cell, the virus 1) binds a receptor and glycan co-receptor on the cell surface, 2) is endocytosed, 3) progresses through the endosomal compartment, 4) escapes the endosome, and 5) traffics to the nucleus. Once inside the nucleus, the virus sheds its coat and its single-stranded genome is converted to a double-stranded one which the host cell can now use as a template for gene expression. The AAV receptor (AAVR, KIAA0319L) has been reported to be essential for AAV entry into cells, however, some recombinant AAVs can enter cells independent of AAVR. Glycosylation is also known to play a role in viral transduction efficiency. Changing the capsid composition of an AAV changes the ability of that AAV to enter cells. There are at least four major techniques currently used towards modifying and improving AAV tropism: rational engineering, directed evolution, evolutionary lineage analysis, and chemical conjugation.

One way to go about modifying a virus's tropism is to generate a large library of peptides to add onto the AAV surface and then characterize the resultant variants to determine their tropism. This method is labor intensive, requiring massive screening efforts as the library of peptides screened are more or less generated randomly so success is based on a numbers game. In a similar technique referred to as directed viral evolution, organs are collected from the first round of viral infection and screened to select viral variants with desired tropism (ex: brain-specific) for subsequent rounds of infection to further select even more specific tropism. However, some of these screening methods are not able to be translated between species.

Instead of using a random library screening, described herein is a rational design method to improve AAV tropism. Rational design strategies for AAV capsid engineering include peptide domain insertions and chemical biology approaches. One can add peptides which are known to specifically interact with cells of interest. By using binding peptides that bind to the protein encoding a target molecule, AAVs described herein have increased tropism towards target molecule expressing cells, generating more targeted strategies.

In some embodiments, the peptide to increase tropism binds to Nav1.7. Peptides to increase Nav 1.7 tropism include: JNJ63955, m3-Huwentoxin-IV, Phlotoxin 1 (PhITx1), Protoxin-II (ProTx-II), Ceratotoxin-1 (CcoTx1), Huwentoxin-IV (HwTx-IV), and those described elsewhere herein, e.g., a peptide of Table 7 or a sequence at least about 90%, 91%, 92%, 93%, 94%, 95%, 96%, 97%, 98%, 99% or 100% identical to a peptide of Table 7.

### Target Molecules

In certain embodiments compositions described herein modulate expression of one or more target molecules associated with a disease or condition in a subject. In some cases, expression of the target molecule is activated. In some cases, expression of the target molecule is repressed.

The one or more target molecules may comprise DNA or RNA. In some embodiments, a target molecule comprises DNA. In some embodiments, a target molecule comprises a coding region of a gene. In some embodiments, a target molecule comprises DNA complementary to non-coding RNA. The non-coding RNA may be associated with a disease or condition described herein (e.g., pain). For example, the non-coding RNA is associated with neuropathic pain such as spinal nerve ligation, spared nerve injury, chronic constriction injury, or diabetic neuropathy, or a combination thereof.

In some embodiments, the non-coding RNA comprises a SON9A natural antisense transcript (NAT), a Kcna2 antisense RNA, H19, Gm21781, MRAK009713, uc.48+, NONRATT021972, BC168687, Speer7-ps, Uc007pbc.1, XLOC_041439, Mlxipl, Rn50_X_0739.1, CCATI, rno circ 0004058, rno_circRNA_007512, or Egr2 antisense RNA, or a combination thereof.

In some embodiments, the one or more target molecules comprises a nucleic acid associated with a disease or condition described here. Non-limiting examples of nucleic acids associated with diseases and conditions described herein are shown in Tables 1-3. In some embodiments, the one or more target molecules comprises a nucleic acid associated with pain. Pain includes neuropathic pain, inflammatory pain, visceral pain, migraine pain, erythromelalgia pain, fibromyalgia pain, idiopathic pain, and somatic pain.

In some embodiments, the one or more target molecules comprises a nucleic acid associated with a channelopathy. In some cases, the one or more target molecules comprises a nucleic acid encoding a channel. The channel may be an ion channel, e.g., sodium channel, potassium channel, calcium channel, and/or chloride channel.

In some embodiments, the one or more target molecules comprises a nucleic acid associated with a neurological disease. In some embodiments, the one or more target molecules comprises a nucleic acid associated with dementia. In some embodiments, the one or more target molecules comprises a nucleic acid associated with Alzheimer's disease. In some embodiments, the one or more target molecules comprises a nucleic acid associated with Parkinson's disease. In some embodiments, the one or more target molecules comprises a nucleic acid associated with Huntington's disease. In some embodiments, the one or more target molecules comprises a nucleic acid associated with schizophrenia. In some embodiments, the one or more target molecules comprises a nucleic acid associated with Amyotrophic lateral sclerosis (ALS). In some embodiments, the one or more target molecules comprises a nucleic acid associated with Multiple Sclerosis. In some embodiments, the one or more target molecules comprises a nucleic acid associated with a central nervous system ailment. In some embodiments, the one or more target molecules comprises a nucleic acid associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

In some embodiments, the one or more target molecules comprises one or more genes of Tables 1-3. In some embodiments, the one or more target molecules comprises SON9A, SON10A, or SON11A, or a combination thereof. In some embodiments, the one or more target molecules comprises SNCA, GBA, or LRRK2, or a combination thereof. In some embodiments, the one or more target molecules comprises GPR52. In some embodiments, the one or more target molecules comprises SOD1, ataxin-2, or SCA2, or a combination thereof. In some embodiments, the one or more target molecules comprises one or more genes selected from the group comprising BED1, FUS, (9orf72, Brain-derived neurotrophic factor, Nerve growth factor, a Neurotrophin, BCL11A, FMR1, DNM2, PrP, UBE3A, GYS1, and GFAP.

### Genes Involved in Pain

The human genome encodes genes that can confer protection to unnecessary pain. Genetic studies have correlated a hereditary loss-of-function mutation in a human-voltage gated sodium channel—Nav1.7 (SON9A)—with a rare genetic disorder, which leads to insensitivity to pain without other neurodevelopmental alterations. Thus, this sodium channel has been an attractive target for developing chronic pain therapies. However, efforts to develop selective small molecule inhibitors have been hampered due to the high sequence identity between Nav subtypes, and in fact, many small-molecule drugs targeting Nav1.7 have failed due to lack of specificity. Antibodies have faced a similar situation since there is a tradeoff between selectivity and potency due to the antibody binding to a specific (open or close) conformation of the channel. Indeed, even commercially available antibodies targeting the human channel are poor for a western blot. Interference RNA (RNAi) has also been utilized to target Nav1.7. As an exogenous system, however, RNAi competes with endogenous machinery such as microRNA or RISC complex function. Thus, RNAi can compete with and impair fundamental homeostatic mechanisms of RNA synthesis and degradation. In addition, due to high RNA turnover, RNAi methods have poorer pharmacokinetics prospects and require higher dosage. It is mainly due to these drawbacks that none of the Nav 1.7-targeting treatments based on these methods have yet succeed to reach the final phase of clinical trials. In contrast, disclosed here is the use of nucleic acid binding domains (e.g., nuclease-inactivated “dead” CRISPR-Cas (dCas)—also known as CRISPR interference or CRISPRi- and Zinc-Finger proteins) and epigenetic modulators (e.g., KRAB repressor) to repress the transcription of SON9A and other genes associated with pain. As permanent ablation of pain is not desired, there is no permanent genome editing using such methods. Instead, these epigenomic engineering methods enable transient modulation of Nav1.7 gene expression. Additionally, this approach may have lower risk of off-target effects than other approaches. Rather than pharmacologically targeting the protein or RNA, this approach targets Nav1.7 at the DNA level. This may result in longer lasting results than methods targeting protein or RNA. With this approach, one can engineer highly specific, long-lasting, and reversible treatments for pain. Treatment duration is important because many pain states resulting from chronic inflammation and nerve injury are enduring conditions which typically require continual re-medication. This genetic approach provides ongoing, controllable regulation of the aberrant pain processing. Further, since the disclosed approach can be easily designed to target several genes, it represents a new paradigm in pain management since it provides a synergistic way of targeting single or multiple sodium channels for more potent pain relief.

In addition to SON9A, Table 2 provides other genes involved in pain. For genes that are upregulated, the methods herein may repress expression; and for genes that are downregulated, the methods herein may activate expression. In some cases, the methods repress and activate expression of one or more genes.

### Genes Involved in Channelopathies

In certain embodiments, the target molecule comprises a gene involved in a channelopathy. The genes may be associated with or encode a channel such as a sodium channel, potassium channel, calcium channel, and/or chloride channel. Non-limiting examples of such genes are provided in Table 1.

### Genes Involved in Neurological Diseases or Conditions

In certain embodiments, the target molecule comprises a gene involved in a neurological disease or condition of the central nervous system. Non-limiting example diseases and conditions include Alzheimer's disease, Parkinson disease, Huntington's disease, Amyotrophic lateral sclerosis (ALS) and Multiple Sclerosis.

Alzheimer's disease (AD) is a progressive disease that causes brain cells to waste away (degenerate) and die. Alzheimer's disease is the most common cause of dementia—a continuous decline in thinking, behavioral and social skills that disrupts a person's ability to function independently. Activation and/or repression of genes could potentially slow the progression or prevent Alzheimer's disease.

Research has shown that the binding of B-Amyloids to Li1rB2 (e.g., UniProt Ref No. Q8N423) is one of the first steps leading to Alzheimer's disease. Thus, repression of Li1rB2 such that it affects the B-Amyloid binding could prevent the onset of Alzheimer's disease. D1 is associated with Uniprot Ref No. P21728. D2 is associated with Uniprot Ref No. 14416. Therefore, provided herein are compositions and methods of repressing expression of Li1rB2.

MS4A4A and TREM2 operate in the microglia, the brain's immune cells. They influence Alzheimer's disease risk by altering levels of TREM2, a protein that helps microglia cells clear excessive amounts of the Alzheimer's proteins amyloid and tau from the brain. Thus, activation of TREM2 in the cerebrospinal fluid (CSF) could help prevent and/or slow the progression of Alzheimer's disease. Therefore, provided herein are compositions and methods of activating expression of TREM2.

Apolipoprotein E4 (apoE4), the most prevalent genetic risk factor of Alzheimer's disease, is expressed in more than half of Alzheimer's disease patients and is thus an important possible therapeutic target. Of the three polymorphic forms of apoE, namely apoE2, apoE3, and apoE4, carriers of apoE4 are more likely to develop Alzheimer's disease. Blocking the apoE4 effect would help the 40-60% of AD patients who carry apoE4, whereas, if all apoE forms are in fact toxic, another approach would be to block all apoE action. In addition, several studies have revealed that apoE4 is also a risk factor for other diseases including cerebral amyloid angiopathy, dementia with Lewy bodies, tauopathy, cerebrovascular disease, multiple sclerosis, and vascular dementia. Accordingly, provided herein are compositions and methods for repressing APOE4 expression.

VEGF (vascular endothelial growth factor), originally described as a key angiogenic factor, has been shown to play an important role in neurogenesis and neuroprotection and to affect neuronal plasticity and repair. Animal model studies revealed that brain levels of VEGF and its receptor (VEGFR-2) were reduced in the hippocampus of apoE4 mice. Thus, upregulation of the levels of hippocampal VEGF may reverse the accumulation of Aβ and hyperphosphorylated tau in hippocampal neurons. Thus, provided herein are compositions and methods for upregulating VEGF expression.

Similarly, ABCA1 (ATP-binding cassette transporter ABCA1) upregulation reverses the apoE4-driven cognitive and brain pathology, and therefore ABCA1 is a target molecule for upregulation and Alzheimer's disease treatment.

Transactive response DNA-binding protein 43 (TDP-43), an RNA-binding protein that functions in axon skipping, has recently been shown to be deposited in AD brain. TDP-43 is present in the brain of 65-80% of AD patients and was shown to be associated with progressive hippocampal atrophy. Therefore, in some embodiments, a target molecule is TDP-43, and compositions described herein repress TDP-43 expression.

Target molecules also include genes having known mutations that lead to early-onset Alzheimer's disease, such as presenilin 1 (PSEN1) and presenilin 2 (PSEN2). Thus, activation of these genes could be another potential avenue for treatment of Alzheimer's disease.

Further non-limiting target molecules involved in neurological diseases such as Alzheimer's disease are shown in Table 3. Such genes may be repressed or upregulated using the compositions and methods described herein.

Additional target molecules include alpha-synuclein, microtubule-associated protein tau, APP, and huntingtin (SNCA, MAPT, APP, and HTT, respectively). Precise transcriptional modulation has been performed through CRISPRa of neurodegenerative disease-related genes in human iPSC-derived neurons. TSS2-2 sgRNA and dCas9-VPR transcriptional activator mediated the activation of alpha-synuclein in normal alpha-synuclein levels (NAS) iPSC-derived neurons from healthy control patient and iPSCs derived from a patient with Parkinson's disease caused by alpha-synuclein triplication (AST). An eightfold activation of endogenous SNCA expression was achieved in the NAS iPSC-derived neurons and through dCas9-KRAB repression, a 40% reduction in alpha-synuclein mRNA levels in the AST iPSC-derived neurons. In addition, targeting the genes close to the TSS region reduced the off-target effects considerably, reducing the negative side effects of using SpCas9. Overall, these findings suggest the possibility of exploiting the tunable CRISPRa/CRISPRi platform for multiplex transcriptional repression of molecular pathological signatures in vivo in the mammalian brain and the possibility for addressing neurodegeneration in the familial and sporadic disease states.

In some embodiments, the target molecule comprises one or more of alpha synuclein (SNCA), glucocerebrosidase (GBA), and/or leucine-rich repeat kinase (LRRK2). In some cases, SNCA degradation is prevented by inhibiting tyrosine kinase c-ABL. In some cases, expression of SNCA is repressed. The target molecule may be repressed for treatment of Parkinson's or another condition of the nervous system.

In some embodiments, the target molecule comprises HTT encoding Huntington's protein. Repression of mutated HTT may be performed to treat Huntington's disease.

In some embodiments, the target molecule comprises GPR52. Modulation of GPR52 may be performed to treat Huntington's disease and/or schizophrenia. GPR52 upregulation may be used for schizophrenia, cognitive impairment, psychiatric disorders, brain malformation and hyperactivity. Repression of GPR52 may be used for the treatment of Huntington's disease, as GPR52 is associated with the abnormal expression of huntingtin that is observed in patients with this disorder.

In some embodiments, the target molecule comprises at least one of superoxide dismutase 1 (SOD1), ataxin-2, and/or transactive response DNA-binding protein 43 (TDP-43). Modulation of the target molecules may be performed to treat ALS, such as repression of SOD1, ataxin-2 or TDP43. Modulation of the target molecules may be performed to treat frontotemporal dementia (FTD) or other neurological diseases.

In some embodiments, the target molecule comprises BFD1, encoding bradyzoite-formation deficient 1, a transcription-factor protein. BFD1 can drive the expression of genes needed for the formation of bradyzoites of Toxoplasma Gondii, and therefore its repression may prevent chronification of this infection and other infections using a similar route of chronification.

A mutation in the C9orf72 gene is the most common genetic cause of two neurodegenerative diseases. Studies of mice and humans suggest a role for loss of the C9orf72 protein in some neurodegenerative disorders as with reduced C9orf72 levels, there is more inflammation mediated by the STING protein in immune and brain cells. Therefore, in some embodiments, the target molecule comprises C9orf72, and compositions herein upregulate C9orf72 expression. In some embodiments, the target molecule comprises the STING gene.

Additional genes that could be upregulated to treat neurological diseases include neurotrophic factors such as Brain-derived neurotrophic factor, Nerve growth factor and Neurotrophins. The DD (′ gene encoding AADC can be upregulated to treat Parkinson's disease.

Genes that could be downregulated to treat the following diseases include: B-thalassemia (BOL11A), Fragile X (FMR1), centronuclear myopathy (DNM2), Prion disease (PrP), Angelman Syndrome (UBE3A), Lafora disease (GYS1), and Alexander disease (GFAP).

### Methods of Treatment

Various embodiments provide for methods of treating a disease or condition in a subject with the compositions described herein. In some embodiments, the composition comprises a nucleic acid described herein encoding a nucleic acid binding domain and an epigenetic modulator. The composition may be delivered via AAV or non-viral vehicles. Non-limiting example compositions include those generally shown in FIGS. 1 and 2.

In example embodiments, the disease or condition comprises pain. Pain includes neuropathic, inflammatory, visceral, migraine, erythromelalgia, fibromyalgia, idiopathic and somatic pain. Inflammatory pain comprises rheumatoid arthritis pain. The disease or condition also includes those where Nav1.7 or other genes involved in pain could be targeted.

In some embodiments, the disease or condition comprises a channelopathy. Channelopathies include Dravet syndrome, Epilepsy syndromes, Familial hemiplegic migraine, Ohtahara syndrome, West syndrome, Lennox-Gastaut syndrome, sodium channel myotonia, autism, Long QT syndrome, Brugada syndrome, Progressive cardiac conduction disease (also called Lenègre disease), and the like. Example diseases and conditions are shown in Table 1.

In some embodiments, the disease or condition comprises a neurological disease or condition. Neurological diseases or conditions include dementia, Alzheimer's disease, Parkinson disease, Huntington's disease, schizophrenia, Amyotrophic lateral sclerosis (ALS) and Multiple Sclerosis. The disease or condition may comprise a central nervous system ailment.

In some embodiments, the disease or condition comprises an inflammatory disease or condition. As a non-limiting example, the inflammatory disease or condition is rheumatoid arthritis.

In some embodiments, the disease or condition comprises an infection.

In some embodiments, the disease or condition comprises Beta-thalassemia, Fragile X, centronuclear myopathy, Prion disease, Angelman Syndrome, Lafora disease, or Alexander disease, or a combination thereof.

In some embodiments, a method of treatment comprises administering a nucleic acid composition described herein and one or more additional active agents. For instance, the additional active agent may be used to complementarily treat the disease or condition.

In some embodiments, a subject refers to any animal, including, but not limited to, humans, non-human primates, rodents, and domestic and game animals. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a human.

In various embodiments, a subject can be one who has been previously diagnosed with or identified as suffering from or having a disease or condition in need of treatment. In various embodiments, the subject previously diagnosed with or identified as suffering from or having the disease or condition may or may not have undergone treatment for a condition. In other embodiments, a subject can also be one who has not been previously diagnosed as having a disease or condition and the therapeutic is used for prevention (prophylactically).

A “subject in need” of treatment for a particular condition can be a subject having that condition, diagnosed as having that condition, or at risk of developing that condition. In some embodiments, the subject is a “patient,” that has been diagnosed with a disease or condition described herein. In some embodiments, the subject is military personnel to prevent or mitigate pain and/or torture.

In some embodiments, the term “therapeutically effective amount” refers to an amount of a composition (e.g., nucleic acid) effective to “treat” a disease or disorder in a subject or mammal. In some cases, a therapeutically effective amount of the composition reduces the severity of symptoms of the disease or condition. In some cases, a therapeutically effective amount of the composition prevents the development of a disease or condition.

In some embodiments, the terms, “treat” or “treating” as used herein refer to both therapeutic treatment and prophylactic or preventative measures (e.g., disease progression), wherein the object is to prevent or slow down (lessen) the targeted pathologic condition. In some aspects provided herein, subjects in need of treatment include those already with a disease or condition, as well as those susceptible to develop the disease or condition.

### Pharmaceutical Compositions, Administration and Dosage

In various embodiments, the compositions herein are formulated for delivery via any route of administration. “Route of administration” may refer to any administration pathway known in the art, including but not limited to intrathecal, epidural, intravenous, transdermal, intranasal, oral, mucosal, or other delivery methods, and/or via single or multiple doses. Example routes of administration for the nucleic acids described herein include lumbar intrathecal puncture, intracisterna magna administration, and intraganglionic administration.

In an example embodiment, the composition is delivered into the spinal intrathecal space using any appropriate delivery method. This approach may be particularly useful when targeting Nav 1.7 because the role played by Nav1.7 is in the nociceptive afferents, and their cell bodies are in the respective segmental dorsal root ganglion (DRG) neurons. Therefore, delivery to the spinal intrathecal space may efficiently deliver compositions targeting Nav1.7 to the DRG neurons, which can minimize the possibility of off target biodistribution and reduce viral load required for transduction.

It is appreciated that actual dosage can vary depending on the route of administration, the delivery system used (e.g., AAV or liposome, etc), the target cell, organ, or tissue, the subject, as well as the degree of effect sought. Size and weight of the tissue, organ, and/or patient can also affect dosing. Doses may further include additional agents, including but not limited to a carrier. Non-limiting examples of suitable carriers are known in the art: for example, water, saline, ethanol, glycerol, lactose, sucrose, dextran, agar, pectin, plant-derived oils, phosphate-buffered saline, and/or diluents.

The pharmaceutical compositions can also contain any pharmaceutically acceptable carrier. “Pharmaceutically acceptable carrier” refers to a pharmaceutically acceptable material, composition, or vehicle that is involved in carrying or transporting a composition from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, provided are pharmaceutical compositions including a pharmaceutically acceptable excipient along with a therapeutically effective amount of a nucleic acid described herein. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain auxiliary substances such as wetting or emulsifying agents, pH buffering agents and the like which enhance or maintain the effectiveness of the active ingredient, or increase the stability of the pharmaceutical product. In addition, if desired, the composition can contain auxiliary substances to modify the density of the pharmaceutical product. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of nucleic acid (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. When the intrathecal route is recommended, the pharmaceutical product may be diluted ex vivo with the subject cerebrospinal fluid prior to administration to achieve an isobaric solution.

### Kits

Further provided is a kit to perform methods described herein. The kit is an assemblage of components, including at least one of the compositions described herein. Thus, in some embodiments, the kit comprises a nucleic acid encoding a nucleic acid binding domain and an epigenetic modulator. The nucleic acid may be combined with, or complexed to, another component such as a vehicle for delivery, or may be unmodified for direct delivery. In some cases, the nucleic acid is complexed with a cationic molecule. In some cases, the nucleic acid is configured for delivery via a viral delivery vehicle such as an AAV capsid protein. For certain kits where the nucleic acid binding domain comprises a dCas protein, the kit comprises one or more guide RNA sequences.

Instructions for use of the components may be included in the kit. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of a composition containing a nucleic acid herein. The packaging material generally has an external label which indicates the contents and/or purpose of the kit and/or its components.

### Numbered Embodiments

1. A method for modulating expression of one or more target molecules associated with a disease or condition in a subject, the method comprising administering to the subject a nucleic acid sequence encoding a nucleic acid binding domain and an epigenetic modulator that regulates transcription of the one or more target molecules.

2. The method of embodiment 1, wherein regulation of the transcription of the one or more target molecules is transient.

3. The method of embodiment 1, wherein the genome of the subject is not edited.

4. The method of any one of embodiments 1-3, wherein the disease or condition comprises pain.

5. The method of embodiment 4, wherein the pain comprises neuropathic pain, inflammatory pain, visceral pain, migraine pain, erythromelalgia pain, fibromyalgia pain, idiopathic pain, or somatic pain, or a combination thereof.

6. The method of any one of embodiments 1-3, wherein the disease or condition comprises a channelopathy.

7. The method of embodiment 6, wherein the channelopathy comprises Dravet syndrome, Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

8. The method of any one of embodiments 1-3, wherein the disease or condition comprises a neurological disease.

9. The method of embodiment 8, wherein the neurological disease is dementia.

10. The method of any one of embodiments 1-3, wherein the disease or condition comprises Alzheimer's disease.

11. The method of any one of embodiments 1-3, wherein the disease or condition comprises Parkinson's disease.

12. The method of any one of embodiments 1-3, wherein the disease or condition comprises Huntington's disease.

13. The method of any one of embodiments 1-3, wherein the disease or condition comprises schizophrenia.

14. The method of any one of embodiments 1-3, wherein the disease or condition comprises Amyotrophic lateral sclerosis (ALS).

15. The method of any one of embodiments 1-3, wherein the disease or condition comprises Multiple Sclerosis.

16. The method of any one of embodiments 1-3, wherein the disease or condition comprises a central nervous system ailment.

17. The method of any one of embodiments 1-3, wherein the disease or condition comprises an infection.

18. The method of any one of embodiments 1-3, wherein the disease or condition comprises Beta-thalassemia, Fragile X, centronuclear myopathy, Prion disease, Angelman Syndrome, Lafora disease, or Alexander disease.

19. The method of any one of embodiments 1-18, wherein the one or more target molecules comprise DNA.

20. The method of any one of embodiments 1-19, wherein the one or more target molecules comprise RNA.

21. The method of any one of embodiments 1-20, wherein the one or more target molecules comprise a coding region of a gene.

22. The method of any one of embodiments 1-21, wherein the one or more target molecules comprise DNA complementary to non-coding RNA.

23. The method of embodiment 22, wherein the non-coding RNA is associated with neuropathic pain.

24. The method of embodiment 23, wherein the neuropathic pain comprises spinal nerve ligation, spared nerve injury, chronic constriction injury, or diabetic neuropathy, or a combination thereof.

25. The method of any one of embodiments 22-24, wherein the non-coding RNA comprises a SCN9A natural antisense transcript (NAT), a Kcna2 antisense RNA, H19, Gm21781, MRAK009713, uc.48+, NONRATT021972, BC168687, Speer7-ps, Uc007pbc. 1, XLOC_041439, Mlxipl, Rn50_X_0739.1, CCATI, rno circ 0004058, mno_circRNA_007512, or Egr2 antisense RNA, or a combination thereof.

26. The method of any one of embodiments 1-21, wherein the one or more target molecules comprises a nucleic acid encoding a channel.

27. The method of any one of embodiments 1-21, wherein the one or more target molecules comprises a nucleic acid associated with a channel.

28. The method of embodiment 26 or embodiment 27, wherein the channel is an ion channel.

29. The method of embodiment 28, wherein the ion channel is a sodium channel.

30. The method of embodiment 28, wherein the ion channel is a potassium channel.

31. The method of embodiment 28, wherein the ion channel is a calcium channel.

32. The method of embodiment 28, wherein the ion channel is a chloride channel.

33. The method of any one of embodiments 1-32, wherein the one or more target molecules is associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

34. The method of any one of embodiments 1-33, wherein the one or more target molecules comprises one or more genes of Table 1.

35. The method of any one of embodiments 1-34, wherein the one or more target molecules comprises SCN9A, SCN10A, or SCN11A, or a combination thereof.

36. The method of any one of embodiments 1-35, wherein the one or more target molecules comprise a natural antisense transcript for SCN9A.

37. The method of any one of embodiments 1-36, wherein the one or more target molecules is associated with pain.

38. The method of claim 37, wherein the pain comprises neuropathic pain, inflammatory pain, visceral pain, migraine pain, erythromelalgia pain, fibromyalgia pain, idiopathic pain, or somatic pain, or a combination thereof.

39. The method of any one of embodiments 1-38, wherein the one or more target molecules comprises one or more genes of Table 2.

40. The method of any one of embodiments 1-39, wherein the one or more target molecules is associated with a neurological disease.

41. The method of embodiment 40, where in the neurological disease comprises dementia.

42. The method of any one of embodiments 1-41, wherein the one or more target molecules is associated with Alzheimer's disease.

43. The method of any one of embodiments 1-42, wherein the one or more target molecules comprises one or more genes of Table 3.

44. The method of any one of embodiments 1-43, wherein the one or more target molecules is associated with Parkinson's disease.

45. The method of any one of embodiments 1-44, wherein the one or more target molecules comprises one or more genes selected from the group comprising SNCA, GBA, and LRRK2.

46. The method of any one of embodiments 1-45, wherein the one or more target molecules is associated with Huntington's disease.

47. The method of any one of embodiments 1-46, wherein the one or more target molecules is associated with schizophrenia.

48. The method of any one of embodiments 1-47, wherein the one or more target molecules comprises GPR52.

49. The method of any one of embodiments 1-48, wherein the one or more target molecules is associated with Amyotrophic lateral sclerosis (ALS).

50. The method of any one of embodiments 1-49, wherein the one or more target molecule comprises one or more genes selected from the group comprising SOD1, ataxin-2, TDP43, FUS, C9ORF72 and SCA2.

51. The method of any one of embodiments 1-50, wherein the one or more target molecules is associated with Multiple Sclerosis.

52. The method of any one of embodiments 1-51, wherein the one or more target molecules is associated with a central nervous system ailment.

53. The method of any one of embodiments 1-52, wherein the one or more target molecules comprise one or more genes selected from the group comprising BFD1, FUS, C9orf72, Brain-derived neurotrophic factor, Nerve growth factor, a Neurotrophin, BCL11A, FMR1, DNM2, PrP, UBE3A, GYS1, STING, and GFAP.

54. The method of any one of embodiments 1-53, wherein the nucleic acid binding domain binds to at least one of the one or more target molecules.

55. The method of any one of embodiments 1-53, wherein the nucleic acid binding domain comprises a nuclease dead Clustered Regularly Interspaced Short Palindromic Repeats associated protein (dCas).

56. The method of embodiment 55, wherein the dCas comprises a mutated Cas protein.

57. The method of embodiment 55, wherein the dCas is a truncated Cas protein.

58. The method of embodiment 55, wherein the dCas is a mutated or truncated Cas protein of Table 4.

59. The method of embodiment 55, wherein the dCas comprises a sequence at least 90% identical to a dCas of Table 4.

60. The method of embodiment 55, wherein the dCas comprises dCas9.

61. The method of embodiment 60, wherein the dCas9 is a truncated or mutated Cas9 protein.

62. The method of embodiment 55, wherein the dCas comprises dCas12.

63. The method of embodiment 62, wherein the dCas12 is a truncated or mutated Cas12 protein.

64. The method of embodiment 60, wherein the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteuriamis, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9.

65. The method of embodiment 55, wherein the dCas has a REC2 domain deletion.

66. The method of embodiment 55, wherein the dCas has a REC3 domain deletion.

67. The method of embodiment 55, wherein the dCas has a HNH deletion.

68. The method of embodiment 55, wherein the dCas has a nuclease (NUC) lobe deletion.

69. The method of any one of embodiments 1-53, wherein the nucleic acid binding domain comprises a zinc finger protein.

70. The method of any one of embodiments 1-53, wherein the nucleic acid binding domain comprises a meganuclease.

71. The method of any one of embodiments 1-53, wherein the nucleic acid binding domain comprises a transcription activator-like effector nucleases (TALENs).

72. The method of any one of embodiments 1-71, wherein the epigenetic modulator comprises a transcription regulatory domain.

73. The method of embodiment 72, wherein the epigenetic modulator comprises a domain having transcription repression activity (repressor domain).

74. The method of embodiment 73, wherein the repressor domain comprises a Krueppel-associated box (KRAB) domain (recruitment of histone methyltransferases and deacetylases). 75. The method of embodiment 72, wherein the epigenetic modulator comprises a domain having transcription activation activity (activator domain).

76. The method of any one of embodiments 1-75, wherein the epigenetic modulator comprises KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, or G9a (EHMT2), or a variant or combination thereof.

77. The method of any one of embodiments 1-76, wherein the epigenetic modulator comprises ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, or ZNF257, or a variant or combination thereof.

78. The method of any one of embodiments 1-77, wherein the epigenetic modulator comprises VP64, Rta, P16, P65, p300, TET1 catalytic domain, TDG, Ldb1 self-association domain, SAM activator (VP64, p65, HSF1), VPR (VP64, p65, Rta), CD, or SunTag, or a variant or combination thereof.

79. The method of any one of embodiments 1-78, wherein the epigenetic modulator comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof.

80. The method of any one of embodiments 1-79, wherein the epigenetic modulator comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb1 self-association domain (recruits enhancer-associated endogenous Ldb1), SAM activator (VP64, p65, HSF1) (recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2) (histone methyltransferase), DNMT3a (DNA methyltransferase), or DNMT3a-DNMT3L (DNA methyltransferase), or a combination thereof.

81. The method of any one of embodiments 1-80, wherein the epigenetic modulator is linked to the nucleic acid binding domain at the N-terminus or C-terminus of the nucleic acid binding domain via a linker.

82. The method of embodiment 81, wherein the linker is a flexible linker.

83. The method of embodiment 81 or embodiment 82, wherein the linker is (GGS)n (SEQ ID NO: 156), (GGGS)n (SEQ ID NO: 157), (GGGGS)n (SEQ ID NO: 158), (G)n (SEQ ID NO: 159), (EAAAK)n (SEQ ID NO: 160), A (EAAAK)nALEA(EAAAK)nA (SEQ ID NO: 161), PAPAP (SEQ ID NO: 162), AEAAAKEAAAKA (SEQ ID NO: 163), (Ala-Pro)x (SEQ ID NO: 164), LE, GlySer-polyPro (Glyc)-polyPro (Glyc)-polyPro (Glyc), GlySer-polyPro-polyPro (Glyc)-polyPro, GlySer-polyPro-GlySer (Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-β2m-GlySer-β2m-polyPro, GlySer-polyPro-Ub-GlySer, GlySer-polyPro-ZAG-polyPro, GlySer-GlySer-ZAG-GlySer-ZAG-polyPro, GlySer (Glyc)-GlySer (Glyc)-polyPro, (G4S) 3-cTPR3-(G4S) 3, (G4S) 3-cTPR6-(G4S) 3, (G4S) 3-cTPR9-(G4S) 3, (G4S) 3-cTPR12-(G4S) 3, or (G4S)n (SEQ ID NO: 158); wherein n is independently selected from 1 to 10, x is 10-34, polyPro is proline-rich hinge sequence from IgA1, polyPro (Glyc) is proline-rich hinge sequence from IgA1 with an embedded potential N-linked glycosylation site (Asn-Ser-Ser), β2m is β2-microglobulin, Ub is ubiquitin, ZAG is Zn-α2-glycoprotein, and cTPRX is consensus tetratricopeptide repeat sequence with X number of repeats.

84. The method of any one of embodiments 1-80, wherein the nucleic acid binding domain and the epigenetic modulator are connected by a disulfide bond.

85. The method of any one of embodiments 1-84, wherein the nucleic acid sequence comprises a nuclear localization sequence (NLS).

86. The method of any one of embodiments 1-85, wherein the nucleic acid sequence comprises a promoter.

87. The method of embodiment 86, wherein the promoter comprises a sequence at least about 90% identical to sequence of Table 5.

88. The method of embodiment 86 or embodiment 87, wherein the promoter comprises a nuclear localization sequence.

89. The method of embodiment 85 or embodiment 88, wherein the NLS drives nuclear import of the nucleic acid.

90. The method of any one of embodiments 86-89, wherein the promoter drives expression of the nucleic acid binding domain.

91. The method of any one of embodiments 86-90, wherein the promoter drives expression of the epigenetic modulator.

92. The method of any one of embodiments 86-91, wherein the promoter is a promoter naturally associated with the target molecule.

93. The method of any one of embodiments 86-92, wherein the promoter is a promoter of a gene from Tables 1-3.

94. The method of any one of embodiments 86-92, wherein the promoter is a SCN9A promoter or SCN10A promoter.

95.

The method of any one of embodiments 86-92, wherein the promoter is a pan-neuronal gene promoter.

96. The method of any one of embodiments 86-92, wherein the promoter is a promoter naturally associated with a gene related to pain.

97. The method of any one of embodiments 86-92, wherein the promoter is a promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, promoter of the NeuN gene (Fox-3, Rbfox3, or Hexaribonucleotide Binding Protein-3), promoter of the α-calcium/calmodulin-dependent protein kinase II [CaMKIIα]), promoter of the Rheb gene (ras homolog enriched in brain), TRKA promoter (Tyrosine Kinase A), or jET.

98. The method of any one of embodiments 86-92, wherein the promoter is a promoter naturally associated with a gene associated with a channel.

99. The method of any one of embodiments 86-92, wherein the promoter is a promoter naturally associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

100. The method of any one of embodiments 86-92, wherein the promoter is a promoter naturally associated with inflammatory pain, visceral pain, migraine pain, erythromelalgia pain, fibromyalgia pain, idiopathic pain, or somatic pain, or a combination thereof.

101. The method of any one of embodiments 86-92, wherein the promoter is a promoter naturally associated with a neurological disease.

102. The method of any one of embodiments 86-92, wherein the promoter is a promoter naturally associated with dementia.

103. The method of any one of embodiments 86-92, wherein the promoter is a promoter naturally associated with Alzheimer's disease.

104. The method of any one of embodiments 86-92, wherein the promoter is a promoter naturally associated with Parkinson's disease.

105. The method of any one of embodiments 86-92, wherein the promoter is a promoter naturally associated with ALS.

106. The method of any one of embodiments 86-92, wherein the promoter is a promoter naturally associated with Multiple Sclerosis.

107. The method of any one of embodiments 86-92, wherein the promoter is a promoter naturally associated with a gene associated with a central nervous system ailment.

108. The method of any one of embodiments 86-92, wherein the promoter is a promoter of a gene selected from SNCA, GBA, and LRRK2, SOD1, ataxin-2, SCA2, BFD1, FUS, C9orf72, Brain-derived neurotrophic factor, Nerve growth factor, a Neurotrophin, BCL11A, FMR1, DNM2, PrP, UBE3A, GYS1, and GFAP.

109. The method of any one of embodiments 86-92, wherein the promoter is a pol II promoter (e.g., Thy1 and H1xb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter.

110. The method of any one of embodiments 86-92, wherein the promoter is controlled by a small molecule.

111. The method of embodiment 110, wherein the promoter is a tetracycline responsive promoter, glucocorticoid responsive promoter, RU-486 responsive promoter, peroxide inducible promoter or tamoxifen induced promoter.

112. The method of any one of embodiments 86-109, wherein expression of the epigenetic modulator and/or transcription regulatory domain occurs upon a natural or physiological induction of the promoter.

113. The method of embodiment 112, wherein the promoter is induced when a pathology arises in the subject.

114. The method of embodiment 113, wherein the pathology is injury and/or inflammation.

115. The method of embodiment 113 or embodiment 114, wherein the promoter is a galanin promoter or NF-κB promoter.

116. The method of any one of embodiments 1-115, wherein the nucleic acid sequence comprises two or more promoters.

117. The method of any one of embodiments 1-116, wherein the nucleic acid sequence comprises tandem promoters.

118. The method of any one of embodiments 1-117, wherein the nucleic acid sequence comprises an enhancer.

119. The method of any one of embodiments 1-118, wherein the nucleic acid sequence comprises an intron.

120. The method of any one of embodiments 1-119, wherein the nucleic acid comprises an inverted terminal repeat (ITR).

121. The method of any one of embodiments 1-120, wherein the nucleic acid comprises a terminator sequence.

122. The method of any one of embodiments 1-121, comprising administering to the subject one or more guide RNA sequences.

123. The method of embodiment 122, wherein the one or more guide RNA sequences is selected from a sequence in Table 6.

124. The method of embodiment 122 or embodiment 123, wherein the one or more guide RNA sequences binds to one or more of the target molecules.

125. The method of embodiment 124, wherein the one or more target molecules is 2, 3, 4, or 5 target molecules.

126. The method of any one of embodiments 122-125, wherein the one or more guide RNA sequences is 2, 3, 4, or 5 guide RNA sequences.

127. The method of embodiment 126, comprising at least two guide RNA sequences, wherein at least two of the guide RNA sequences are different.

128. The method of any one of embodiments 122-127, wherein at least one or more guide RNA sequences target the promoter of embodiments 86-115, creating a regulatory feedback loop to control expression levels.

129. The method of any one of embodiments 1-128, wherein the nucleic acid is delivered as a naked (or unmodified) nucleic acid.

130. The method of any one of embodiments 1-128, wherein the nucleic acid is delivered complexed with cationic molecules.

131. The method of any one of embodiments 1-128, wherein the nucleic acid is delivered to the subject via a vehicle (e.g., viral delivery vehicle (e.g., retroviral vector, lentiviral vector, or adenoviral vector), liposome, nanoparticle, or exosome).

132. The method of embodiment 131, wherein the vehicle is a viral delivery vehicle.

133. The method of embodiment 132, wherein the viral delivery vehicle is a retroviral vector, lentiviral vector, or adenoviral vector.

134. The method of embodiment 133, wherein the viral delivery vehicle is a recombinant adeno-associated virus (AAV).

135. The method of embodiment 134, wherein the AAV is AAV1, AAV2, AAV3, AAV4, AAV5, AAV6, AAV7, AAV8, AAV9, AAV10, AAVhu68, AAVrh. 10, AAVrh74 or AAVDJ, or a combination thereof.

136. The method of embodiment 135, wherein the AAV is AAV9.

137. The method of any one of embodiments 134-136, wherein the AAV has a recombinant capsid.

138. The method of embodiment 137, wherein the recombinant capsid encodes a targeting moiety.

139. The method of any one of embodiments 1-137, wherein the nucleic acid comprises a sequence encoding a targeting moiety.

140. The method of embodiment 131, wherein the vehicle is a liposome, lipid nanoparticle, nanocapsule, or exosome.

141. The method of embodiment 140, wherein the vehicle comprises or is connected to a targeting moiety.

142. The method of any one of embodiments 138-141, wherein the targeting moiety targets a cell comprising the target molecule in the subject.

143. The method of any one of embodiments 138-142, wherein the targeting moiety binds to a peptide product of the target molecule.

144. The method of embodiment 142, wherein the target molecule is present on a target cell.

145. The method of embodiment 143, wherein the target cell is associated with the disease or condition in the subject.

146. The method of any one of embodiments 138-145, wherein the targeting moiety comprises a peptide.

147. The method of embodiment 146, wherein the peptide comprises JNJ63955, m3-Huwentoxin-IV, Phlotoxin 1 (PhlTx1), Protoxin-II (ProTx-II), or Ceratotoxin-1 (CcoTx1), or a variant thereof.

148. The method of embodiment 146, wherein the peptide comprises a sequence at least about 90% identical to a peptide of Table 7.

149. The method of any one of embodiments 1-147, further comprising administering to the subject an additional therapeutic agent.

150. The method of any one of embodiments 1-149, wherein the nucleic acid is administered via lumbar intrathecal puncture, epidural, intravenous, transdermal, intranasal, oral, mucosal, intracisterna magna administration, or intraganglionic administration.

151. A nucleic acid sequence encoding a nucleic acid binding domain and an epigenetic modulator that regulates transcription of the one or more target molecules.

The nucleic acid sequence of embodiment 151, wherein regulation of the transcription of 152. the one or more target molecules is transient.

153. The nucleic acid sequence of embodiment 151, wherein the genome of the subject is not edited.

154. The nucleic acid sequence of any one of embodiments 151-153, wherein the one or more target molecules comprise DNA.

155. The nucleic acid sequence of any one of embodiments 151-154, wherein the one or more target molecules comprise RNA.

156. The nucleic acid sequence of any one of embodiments 151-155, wherein the one or more target molecules comprise a coding region of a gene.

157. The nucleic acid sequence of any one of embodiments 151-156, wherein the one or more target molecules comprise DNA complementary to non-coding RNA.

158. The nucleic acid sequence of embodiment 157, wherein the non-coding RNA is associated with neuropathic pain.

159. The nucleic acid sequence of embodiment 158, wherein the neuropathic pain comprises spinal nerve ligation, spared nerve injury, chronic constriction injury, or diabetic neuropathy, or a combination thereof.

160. The nucleic acid sequence of any one of embodiments 157-159, wherein the non-coding RNA comprises a SCN9A natural antisense transcript (NAT), a Kcna2 antisense RNA, H19, Gm21781, MRAK009713, uc.48+, NONRATT021972, BC168687, Speer7-ps, Uc007pbc.1, XLOC_041439, Mlxipl, Rn50_X_0739.1, CCATI, mno circ 0004058, rno_circRNA_007512, or Egr2 antisense RNA, or a combination thereof.

161. The nucleic acid sequence of any one of embodiments 151-160, wherein the one or more target molecules comprises a nucleic acid encoding a channel.

162. The nucleic acid sequence of any one of embodiments 151-160, wherein the one or more target molecules comprises a nucleic acid associated with a channel.

163. The nucleic acid sequence of embodiment 161 or embodiment 162, wherein the channel is an ion channel.

164. The nucleic acid sequence of embodiment 163, wherein the ion channel is a sodium channel.

165. The nucleic acid sequence of embodiment 163, wherein the ion channel is a potassium channel.

166. The nucleic acid sequence of embodiment 163, wherein the ion channel is a calcium channel.

167. The nucleic acid sequence of embodiment 163, wherein the ion channel is a chloride channel.

168. The nucleic acid sequence of any one of embodiments 151-167, wherein the one or more target molecules is associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

169. The nucleic acid sequence of any one of embodiments 151-168, wherein the one or more target molecules comprises one or more genes of Table 1.

170. The nucleic acid sequence of any one of embodiments 151-169, wherein the one or more target molecules comprises SCN9A, SCN10A, or SCN11A, or a combination thereof.

171. The nucleic acid sequence of any one of embodiments 151-170, wherein the one or more target molecules comprise a natural antisense transcript for SCN9A.

172. The nucleic acid sequence of any one of embodiments 151-171, wherein the one or more target molecules is associated with pain.

173. The nucleic acid sequence of claim 172, wherein the pain comprises neuropathic pain, inflammatory pain, visceral pain, migraine pain, erythromelalgia pain, fibromyalgia pain, idiopathic pain, or somatic pain, or a combination thereof.

174. The nucleic acid sequence of any one of embodiments 151-173, wherein the one or more target molecules comprises one or more genes of Table 2.

175. The nucleic acid sequence of any one of embodiments 151-174, wherein the one or more target molecules is associated with a neurological disease.

176. The nucleic acid sequence of embodiment 175, where in the neurological disease comprises dementia.

177. The nucleic acid sequence of any one of embodiments 151-176, wherein the one or more target molecules is associated with Alzheimer's disease.

178. The nucleic acid sequence of any one of embodiments 151-177, wherein the one or more target molecules comprises one or more genes of Table 3.

179.

The nucleic acid sequence of any one of embodiments 151-178, wherein the one or more target molecules is associated with Parkinson's disease.

180. The nucleic acid sequence of any one of embodiments 151-179, wherein the one or more target molecules comprises one or more genes selected from the group comprising SNCA, GBA, and LRRK2.

181. The nucleic acid sequence of any one of embodiments 151-180, wherein the one or more target molecules is associated with Huntington's disease.

182. The nucleic acid sequence of any one of embodiments 151-181, wherein the one or more target molecules is associated with schizophrenia.

183. The nucleic acid sequence of any one of embodiments 151-182, wherein the one or more target molecules comprises GPR52.

184. The nucleic acid sequence of any one of embodiments 151-183, wherein the one or more target molecules is associated with Amyotrophic lateral sclerosis (ALS).

185. The nucleic acid sequence of any one of embodiments 151-184, wherein the one or more target molecule comprises one or more genes selected from the group comprising SOD1, ataxin-2, TDP43, C9ORF72, FUS and SCA2.

186. The nucleic acid sequence of any one of embodiments 151-185, wherein the one or more target molecules is associated with Multiple Sclerosis.

187. The nucleic acid sequence of any one of embodiments 151-186, wherein the one or more target molecules is associated with a central nervous system ailment.

188. The nucleic acid sequence of any one of embodiments 151-187, wherein the one or more target molecules comprise one or more genes selected from the group comprising BFD1, C9orf72, FUS, SOD1, TPD43, Brain-derived neurotrophic factor, Nerve growth factor, a Neurotrophin, BCL11A, FMR1, DNM2, PrP, UBE3A, GYS1, STING, and GFAP.

189. The nucleic acid sequence of any one of embodiments 151-188, wherein the nucleic acid binding domain binds to at least one of the one or more target molecules.

190. The nucleic acid sequence of any one of embodiments 151-189, wherein the nucleic acid binding domain comprises a nuclease dead Clustered Regularly Interspaced Short Palindromic Repeats associated protein (dCas).

191. The nucleic acid sequence of embodiment 190, wherein the dCas comprises a mutated Cas protein.

192. The nucleic acid sequence of embodiment 190, wherein the dCas is a truncated Cas protein.

193. The nucleic acid sequence of embodiment 190, wherein the dCas is a mutated or truncated Cas protein of Table 4.

194. The nucleic acid sequence of embodiment 190, wherein the dCas comprises a sequence at least 90% identical to a dCas of Table 4.

195. The nucleic acid sequence of embodiment 190, wherein the dCas comprises dCas9.

196. The nucleic acid sequence of embodiment 195, wherein the dCas9 is a truncated or mutated Cas9 protein.

197. The nucleic acid sequence of embodiment 195, wherein the dCas comprises dCas12.

198. The nucleic acid sequence of embodiment 197, wherein the dCas12 is a truncated or mutated Cas12 protein.

199. The nucleic acid sequence of embodiment 190, wherein the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteurianus, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9.

200. The nucleic acid sequence of embodiment 190, wherein the dCas has a REC2 domain deletion.

201. The nucleic acid sequence of embodiment 190, wherein the dCas has a REC3 domain deletion.

202. The nucleic acid sequence of embodiment 190, wherein the dCas has a HNH deletion.

203. The nucleic acid sequence of embodiment 190, wherein the dCas has a nuclease (NUC) lobe deletion.

204. The nucleic acid sequence of any one of embodiments 151-189, wherein the nucleic acid binding domain comprises a zinc finger protein.

205. The nucleic acid sequence of any one of embodiments 151-189, wherein the nucleic acid binding domain comprises a meganuclease.

206. The nucleic acid sequence of any one of embodiments 151-189, wherein the nucleic acid binding domain comprises a transcription activator-like effector nucleases (TALENs).

207. The nucleic acid sequence of any one of embodiments 151-206, wherein the epigenetic modulator comprises a transcription regulatory domain.

208. The nucleic acid sequence of embodiment 207, wherein the epigenetic modulator comprises a domain having transcription repression activity (repressor domain).

209. The nucleic acid sequence of embodiment 208, wherein the repressor domain comprises a Krueppel-associated box (KRAB) domain (recruitment of histone methyltransferases and deacetylases).

210. The nucleic acid sequence of embodiment 207, wherein the epigenetic modulator comprises a domain having transcription activation activity (activator domain).

211. The nucleic acid sequence of any one of embodiments 151-210, wherein the epigenetic modulator comprises KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, or G9a (EHMT2), or a variant or combination thereof.

212. The nucleic acid sequence of any one of embodiments 151-211, wherein the epigenetic modulator comprises ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, Nluc, ZFP28-2, ZNF224, or ZNF257, or a variant or combination thereof.

213. The nucleic acid sequence of any one of embodiments 151-212, wherein the epigenetic modulator comprises VP64, Rta, P16, P65, p300, TET1 catalytic domain, TDG, Ldb1 self-association domain, SAM activator (VP64, p65, HSF1), VPR (VP64, p65, Rta), CD, or SunTag, or a variant or combination thereof.

214. The nucleic acid sequence of any one of embodiments 151-213, wherein the epigenetic modulator comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof.

215. The nucleic acid sequence of any one of embodiments 151-214, wherein the epigenetic modulator comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb1 self-association domain (recruits enhancer-associated endogenous Ldb1), SAM activator (VP64, p65, HSF1) (recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2) (histone methyltransferase), DNMT3a (DNA methyltransferase), or DNMT3a-DNMT3L (DNA methyltransferase), or a combination thereof.

216. The nucleic acid sequence of any one of embodiments 151-215, wherein the epigenetic modulator is linked to the nucleic acid binding domain at the N-terminus or C-terminus of the nucleic acid binding domain via a linker.

217. The nucleic acid sequence of embodiment 216, wherein the linker is a flexible linker.

218. The nucleic acid sequence of embodiment 216 or embodiment 217, wherein the linker is (GGS)n (SEQ ID NO: 156), (GGGS)n (SEQ ID NO: 157), (GGGGS)n (SEQ ID NO: 158), (G)n (SEQ ID NO: 159), (EAAAK)n (SEQ ID NO: 160), A (EAAAK)nALEA(EAAAK)nA (SEQ ID NO: 161), PAPAP (SEQ ID NO: 162), AEAAAKEAAAKA (SEQ ID NO: 163), (Ala-Pro)x (SEQ ID NO: 164), LE, GlySer-polyPro (Glyc)-polyPro (Glyc)-polyPro (Glyc), GlySer-polyPro-polyPro (Glyc)-polyPro, Gly Ser-polyPro-Gly Ser (Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-β2m-GlySer-β2m-polyPro, GlySer-polyPro-Ub-GlySer, GlySer-polyPro-ZAG-polyPro, GlySer-GlySer-ZAG-GlySer-ZAG-polyPro, GlySer (Glyc)-GlySer (Glyc)-polyPro, (G4S) 3-cTPR3-(G4S) 3, (G4S) 3-cTPR6-(G4S) 3, (G4S) 3-cTPR9-(G4S) 3, (G4S) 3-cTPR12-(G4S) 3, or (G4S)n (SEQ ID NO: 158); wherein n is independently selected from 1 to 10, x is 10-34, polyPro is proline-rich hinge sequence from IgA1, polyPro (Glyc) is proline-rich hinge sequence from IgA1 with an embedded potential N-linked glycosylation site (Asn-Ser-Ser), β2m is β2-microglobulin, Ub is ubiquitin, ZAG is Zn-α2-glycoprotein, and cTPRX is consensus tetratricopeptide repeat sequence with X number of repeats.

219. The nucleic acid sequence of any one of embodiments 151-218, wherein the nucleic acid binding domain and the epigenetic modulator are connected by a disulfide bond.

220. The nucleic acid sequence of any one of embodiments 151-219, wherein the nucleic acid sequence comprises a nuclear localization sequence (NLS).

221. The nucleic acid sequence of any one of embodiments 151-220, wherein the nucleic acid sequence comprises a promoter.

222. The nucleic acid sequence of embodiment 221, wherein the promoter comprises a sequence at least about 90% identical to sequence of Table 5.

223. The nucleic acid sequence of embodiment 221 or embodiment 222, wherein the promoter comprises a nuclear localization sequence.

224. The nucleic acid sequence of embodiment 220 or embodiment 223, wherein the NLS drives nuclear import of the nucleic acid.

225. The nucleic acid sequence of any one of embodiments 221-224, wherein the promoter drives expression of the nucleic acid binding domain.

226. The nucleic acid sequence of any one of embodiments 221-225, wherein the promoter drives expression of the epigenetic modulator.

227. The nucleic acid sequence of any one of embodiments 221-226, wherein the promoter is a promoter naturally associated with the target molecule.

228. The nucleic acid sequence of any one of embodiments 221-227, wherein the promoter is a promoter of a gene from Tables 1-3.

229. The nucleic acid sequence of any one of embodiments 221-227, wherein the promoter is a SCN9A promoter or SON10A promoter.

230. The nucleic acid sequence of any one of embodiments 221-227, wherein the promoter is a pan-neuronal gene promoter.

231. The nucleic acid sequence of any one of embodiments 221-227, wherein the promoter is a promoter naturally associated with a gene related to pain.

232. The nucleic acid sequence of any one of embodiments 221-227, wherein the promoter is a promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, promoter of the NeuN gene (Fox-3, Rbfox3, or Hexaribonucleotide Binding Protein-3), promoter of the α-calcium/calmodulin-dependent protein kinase II [CaMKIIα]), promoter of the Rheb gene (ras homolog enriched in brain), TRKA promoter (Tyrosine Kinase A), or jET.

233. The nucleic acid sequence of any one of embodiments 221-227, wherein the promoter is a promoter naturally associated with a gene associated with a channel.

234. The nucleic acid sequence of any one of embodiments 221-227, wherein the promoter is a promoter naturally associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

235. The nucleic acid sequence of any one of embodiments 221-227, wherein the promoter is a promoter naturally associated with inflammatory pain, visceral pain, migraine pain, erythromelalgia pain, fibromyalgia pain, idiopathic pain, or somatic pain, or a combination thereof.

236. The nucleic acid sequence of any one of embodiments 221-227, wherein the promoter is a promoter naturally associated with a neurological disease.

237. The nucleic acid sequence of any one of embodiments 221-227, wherein the promoter is a promoter naturally associated with dementia.

238. The nucleic acid sequence of any one of embodiments 221-227, wherein the promoter is a promoter naturally associated with Alzheimer's disease.

239. The nucleic acid sequence of any one of embodiments 221-227, wherein the promoter is a promoter naturally associated with Parkinson's disease.

240. The nucleic acid sequence of any one of embodiments 221-227, wherein the promoter is a promoter naturally associated with ALS.

241. The nucleic acid sequence of any one of embodiments 221-227, wherein the promoter is a promoter naturally associated with Multiple Sclerosis.

242. The nucleic acid sequence of any one of embodiments 221-227, wherein the promoter is a promoter naturally associated with a gene associated with a central nervous system ailment.

243. The nucleic acid sequence of any one of embodiments 221-227, wherein the promoter is a promoter of a gene selected from SNCA, GBA, and LRRK2, SOD1, ataxin-2, SCA2, BFD1, FUS, TDP43, (9orf72, Brain-derived neurotrophic factor, Nerve growth factor, a Neurotrophin, BOL11A, FMR1, DNM2, PrP, UBE3A, GYS1, and GFAP.

244. The nucleic acid sequence of any one of embodiments 221-227, wherein the promoter is a pol II promoter (e.g., Thy1 and H1xb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter.

245. The nucleic acid sequence of any one of embodiments 221-227, wherein the promoter is controlled by a small molecule.

246. The nucleic acid sequence of embodiment 245, wherein the promoter is a tetracycline responsive promoter, glucocorticoid responsive promoter, RU-486 responsive promoter, peroxide inducible promoter or tamoxifen induced promoter.

247. The nucleic acid sequence of any one of embodiments 221-244, wherein expression of the epigenetic modulator and/or transcription regulatory domain occurs upon a natural or physiological induction of the promoter.

248. The nucleic acid sequence of embodiment 247, wherein the promoter is induced when a pathology arises in the subject.

249. The nucleic acid sequence of embodiment 248, wherein the pathology is injury and/or inflammation.

250. The nucleic acid sequence of embodiment 248 or embodiment 249, wherein the promoter is a galanin promoter or NF-κB promoter.

251. The nucleic acid sequence of any one of embodiments 151-250, wherein the nucleic acid sequence comprises two or more promoters.

252. The nucleic acid sequence of any one of embodiments 151-251, wherein the nucleic acid sequence comprises tandem promoters.

253. The nucleic acid sequence of any one of embodiments 151-252, wherein the nucleic acid sequence comprises an enhancer.

254. The nucleic acid sequence of any one of embodiments 151-253, wherein the nucleic acid sequence comprises an intron.

255. The nucleic acid sequence of any one of embodiments 151-254, wherein the nucleic acid comprises an inverted terminal repeat (ITR).

256. The nucleic acid sequence of any one of embodiments 151-255, wherein the nucleic acid comprises a terminator sequence.

257. The nucleic acid sequence of any one of embodiments 151-256, comprising administering to the subject one or more guide RNA sequences.

258. The nucleic acid sequence of embodiment 257, wherein the one or more guide RNA sequences is selected from a sequence in Table 6.

259. The nucleic acid sequence of embodiment 257 or embodiment 258, wherein the one or more guide RNA sequences binds to one or more of the target molecules.

260. The nucleic acid sequence of embodiment 259, wherein the one or more target molecules is 2, 3, 4, or 5 target molecules.

261. The nucleic acid sequence of any one of embodiments 257-260, wherein the one or more guide RNA sequences is 2, 3, 4, or 5 guide RNA sequences.

262. The nucleic acid sequence of embodiment 261, comprising at least two guide RNA sequences, wherein at least two of the guide RNA sequences are different.

263. The nucleic acid sequence of any one of embodiments 257-262, wherein at least one or more guide RNA sequences target the promoter of embodiments 221-252, creating a regulatory feedback loop to control expression levels.

264. The nucleic acid sequence of any one of embodiments 151-263, wherein the nucleic acid is delivered as a naked (or unmodified) nucleic acid.

265. The nucleic acid sequence of any one of embodiments 151-263, wherein the nucleic acid is delivered complexed with cationic molecules.

266. The nucleic acid sequence of any one of embodiments 151-263, wherein the nucleic acid is delivered to the subject via a vehicle (e.g., viral delivery vehicle (e.g., retroviral vector, lentiviral vector, or adenoviral vector), liposome, nanoparticle, or exosome).

267. The nucleic acid sequence of embodiment 266, wherein the vehicle is a viral delivery vehicle.

268. The nucleic acid sequence of embodiment 267, wherein the viral delivery vehicle is a retroviral vector, lentiviral vector, or adenoviral vector.

269. The nucleic acid sequence of embodiment 268, wherein the viral delivery vehicle is a recombinant adeno-associated virus (AAV).

270. The nucleic acid sequence of embodiment 269, wherein the AAVis AAV1, AAV2, AAV3, AAV4, AAV5, AAV6, AAV7, AAV8, AAV9, AAV10, AAVhu68, AAVrh. 10, AAVrh74 or AAVDJ, or a combination thereof.

271. The nucleic acid sequence of embodiment 269, wherein the AAV is AAV9.

272. The nucleic acid sequence of any one of embodiments 269-271, wherein the AAV has a recombinant capsid.

273. The nucleic acid sequence of embodiment 272, wherein the recombinant capsid encodes a targeting moiety.

274. The nucleic acid sequence of any one of embodiments 151-273, wherein the nucleic acid comprises a sequence encoding a targeting moiety.

275. The nucleic acid sequence of embodiment 266, wherein the vehicle is a liposome, lipid nanoparticle, nanocapsule, or exosome.

276. The nucleic acid sequence of embodiment 275, wherein the vehicle comprises or is connected to a targeting moiety.

277. The nucleic acid sequence of any one of embodiments 273-276, wherein the targeting moiety targets a cell comprising the target molecule in the subject.

278. The nucleic acid sequence of any one of embodiments 273-277, wherein the targeting moiety binds to a peptide product of the target molecule.

279. The nucleic acid sequence of embodiment 278, wherein the target molecule is present on a target cell.

280. The nucleic acid sequence of embodiment 279, wherein the target cell is associated with the disease or condition in the subject.

281. The nucleic acid sequence of any one of embodiments 273-280, wherein the targeting moiety comprises a peptide.

282. The nucleic acid sequence of embodiment 281, wherein the peptide comprises JNJ63955, m3-Huwentoxin-IV, Phlotoxin 1 (PhlTx1), Protoxin-II (ProTx-II), or Ceratotoxin-1 (CcoTx1), or a variant thereof.

283. The nucleic acid sequence of embodiment 281, wherein the peptide comprises a sequence at least about 90% identical to a peptide of Table 7.

284. A combination comprising the nucleic acid sequence of any one of embodiments 151-283, and an additional therapeutic agent.

### Certain Definitions

Percent (%) sequence identity with respect to a reference polypeptide or polynucleotide sequence is the percentage of amino acid or nucleotide residues in a candidate sequence that are identical with the amino acid or nucleotide residues in the reference polypeptide or polynucleotide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid or polynucleotide sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid or polynucleotide sequence comparisons, the % amino acid or polynucleotide sequence identity of a given sequence A to, with, or against a given sequence B (which can alternatively be phrased as a given sequence A that has or comprises a certain % sequence identity to, with, or against a given sequence B) is calculated as follows: 100 times the fraction X/Y, where X is the number of residues scored as identical matches by the sequence alignment program ALIGN-2 in that program's alignment of A and B, and where Y is the total number of residues in B. It will be appreciated that where the length of sequence A is not equal to the length of sequence B, the % sequence identity of A to B will not equal the % sequence identity of B to A. Unless specifically stated otherwise, all % sequence identity values used herein are obtained as described in the immediately preceding paragraph using the ALIGN-2 computer program.

In some embodiments, the term “about” means within 10% of the stated amount. For instance, a peptide comprising about 80% identity to a reference peptide may comprise 72% to 88% identity to the reference peptide sequence.

### Examples

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Preparation of Compositions Comprising Nav1.7-Binding Peptides

Peptide binders of sodium channels are known in the art. For instance, some venomous animals, such as spiders, make molecules in their venom that can bind and inhibit sodium channels of their prey. In particular, spider toxin peptides such as Protoxin-II (ProTx-II), Huwentoxin-IV (HwTx-IV), m3-Huwentoxin-IV (variant m3-HwTX-IV), Ceratotoxin-1 (CcoTx1), and Phlotoxin 1 (PhlTx1) bind Nav1.7.

In this experiment, each of the following peptides are attached to the surface of a different AAV9 packaging mCherry reporter construct: the peptides of Table 7, including ProTx-II, ProTx-III, the ProTx-II variant JNJ63955, HwTx-IV, variant m3-HwTx-IV, CcoTx1 variant 2670, PhlTx1 variant D7A-PhlTx1, and JzTx-V variant AM-0422. These peptides were chosen because they are established Nav 1.7-binding ligands and because they represent a varied selection of peptides with different sequences and differential interactions with the Nav1.7 channel. Peptide attachment to the AAV is performed by both C-term NHS modification of the peptide and N-term Benzoyl-NH modification to bind with the nine lysine residues on the AAV9 surface. Crosslinking the peptides at the N- and C-terminals will increase the chances that at least one them will keep the peptide conformation to bind Nav1.7.

### Example 2: Preparation of Compositions Encoding Nav1.7-Binding Peptides

As attachment of peptides onto AAV9 is not an economically feasible option in terms of scaled-up manufacturing for therapeutic use, sequences encoding the peptides of Example 1 are incorporated into the viral genome to be expressed on viral protein loops. AAV viral capsids are composed of three proteins, VP1, VP2 and VP3. The N terminus of VP2 capsid protein accepts large-peptide insertions as well as proteins, thus, insertion of targeting peptides at VP2 re-targets vector tropism. Other potential sites to insert these target peptides are found in VP3.

In a first experiment, a sequence encoding a peptide of Example 1 is cloned in frame within a-GS-flanking linker to be expressed in different loops of the AAV9 capsid. In this example, there are five inserting positions: 453, 447, 588, 589, and downstream of the N-terminal methionine of the VP2 start codon. Transduction efficiency is determined by mCherry reporter expression in the Nav1.7-expressing cell lines HuH7 and Neuro2a.

Quality assessment tests are conducted to check that the surface modifications do not negatively affect packaging and capsid structure. This assessment focuses on viral titers since yield is usually the trait most affected after capsid modifications. Titers are measured by RT-qPCR. Ratios of viral proteins VP1, VP2, and VP3 are analyzed by protein gels.

### Example 3: Biodistribution and Immunogenicity In Vivo

An unmodified AAV9-mCherry and AAV9 modified with the sequence encoding Nav1.7 binding peptide as described in Example 2 are injected IT into male C57BL/6 mice (n=6/group) with a low viral titer load 1·10¹¹ vg/mouse. As higher titers transduce high amounts of neurons, the lower titer provides for better differentiation of transduction efficacy in Nav1.7 expressing cells. Organs (brain, spinal cord, dorsal root ganglia, eyes, lungs, heart, liver, spleen, and gonads) and blood are harvested for biodistribution and immunology analyses. Importantly, as transduction of sensory neurons in DRG is important to ensure efficacy of future studies that focus on repression of Nav 1.7, which subset of neurons in the DRG are transduced via RNA-scope is analyzed using probes against mCherry and Nav1.7 simultaneously. To define peptidergic and non-peptidergic nociceptor subpopulations, probes against calcitonin gene-related peptide (CGRP) and P2X purinergic ion channel type 3 receptor (P2X3R) are also used. Viral genomes are also be quantified via RT-qPCR. Viral tropism as indicated by mCherry expression is determined from images captured by confocal microscopy and RT-qPCR.

### Example 4: Design of Nav1.7 Promoter Regions

Different Nav 1.7 promoters (#1, #2, and #7) are investigated for expression of mCherry in Nav1.7 expressing cell lines, and non-Nav1.7 expressing cell lines as negative controls, to determine specificity.

Design of minimal human Nav1.7 promoter regions: The promoters tested vary between 700 bp-1.2 kbp in length. Promoters of various lengths are tested to enable their packaging into a single AAV. In addition, Nav1.7 gene enhancers as described in the EPD website are tested even if they are not part of the promoter region (https://epd.epfl.ch//index.php). These new sequences are cloned in a mCherry expressing vector with the CMV removed as previously performed.

In vitro testing of human Nav1.7 promoters: All cloned sequences are tested in human cell lines, and mCherry expression is compared to the vector harboring the CMV promoter. High Nav1.7 expressing lines HuH7 and IMR-90 are utilized. The negative control is the non-Nav1.7-expressing cell line MCF-7. RT-qPCR, FACS-sorting, and imaging analysis is performed to determine the expression (activity and intensity) levels of the transgene using these promoters.

iPCS testing of human Nar1.7 promoters: The promoters that are specific (high expression in Nav1.7 expressing cells and no expression in MCF-7 negative control) are tested in nociceptor-like iPCS cells. RT-qPCR, FACS-sorting, and imaging analysis is performed to determine the expression (activity and intensity) levels of mCherry using these promoters.

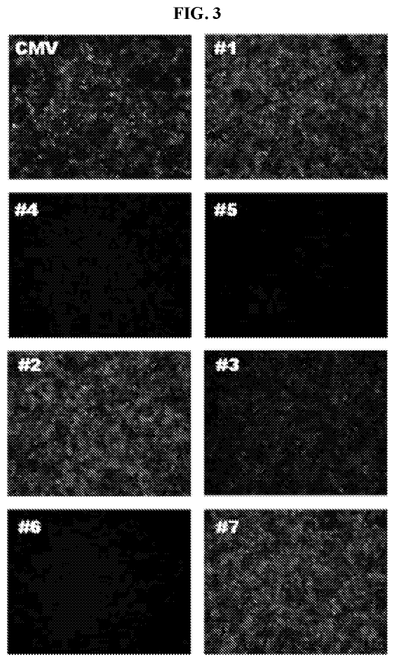

As shown in FIG. 3, seven Nav 1.7 promoters driving the expression of mCherry were transfected into a high Nav1.7-expressing human cell line, HuH7. Three promoters had high transgene expression.

### Example 5: Pilot Testing of Human Nav1.7 Promoter in Mice to Determine Biodistribution

This study is to determine the activity and biodistribution of the human Nav1.7 promoter optimized in the previous example in mice. There is low sequence homology between the promoter region of Nav1.7 in mouse and human. However, our preliminary assays indicate promoters #1, #2 and #7 also work in Neuro2a (cells with high expression of mouse Nav1.7). In addition, there are other examples of promoters with low homology between mouse and human that are able to have similar expression properties in rodents, such as the human synapsin 1 (hSYN1) promoter. Therefore, the human Nav1.7 promoter is tested in mice, and compared to CMV and hSyn1 promoters for activity and biodistribution. Importantly, this will confirm the expression of the transgene (mCherry) in nociceptors. This pilot study will help identify promoters for dose-range studies.

C57BL/6 male mice are injected IT (n=6/group) with 1·10¹² vg of AAV9-hNav1.7promoter-mCherry, AAV9-CMV-mCherry, AAV9-hSyn1-mCherry, or with saline. Biodistribution is determined by protein immunostaining, qPCR and RNA-scope. After three weeks, n=3 mice/group are perfused, while n=3 mice/group are euthanized and tissues are placed in RNAlater at the time of harvesting to preserve RNA. Organs (brain, spinal cord, dorsal root ganglia, eyes, lungs, heart, liver, kidney, skeletal muscle, spleen, and gonads) and blood are harvested. Immunostaining of mCherry (ab167453), and subsequent confocal analysis is performed. Additionally, mCherry mRNA is quantified via RT-qPCR. Importantly, as transduction of sensory neurons in DRG is important to ensure efficacy, which subset of neurons in the DRG are transduced via RNA-scope is analyzed using probes against mCherry and Nav1.7 simultaneously. To define peptidergic and non-peptidergic nociceptor subpopulations, probes against calcitonin gene-related peptide (CGRP) and P2X purinergic ion channel type 3 receptor (P2X3R) are also used. An ELISA and ELISPOT analysis are performed to determine whether any immune reaction to the transgene is detected and if it varies between promoters.

### Example 6: Nav1.7 Gene Repression Utilizing a ZFP-KRAB Repressor

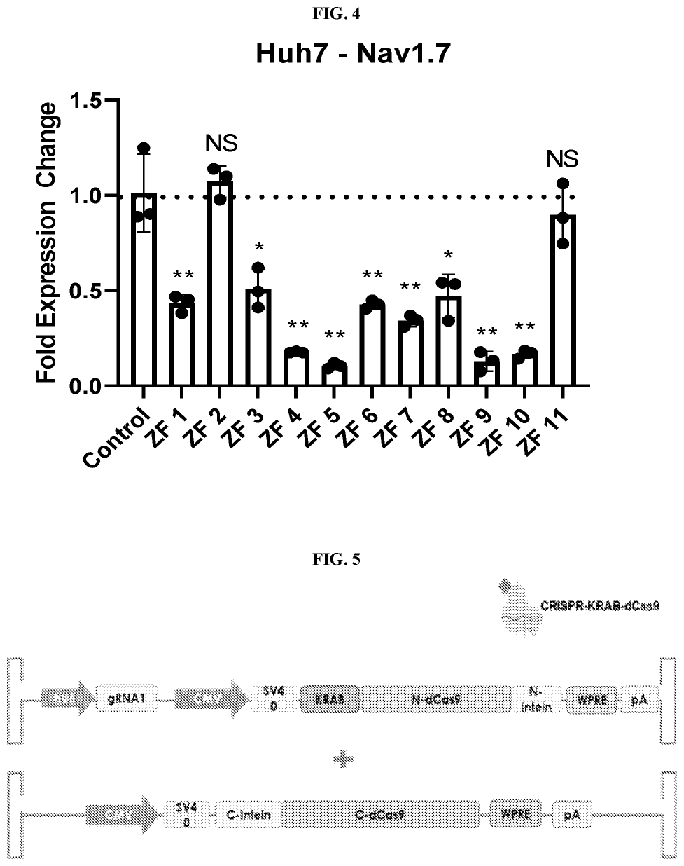

HuH7 cells were transfected with a nucleic acid encoding one of ZFPs 1 to 11 (see Table 8 for ZFP sequences) and the KRAB repressor domain using standard Lipofectamine transfection. Nav1.7 levels were compared with an mCherry control three days post-transfection using qPCR. FIG. 4 shows fold expression change in Nav1.7 after transfection of the ZFP-KRAB.

### Example 7: Nav1.7 and Nav1.8 Repression Using a ZFP-KRAB Repressor

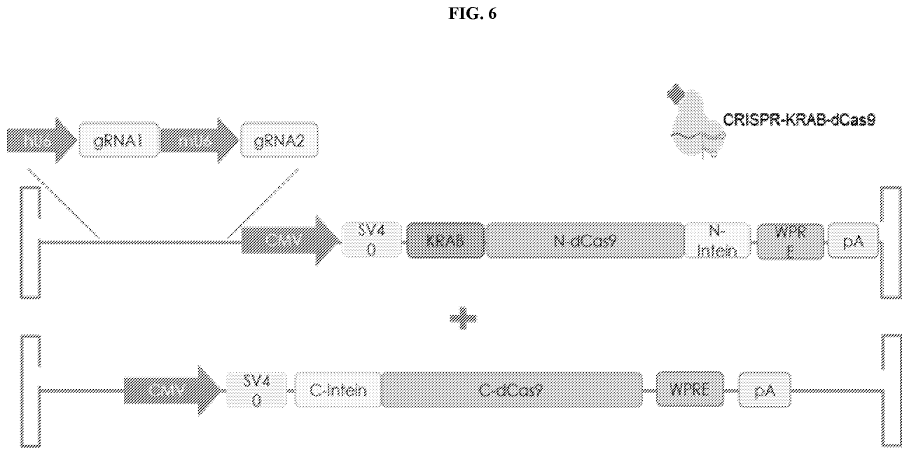

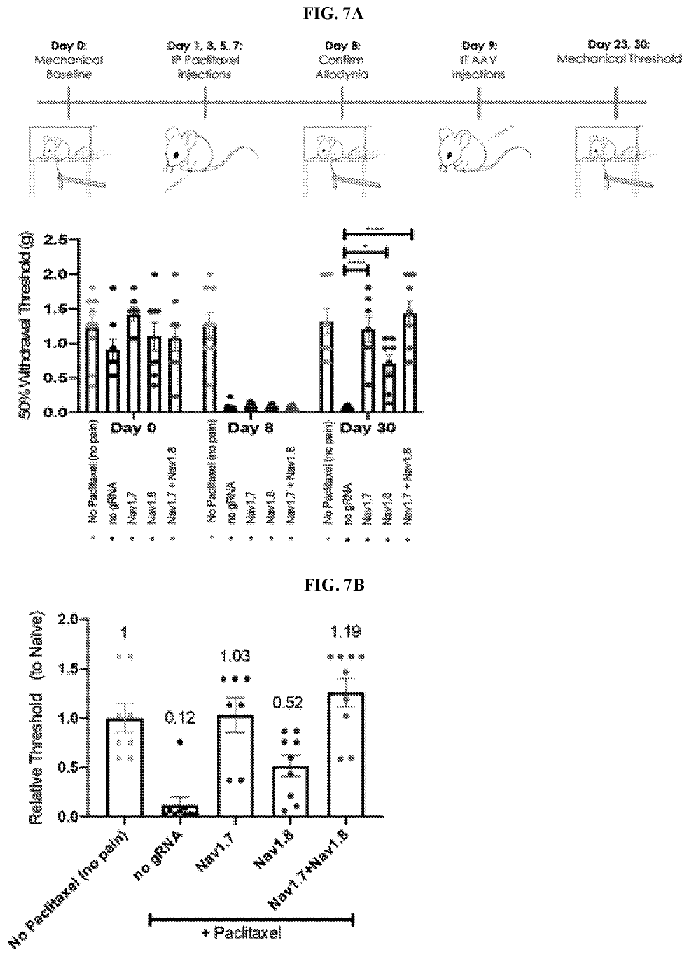

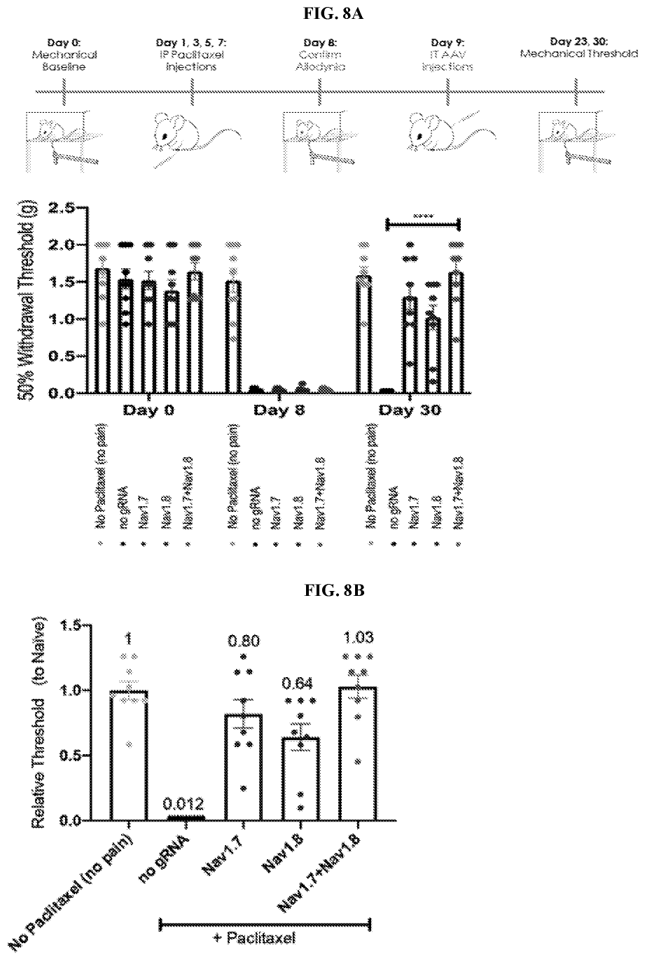

Single-target (Nav1.7 or Nav1.8) and dual-target (Nav1.7 and Nav1.8) compositions were designed as shown in the schematics of FIGS. 5-6. Mice were treated with paclitaxel to induce allodynia, then treated with the single- or dual-target compositions to identify improvements in reversing allodynia. For male mice with mechanical allodynia, as shown in FIGS. 7A-7B, there was about a 50% increase in relative threshold when repressing Nav1.7 versus Nav1.8, and about a 13.4% increase in relative threshold with the dual-target as compared to targeting Nav1.7. For female mice with mechanical allodynia, as shown in FIGS. 8A-8B, there was about a 20% increase in relative threshold when repressing Nav1.7 versus Nav1.8, and about a 22% increase in relative threshold with the dual-target as compared to targeting Nav1.7. These results show that dual Nav1.7 and Nav1.8 repression was more efficacious as compared to just Nav1.7 repression in female and male mice. In addition, there were no significant changes seen in grip strength or rotarod studies, and no neuronal loss, axonopathy, or apoptosis was observed.

### Example 8: Chemical Conjugation of Nav1.7-Binding Peptides onto the Surface of AAV9 and Transduction In Vitro

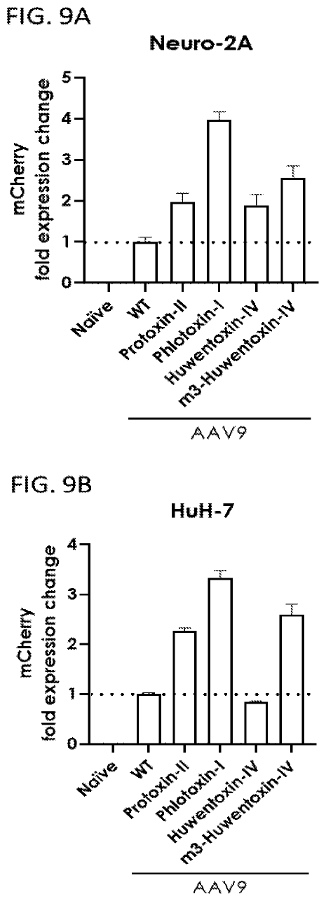

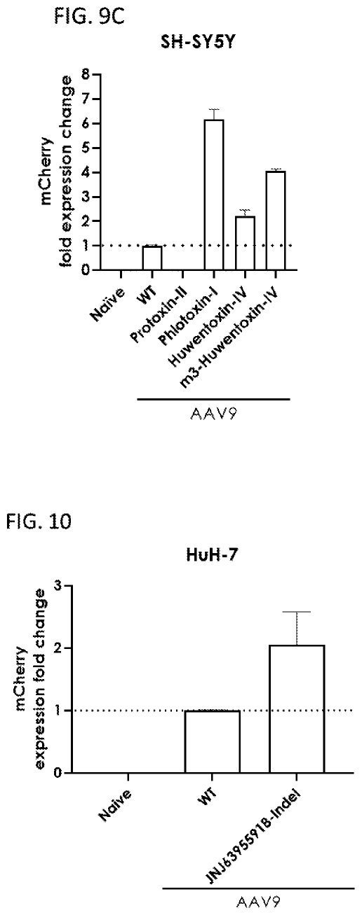

Nav1.7-binding peptides (Protoxin-II, Phlotoxin-I, Huwentoxin-IV, m3-Huwentoxin-IV, see Table 7) were bound to the surface of AAV9 CMV mCherry by chemical crosslinking. The viruses were tested in 3 cell lines which express Nav1.7. The cells were incubated with virus (MOI Neuro-2A: 50,000; HuH-7:100,000; SH-SY5Y: 100,000) for 72 h. Naïve cells did not receive any virus. qRT-PCR was used to measure the expression of the transgene reporter, mCherry. Expression of mCherry was quantified relative to housekeeping gene, glyceraldehyde 3-phosphate dehydrogenase (GAPDH). Fold expression change of mCherry was calculated relative to expression of mCherry in the unmodified AAV9 (WT) condition. Error bars show SD. FIGS. 9A-9C shows that chemical conjugation of Nav1.7-binding peptides onto the surface of AAV9 increases transduction in Neuro-2A, HuH-7, and SH-SY5Y cell lines which express Nav1.7. Some capsid modifications increased transduction roughly 2-6x compared to the WT.

### Example 9: Expression of a Nav1.7-Binding Peptide in Capsid of AAV9 Increases Viral Transduction in a Nav1.7-Binding Cell Line

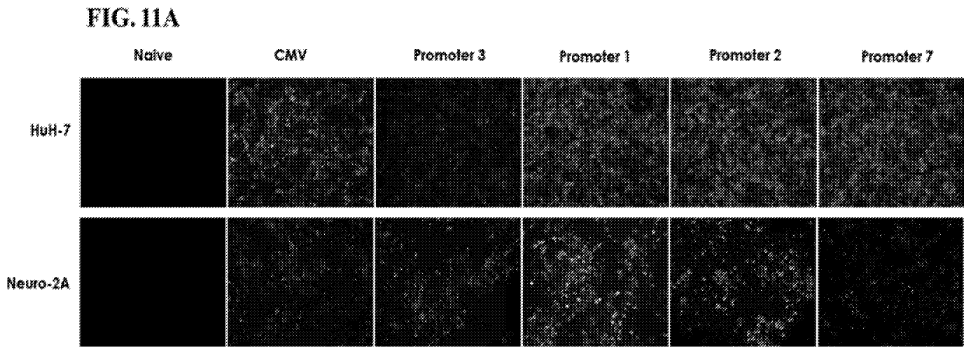

Peptide JNJ63955918-Indel and a short 6 bp linker was expressed in the viral protein VR-III region of the AAV9 capsid. Virus was made using WT capsid or the mutated capsid including the peptide JNJ63955918 (SEQ ID NO: 97). A mCherry reporter was packaged into all viruses. Virus was incubated with cells at an MOI of 10,000 for 72 h. Naïve cells did not receive any virus. The cells were analyzed by qPCR for mCherry expression. Expression of mCherry was quantified relative to housekeeping gene, glyceraldehyde 3-phosphate dehydrogenase (GAPDH). Fold expression change of mCherry was calculated relative to expression of mCherry in the unmodified AAV9 (WT) condition. Error bars show SD. FIG. 10 shows that the expression of JNJ63955918-Indel on the surface of AAV9 facilitates transduction of a cell line, HuH-7, that expresses Nav 1.7, therefore increasing transduction 2x.

### Example 10: Use of Nav1.7 Promoters in Cell Lines that Express Nav1.7 and Transgene Expression

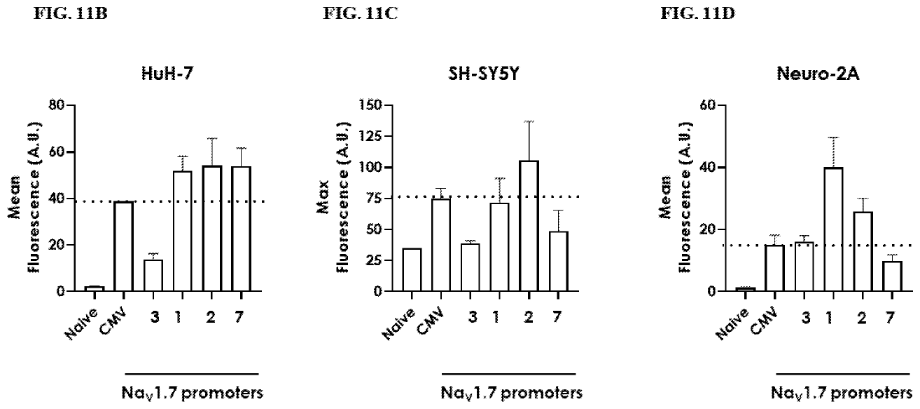

Cell lines are transfected with plasmids with promoters driving mCherry reporter expression. Naïve is the non-transfected condition. The cytomegalovirus (CMV) promoter an industry standard universal promoter. Promoter 3, promoter 1, promoter 2, and promoter 7 (see Table 5) are Nav1.7-specific promoters. Three cell lines tested were HuH-7, SH-SY5Y, and Neuro-2A. After 72 h, cells are imaged with a fluorescent microscope. Red fluorescence intensity was measured and averaged from 3 non-overlapping images with the Histogram tool in ImageJ. Error bars show SD. FIG. 11A shows images from fluorescent microscope on mCherry expression driven by different Nav 1.7-specific promoters tested in HuH-7 and Neuro-2A cell lines. FIGS. 11B-11D show that Nav 1.7-specific promoters can increase transgene expression in cell lines that express Nav1.7.

### Example 11: A Nav1.7-Specific Promoter and Transgene Expression in Nav1.7-Expressing Neurons of Mouse Dorsal Root Ganglia (DRG)

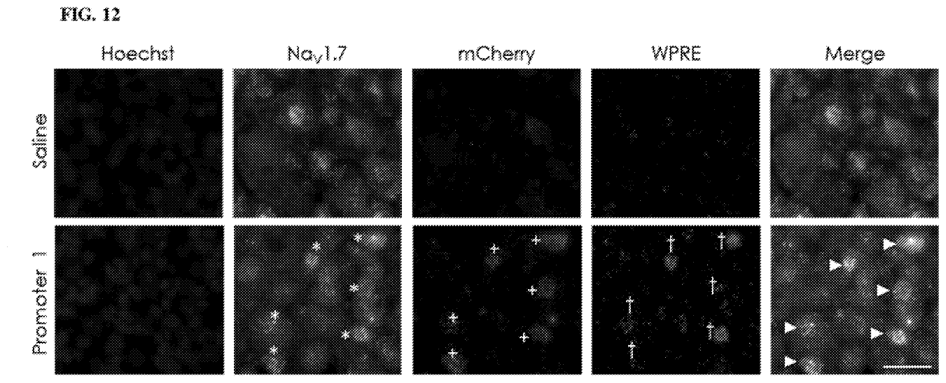

C57BL/6 mice were intrathecally injected with 1×10¹² gc of AAV9 virus packaged with an mCherry reporter driven by a Nav1.7-specific promoter, promoter 1. 4 weeks post-injection, the mice were sacrificed and lumbar DRG were harvested, fixed, and cryoprotected. The DRG were sectioned (10 μm) and labeled using RNAScope Multiplex Fluorescent (Advanced Cell Diagnostics, Inc) reagents with probes targeting Nav1.7, mCherry, and WPRE (marker for viral transgene). A Leica DMi8 fluorescent microscope with a 20× objective was used to capture a Z-stack from each sample. ImageJ was used to create maximum intensity projection images from each Z-stack. Hoechst (blue) was used for nuclear localization. FIG. 12 shows that the promoter 1 Nav1.7-specific promoter drives mCherry expression (red) in target Nav1.7-expressing neuron populations (green). Colocalization of Nav1.7 (asterisks), mCherry (plus signs), and WPRE (daggers) show that the virus transduced Nav 1.7-expressing and the reporter is expressed in these cells (arrowheads). Each punctate red dot in the mCherry channel indicates a single mCherry mRNA molecule, and many punctate dots are observed in several cells, indicating that the Nav1.7-specific promoter is able to drive relatively high expression of the transgene in neurons of mouse DRG. Scale bar shows 50 μm.

### Tables

TABLE 1
Genes involved in channelopathies

| Channels | Potassium | Sodium | Calcium | Chloride |
|---|---|---|---|---|
| Epileptic syndromes | KCNA1, KCNA2, | SCN1A, | CACNA1H | CLCN2, |
| | KCNQ2/3, KCNMA1, | SCN1B, | | GLIALCAM |
| | KCNT1, KCND2, | SCN2A, | | |
| | KCNH5, KCNJ10, | SCN3A, | | |
| | KCNJ11, | SCN8A, | | |
| Ataxia syndromes | KCNA1, KCNC3, | | CACNA1A | |
| | KCND3 | | | |
| Familial Hemiplegic | | SCN1A | CACNA1A | |
| Migraine | | | | |
| Pain syndromes and | KCNQ2 | SCN9A | TRPA1 | — |
| neuropathies | | | | |
| Bartter's syndrome | ROMK1 | | | CLCNKB, BSDN |
| Dent disease | | | | CLCN5 |
| EAST/SESAME | KCNJ11 | | | |
| syndrome | | | | |
| Long QT and Short | KCNQ1, KCNH2, | SCN5A | CACNA1C | |
| QT syndromes | KCNE1, KCNJ2 | | | |
| Brugada syndromes | KCNE3, HCN4 | SCN5a, | CACNA1C | |
| | | SCN1B, | | |
| | | SCN3B | | |
| Catecholaminergic | | | RYR2 | |
| polymorphic | | | | |
| ventricular | | | | |
| tachycardia | | | | |
| Familial Congenital | KCNJ11, ABCC8 | | | |
| hyperinsulinism and | | | | |
| neonatal diabetes | | | | |
| mellitus | | | | |
| Non dystrophic | | SCN4A | | CLCN1 |
| myotonias | | | | |
| Periodic paralysis | KCNJ2, KCNJ18 | SCN4A | CACNA1S | |
| Osteopetrosis | | | | CLCN7, OSTM1 |

TABLE 2
Genes involved in pain

| | | Up/downregulated | Activate or Repress |
|---|---|---|---|
| Gene name | Biological process | in response to pain | with Gene Therapy |
| Cxcl13 | Chemokine-mediated signaling | Upregulated | Repress |
| | pathway | | |
| C1qc | Innate immune response | Upregulated | Repress |
| Ccl2 | Regulation of sensory perception | Upregulated | Repress |
| | of pain | | |
| Fcgr3a | Regulation of sensory perception | Upregulated | Repress |
| | of pain | | |
| Ngfr | Sensory perception of pain | Upregulated | Repress |
| Ltc4s | Response to axon injury | Upregulated | Repress |
| Rpe65 | Cellular response to electrical | Upregulated | Repress |
| | stimulus | | |
| Aoah | Inflammatory response | Upregulated | Repress |
| Aif1 | Response to axon injury | Upregulated | Repress |
| Capn11 | Calcium-dependent cysteine protease | Upregulated | Repress |
| HDAC5, | Regulates neuropathic pain through | Upregulated | Repress |
| HDAC5, | SRY-related HMG-box 10 (SOX10) | | |
| HDAC7 | | | |
| P2 × 3 | Purinergic receptor | Upregulated | Repress |
| P2 × 4 | Purinergic receptor | Upregulated | Repress |
| NMDA | Subunit of the N-methyl-D-aspartate | Upregulated | Repress |
| receptor | (NMDA) receptor is an example of a | | |
| NR2B | heteromeric ligand-gated ion channel | | |
| | that interacts with multiple | | |
| | intracellular proteins by way of | | |
| | different subunits. | | |
| NMDA | Subunit of the N-methyl-D-aspartate | Upregulated | Repress |
| receptor | (NMDA) receptor is an example of a | | |
| NR1 | heteromeric ligand-gated ion channel | | |
| | that interacts with multiple | | |
| | intracellular proteins by way of | | |
| | different subunits. | | |
| MMP-2 & | Metalloprotease-9 and -2 | Upregulated | Repress |
| MMP-9 | | | |
| K channel | Potassium channel | Downregulated | Activate |
| Kir4 | | | |
| TRPV1/2/3/4 | Capsaicin receptor | Upregulated | Both |
| EP | Prostaglandin E(2) (PGE(2)) is both | Upregulated | Repress |
| receptor | an inflammatory mediator released | | |
| EP4 | at the site of tissue inflammation | | |
| | and a neuromodulator that alters | | |
| | neuronal excitability and synaptic | | |
| | processing | | |
| Nav1.1 | Voltage-gated sodium channel | Upregulated | Repress |
| (SCN1A) | | | |
| Nav1.3 | Voltage-gated sodium channel | Upregulated | Repress |
| (SCN3A) | | | |
| Nav1.6 | Voltage-gated sodium channel | Upregulated | Repress |
| (SCN8A) | | | |
| Nav1.7 | Voltage-gated sodium channel | Upregulated | Repress |
| (SCN9A) | | | |
| Nav1.8 | Voltage-gated sodium channel | Upregulated | Repress |
| (SCN10A) | | | |
| Nav1.9 | Voltage-gated sodium channel | Upregulated | Both |
| (SCN11A) | | | |
| KCNQ | Voltage-gated potassium channels | Downregulated/ | Activate |
| (KCNQ2, | | Upregulated | |
| KCNQ3, | | depending on | |
| KCNQ4, | | pain phenotype | |
| KCNQ5) | | (KCNQ2 up) | |
| TRPM8 | Transient receptor potential | Downregulated/ | Repress/ |
| | melastatin 8 | Upregulated | Activate |
| | | depending on | depending |
| | | pain phenotype | on pain type |
| TRPA1 | Transient receptor potential cation | Upregulated | Repress |
| | channel | | |
| CTNNB1 | Encodes an important cytoplasmic | Upregulated | Repress |
| | component of the classical cadherin | | |
| | adhesion complex, also involved in | | |
| | Wnt signaling | | |
| Wnt3a | Wnt signaling | Upregulated | Repress |
| IRS1 | Insulin receptor substrate | Downregulated | Activate |
| mTORC1 | Mammalian target of rapamycin | Downregulated | Activate |
| | complex 1 | | |
| ZFHX2 | Putative transcription factor | Downregulated | Activate |
| TAK1 | Tumor necrosis factor-α | Upregulated | Repress |
| TRPC5 | Transient receptor potential | Downregulated | Activate |
| | canonical 5 | | |
| CDK6 | Cell division protein kinase 6 | Upregulated | Repress |
| PKIA-AS1 | lncRNA | Upregulated | Repress |
| GSK3B | Serine/threonine kinase | Upregulated | Repress |
| OAT | Ornithine Aminotransferase | Upregulated | Repress |
| SHANK3 | Connects neurotransmitter receptors, | Upregulated | Repress |
| | ion channels, and other membrane | | |
| | proteins to the actin cytoskeleton | | |
| | and G-protein-coupled signaling | | |
| | pathways. | | |
| ODC1 | Ornithine decarboxylase | Downregulated | Activate |
| TRPM1 | Transient Receptor Potential Cation | Downregulated | Activate |
| | Channel Subfamily M Member 1) | | |
| TLR4 | Toll Like Receptor 4 | Upregulated | Repress |
| ARRB2 | Beta-arrestin-2 | Downregulated | Activate |
| PKC | Protein Kinase C | Upregulated | Repress |
| CACNA1A | Calcium Voltage-Gated Channel | Downregulated | Activate |
| | Subunit Alpha1 A | (mutations | |
| | | involved in | |
| | | familial | |
| | | migraine) | |
| AIBP | Apolipoprotein A-I binding protein | Downregulated | Activate |
| IL-10 | Pleiotropic cytokine | Downregulated | Activate |
| POMC | Proopiomelanocortin | Downregulated | Activate |
| Cav2.2 | Voltage-gated calcium channel | Upregulated | Repress |
| Cav3.1 | Voltage-gated calcium channel | Upregulated | Repress |
| Cav3.2 | Voltage-gated calcium channel | Upregulated | Repress |
| Kelch-like | A structural protein that binds to | | Repress |
| 1 (KLHL1) | Caᵥ3.2 and actin | | |
| mTOR | member of the phosphatidylinositol | Upregulated | Repress |
| | 3-kinase-related kinase family of | | |
| | protein kinases | | |
| KCNA2 | voltage-gated potassium channel | Downregulated | Activate |
| (Kv1.2) | | | |
| NK1R | Neurokinin 1 receptor (NK1R) | Activated in | Repress |
| | antagonist possesses antidepressant, | pain conditions | |
| | anxiolytic, and antiemetic properties. | as well as anxiety, | |
| | They boost the efficacy of 5-HT3 | depression via | |
| | antagonists to prevent nausea and | substance P. | |
| | vomiting. | | |
| STING1 | Stimulator of Interferon Response | Downregulated | Activate |
| (TMEM- | cGAMP Interactor-1. Stimulator of | | |
| 173) | interferon genes. | | |
| HCN2 | Hyperpolarization-activated cyclic | Upregulated | Repress |
| | nucleotide-gated ion channel | | |
| HCN3 | Hyperpolarization-activated cyclic | Activated | Repress |
| | nucleotide-gated ion channel | | |
| ASIC3 | Acid-sensing ion channel. A sensor | Activated | Repress |
| | of acidic and primary inflammatory | | |
| | pain. | | |
| Caveolin-1 | Cholesterol binding and resident | Downregulated | Activate |
| | protein. Organizes and targets | | |
| | synaptic components of the | | |
| | neurotransmitter and neurotrophic | | |
| | receptor signaling pathways. | | |

TABLE 3
Genes involved in Neurological diseases

| | | Activate or Repress with |
|---|---|---|
| | | Gene Therapy (activation |
| | | or repression may be desired |
| | | depending on the pathology, |
| | | example modulations |
| | | in this column are provided |
| Gene name | Biological process | for Alzheimer's disease) |
| Triggering receptor | Operate Microglia | Activate |
| expressed on myeloid | | |
| cells 2 TREM2 | | |
| Membrane-Spanning 4- | Operate Microglia | Activate |
| domains subfamily A | | |
| (MS₄A) | | |
| LilrB2 | | Repress |
| APOE4 | Lipid metabolism | Repress |
| Presenilins PSEN1, PSEN2 | | Activate |
| Vascular endothelial growth | Neurogenesis and | Upregulation |
| factor VEGF | neuroprotection | |
| Vascular endothelial growth | Neurogenesis and | Upregulation |
| factor Receptor VEGFR | neuroprotection | |
| RANBP17 | Nuclear transport | Repress |
| | receptor (importin- | |
| | beta superfamily) | |
| LAMA3 | Human laminin, alpha 3 | Repress |
| PCDH10 | Human protocadherin 10 | Repress |
| MAPT (encodes Tau protein) | microtubule-associated | Repress |
| | protein in neuro | |
| SOD1 (superoxide dismutase 1) | It is implicated in | Repress |
| | apoptosis and familial | |
| | amyotrophic lateral | |
| | sclerosis. | |
| C9orf72 (chromosome 9 open | It is the most common | Repress |
| reading frame 72) | mutation affected gene | |
| | that is associated with | |
| | familial frontotemporal | |
| | dementia (FTD) and/or | |
| | amyotrophic lateral | |
| | sclerosis (ALS). | |
| TDP-43 (transactive response | Correlated with ALS. | Repress |
| DNA binding protein 43 kDa) | | |
| FUS (FUsed in Sarcoma) | RNA-binding protein. | Repress |
| | Pathological link to ALS. | |

TABLE 4
CRISPR Cas orthologs

| Accession | | Gene | |
|---|---|---|---|
| ID | Name | family | Sequence |
| AAK33936.1 | Streptococcus | Cas9 | MDKKYSIGLDIGTNSVGWAVITDEYKVPSKKFKVLGNTDRHSIKKNL |
| | pyogenes | | IGALLFDSGETAEATRLKRTARRRYTRRKNRICYLQEIFSNEMAKVD |
| | M1 | | DSFFHRLEESFLVEEDKKHERHPIFGNIVDEVAYHEKYPTIYHLRKKL |
| | GAS | | VDSTDKADLRLIYLALAHMIKFRGHFLIEGDLNPDNSDVDKLFIQLV |
| | | | QTYNQLFEENPINASGVDAKAILSARLSKSRRLENLIAQLPGEKKNGL |
| | | | FGNLIALSLGLTPNFKSNFDLAEDAKLQLSKDTYDDDLDNLLAQIGD |
| | | | QYADLFLAAKNLSDAILLSDILRVNTEITKAPLSASMIKRYDEHHQDL |
| | | | TLLKALVRQQLPEKYKEIFFDQSKNGYAGYIDGGASQEEFYKFIKPIL |
| | | | EKMDGTEELLVKLNREDLLRKQRTFDNGSIPHQIHLGELHAILRRQE |
| | | | DFYPFLKDNREKIEKILTFRIPYYVGPLARGNSRFAWMTRKSEETITP |
| | | | WNFEEVVDKGASAQSFIERMTNFDKNLPNEKVLPKHSLLYEYFTVY |
| | | | NELTKVKYVTEGMRKPAFLSGEQKKAIVDLLFKTNRKVTVKQLKED |
| | | | YFKKIECFDSVEISGVEDRFNASLGTYHDLLKIIKDKDFLDNEENEDIL |
| | | | EDIVLTLTLFEDREMIEERLKTYAHLFDDKVMKQLKRRRYTGWGRL |
| | | | SRKLINGIRDKQSGKTILDFLKSDGFANRNFMQLIHDDSLTFKEDIQK |
| | | | AQVSGQGDSLHEHIANLAGSPAIKKGILQTVKVVDELVKVMGRHKP |
| | | | ENIVIEMARENQTTQKGQKNSRERMKRIEEGIKELGSQILKEHPVENT |
| | | | QLQNEKLYLYYLQNGRDMYVDQELDINRLSDYDVDHIVPQSFLKDD |
| | | | SIDNKVLTRSDKNRGKSDNVPSEEVVKKMKNYWRQLLNAKLITQRK |
| | | | FDNLTKAERGGLSELDKAGFIKRQLVETRQITKHVAQILDSRMNTKY |
| | | | DENDKLIREVKVITLKSKLVSDFRKDFQFYKVREINNYHHAHDAYLN |
| | | | AVVGTALIKKYPKLESEFVYGDYKVYDVRKMIAKSEQEIGKATAKY |
| | | | FFYSNIMNFFKTEITLANGEIRKRPLIETNGETGEIVWDKGRDFATVR |
| | | | KVLSMPQVNIVKKTEVQTGGFSKESILPKRNSDKLIARKKDWDPKKY |
| | | | GGFDSPTVAYSVLVVAKVEKGKSKKLKSVKELLGITIMERSSFEKNPI |
| | | | DFLEAKGYKEVKKDLIIKLPKYSLFELENGRKRMLASAGELQKGNEL |
| | | | ALPSKYVNFLYLASHYEKLKGSPEDNEQKQLFVEQHKHYLDEIIEQIS |
| | | | EFSKRVILADANLDKVLSAYNKHRDKPIREQAENIIHLFTLTNLGAPA |
| | | | AFKYFDTTIDRKRYTSTKEVLDATLIHQSITGLYETRIDLSQLGGD |
| | | | (SEQ ID NO 1) |
| ADC31648.1 | Mycoplasma | Cas9 | MNNSIKSKPEVTIGLDLGVGSVGWAIVDNETNIIHHLGSRLFSQAKTA |
| | gallisepticum | | EDRRSFRGVRRLIRRRKYKLKRFVNLIWKYNSYFGFKNKEDILNNYQ |
| | str. F | | EQQKLHNTVLNLKSEALNAKIDPKALSWILHDYLKNRGHFYEDNRD |
| | | | FNVYPTKELAKYFDKYGYYKGIIDSKEDNDNKLEEELTKYKFSNKH |
| | | | WLEEVKKVLSNQTGLPEKFKEEYESLFSYVRNYSEGPGSINSVSPYGI |
| | | | YHLDEKEGKVVQKYNNIWDKTIGKCNIFPDEYRAPKNSPIAMIFNEIN |
| | | | ELSTIRSYSIYLTGWFINQEFKKAYLNKLLDLLIKTNGEKPIDARQFKK |
| | | | LREETIAESIGKETLKDVENEEKLEKEDHKWKLKGLKLNTNGKIQYN |
| | | | DLSSLAKFVHKLKQHLKLDFLLEDQYATLDKINFLQSLFVYLGKHLR |
| | | | YSNRVDSANLKEFSDSNKLFERILQKQKDGLFKLFEQTDKDDEKILA |
| | | | QTHSLSTKAMLLAITRMTNLDNDEDNQKNNDKGWNFEAIKNFDQK |
| | | | FIDITKKNNNLSLKQNKRYLDDRFINDAILSPGVKRILREATKVFNAIL |
| | | | KQFSEEYDVTKVVIELARELSEEKELENTKNYKKLIKKNGDKISEGL |
| | | | KALGISEDEIKDILKSPTKSYKFLLWLQQDHIDPYSLKEIAFDDIFTKT |
| | | | EKFEIDHIIPYSISFDDSSSNKLLVLAESNQAKSNQTPYEFISSGNAGIK |
| | | | WEDYEAYCRKFKDGDSSLLDSTQRSKKFAKMMKTDTSSKYDIGFLA |
| | | | RNLNDTRYATIVFRDALEDYANNHLVEDKPMFKVVCINGSVTSFLR |
| | | | KNFDDSSYAKKDRDKNIHHAVDASIISIFSNETKTLFNQLTQFADYKL |
| | | | FKNTDGSWKKIDPKTGVVTEVTDENWKQIRVRNQVSEIAKVIEKYIQ |
| | | | DSNIERKARYSRKIENKTNISLFNDTVYSAKKVGYEDQIKRKNLKTL |
| | | | DIHESAKENKNSKVKRQFVYRKLVNVSLLNNDKLADLFAEKEDILM |
| | | | YRANPWVINLAEQIFNEYTENKKIKSQNVFEKYMLDLTKEFPEKFSE |
| | | | FLVKSMLRNKTAIIYDDKKNIVHRIKRLKMLSSELKENKLSNVIIRSK |
| | | | NQSGTKLSYQDTINSLALMIMRSIDPTAKKQYIRVPLNTLNLHLGDH |
| | | | DFDLHNMDAYLKKPKFVKYLKANEIGDEYKPWRVLTSGTLLIHKKD |
| | | | KKLMYISSFQNLNDVIEIKNLIETEYKENDDSDSKKKKKANRFLMTL |
| | | | STILNDYILLDAKDNFDILGLSKNRIDEILNSKLGLDKIVK (SEQ ID NO |
| | | | 2) |
| ADX75954.1 | Staphylococcus | Cas9 | MGRKPYILSLDIGTGSVGYACMDKGFNVLKYHDKDALGVYLFDGA |
| | pseudinter- | | LTAQERRQFRTSRRRKNRRIKRLGLLQELLAPLVQNPNFYQFQRQFA |
| | medius | | WKNDNMDFKNKSLSEVLSFLGYESKKYPTIYHLQEALLLKDEKFDP |
| | ED99 | | ELIYMALYHLVKYRGHFLFDHLKIENLTNNDNMHDFVELIETYENLN |
| | | | NIKLNLDYEKTKVIYEILKDNEMTKNDRAKRVKNMEKKLEQFSIML |
| | | | LGLKFNEGKLFNHADNAEELKGANQSHTFADNYEENLTPFLTVEQS |
| | | | EFIERANKIYLSLTLQDILKGKKSMAMSKVAAYDKFRNELKQVKDIV |
| | | | YKADSTRTQFKKIFVSSKKSLKQYDATPNDQTFSSLCLFDQYLIRPKK |
| | | | QYSLLIKELKKIIPQDSELYFEAENDTLLKVLNTTDNASIPMQINLYEA |
| | | | ETILRNQQKYHAEITDEMIEKVLSLIQFRIPYYVGPLVNDHTASKFGW |
| | | | MERKSNESIKPWNFDEVVDRSKSATQFIRRMTNKCSYLINEDVLPKN |
| | | | SLLYQEMEVLNELNATQIRLQTDPKNRKYRMMPQIKLFAVEHIFKKY |
| | | | KTVSHSKFLEIMLNSNHRENFMNHGEKLSIFGTQDDKKFASKLSSYQ |
| | | | DMTKIFGDIEGKRAQIEEIIQWITIFEDKKILVQKLKECYPELTSKQINQ |
| | | | LKKLNYSGWGRLSEKLLTHAYQGHSIIELLRHSDENFMEILTNDVYG |
| | | | FQNFIKEENQVQSNKIQHQDIANLTTSPALKKGIWSTIKLVRELTSIFG |
| | | | EPEKIIMEFATEDQQKGKKQKSRKQLWDDNIKKNKLKSVDEYKYIID |
| | | | VANKLNNEQLQQEKLWLYLSQNGKCMYSGQSIDLDALLSPNATKH |
| | | | YEVDHIFPRSFIKDDSIDNKVLVIKKMNQTKGDQVPLQFIQQPYERIA |
| | | | YWKSLNKAGLISDSKLHKLMKPEFTAMDKEGFIQRQLVETRQISVHV |
| | | | RDFLKEEYPNTKVIPMKAKMVSEFRKKFDIPKIRQMNDAHHAIDAYL |
| | | | NGVVYHGAQLAYPNVDLFDFNFKWEKVREKWKALGEFNTKQKSRE |
| | | | LFFFKKLEKMEVSQGERLISKIKLDMNHFKINYSRKLANIPQQFYNQT |
| | | | AVSPKTAELKYESNKSNEVVYKGLTPYQTYVVAIKSVNKKGKEKME |
| | | | YQMIDHYVFDFYKFQNGNEKELALYLAQRENKDEVLDAQIVYSLNK |
| | | | GDLLYINNHPCYFVSRKEVINAKQFELTVEQQLSLYNVMNNKETNV |
| | | | EKLLIEYDFIAEKVINEYHHYLNSKLKEKRVRTFFSESNQTHEDFIKA |
| | | | LDELFKVVTASATRSDKIGSRKNSMTHRAFLGKGKDVKIAYTSISGL |
| | | | KTTKPKSLFKLAESRNEL (SEQ ID NO 3) |
| AEX66236.1 | Corynebacterium | Cas9 | MKYHVGIDVGTFSVGLAAIEVDDAGMPIKTLSLVSHIHDSGLDPDKI |
| | diphtheriae | | KSAVTRLASSGIARRTRRLYRRKRRRLQQLDKFIQRQGWPVIELEDY |
| | C7 | | SDPLYPWKVRAELAASYIADEKERGEKLSVALRHIARHRGWRNPYA |
| | | | KVSSLYLPDEPSDAFKAIREEIKRASGQPVPETATVGQMVTLCELGTL |
| | | | KLRGEGGVLSARLQQSDHAREIQEICRMQEIGQELYRKIIDVVFAAES |
| | | | PKGSASSRVGKDPLQPGKNRALKASDAFQRYRIAALIGNLRVRVDGE |
| | | | KRILSVEEKNLVFDHLVNLAPKKEPEWVTIAEILGIDRGQLIGTATMT |
| | | | DDGERAGARPPTHDTNRSIVNSRIAPLVDWWKTASALEQHAMVKAL |
| | | | SNAEVDDFDSPEGAKVQAFFADLDDDVHAKLDSLHLPVGRAAYSED |
| | | | TLVRLTRRMLADGVDLYTARLQEFGIEPSWTPPAPRIGEPVGNPAVD |
| | | | RVLKTVSRWLESATKTWGAPERVIIEHVREGFVTEKRAREMDGDMR |
| | | | RRAARNAKLFQEMQEKLNVQGKPSRADLWRYQSVQRQNCQCAYC |
| | | | GSPITFSNSEMDHIVPRAGQGSTNTRENLVAVCHRCNQSKGNTPFAI |
| | | | WAKNTSIEGVSVKEAVERTRHWVTDTGMRSTDFKKFTKAVVERFQ |
| | | | RATMDEEIDARSMESVAWMANELRSRVAQHFASHGTTVRVYRGSL |
| | | | TAEARRASGISGKLEFLDGVGKSRLDRRHHAIDAAVIAFTSDYVAET |
| | | | LAVRSNLKQSQAHRQEAPQWREFTGKDAEHRAAWRVWCQKMEKL |
| | | | SALLTEDLRDDRVVVMSNVRLRLGNGSAHEETIGKLSKVKLGSQLS |
| | | | VSDIDKASSEALWCALTREPDFDPKDGLPANPERHIRVNGTHVYAGD |
| | | | NIGLFPVSAGSIALRGGYAELGSSFHHARVYKITSGKKPAFAMLRVY |
| | | | TIDLLPYRNQDLFSVELKPQTMSMRQAEKKLRDALATGNAEYLGWL |
| | | | VVDDELVVDTSKIATDQVKAVEAELGTIRRWRVDGFFGDTRLRLRP |
| | | | LQMSKEGIKKESAPELSKIIDRPGWLPAVNKLFSEGNVTVVRRDSLG |
| | | | RVRLESTAHLPVTWKVQ (SEQ ID NO 4) |
| APG80630.1 | Candidatus | Cas9 | MLGSSRYLRYNLTSFEGKEPFLIMGYYKEYNKELSSKAQKEFNDQIS |
| | Parvarchaeum | | EFNSYYKLGIDLGDKTGIAIVKGNKIILAKTLIDLHSQKLDKRREARR |
| | acidiphilum | | NRRTRLSRKKRLARLRSWVMRQKVGNQRLPDPYKIMHDNKYWSIY |
| | ARMAN-4 | | NKSNSANKKNWIDLLIHSNSLSADDFVRGLTIIFRKRGYLAFKYLSRL |
| | | | SDKEFEKYIDNLKPPISKYEYDEDLEELSSRVENGEIEEKKFEGLKNK |
| | | | LDKIDKESKDFQVKQREEVKKELEDLVDLFAKSVDNKIDKARWKRE |
| | | | LNNLLDKKVRKIRFDNRFILKCKIKGCNKNTPKKEKVRDFELKMVLN |
| | | | NARSDYQISDEDLNSFRNEVINIFQKKENLKKGELKGVTIEDLRKQLN |
| | | | KTFNKAKIKKGIREQIRSIVFEKISGRSKFCKEHLKEFSEKPAPSDRINY |
| | | | GVNSAREQHDFRVLNFIDKKIFKDKLIDPSKLRYITIESPEPETEKLEK |
| | | | GQISEKSFETLKEKLAKETGGIDIYTGEKLKKDFEIEHIFPRARMGPSI |
| | | | RENEVASNLETNKEKADRTPWEWFGQDEKRWSEFEKRVNSLYSKK |
| | | | KISERKREILLNKSNEYPGLNPTELSRIPSTLSDFVESIRKMFVKYGYE |
| | | | EPQTLVQKGKPIIQVVRGRDTQALRWRWHALDSNIIPEKDRKSSFNH |
| | | | AEDAVIAACMPPYYLRQKIFREEAKIKRKVSNKEKEVTRPDMPTKKI |
| | | | APNWSEFMKTRNEPVIEVIGKVKPSWKNSIMDQTFYKYLLKPFKDNL |
| | | | IKIPNVKNTYKWIGVNGQTDSLSLPSKVLSISNKKVDSSTVLLVHDKK |
| | | | GGKRNWVPKSIGGLLVYITPKDGPKRIVQVKPATQGLLIYRNEDGRV |
| | | | DAVREFINPVIEMYNNGKLAFVEKENEEELLKYFNLLEKGQKFERIR |
| | | | RYDMITYNSKFYYVTKINKNHRVTIQEESKIKAESDKVKSSSGKEYT |
| | | | RKETEELSLQKLAELISI (SEQ ID NO 5) |
| BAK69486.1 | Campylobacter | Cas9 | MRILGFDIGINSIGWAFVENDELKDCGVRIFTKAENPKNKESLALPRR |
| | lari | | NARSSRRRLKRRKARLIAIKRILAKELKLNYKDYVAADGELPKAYEG |
| | | | SLASVYELRYKALTQNLETKDLARVILHIAKHRGYMNKNEKKSNDA |
| | | | KKGKILSALKNNALKLENYQSVGEYFYKEFFQKYKKNTKNFIKIRNT |
| | | | KDNYNNCVLSSDLEKELKLILEKQKEFGYNYSEDFINEILKVAFFQRP |
| | | | LKDFSHLVGACTFFEEEKRACKNSYSAWEFVALTKIINEIKSLEKISGE |
| | | | IVPTQTINEVLNLILDKGSITYKKFRSCINLHESISFKSLKYDKENAEN |
| | | | AKLIDFRKLVEFKKALGVHSLSRQELDQISTHITLIKDNVKLKTVLEK |
| | | | YNLSNEQINNLLEIEFNDYINLSFKALGMILPLMREGKRYDEACEIAN |
| | | | LKPKTVDEKKDFLPAFCDSIFAHELSNPVVNRAISEYRKVLNALLKK |
| | | | YGKVHKIHLELARDVGLSKKAREKIEKEQKENQAVNAWALKECENI |
| | | | GLKASAKNILKLKLWKEQKEICIYSGNKISIEHLKDEKALEVDHIYPY |
| | | | SRSFDDSFINKVLVFTKENQEKLNKTPFEAFGKNIEKWSKIQTLAQNL |
| | | | PYKKKNKILDENFKDKQQEDFISRNLNDTRYIATLIAKYTKEYLNFLL |
| | | | LSENENANLKSGEKGSKIHVQTISGMLTSVLRHTWGFDKKDRNNHL |
| | | | HHALDAIIVAYSTNSIIKAFSDFRKNQELLKARFYAKELTSDNYKHQV |
| | | | KFFEPFKSFREKILSKIDEIFVSKPPRKRARRALHKDTFHSENKIIDKCS |
| | | | YNSKEGLQIALSCGRVRKIGTKYVENDTIVRVDIFKKQNKFYAIPIYA |
| | | | MDFALGILPNKIVITGKDKNNNPKQWQTIDESYEFCFSLYKNDLILLQ |
| | | | KKNMQEPEFAYYNDFSISTSSICVEKHDNKFENLTSNQKLLFSNAKE |
| | | | GSVKVESLGIQNLKVFEKYIITPLGDKIKADFQPRENISLKTSKKYGLR |
| | | | (SEQ ID NO 6) |
| CBK78998.1 | Coprococcus | Cas9 | MKQEYFLGLDMGTGSLGWAVTDSTYQVMRKHGKALWGTRLFESA |
| | catus | | STAEERRMFRTARRRLDRRNWRIQVLQEIFSEEISKVDPGFFLRMKES |
| | GD/7 | | KYYPEDKRDAEGNCPELPYALFVDDNYTDKNYHKDYPTIYHLRKM |
| | | | LMETTEIPDIRLVYLVLHHMMKHRGHFLLSGDISQIKEFKSTFEQLIQ |
| | | | NIQDEELEWHISLDDAAIQFVEHVLKDRNLTRSTKKSRLIKQLNAKS |
| | | | ACEKAILNLLSGGTVKLSDIFNNKELDESERPKVSFADSGYDDYIGIV |
| | | | EAELAEQYYIIASAKAVYDWSVLVEILGNSVSISEAKIKVYQKHQAD |
| | | | LKTLKKIVRQYMTKEDYKRVFVDTEEKLNNYSAYIGMTKKNGKKV |
| | | | DLKSKQCTQADFYDFLKKNVIKVIDHKEITQEIESEIEKENFLPKQVT |
| | | | KDNGVIPYQVHDYELKKILDNLGTRMPFIKENAEKIQQLFEFRIPYYV |
| | | | GPLNRVDDGKDGKFTWSVRKSDARIYPWNFTEVIDVEASAEKFIRR |
| | | | MTNKCTYLVGEDVLPKDSLVYSKFMVLNELNNLRLNGEKISVELKQ |
| | | | RIYEELFCKYRKVTRKKLERYLVIEGIAKKGVEITGIDGDFKASLTAY |
| | | | HDFKERLTDVQLSQRAKEAIVLNVVLFGDDKKLLKQRLSKMYPNLT |
| | | | TGQLKGICSLSYQGWGRLSKTFLEEITVPAPGTGEVWNIMTALWQTN |
| | | | DNLMQLLSRNYGFTNEVEEFNTLKKETDLSYKTVDELYVSPAVKRQI |
| | | | WQTLKVVKEIQKVMGNAPKRVFVEMAREKQEGKRSDSRKKQLVEL |
| | | | YRACKNEERDWITELNAQSDQQLRSDKLFLYYIQKGRCMYSGETIQL |
| | | | DELWDNTKYDIDHIYPQSKTMDDSLNNRVLVKKNYNAIKSDTYPLS |
| | | | LDIQKKMMSFWKMLQQQGFITKEKYVRLVRSDELSADELAGFIERQI |
| | | | VETRQSTKAVATILKEALPDTEIVYVKAGNVSNFRQTYELLKVREMN |
| | | | DLHHAKDAYLNIVVGNAYFVKFTKNAAWFIRNNPGRSYNLKRMFEF |
| | | | DIERSGEIAWKAGNKGSIVTVKKVMQKNNILVTRKAYEVKGGLFDQ |
| | | | QIMKKGKGQVPIKGNDERLADIEKYGGYNKAAGTYFMLVKSLDKK |
| | | | GKEIRTIEFVPLYLKNQIEINHESAIQYLAQERGLNSPEILLSKIKIDTLF |
| | | | KVDGFKMWLSGRTGNQLIFKGANQLILSHQEAAILKGVVKYVNRKN |
| | | | ENKDAKLSERDGMTEEKLLQLYDTFLDKLSNTVYSIRLSAQIKTLTE |
| | | | KRAKFIGLSNEDQCIVLNEILHMFQCQSGSANLKLIGGPGSAGILVMN |
| | | | NNITACKQISVINQSPTGIYEKEIDLIKL (SEQ ID NO 7) |
| EEZ71796.1 | Neisseria | Cas9 | MAAFKPNPMNYILGLDIGIASVGWAIVEIDEEENPIRLIDLGVRVFER |
| | cinerea | | AEVPKTGDSLAAARRLARSVRRLTRRRAHRLLRARRLLKREGVLQA |
| | ATCC 14685 | | ADFDENGLIKSLPNTPWQLRAAALDRKLTPLEWSAVLLHLIKHRGYL |
| | | | SQRKNEGETADKELGALLKGVADNTHALQTGDFRTPAELALNKFEK |
| | | | ESGHIRNQRGDYSHTFNRKDLQAELNLLFEKQKEFGNPHVSDGLKE |
| | | | GIETLLMTQRPALSGDAVQKMLGHCTFEPTEPKAAKNTYTAERFVW |
| | | | LTKLNNLRILEQGSERPLTDTERATLMDEPYRKSKLTYAQARKLLDL |
| | | | DDTAFFKGLRYGKDNAEASTLMEMKAYHAISRALEKEGLKDKKSPL |
| | | | NLSPELQDEIGTAFSLFKTDEDITGRLKDRVQPEILEALLKHISFDKFV |
| | | | QISLKALRRIVPLMEQGNRYDEACTEIYGDHYGKKNTEEKIYLPPIPA |
| | | | DEIRNPVVLRALSQARKVINGVVRRYGSPARIHIETAREVGKSFKDR |
| | | | KEIEKRQEENRKDREKSAAKFREYFPNFVGEPKSKDILKLRLYEQQH |
| | | | GKCLYSGKEINLGRLNEKGYVEIDHALPFSRTWDDSFNNKVLALGSE |
| | | | NQNKGNQTPYEYFNGKDNSREWQEFKARVETSRFPRSKKQRILLQK |
| | | | FDEDGFKERNLNDTRYINRFLCQFVADHMLLTGKGKRRVFASNGQI |
| | | | TNLLRGFWGLRKVRAENDRHHALDAVVVACSTIAMQQKITRFVRY |
| | | | KEMNAFDGKTIDKETGEVLHQKAHFPQPWEFFAQEVMIRVFGKPDG |
| | | | KPEFEEADTPEKLRTLLAEKLSSRPEAVHKYVTPLFISRAPNRKMSGQ |
| | | | GHMETVKSAKRLDEGISVLRVPLTQLKLKDLEKMVNREREPKLYEA |
| | | | LKARLEAHKDDPAKAFAEPFYKYDKAGNRTQQVKAVRVEQVQKTG |
| | | | VWVHNHNGIADNATIVRVDVFEKGGKYYLVPIYSWQVAKGILPDRA |
| | | | VVQGKDEEDWTVMDDSFEFKFVLYANDLIKLTAKKNEFLGYFVSLN |
| | | | RATGAIDIRTHDTDSTKGKNGIFQSVGVKTALSFQKYQIDELGKEIRP |
| | | | CRLKKRPPVR (SEQ ID NO 8) |
| EFT93846.1 | Enterococcus | Cas9 | MYSIGLDLGISSVGWSVIDERTGNVIDLGVRLFSAKNSEKNLERRTNR |
| | faecalis | | GGRRLIRRKTNRLKDAKKILAAVGFYEDKSLKNSCPYQLRVKGLTEP |
| | TX0012 | | LSRGEIYKVTLHILKKRGISYLDEVDTEAAKESQDYKEQVRKNAQLL |
| | | | TKYTPGQIQLQRLKENNRVKTGINAQGNYQLNVFKVSAYANELATIL |
| | | | KTQQAFYPNELTDDWIALFVQPGIAEEAGLIYRKRPYYHGPGNEANN |
| | | | SPYGRWSDFQKTGEPATNIFDKLIGKDFQGELRASGLSLSAQQYNLL |
| | | | NDLTNLKIDGEVPLSSEQKEYILTELMTKEFTRFGVNDVVKLLGVKK |
| | | | ERLSGWRLDKKGKPEIHTLKGYRNWRKIFAEAGIDLATLPTETIDCL |
| | | | AKVLTLNTEREGIENTLAFELPELSESVKLLVLDRYKELSQSISTQSW |
| | | | HRFSLKTLHLLIPELMNATSEQNTLLEQFQLKSDVRKRYSEYKKLPT |
| | | | KDVLAEIYNPTVNKTVSQAFKVIDALLVKYGKEQIRYITIEMPRDDN |
| | | | EEDEKKRIKELHAKNSQRKNDSQSYFMQKSGWSQEKFQTTIQKNRR |
| | | | FLAKLLYYYEQDGICAYTGLPISPELLVSDSTEIDHIIPISISLDDSINNK |
| | | | VLVLSKANQVKGQQTPYDAWMDGSFKKINGKFSNWDDYQKWVES |
| | | | RHFSHKKENNLLETRNIFDSEQVEKFLARNLNDTRYASRLVLNTLQS |
| | | | FFTNQETKVRVVNGSFTHTLRKKWGADLDKTRETHHHHAVDATLC |
| | | | AVTSFVKVSRYHYAVKEETGEKVMREIDFETGEIVNEMSYWEFKKS |
| | | | KKYERKTYQVKWPNFREQLKPVNLHPRIKFSHQVDRKANRKLSDAT |
| | | | IYSVREKTEVKTLKSGKQKITTDEYTIGKIKDIYTLDGWEAFKKKQD |
| | | | KLLMKDLDEKTYERLLSIAETTPDFQEVEEKNGKVKRVKRSPFAVYC |
| | | | EENDIPAIQKYAKKNNGPLIRSLKYYDGKLNKHINITKDSQGRPVEKT |
| | | | KNGRKVTLQSLKPYRYDIYQDLETKAYYTVQLYYSDLRFVEGKYGI |
| | | | TEKEYMKKVAEQTKGQVVRFCFSLQKNDGLEIEWKDSQRYDVRFY |
| | | | NFQSANSINFKGLEQEMMPAENQFKQKPYNNGAINLNIAKYGKEGK |
| | | | KLRKFNTDILGKKHYLFYEKEPKNIIK (SEQ ID NO 9) |
| EIE39736.1 | Mycoplasma | Cas9 | MEKKRKVTLGFDLGIASVGWAIVDSETNQVYKLGSRLFDAPDTNLE |
| | canis PG | | RRTQRGTRRLLRRRKYRNQKFYNLVKRTEVFGLSSREAIENRFRELSI |
| | 14 | | KYPNIIELKTKALSQEVCPDEIAWILHDYLKNRGYFYDEKETKEDFD |
| | | | QQTVESMPSYKLNEFYKKYGYFKGALSQPTESEMKDNKDLKEAFFF |
| | | | DFSNKEWLKEINYFFNVQKNILSETFIEEFKKIFSFTRDISKGPGSDNM |
| | | | PSPYGIFGEFGDNGQGGRYEHIWDKNIGKCSIFTNEQRAPKYLPSALI |
| | | | FNFLNELANIRLYSTDKKNIQPLWKLSSVDKLNILLNLFNLPISEKKK |
| | | | KLTSTNINDIVKKESIKSIMISVEDIDMIKDEWAGKEPNVYGVGLSGL |
| | | | NIEESAKENKFKFQDLKILNVLINLLDNVGIKFEFKDRNDIIKNLELLD |
| | | | NLYLFLIYQKESNNKDSSIDLFIAKNESLNIENLKLKLKEFLLGAGNEF |
| | | | ENHNSKTHSLSKKAIDEILPKLLDNNEGWNLEAIKNYDEEIKSQIEDN |
| | | | SSLMAKQDKKYLNDNFLKDAILPPNVKVTFQQAILIFNKIIQKFSKDF |
| | | | EIDKVVIELAREMTQDQENDALKGIAKAQKSKKSLVEERLEANNIDK |
| | | | SVENDKYEKLIYKIFLWISQDFKDPYTGAQISVNEIVNNKVEIDHIIPY |
| | | | SLCFDDSSANKVLVHKQSNQEKSNSLPYEYIKQGHSGWNWDEFTKY |
| | | | VKRVFVNNVDSILSKKERLKKSENLLTASYDGYDKLGFLARNLNDT |
| | | | RYATILFRDQLNNYAEHHLIDNKKMFKVIAMNGAVTSFIRKNMSYD |
| | | | NKLRLKDRSDFSHHAYDAAIIALFSNKTKTLYNLIDPSLNGIISKRSEG |
| | | | YWVIEDRYTGEIKELKKEDWTSIKNNVQARKIAKEIEEYLIDLDDEVF |
| | | | FSRKTKRKTNRQLYNETIYGIATKTDEDGITNYYKKEKFSILDDKDIY |
| | | | LRLLREREKFVINQSNPEVIDQIIEIIESYGKENNIPSRDEAINIKYTKNK |
| | | | INYNLYLKQYMRSLTKSLDQFSEEFINQMIANKTFVLYNPTKNTTRKI |
| | | | KFLRLVNDVKINDIRKNQVINKFNGKNNEPKAFYENINSLGAIVFKNS |
| | | | ANNFKTLSINTQIAIFGDKNWDIEDFKTYNMEKIEKYKEIYGIDKTYN |
| | | | FHSFIFPGTILLDKQNKEFYYISSIQTVRDIIEIKFLNKIEFKDENKNQDT |
| | | | SKTPKRLMFGIKSIMNNYEQVDISPFGINKKIFE (SEQ ID NO 10) |
| RLP87161.1 | Geobacillus | Cas9 | MRYKIGLDIGITSVGWAVMNLDIPRIEDLGVRIFDRAENPQTGESLAL |
| | stearo- | | PRRLARSARRRLRRRKHRLERIRRLVIREGILTKEELDKLFEEKHEIDV |
| | thermophilus | | WQLRVEALDRKLNNDELARVLLHLAKRRGFKSNRKSERSNKENST |
| | Cas9 | | MLKHIEENRAILSSYRTVGEMIVKDPKFALHKRNKGENYTNTIARDD |
| | | | LEREIRLIFSKQREFGNMSCTEEFENEYIAIWASQRPVASKDDIEKKV |
| | | | GFCAFEPKEKRAPKATYTFQSFIAWEHINKLRLISPSGARGLTDEERR |
| | | | LLYEQAFQKNKITYHDIRTLLHLPDDTYFKGIVYDRGESRKQNENIRF |
| | | | LELDAYHQIRKAVDKVYGKEKSSSFLPIDFDTFGYALTLFKDDADIHS |
| | | | YLRNEYEQNGKRMPNLANKVYDNELIEELLNLSFTKFGHLSLKALRS |
| | | | ILPYMEQGEVYSSACERAGYTFTGPKKKQKTMLLPNIPPIANPVVMR |
| | | | ALTQARKVVNAIIKKYGSPVSIHIELARDLSQTFDERRKTKKEQDENR |
| | | | KKNETAIRQLMEYGLTLNPTGHDIVKFKLWSEQNGRCAYSLQPIEIE |
| | | | RLLEPGYVEVDHVIPYSRSLDDSYTNKVLVLTRENREKGNRIPAEYL |
| | | | GVGTERWQQFETFVLTNKQFSKKKRDRLLRLHYDENEETEFKNRNL |
| | | | NDTRYISRFFANFIREHLKFAESDDKQKVYTVNGRVTAHLRSRWEFN |
| | | | KNREESDLHHAVDAVIVACTTPSDIAKVTAFYQRREQNKELAKKTEP |
| | | | HFPQPWPHFADELRARLSKHPKESIKALNLGNYDDQKLESLQPVFVS |
| | | | RMPKRSVTGAAHQETLRRYVGIDERSGKIQTVVKTKLSEIKLDASGH |
| | | | FPMYGKESDPRTYEAIRQRLLEHNNDPKKAFQEPLYKPKKNGEPGPV |
| | | | IRTVKIIDTKNQVIPLNDGKTVAYNSNIVRVDVFEKDGKYYCVPVYT |
| | | | MDIMKGILPNKAIEPNKPYSEWKEMTEDYTFRFSLYPNDLIRIELPRE |
| | | | KTVKTAAGEEINVKDVFVYYKTIDSANGGLELISHDHRFSLRGVGSR |
| | | | TLKRFEKYQVDVLGNIYKVRGEKRVGLASSAHSKPGKTIRPLQSTRD |
| | | | (SEQ ID NO 11) |
| J7RUA5.1 | Staphylococcus | Cas9 | MKRNYILGLDIGITSVGYGIIDYETRDVIDAGVRLFKEANVENNEGRR |
| | aureus | | SKRGARRLKRRRRHRIQRVKKLLFDYNLLTDHSELSGINPYEARVKG |
| | | | LSQKLSEEEFSAALLHLAKRRGVHNVNEVEEDTGNELSTKEQISRNS |
| | | | KALEEKYVAELQLERLKKDGEVRGSINRFKTSDYVKEAKQLLKVQK |
| | | | AYHQLDQSFIDTYIDLLETRRTYYEGPGEGSPFGWKDIKEWYEMLM |
| | | | GHCTYFPEELRSVKYAYNADLYNALNDLNNLVITRDENEKLEYYEK |
| | | | FQIIENVFKQKKKPTLKQIAKEILVNEEDIKGYRVTSTGKPEFTNLKV |
| | | | YHDIKDITARKEIIENAELLDQIAKILTIYQSSEDIQEELTNLNSELTQE |
| | | | EIEQISNLKGYTGTHNLSLKAINLILDELWHTNDNQIAIFNRLKLVPK |
| | | | KVDLSQQKEIPTTLVDDFILSPVVKRSFIQSIKVINAIIKKYGLPNDIIIE |
| | | | LAREKNSKDAQKMINEMQKRNRQTNERIEEIIRTTGKENAKYLIEKIK |
| | | | LHDMQEGKCLYSLEAIPLEDLLNNPFNYEVDHIIPRSVSFDNSFNNKV |
| | | | LVKQEENSKKGNRTPFQYLSSSDSKISYETFKKHILNLAKGKGRISKT |
| | | | KKEYLLEERDINRFSVQKDFINRNLVDTRYATRGLMNLLRSYFRVNN |
| | | | LDVKVKSINGGFTSFLRRKWKFKKERNKGYKHHAEDALIIANADFIF |
| | | | KEWKKLDKAKKVMENQMFEEKQAESMPEIETEQEYKEIFITPHQIKH |
| | | | IKDFKDYKYSHRVDKKPNRELINDTLYSTRKDDKGNTLIVNNLNGLY |
| | | | DKDNDKLKKLINKSPEKLLMYHHDPQTYQKLKLIMEQYGDEKNPLY |
| | | | KYYEETGNYLTKYSKKDNGPVIKKIKYYGNKLNAHLDITDDYPNSR |
| | | | NKVVKLSLKPYRFDVYLDNGVYKFVTVKNLDVIKKENYYEVNSKC |
| | | | YEEAKKLKKISNQAEFIASFYNNDLIKINGELYRVIGVNNDLLNRIEV |
| | | | NMIDITYREYLENMNDKRPPRIIKTIASKTQSIKKYSTDILGNLYEVKS |
| | | | KKHPQIIKKG (SEQ ID NO 12) |
| NP_246064.1 | Pasteurella | Cas9 | MQTTNLSYILGLDLGIASVGWAVVEINENEDPIGLIDVGVRIFERAEV |
| | multocida | | PKTGESLALSRRLARSTRRLIRRRAHRLLLAKRFLKREGILSTIDLEKG |
| | subsp. | | LPNQAWELRVAGLERRLSAIEWGAVLLHLIKHRGYLSKRKNESQTN |
| | multocida | | NKELGALLSGVAQNHQLLQSDDYRTPAELALKKFAKEEGHIRNQRG |
| | str. Pm70 | | AYTHTFNRLDLLAELNLLFAQQHQFGNPHCKEHIQQYMTELLMWQ |
| | | | KPALSGEAILKMLGKCTHEKNEFKAAKHTYSAERFVWLTKLNNLRI |
| | | | LEDGAERALNEEERQLLINHPYEKSKLTYAQVRKLLGLSEQAIFKHL |
| | | | RYSKENAESATFMELKAWHAIRKALENQGLKDTWQDLAKKPDLLD |
| | | | EIGTAFSLYKTDEDIQQYLTNKVPNSVINALLVSLNFDKFIELSLKSLR |
| | | | KILPLMEQGKRYDQACREIYGHHYGEANQKTSQLLPAIPAQEIRNPV |
| | | | VLRTLSQARKVINAIIRQYGSPARVHIETGRELGKSFKERREIQKQQE |
| | | | DNRTKRESAVQKFKELFSDFSSEPKSKDILKFRLYEQQHGKCLYSGK |
| | | | EINIHRLNEKGYVEIDHALPFSRTWDDSFNNKVLVLASENQNKGNQT |
| | | | PYEWLQGKINSERWKNFVALVLGSQCSAAKKQRLLTQVIDDNKFID |
| | | | RNLNDTRYIARFLSNYIQENLLLVGKNKKNVFTPNGQITALLRSRWG |
| | | | LIKARENNNRHHALDAIVVACATPSMQQKITRFIRFKEVHPYKIENRY |
| | | | EMVDQESGEIISPHFPEPWAYFRQEVNIRVFDNHPDTVLKEMLPDRP |
| | | | QANHQFVQPLFVSRAPTRKMSGQGHMETIKSAKRLAEGISVLRIPLT |
| | | | QLKPNLLENMVNKEREPALYAGLKARLAEFNQDPAKAFATPFYKQG |
| | | | GQQVKAIRVEQVQKSGVLVRENNGVADNASIVRTDVFIKNNKFFLV |
| | | | PIYTWQVAKGILPNKAIVAHKNEDEWEEMDEGAKFKFSLFPNDLVE |
| | | | LKTKKEYFFGYYIGLDRATGNISLKEHDGEISKGKDGVYRVGVKLAL |
| | | | SFEKYQVDELGKNRQICRPQQRQPVR (SEQ ID NO 13) |
| NP_721764.1 | Streptococcus | Cas9 | MKKPYSIGLDIGTNSVGWAVVTDDYKVPAKKMKVLGNTDKSHIEK |
| | mutans | | NLLGALLFDSGNTAEDRRLKRTARRRYTRRRNRILYLQEIFSEEMGK |
| | UA159 | | VDDSFFHRLEDSFLVTEDKRGERHPIFGNLEEEVKYHENFPTIYHLRQ |
| | | | YLADNPEKVDLRLVYLALAHIIKFRGHFLIEGKFDTRNNDVQRLFQE |
| | | | FLAVYDNTFENSSLQEQNVQVEEILTDKISKSAKKDRVLKLFPNEKS |
| | | | NGRFAEFLKLIVGNQADFKKHFELEEKAPLQFSKDTYEEELEVLLAQI |
| | | | GDNYAELFLSAKKLYDSILLSGILTVTDVGTKAPLSASMIQRYNEHQ |
| | | | MDLAQLKQFIRQKLSDKYNEVFSDVSKDGYAGYIDGKTNQEAFYKY |
| | | | LKGLLNKIEGSGYFLDKIEREDFLRKQRTFDNGSIPHQIHLQEMRAIIR |
| | | | RQAEFYPFLADNQDRIEKLLTFRIPYYVGPLARGKSDFAWLSRKSAD |
| | | | KITPWNFDEIVDKESSAEAFINRMTNYDLYLPNQKVLPKHSLLYEKF |
| | | | TVYNELTKVKYKTEQGKTAFFDANMKQEIFDGVFKVYRKVTKDKL |
| | | | MDFLEKEFDEFRIVDLTGLDKENKVFNASYGTYHDLCKILDKDFLDN |
| | | | SKNEKILEDIVLTLTLFEDREMIRKRLENYSDLLTKEQVKKLERRHYT |
| | | | GWGRLSAELIHGIRNKESRKTILDYLIDDGNSNRNFMQLINDDALSFK |
| | | | EEIAKAQVIGETDNLNQVVSDIAGSPAIKKGILQSLKIVDELVKIMGH |
| | | | QPENIVVEMARENQFTNQGRRNSQQRLKGLTDSIKEFGSQILKEHPV |
| | | | ENSQLQNDRLFLYYLQNGRDMYTGEELDIDYLSQYDIDHIIPQAFIKD |
| | | | NSIDNRVLTSSKENRGKSDDVPSKDVVRKMKSYWSKLLSAKLITQR |
| | | | KFDNLTKAERGGLTDDDKAGFIKRQLVETRQITKHVARILDERENTE |
| | | | TDENNKKIRQVKIVTLKSNLVSNFRKEFELYKVREINDYHHAHDAYL |
| | | | NAVIGKALLGVYPQLEPEFVYGDYPHFHGHKENKATAKKFFYSNIM |
| | | | NFFKKDDVRTDKNGEIIWKKDEHISNIKKVLSYPQVNIVKKVEEQTG |
| | | | GFSKESILPKGNSDKLIPRKTKKFYWDTKKYGGFDSPIVAYSILVIADI |
| | | | EKGKSKKLKTVKALVGVTIMEKMTFERDPVAFLERKGYRNVQEENII |
| | | | KLPKYSLFKLENGRKRLLASARELQKGNEIVLPNHLGTLLYHAKNIH |
| | | | KVDEPKHLDYVDKHKDEFKELLDVVSNFSKKYTLAEGNLEKIKELY |
| | | | AQNNGEDLKELASSFINLLTFTAIGAPATFKFFDKNIDRKRYTSTTEIL |
| | | | NATLIHQSITGLYETRIDLNKLGGD (SEQ ID NO 14) |
| WP_ | Streptococcus | Cas9 | MNKPYSIGLDIGTNSVGWSIITDDYKVPAKKMRVLGNTDKEYIKKNL |
| 001040103.1 | agalactiae | | IGALLFDGGNTAADRRLKRTARRRYTRRRNRILYLQEIFAEEMSKVD |
| | NEM316 | | DSFFHRLEDSFLVEEDKRGSKYPIFATMQEEKYYHEKFPTIYHLRKEL |
| | | | ADKKEKADLRLVYLALAHIIKFRGHFLIEDDRFDVRNTDIQKQYQAF |
| | | | LEIFDTTFENNHLLSQNVDVEAILTDKISKSAKKDRILAQYPNQKSTGI |
| | | | FAEFLKLIVGNQADFKKHFNLEDKTPLQFAKDSYDEDLENLLGQIGD |
| | | | EFADLFSVAKKLYDSVLLSGILTVTDLSTKAPLSASMIQRYDEHHEDL |
| | | | KHLKQFVKASLPENYREVFADSSKDGYAGYIEGKTNQEAFYKYLLK |
| | | | LLTKQEGSEYFLEKIKNEDFLRKQRTFDNGSIPHQVHLTELRAIIRRQS |
| | | | EYYPFLKENQDRIEKILTFRIPYYVGPLAREKSDFAWMTRKTDDSIRP |
| | | | WNFEDLVDKEKSAEAFIHRMTNNDLYLPEEKVLPKHSLIYEKFTVYN |
| | | | ELTKVRFLAEGFKDFQFLNRKQKETIFNSLFKEKRKVTEKDIISFLNK |
| | | | VDGYEGIAIKGIEKQFNASLSTYHDLKKILGKDFLDNTDNELILEDIV |
| | | | QTLTLFEDREMIKKCLDIYKDFFTESQLKKLYRRHYTGWGRLSAKLI |
| | | | NGIRNKENQKTILDYLIDDGSANRNFMQLINDDDLSFKPIIDKARTGS |
| | | | HSDNLKEVVGELAGSPAIKKGILQSLKIVDELVKVMGYEPEQIVVEM |
| | | | ARENQTTAKGLSRSRQRLTTLRESLANLKSNILEEKKPKYVKDQVEN |
| | | | HHLSDDRLFLYYLQNGRDMYTKKALDIDNLSQYDIDHIIPQAFIKDD |
| | | | SIDNRVLVSSAKNRGKSDDVPSIEIVKARKMFWKNLLDAKLMSQRK |
| | | | YDNLTKAERGGLTSDDKARFIQRQLVETRQITKHVARILDERFNNEV |
| | | | DNGKKICKVKIVTLKSNLVSNFRKEFGFYKIREVNDYHHAHDAYLN |
| | | | AVVAKAILTKYPQLEPEFVYGMYRQKKLSKIVHEDKEEKYSEATRK |
| | | | MFFYSNLMNMFKRVVRLADGSIVVRPVIETGRYMRKTAWDKKKHF |
| | | | ATVRKVLSYPQNNIVKKTEIQTGGFSKESILAHGNSDKLIPRKTKDIY |
| | | | LDPKKYGGFDSPIVAYSVLVVADIKKGKAQKLKTVTELLGITIMERS |
| | | | RFEKNPSAFLESKGYLNIRDDKLMILPKYSLFELENGRRRLLASAGEL |
| | | | QKGNELALPTQFMKFLYLASRYNESKGKPEEIEKKQEFVNQHVSYFD |
| | | | DILQLINDFSKRVILADANLEKINKLYQDNKENIPVDELANNIINLFTF |
| | | | TSLGAPAAFKFFDKIVDRKRYTSTKEVLNSTLIHQSITGLYETRIDLGK |
| | | | LGED (SEQ ID NO 15) |
| WP_ | Streptococcus | Cas9 | MTNGKILGLDIGIASVGVGIIEAKTGKVVHANSRLFSAANAENNAER |
| 013852048.1 | pasteurianus | | RGFRGSRRLNRRKKHRVKRVRDLFEKYGIVTDFRNLNLNPYELRVK |
| | | | GLTEQLKNEELFAALRTISKRRGISYLDDAEDDSTGSTDYAKSIDENR |
| | | | RLLKNKTPGQIQLERLEKYGQLRGNFTVYDENGEAHRLINVFSTSDY |
| | | | EKEARKILETQADYNKKITAEFIDDYVEILTQKRKYYHGPGNEKSRT |
| | | | DYGRFRTDGTTLENIFGILIGKCNFYPDEYRASKASYTAQEYNFLNDL |
| | | | NNLKVSTETGKLSTEQKESLVEFAKNTATLGPAKLLKEIAKILDCKV |
| | | | DEIKGYREDDKGKPDLHTFEPYRKLKFNLESINIDDLSREVIDKLADIL |
| | | | TLNTEREGIEDAIKRNLPNQFTEEQISEIIKVRKSQSTAFNKGWHSFSA |
| | | | KLMNELIPELYATSDEQMTILTRLEKFKVNKKSSKNTKTIDEKEVTDE |
| | | | IYNPVVAKSVRQTIKIINAAVKKYGDFDKIVIEMPRDKNADDEKKFID |
| | | | KRNKENKKEKDDALKRAAYLYNSSDKLPDEVFHGNKQLETKIRLW |
| | | | YQQGERCLYSGKPISIQELVHNSNNFEIDHILPLSLSFDDSLANKVLVY |
| | | | AWTNQEKGQKTPYQVIDSMDAAWSFREMKDYVLKQKGLGKKKRD |
| | | | YLLTTENIDKIEVKKKFIERNLVDTRYASRVVLNSLQSALRELGKDTK |
| | | | VSVVRGQFTSQLRRKWKIDKSRETYHHHAVDALIIAASSQLKLWEK |
| | | | QDNPMFVDYGKNQVVDKQTGEILSVSDDEYKELVFQPPYQGFVNTI |
| | | | SSKGFEDEILFSYQVDSKYNRKVSDATIYSTRKAKIGKDKKEETYVL |
| | | | GKIKDIYSQNGFDTFIKKYNKDKTQFLMYQKDSLTWENVIEVILRDY |
| | | | PTTKKSEDGKNDVKCNPFEEYRRENGLICKYSKKGKGTPIKSLKYYD |
| | | | KKLGNCIDITPEESRNKVILQSINPWRADVYFNPETLKYELMGLKYSD |
| | | | LSFEKGTGNYHISQEKYDAIKEKEGIGKKSEFKFTLYRNDLILIKDIAS |
| | | | GEQEIYRFLSRTMPNVNHYVELKPYDKEKFDNVQELVEALGEADKV |
| | | | GRCIKGLNKPNISIYKVRTDVLGNKYFVKKKGDKPKLDFKNNKK |
| | | | (SEQ ID NO 16) |
| WP_ | Neisseria | Cas9 | MAAFKPNPMNYILGLDIGIASVGWAMVEVDEEENPIRLIDLGVRVFE |
| 036475267.1 | lactamica] | | RAEVPKTGDSLAMARRLARSVRRLTRRRAHRLLRARRLLKREGVLQ |
| | | | DADFDENGLVKSLPNTPWQLRAAALDRKLTCLEWSAVLLHLVKHR |
| | | | GYLSQRKNEGETADKELGALLKGVADNAHALQTGDFRTPAELALN |
| | | | KFEKESGHIRNQRGDYSHTFSRKDLQAELNLLFEKQKEFGNPHVSDG |
| | | | LKEDIETLLMAQRPALSGDAVQKMLGHCTFEPAEPKAAKNTYTAER |
| | | | FIWLTKLNNLRILEQGSERPLTDTERATLMDEPYRKSKLTYAQARKL |
| | | | LGLEDTAFFKGLRYGKDNAEASTLMEMKAYHAISRALEKEGLKDKK |
| | | | SPLNLSTELQDEIGTAFSLFKTDKDITGRLKDRVQPEILEALLKHISFD |
| | | | KFVQISLKALRRIVPLMEQGKRYDEACAEIYGDHYCKKNAEEKIYLP |
| | | | PIPADEIRNPVVLRALSQARKVINCVVRRYGSPARIHIETAREVGKSF |
| | | | KDRKEIEKRQEENRKDREKAAAKFREYFPNFVGEPKSKDILKLRLYE |
| | | | QQHGKCLYSGKEINLVRLNEKGYVEIDHALPFSRTWDDSFNNKVLV |
| | | | LGSENQNKGNQTPYEYFNGKDNSREWQEFKARVETSRFPRSKKQRI |
| | | | LLQKFDEEGFKERNLNDTRYVNRFLCQFVADHILLTGKGKRRVFAS |
| | | | NGQITNLLRGFWGLRKVRTENDRHHALDAVVVACSTVAMQQKITR |
| | | | FVRYKEMNAFDGKTIDKETGEVLHQKAHFPQPWEFFAQEVMIRVFG |
| | | | KPDGKPEFEEADTPEKLRTLLAEKLSSRPEAVHEYVTPLFVSRAPNRK |
| | | | MSGQGHMETVKSAKRLDEGISVLRVPLTQLKLKGLEKMVNREREPK |
| | | | LYDALKAQLETHKDDPAKAFAEPFYKYDKAGSRTQQVKAVRIEQV |
| | | | QKTGVWVRNHNGIADNATMVRVDVFEKGGKYYLVPIYSWQVAKGI |
| | | | LPDRAVVAFKDEEDWTVMDDSFEFRFVLYANDLIKLTAKKNEFLGY |
| | | | FVSLNRATGAIDIRTHDTDSTKGKNGIFQSVGVKTALSFQKNQIDELG |
| | | | KEIRPCRLKKRPPVR (SEQ ID NO 17) |
| WP_ | Francisella | Cas9 | MNVKILPIAIDLDVKNTGVFSAFYQKGTSLEKLDNKNGKVYELSKDS |
| 003026687.1 | tularensis | | YTLLMNNRTARRHQRRGIDRKQLVKRLFKLVWTEQLNLEWDKDTQ |
| | subsp. | | QAISFLFNRRGFSFITDGYSTEYLNIVPEQVKAILMDIFDDYNGEDDL |
| | tularensis | | DSYLKLATEQESKISEIYNKLMQKILEFKLRKLCTDIKDDKVSTKTLK |
| | WY96-3418 | | EITSYEFELLADYLANYSESLKIQKFSYTDKQGNLKELSYYHHDKYNI |
| | | | QEFLKRHATINDEILDTLLTDDFDIWNFNFEKFDFDKNEEKLQNQED |
| | | | KDHTQAHLHHFVFAVNKIKSEMASGGRHRSQYFQEITNVLDENNHQ |
| | | | EGYLKNFCENLHNKKYSNLSVKNLVNLVGNLSNLELKPLRKYFNDK |
| | | | IHAKADHWDEQKFTETYCHWILGEWRVGVKDQDKKDGAKYSYKD |
| | | | LCNELKQKVTKAGLIDFLLELDPCRTIPPYLDNNNRKPPKCQSLILNP |
| | | | KFLDNQYPNWQQYLQELKKLQSIQDYLDSFETDLKDLKSSKDQPYF |
| | | | VEYKSSNQQMASGQRDYKDLDARILQFIFDRVKASDELLLNEIYFQA |
| | | | KKLKQKASSELEKLESSKKLDEVIANSQLSQILKSQHTNGIFEQGTFL |
| | | | HLVCKYYKQRQRARDSRLYIMPEYRYDKKLDKYNNTGRFDDNNQL |
| | | | LTYCNHKPRQKRYQLLNDLAGVLQVSRNQLLSSVEEWFQQAQRVG |
| | | | EISKSQDEQIFEWLKSFKIASYCKAAVEMQKQYRGTLKNAIQTAIFRQ |
| | | | SENINKNKNKNTGNQQQALSENSKDVKSLTADEKKLLKLIENIAKAS |
| | | | QKIGESLGLNDKQIKKFNSIYSFAQIQQIAFAERKGNANTCAVCSADN |
| | | | AHRMQQIKITELVEDNKDNIILSAKAQRLPAIPTRIVDGAVKKMATIL |
| | | | AKNIVDDNWQNIKQVLSAKHQLHIPIITESNAFEFEPALADVKGKSLK |
| | | | DRRKKALERISPENIFKDKNNRIKEFAKGISAYSGANLTDGDFDGAKE |
| | | | ELDHIIPRSHKKYGTLNDEANLICVTRDDNKNIFAIDTSKWFEIETPSD |
| | | | LRDIGVATIQYKIDNNSRPKVRVKLDYVIDDDSKINYFMNHSLLKSR |
| | | | YPDKVLEILKQSTIIEFESSGFNKTIKEMLGMTLAGIYNETSNN (SEQ |
| | | | ID NO 18) |
| WP_ | Bradyrhizobium | Cas9 | MKRTSLRAYRLGVDLGANSLGWFVVWLDDHGQPEGLGPGGVRIFP |
| 012044026.1 | sp. | | DGRNPQSKQSNAAGRRLARSARRRRDRYLQRRGKLMGLLVKHGLM |
| | BTAi1 | | PADEPARKRLECLDPYGLRAKALDEVLPLHHVGRALFHLNQRRGLF |
| | | | ANRAIEQGDKDASAIKAAAGRLQTSMQACGARTLGEFLNRRHQLRA |
| | | | TVRARSPVGGDVQARYEFYPTRAMVDAEFEAIWAAQAPHHPTMTA |
| | | | EAHDTIREAIFSQRAMKRPSIGKCSLDPATSQDDVDGFRCAWSHPLA |
| | | | QRFRIWQDVRNLAVVETGPTSSRLGKEDQDKVARALLQTDQLSFDEI |
| | | | RGLLGLPSDARFNLESDRRDHLKGDATGAILSARRHFGPAWHDRSL |
| | | | DRQIDIVALLESALDEAAIIASLGTTHSLDEAAAQRALSALLPDGYCR |
| | | | LGLRAIKRVLPLMEAGRTYAEAASAAGYDHALLPGGKLSPTGYLPY |
| | | | YGQWLQNDVVGSDDERDTNERRWGRLPNPTVHIGIGQLRRVVNELI |
| | | | RWHGPPAEITVELTRDLKLSPRRLAELEREQAENQRKNDKRTSLLRK |
| | | | LGLPASTHNLLKLRLWDEQGDVASECPYTGEAIGLERLVSDDVDIDH |
| | | | LIPFSISWDDSAANKVVCMRYANREKGNRTPFEAFGHRQGRPYDWA |
| | | | DIAERAARLPRGKRWRFGPGARAQFEELGDFQARLLNETSWLARVA |
| | | | KQYLAAVTHPHRIHVLPGRLTALLRATWELNDLLPGSDDRAAKSRK |
| | | | DHRHHAIDALVAALTDQALLRRMANAHDDTRRKIEVLLPWPTFRID |
| | | | LETRLKAMLVSHKPDHGLQARLHEDTAYGTVEHPETEDGANLVYR |
| | | | KTFVDISEKEIDRIRDRRLRDLVRAHVAGERQQGKTLKAAVLSFAQR |
| | | | RDIAGHPNGIRHVRLTKSIKPDYLVPIRDKAGRIYKSYNAGENAFVDI |
| | | | LQAESGRWIARATTVFQANQANESHDAPAAQPIMRVFKGDMLRIDH |
| | | | AGAEKFVKIVRLSPSNNLLYLVEHHQAGVFQTRHDDPEDSFRWLFAS |
| | | | FDKLREWNAELVRIDTLGQPWRRKRGLETGSEDATRIGWTRPKKWP |
| | | | (SEQ ID NO 19) |
| WP_ | Streptococcus | Cas9 | MNGLVLGLDIGIASVGVGILEKDTGKIIHASSRLFPAATADNNVERRS |
| 012130469.1 | gordonii | | NRQGRRLNRRKKHRSVRLQDLFEGYGLLTDFSKVSMNLNPYQLRVQ |
| | str. | | GMENQLTNEELFVALKNIVKRRGISYLDDASEDGGTVSSDYGKAVE |
| | Challis | | ENRKLLAEKTPGQIQLERFEKYGQLRGDFTVEENGEKHRLINVFSTS |
| | substr. | | AYRKEAERILRKQQEFNSKITDEFIEDYLIILTGKRKYYHGPGNEKSR |
| | CH1 | | TDYGRFRTDGTTLDNIFGILIGKCTFYTEEYRASKASYTAQEFNLLND |
| | | | LNNLTVPTETKKLSEEQKKLIIEYAKSAKTLGASTLLKYIAKMIDASV |
| | | | DQIRGYRVDVNNKPEMHTFEVYRKMQSLETIKVEELPRKVLDELAHI |
| | | | LTLNTEREGIEEAINSKLKDIFNRDQVLELVQFRKNNSSLFSKGWHNF |
| | | | SIKLMMELIPELYETSEEQMTILTRLGKQRSKETSKRTKYIDEKELTEE |
| | | | IYNPVVAKSVRQAIKIINEATKKYGIFDNIVIEMARENNEEDAKKDYI |
| | | | KRQKANQDEKNAAMEKAAFQYNGKKELPDNIFHGHKELTTKIRLW |
| | | | HQQGEKCLYTGKNIPISDLIHNQYKYEIDHILPLSLSFDDSLSNKVLVL |
| | | | ATANQEKGQRTPFQALDSMDDAWSYREFKSYVKDSKLLSNKKKDY |
| | | | LLTEEDISKIEVKQKFIERNLVDTRYSSRVVLNALQDFYKSHQLDTTIS |
| | | | VVRGQFTSQLRRKWGIEKSRETYHHHAVDALIIAASSQLRLWKKHS |
| | | | NPLIAYKEGQFVDSETGEIVSLSDEEYKELVFKAPYDHFVDTLRSKKF |
| | | | EDSILFSYQVDSKYNRKISDATIYATRKAKLDKEKKEYTYTLGKIKDI |
| | | | YALGTKTPSKTGFYKFLDLYKTDKSQFLMYQKDRKTWDEVIEKIIEQ |
| | | | YRPFKEYDKNGKEVDFNPFEKYRIGNGPIRKYSKKGNGPEIKSLKYY |
| | | | DILLGKHKNITPDGSRNTVALLSLNPWRTDVYYNSETKKYEFLGLKY |
| | | | ADLCFEEGGAYGISEVKYKKIREKEGIGKNSEFKFTLYKNDLILIKDT |
| | | | ETNCQQFFRFWSRTGKDNPKSFEKHKIELKPYEKAKFEKGEELKVLG |
| | | | KVPPSSNQFQKNMQIENLSIYKVKTDILGNKHFIKKEGDEPKLKFKK |
| | | | (SEQ ID NO 20) |
| WP_ | Campylobacter | Cas9 | MARILAFDIGISSIGWAFSENDELKDCGVRIFTKAENPKTGESLALPRR |
| 012006786.1 | jejuni | | LARSARKRLARRKARLNHLKHLIANEFKLNYEDYQSFDESLAKAYK |
| | subsp. | | GSLISPYELRFRALNELLSKQDFARVILHIAKRRGYDDIKNNGDEEKS |
| | jejuni | | EILKAIKQNEEKLVNYQSVGEYLYKEYFQKFKENSKEFINVRNKKES |
| | 81116 | | YERCITQSFLKDELKLIFQKQREFGFSFSKKFEEEVLSVAFYKRALKD |
| | | | FSHLVGNCSFFTDEKRAPKNSPLAFMFVALTRIINILNNLKNTEGILYT |
| | | | KDDLNALLNEVLKNGTLTYKQTKKLLGLSDDYEFKGEKGTYFIEFK |
| | | | KYKEFIKALGEHNLSQDDLNEIAKDITLIKDEIKLKKALAKYDLNQN |
| | | | QIDSLSKLEFKDHLNISFKALKLITPLMLEGKKYDEACNELNLKVAIN |
| | | | EDKKDFLPAFNETYYKDEVTNPVVLRAIKEYRKILNALLKKYGKVH |
| | | | KINIELAREVGKNHSQRAKIEKEQNENYKAKKDAEIECEKLGLKINS |
| | | | KNILKLRLFKEQKEFCAYSGEKIKLSDLQDEKMLEIDHIYPYSRSFDD |
| | | | SYMNKVLVFTKQNQEKLNKTPFEAFGNDSTKWQKIEVLAKNLPEKK |
| | | | QKRILDKNYKDKEQKDFKDRNLNDTRYIARLVLNYTKDYLDFLPLS |
| | | | DDENTKLNDTQKGSKVHVEAKSGMLTSALRHTWGFSAKDRNNHLH |
| | | | HAIDAAIIAYANNSIVKAFSDFKKEQESNSVELYAKKISELDYKNKRK |
| | | | FFEPFSGFRQKVLDKIDEIFVSKPERKKPSGALHEETFRKEEEFYQSYG |
| | | | GKEGVLKALELGKIRKVNGKIVKNGDMFRVDIFKHKKTNKFYAVPI |
| | | | YTMDFALKVLPNKAVARSKKGEIKDWILMDENYEFCFSLYKDSLILI |
| | | | QTKDMQEPELVYFNAFTSSTVSLIVSKHDNKFETLSKNQKILFKNAN |
| | | | EKEVIAKSIGIQNLKVFEKYIVSALGEVTKAEFRQREDFKK (SEQ ID |
| | | | NO 21) |
| WP_ | Dinoroseo- | Cas9 | MRLGLDIGTSSIGWWLYETDGAGSDARITGVVDGGVRIFSDGRDPKS |
| 012177079.1 | bacter | | GASLAVDRRAARAMRRRRDRYLRRRATLMKVLAETGLMPADPAEA |
| | shibae | | KALEALDPFALRAAGLDEPLPLPHLGRALFHLNQRRGFKSNRKTDRG |
| | | | DNESGKIKDATARLDMEMMANGARTYGEFLHKRRQKATDPRHVPS |
| | | | VRTRLSIANRGGPDGKEEAGYDFYPDRRHLEEEFHKLWAAQGAHHP |
| | | | ELTETLRDLLFEKIFFQRPLKEPEVGLCLFSGHHGVPPKDPRLPKAHP |
| | | | LTQRRVLYETVNQLRVTADGREARPLTREERDQVIHALDNKKPTKS |
| | | | LSSMVLKLPALAKVLKLRDGERFTLETGVRDAIACDPLRASPAHPDR |
| | | | FGPRWSILDADAQWEVISRIRRVQSDAEHAALVDWLTEAHGLDRAH |
| | | | AEATAHAPLPDGYGRLGLTATTRILYQLTADVVTYADAVKACGWH |
| | | | HSDGRTGECFDRLPYYGEVLERHVIPGSYHPDDDDITRFGRITNPTVH |
| | | | IGLNQLRRLVNRIIETHGKPHQIVVELARDLKKSEEQKRADIKRIRDT |
| | | | TEAAKKRSEKLEELEIEDNGRNRMLLRLWEDLNPDDAMRRFCPYTG |
| | | | TRISAAMIFDGSCDVDHILPYSRTLDDSFPNRTLCLREANRQKRNQTP |
| | | | WQAWGDTPHWHAIAANLKNLPENKRWRFAPDAMTRFEGENGFLD |
| | | | RALKDTQYLARISRSYLDTLFTKGGHVWVVPGRFTEMLRRHWGLNS |
| | | | LLSDAGRGAVKAKNRTDHRHHAIDAAVIAATDPGLLNRISRAAGQG |
| | | | EAAGQSAELIARDTPPPWEGFRDDLRVRLDRIIVSHRADHGRIDHAA |
| | | | RKQGRDSTAGQLHQETAYSIVDDIHVASRTDLLSLKPAQLLDEPGRS |
| | | | GQVRDPQLRKALRVATGGKTGKDFENALRYFASKPGPYQAIRRVRII |
| | | | KPLQAQARVPVPAQDPIKAYQGGSNHLFEIWRLPDGEIEAQVITSFEA |
| | | | HTLEGEKRPHPAAKRLLRVHKGDMVALERDGRRVVGHVQKMDIAN |
| | | | GLFIVPHNEANADTRNNDKSDPFKWIQIGARPAIASGIRRVSVDEIGR |
| | | | LRDGGTRPI (SEQ ID NO 22) |
| WP_ | Neisseria | Cas9 | MAAFKPNPINYILGLDIGIASVGWAMVEIDEDENPICLIDLGVRVFER |
| 012221298.1 | meningitidis | | AEVPKTGDSLAMARRLARSVRRLTRRRAHRLLRARRLLKREGVLQA |
| | 053442 | | ADFDENGLIKSLPNTPWQLRAAALDRKLTPLEWSAVLLHLIKHRGYL |
| | | | SQRKNEGETADKELGALLKGVADNAHALQTGDFRTPAELALNKFEK |
| | | | ESGHIRNQRGDYSHTFSRKDLQAELNLLFEKQKEFGNPHISDGLKEGI |
| | | | ETLLMTQRPALSGDAVQKMLGHCTFEPAEPKAAKNTYTAERFIWLT |
| | | | KLNNLRILEQGSERPLTDTERTTLIDEPYRKSKLTYAQARKLLELDNT |
| | | | AFFKGLRYGKDNAEASTLMEMKAYHAISRALEKEGLKDKKSPLNLS |
| | | | PELQDEIGTAFSLFKTDEDITGRLKDRVQPEILEALLKHISFDKFVQISL |
| | | | KALRRIVPLMEQGKRYDEACAEIYGDHYGKKNTEEKIDLPPIPADEIR |
| | | | NPVVLRALSQARKVINGVVRRYGSPARIHIETAREVGKSFKDRKEIEK |
| | | | RQEENRKDREKAATKFREYFPNFVGEPKSKDILKLRLYEQQHGKCL |
| | | | YSGKEINLGRLNEKGYVEIDHALPFSRTWDDSFNNKVLVLGSENQN |
| | | | KGNQTPYEYFNGKDNSREWQEFKARVETSRFPRSKKQRILLQKFDEE |
| | | | GFKERNLNDTRYVNRFLCQFVADHMLLTGKGKRRVFASNGQITNLL |
| | | | RGFWGLRKVRAENDRHHALDAVVVACSTVAMQQKITRFVRYKEM |
| | | | NAFDGKTIDKETGEVLHQKTHFPQPWEFFAQEVMIRVFGKPDGKPEF |
| | | | EEADTPEKLRTLLAEKLSSRPEAVHEYVTPLFVSRAPNRKMSGQGH |
| | | | METVKSAKRLDEGVSVLRVPLTQLKLKDLEKMVNREREPKLYEALK |
| | | | ARLEAHKDDPAKAFAEPFYKYDKAGNRTQQVKAVRVEQVQKTGV |
| | | | WVRNHNGIADNATMVRVDVFEKGDKYYLVPIYSWQVAKGILPDRA |
| | | | VVQGKDEEDWQLIDDSFNFKFSLHPNDLVEVITKKARMFGYFASCH |
| | | | RGTGNINIRIHDLDHKIGKNGILEGIGVKTALSFQKYQIDELGKEIRPC |
| | | | RLKKRPPVR (SEQ ID NO 23) |
| WP_ | Elusimicrobium | Cas9 | MQKNINTKQNHIYIKQAQKIKEKLGDKPYRIGLDLGVGSIGFAIVSME |
| 012414420.1 | minutum | | ENDGNVLLPKEIIMVGSRIFKASAGAADRKLSRGQRNNHRHTRERM |
| | Pei191 | | RYLWKVLAEQKLALPVPADLDRKENSSEGETSAKRFLGDVLQKDIY |
| | | | ELRVKSLDERLSLQELGYVLYHIAGHRGSSAIRTFENDSEEAQKENTE |
| | | | NKKIAGNIKRLMAKKNYRTYGEYLYKEFFENKEKHKREKISNAANN |
| | | | HKFSPTRDLVIKEAEAILKKQAGKDGFHKELTEEYIEKLTKAIGYESE |
| | | | KLIPESGFCPYLKDEKRLPASHKLNEERRLWETLNNARYSDPIVDIVT |
| | | | GEITGYYEKQFTKEQKQKLFDYLLTGSELTPAQTKKLLGLKNTNFED |
| | | | IILQGRDKKAQKIKGYKLIKLESMPFWARLSEAQQDSFLYDWNSCPD |
| | | | EKLLTEKLSNEYHLTEEEIDNAFNEIVLSSSYAPLGKSAMLIILEKIKN |
| | | | DLSYTEAVEEALKEGKLTKEKQAIKDRLPYYGAVLQESTQKIIAKGF |
| | | | SPQFKDKGYKTPHTNKYELEYGRIANPVVHQTLNELRKLVNEIIDILG |
| | | | KKPCEIGLETARELKKSAEDRSKLSREQNDNESNRNRIYEIYIRPQQQ |
| | | | VIITRRENPRNYILKFELLEEQKSQCPFCGGQISPNDIINNQADIEHLFPI |
| | | | AESEDNGRNNLVISHSACNADKAKRSPWAAFASAAKDSKYDYNRIL |
| | | | SNVKENIPHKAWRFNQGAFEKFIENKPMAARFKTDNSYISKVAHKY |
| | | | LACLFEKPNIICVKGSLTAQLRMAWGLQGLMIPFAKQLITEKESESFN |
| | | | KDVNSNKKIRLDNRHHALDAIVIAYASRGYGNLLNKMAGKDYKINY |
| | | | SERNWLSKILLPPNNIVWENIDADLESFESSVKTALKNAFISVKHDHS |
| | | | DNGELVKGTMYKIFYSERGYTLTTYKKLSALKLTDPQKKKTPKDFL |
| | | | ETALLKFKGRESEMKNEKIKSAIENNKRLFDVIQDNLEKAKKLLEEE |
| | | | NEKSKAEGKKEKNINDASIYQKAISLSGDKYVQLSKKEPGKFFAISKP |
| | | | TPTTTGYGYDTGDSLCVDLYYDNKGKLCGEIIRKIDAQQKNPLKYKE |
| | | | QGFTLFERIYGGDILEVDFDIHSDKNSFRNNTGSAPENRVFIKVGTFTE |
| | | | ITNNNIQIWFGNIIKSTGGQDDSFTINSMQQYNPRKLILSSCGFIKYRSP |
| | | | ILKNKEG (SEQ ID NO 24) |
| WP_ | Akkermansia | Cas9 | MSRSLTFSFDIGYASIGWAVIASASHDDADPSVCGCGTVLFPKDDCQ |
| 012421034.1 | muciniphila | | AFKRREYRRLRRNIRSRRVRIERIGRLLVQAQIITPEMKETSGHPAPFY |
| | ATCC BAA-835 | | LASEALKGHRTLAPIELWHVLRWYAHNRGYDNNASWSNSLSEDGG |
| | | | NGEDTERVKHAQDLMDKHGTATMAETICRELKLEEGKADAPMEVS |
| | | | TPAYKNLNTAFPRLIVEKEVRRILELSAPLIPGLTAEIIELIAQHHPLTT |
| | | | EQRGVLLQHGIKLARRYRGSLLFGQLIPRFDNRIISRCPVTWAQVYEA |
| | | | ELKKGNSEQSARERAEKLSKVPTANCPEFYEYRMARILCNIRADGEP |
| | | | LSAEIRRELMNQARQEGKLTKASLEKAISSRLGKETETNVSNYFTLHP |
| | | | DSEEALYLNPAVEVLQRSGIGQILSPSVYRIAANRLRRGKSVTPNYLL |
| | | | NLLKSRGESGEALEKKIEKESKKKEADYADTPLKPKYATGRAPYART |
| | | | VLKKVVEEILDGEDPTRPARGEAHPDGELKAHDGCLYCLLDTDSSV |
| | | | NQHQKERRLDTMTNNHLVRHRMLILDRLLKDLIQDFADGQKDRISR |
| | | | VCVEVGKELTTFSAMDSKKIQRELTLRQKSHTDAVNRLKRKLPGKA |
| | | | LSANLIRKCRIAMDMNWTCPFTGATYGDHELENLELEHIVPHSFRQS |
| | | | NALSSLVLTWPGVNRMKGQRTGYDFVEQEQENPVPDKPNLHICSLN |
| | | | NYRELVEKLDDKKGHEDDRRRKKKRKALLMVRGLSHKHQSQNHE |
| | | | AMKEIGMTEGMMTQSSHLMKLACKSIKTSLPDAHIDMIPGAVTAEV |
| | | | RKAWDVFGVFKELCPEAADPDSGKILKENLRSLTHLHHALDACVLG |
| | | | LIPYIIPAHHNGLLRRVLAMRRIPEKLIPQVRPVANQRHYVLNDDGR |
| | | | MMLRDLSASLKENIREQLMEQRVIQHVPADMGGALLKETMQRVLS |
| | | | VDGSGEDAMVSLSKKKDGKKEKNQVKASKLVGVFPEGPSKLKALK |
| | | | AAIEIDGNYGVALDPKPVVIRHIKVFKRIMALKEQNGGKPVRILKKG |
| | | | MLIHLTSSKDPKHAGVWRIESIQDSKGGVKLDLQRAHCAVPKNKTH |
| | | | ECNWREVDLISLLKKYQMKRYPTSYTGTPR (SEQ ID NO 25) |
| WP_ | Streptococcus | Cas9 | MDKKYSIGLDIGTNSVGWAVITDDYKVPSKKFKVLGNTDRHSIKKN |
| 012560673.1 | pyogenes | | LIGALLFDSGETAEATRLKRTARRRYTRRKNRICYLQEIFSNEIAKVD |
| | NZ131 | | DSFFHRLEESFLVEEDKKHERHPIFGNIVDEVAYHEKYPTIYHLRKKL |
| | | | ADSTDKADLRLIYLALAHMIKFRGHFLIEGDLNPDNSDVDKLFIQLV |
| | | | QTYNQLFEENPINASRVDAKAILSARLSKSRRLENLIAQLPGEKRNGL |
| | | | FGNLIALSLGLTPNFKSNFDLAEDAKLQLSKDTYDDDLDNLLAQIGD |
| | | | QYADLFLAAKNLSDAILLSDILRVNTEITKAPLSASMIKRYDEHHQDL |
| | | | TLLKALVRQQLPEKYKEIFFDQSKNGYAGYIDGGASQEEFYKFIKPIL |
| | | | EKMDGTEELLAKLNREDLLRKQRTFDNGSIPHQIHLGELHAILRRQE |
| | | | DFYPFLKDNREKIEKILTFRIPYYVGPLARGNSRFAWMTRKSEETITP |
| | | | WNFEEVVDKGASAQSFIERMTNFDKNLPNEKVLPKHSLLYEYFTVY |
| | | | NELTKVKYVTEGMRKPAFLSGEQKKAIVDLLFKTNRKVTVKQLKED |
| | | | YFKKIECFDSVEISGVEDRFNASLGTYHDLLKIIKDKDFLDNEENEDIL |
| | | | EDIVLTLTLFEDREMIEERLKTYAHLFDDKVMKQLKRRRYTGWGRL |
| | | | SRKLINGIRDKQSGKTILDFLKSDGFANRNFMQLIHDDSLTFKEDLQK |
| | | | AQVSGQGDSLHEHIANLAGSPAIKKGILQTVKVVDELVKVMGRHKP |
| | | | ENIVIEMARENQTTQKGQKNSRERMKRIEEGIKELGSDILKEYPVETT |
| | | | QLQNEKLYLYYLQNGRDMYVDQELDINRLSDYDVDHIVPQSFLKDD |
| | | | SIDNKVLTRSDKNRGKSDNVPSEEVVKKMKNYWKQLLNAKLITQRK |
| | | | FDNLTKAERGGLSELDKAGFIKRQLVETRQITKHVAQILDSRMNTKY |
| | | | DENDKLIREVRVITLKSKLVSDFRKDFQFYKVREINNYHHAHDAYLN |
| | | | AVVGTALIKKYPKLESEFVYGDYKVYDIRKMIAKSEQEIGKATAKYF |
| | | | FYSNIMNFFKTEITLANGEIRKRPLIETNGETGEIVWDKGRDFATVRK |
| | | | VLSMPQVNIVKKTEVQTGGFSKESILPKRNSDKLIARKKDWDPKKYG |
| | | | GFDSPTVAYSVLVVAKVEKGKSKKLKSVKELVGITIMERSSFEKDPV |
| | | | DFLEAKGYKEVRKDLIIKLPKYSLFELENGRKRMLASAGELQKGNEL |
| | | | ALPSKYVNFLYLASHYEKLKGSPEDNEQKQLFVEQHKHYLDEIIEQIS |
| | | | EFSKRVILADANLDKVLSAYNKHRDKPIREQAENIIHLFTLTNLGAPA |
| | | | AFKCFDTTIGRNRYKSIKEVLDATLIHQSITGLYETRIDLSQLGGD |
| | | | (SEQ ID NO 26) |
| WP_ | Neisseria | Cas9 | MAAFKPNPINYILGLDIGIASVGWAMVEIDEDENPICLIDLGVRVFER |
| 002235162.1 | meningitidis | | AEVPKTGDSLAMARRLARSVRRLTRRRAHRLLRARRLLKREGVLQA |
| | Z2491 | | ADFDENGLIKSLPNTPWQLRAAALDRKLTPLEWSAVLLHLIKHRGYL |
| | | | SQRKNEGETADKELGALLKGVADNAHALQTGDFRTPAELALNKFEK |
| | | | ESGHIRNQRGDYSHTFSRKDLQAELILLFEKQKEFGNPHVSGGLKEGI |
| | | | ETLLMTQRPALSGDAVQKMLGHCTFEPAEPKAAKNTYTAERFIWLT |
| | | | KLNNLRILEQGSERPLTDTERATLMDEPYRKSKLTYAQARKLLGLED |
| | | | TAFFKGLRYGKDNAEASTLMEMKAYHAISRALEKEGLKDKKSPLNL |
| | | | SPELQDEIGTAFSLFKTDEDITGRLKDRIQPEILEALLKHISFDKFVQIS |
| | | | LKALRRIVPLMEQGKRYDEACAEIYGDHYGKKNTEEKIYLPPIPADEI |
| | | | RNPVVLRALSQARKVINGVVRRYGSPARIHIETAREVGKSFKDRKEIE |
| | | | KRQEENRKDREKAAAKFREYFPNFVGEPKSKDILKLRLYEQQHGKC |
| | | | LYSGKEINLGRLNEKGYVEIDHALPFSRTWDDSFNNKVLVLGSENQN |
| | | | KGNQTPYEYFNGKDNSREWQEFKARVETSRFPRSKKQRILLQKFDE |
| | | | DGFKERNLNDTRYVNRFLCQFVADRMRLTGKGKKRVFASNGQITNL |
| | | | LRGFWGLRKVRAENDRHHALDAVVVACSTVAMQQKITRFVRYKE |
| | | | MNAFDGKTIDKETGEVLHQKTHFPQPWEFFAQEVMIRVFGKPDGKP |
| | | | EFEEADTPEKLRTLLAEKLSSRPEAVHEYVTPLFVSRAPNRKMSGQG |
| | | | HMETVKSAKRLDEGVSVLRVPLTQLKLKDLEKMVNREREPKLYEAL |
| | | | KARLEAHKDDPAKAFAEPFYKYDKAGNRTQQVKAVRVEQVQKTG |
| | | | VWVRNHNGIADNATMVRVDVFEKGDKYYLVPIYSWQVAKGILPDR |
| | | | AVVQGKDEEDWQLIDDSFNFKFSLHPNDLVEVITKKARMFGYFASC |
| | | | HRGTGNINIRIHDLDHKIGKNGILEGIGVKTALSFQKYQIDELGKEIRP |
| | | | CRLKKRPPVR (SEQ ID NO 27) |
| YP_ | Campylobacter | Cas9 | MARILAFDIGISSIGWAFSENDELKDCGVRIFTKVENPKTGESLALPRR |
| 002344900.1 | jejuni | | LARSARKRLARRKARLNHLKHLIANEFKLNYEDYQSFDESLAKAYK |
| | subsp. | | GSLISPYELRFRALNELLSKQDFARVILHIAKRRGYDDIKNSDDKEKG |
| | jejuni | | AILKAIKQNEEKLANYQSVGEYLYKEYFQKFKENSKEFTNVRNKKES |
| | NCTC | | YERCIAQSFLKDELKLIFKKQREFGFSFSKKFEEEVLSVAFYKRALKD |
| | 11168 = | | FSHLVGNCSFFTDEKRAPKNSPLAFMFVALTRIINLLNNLKNTEGILY |
| | ATCC | | TKDDLNALLNEVLKNGTLTYKQTKKLLGLSDDYEFKGEKGTYFIEF |
| | 700819 | | KKYKEFIKALGEHNLSQDDLNEIAKDITLIKDEIKLKKALAKYDLNQ |
| | | | NQIDSLSKLEFKDHLNISFKALKLVTPLMLEGKKYDEACNELNLKVA |
| | | | INEDKKDFLPAFNETYYKDEVTNPVVLRAIKEYRKVLNALLKKYGK |
| | | | VHKINIELAREVGKNHSQRAKIEKEQNENYKAKKDAELECEKLGLKI |
| | | | NSKNILKLRLFKEQKEFCAYSGEKIKISDLQDEKMLEIDHIYPYSRSFD |
| | | | DSYMNKVLVFTKQNQEKLNQTPFEAFGNDSAKWQKIEVLAKNLPTK |
| | | | KQKRILDKNYKDKEQKNFKDRNLNDTRYIARLVLNYTKDYLDFLPL |
| | | | SDDENTKLNDTQKGSKVHVEAKSGMLTSALRHTWGFSAKDRNNHL |
| | | | HHAIDAVIIAYANNSIVKAFSDFKKEQESNSAELYAKKISELDYKNKR |
| | | | KFFEPFSGFRQKVLDKIDEIFVSKPERKKPSGALHEETFRKEEEFYQSY |
| | | | GGKEGVLKALELGKIRKVNGKIVKNGDMFRVDIFKHKKTNKFYAVP |
| | | | IYTMDFALKVLPNKAVARSKKGEIKDWILMDENYEFCFSLYKDSLILI |
| | | | QTKDMQEPEFVYYNAFTSSTVSLIVSKHDNKFETLSKNQKILFKNAN |
| | | | EKEVIAKSIGIQNLKVFEKYIVSALGEVTKAEFRQREDFKK (SEQ ID |
| | | | NO 28) |
| WP_ | Acidovorax | Cas9 | MAQHVFGLDIGIASVGWAILGEQRIIDLGVRCFDKAETAKEGDPLNL |
| 012655176.1 | ebreus | | TRRQARLLRRRLYRRAWRLTQLSRLLKRKGLIADAKLFAKAPSYGD |
| | TPSY] | | SAWELRRQGLDRLLTPLEWARVIYHQCKHRGFHWTSKAEEAKADS |
| | | | DAEGGRVKQGLAHTKALMQAKNYRSAAEMVLAEFPDAQRNKRGQ |
| | | | YDKALSRVLLGEELALLFATQRRLGNPHASDFFEKLILGDGDRKSGL |
| | | | FWQQKPALSGADLLKMLGKCTFEKGEYRAPKASFSVERHVWLTRL |
| | | | NNLRIVVDGRSRPLNEAERQAALLLPYQTETSKYKTLKNAFIKAGLW |
| | | | GDGVRFGGLAYPSQAQIDAEKTKDPEDQFLVKLPAWHELRKAFKAA |
| | | | GHEALWQQISTPALDGDPTLLDQIATVLSVYKDGAEVVQQLRQLAL |
| | | | PEPAASIAVLEKISFDKFSSLSLKALRRIVPLMQSGLRYDEAVAQIPEY |
| | | | GHHSQRIEPGAAKHLYLPPFYEAQRKYAGKGDHIGSMQFRDDADIPR |
| | | | NPVVLRALNQARKVVNALIREYGSPIAVNIEMARDLSRPLDERNKVK |
| | | | RAQEEFRDRNDRARSEFERDFGYKPKAAAFEKWMLYREQLGQCAY |
| | | | SQQPLDIQRVLDDHNYAQVDHALPYSRSYDDSKNNKVLVLTHENQN |
| | | | KGNRTAFEYLTSFPDGEDGERWRTFVAWVQGNKAYRMAKRNRLLR |
| | | | KNYGVDESKGFIDRNLNDTRYICKFFKNYVEEHLQLAARADGDTAR |
| | | | RCVVVNGQLTAFLRARWGLTKVRGDSDRHHALDAAVVAACTHGM |
| | | | VKALADYSRRKEISFLQEGFPDPETGEILNPAAFDRARQHFPEPWTHF |
| | | | AHELKARLFTDDLAALREDMQRLGSYTTEDLGRLRTLFVSRAPQRR |
| | | | SGGAVHKETIYAQPESLKQQGGVIEKILLTSLKLQDFDKLLNPESNDH |
| | | | FVEPHRNERLYAAIRQRLEQFGGRADKAFGPDNLFHKPDKNNQPTG |
| | | | PVVRSIKLVRGKQTGIPIRGGLAKNDSMLRVDIFTKAGKFHLVPVYV |
| | | | HHRVTGLPNRAIVAFKDEDEWTLIDESFAFLFSVYPNDYVKVTLKKE |
| | | | QQSGYYSGADRSTGAMNLWAHDRAASVGKDGLIRGIGVKTALSVE |
| | | | KFNVDVLGRIYLAPPETRSGLA (SEQ ID NO 29) |
| WP_ | Neisseria | Cas9 | MAAFKPNPINYILGLDIGIASVGWAMVEIDEEENPIRLIDLGVRVFER |
| 015815286.1 | meningitidis | | AEVPKTGDSLAMVRRLARSVRRLTRRRAHRLLRARRLLKREGVLQA |
| | alpha14 | | ADFDENGLIKSLPNTPWQLRAAALDRKLTPLEWSAVLLHLIKHRGYL |
| | | | SQRKNEGETADKELGALLKGVADNAHALQTGDFRTPAELALNKFEK |
| | | | ESGHIRNQRGDYSHTFSRKDLQAELILLFEKQKEFGNPHISGGLKEGI |
| | | | ETLLMTQRPALSGDAVQKMLGHCTFEPAEPKAAKNTYTAERFIWLT |
| | | | KLNNLRILEQGSERPLTDTERATLMDEPYRKSKLTYAQARKLLGLED |
| | | | TAFFKGLRYGKDNAEASTLMEMKAYHAISRALEKEGLKDKKSPLNL |
| | | | SPELQDEIGTAFSLFKTDEDITGRLKDRIQPEILEALLKHISFDKFVQIS |
| | | | LKALRRIVPLMEQGKRYDEACAEIYGDHYGKKNTEEKIYLPPIPADEI |
| | | | RNPVVLRALSQARKVINGVVRRYGSPARIHIETAREVGKSFKDRKEIE |
| | | | KRQEENRKDREKAAAKFREYFPNFVGEPKSKDILKLRLYEQQHGKC |
| | | | LYSGKEINLGRLNEKGYVEIDHALPFSRTWDDSFNNKVLVLGSENQN |
| | | | KGNQTPYEYFNGKDNSREWQEFKARVETSRFPRSKKQRILLQKFDE |
| | | | DGFKERNLNDTRYVNRFLCQFVADRMRLTGKGKKRVFASNGQITNL |
| | | | LRGFWGLRKVRAENDRHHALDAVVVACSTVAMQQKITRFVRYKE |
| | | | MNAFDGKTIDKETGEVLHQKTHFPQPWEFFAQEVMIRVFGKPDGKP |
| | | | EFEEADTPEKLRTLLAEKLSSRPEAVHEYVTPLFVSRAPNRKMSGQG |
| | | | HMETVKSAKRLDEGVSVLRVPLTQLKLKDLEKMVNREREPKLYEAL |
| | | | KARLEAHKDDPAKAFAEPFYKYDKAGNRTQQVKAVRVEQVQKTG |
| | | | VWVRNHNGIADNATMVRVDVFEKGDKYYLVPIYSWQVAKGILPDR |
| | | | AVVQGKDEEDWQLIDDSFNFKFSLHPNDLVEVITKKARMFGYFASC |
| | | | HRGTGNINIRIHDLDHKIGKNGILEGIGVKTALSFQKYQIDELGKEIRP |
| | | | CRLKKRPPVR (SEQ ID NO 30) |
| WP_ | Lactobacillus | Cas9 | MTKLNQPYGIGLDIGSNSIGFAVVDANSHLLRLKGETAIGARLFREG |
| 014569977.1 | rhamnosus | | QSAADRRGSRTTRRRLSRTRWRLSFLRDFFAPHITKIDPDFFLRQKYS |
| | GG | | EISPKDKDRFKYEKRLFNDRTDAEFYEDYPSMYHLRLHLMTHTHKA |
| | | | DPREIFLAIHHILKSRGHFLTPGAAKDFNTDKVDLEDIFPALTEAYAQ |
| | | | VYPDLELTFDLAKADDFKAKLLDEQATPSDTQKALVNLLLSSDGEK |
| | | | EIVKKRKQVLTEFAKAITGLKTKFNLALGTEVDEADASNWQFSMGQ |
| | | | LDDKWSNIETSMTDQGTEIFEQIQELYRARLLNGIVPAGMSLSQAKV |
| | | | ADYGQHKEDLELFKTYLKKLNDHELAKTIRGLYDRYINGDDAKPFL |
| | | | REDFVKALTKEVTAHPNEVSEQLLNRMGQANFMLKQRTKANGAIPI |
| | | | QLQQRELDQIIANQSKYYDWLAAPNPVEAHRWKMPYQLDELLNFHI |
| | | | PYYVGPLITPKQQAESGENVFAWMVRKDPSGNITPYNFDEKVDREA |
| | | | SANTFIQRMKTTDTYLIGEDVLPKQSLLYQKYEVLNELNNVRINNEC |
| | | | LGTDQKQRLIREVFERHSSVTIKQVADNLVAHGDFARRPEIRGLADE |
| | | | KRFLSSLSTYHQLKEILHEAIDDPTKLLDIENIITWSTVFEDHTIFETKL |
| | | | AEIEWLDPKKINELSGIRYRGWGQFSRKLLDGLKLGNGHTVIQELML |
| | | | SNHNLMQILADETLKETMTELNQDKLKTDDIEDVINDAYTSPSNKKA |
| | | | LRQVLRVVEDIKHAANGQDPSWLFIETADGTGTAGKRTQSRQKQIQ |
| | | | TVYANAAQELIDSAVRGELEDKIADKASFTDRLVLYFMQGGRDIYT |
| | | | GAPLNIDQLSHYDIDHILPQSLIKDDSLDNRVLVNATINREKNNVFAS |
| | | | TLFAGKMKATWRKWHEAGLISGRKLRNLMLRPDEIDKFAKGFVAR |
| | | | QLVETRQIIKLTEQIAAAQYPNTKIIAVKAGLSHQLREELDFPKNRDV |
| | | | NHYHHAFDAFLAARIGTYLLKRYPKLAPFFTYGEFAKVDVKKFREF |
| | | | NFIGALTHAKKNIIAKDTGEIVWDKERDIRELDRIYNFKRMLITHEVY |
| | | | FETADLFKQTIYAAKDSKERGGSKQLIPKKQGYPTQVYGGYTQESGS |
| | | | YNALVRVAEADTTAYQVIKISAQNASKIASANLKSREKGKQLLNEIV |
| | | | VKQLAKRRKNWKPSANSFKIVIPRFGMGTLFQNAKYGLFMVNSDTY |
| | | | YRNYQELWLSRENQKLLKKLFSIKYEKTQMNHDALQVYKAIIDQVE |
| | | | KFFKLYDINQFRAKLSDAIERFEKLPINTDGNKIGKTETLRQILIGLQA |
| | | | NGTRSNVKNLGIKTDLGLLQVGSGIKLDKDTQIVYQSPSGLFKRRIPL |
| | | | ADL (SEQ ID NO 31) |
| WP_ | Streptobacillus | Cas9 | MKYILGLDLGVASIGWAVTEINEKGEPVGLIDANSVIFKPLDNDKGK |
| 012859073.1 | moniliformis | | LYNVERRDKRGSRRILRRKKHRVERTKMLLVNSSFLTNEEIDNLYVG |
| | DSM | | KLDNIYEVRLKGLKEQLSKNEIARLMIYYCKNRGFKSNRKTEDLEIL |
| | 12112 | | KSLGEKISEKDSDEKKLKPIIAKNTELIESKGLTPIEVIYMIRENDNSIL |
| | | | GFKNKEGNYKFGFKRNQVIDEVRKILGTQQILSKEIVDEYIDILSSQR |
| | | | DFSDGPGGDSKYKIDYTKLAGKCKYTGEVRAVKSAPSYEIFTMLQK |
| | | | LNDIRYVKFTSEDKIEKKKLSKEVIHKLYDLVVEKNKTLTYDLIEKSI |
| | | | DEENIKLLNIPKLSKSKYVELRKSYFKERNDNENLTEEEINAFNEKLN |
| | | | NERMKQELIRNNLKAYKELKSKFKKNNIDENRIKELGGIYFLDLVAE |
| | | | LLTYSKTDEKLEYFVENVERYSLFKEQRDIVEIIKTFPNYTKNGNLSL |
| | | | SLVRELNKLLMEGHDYESSLSSLNYYIDTDVDWEKFPTISEIEDSLST |
| | | | KITNPNVKHILVILRKLYNTLLFKYGRPEKVHLELSRDFSNDFSTRNKI |
| | | | QKEQLENKVRREVAAFEMYGANKDIVAGKDRLSNDDFVRIKLWEE |
| | | | QNKVCMYSGRTIEKYQLTSAEVQIDHILPYSKSFDNSYSNKVLVFSN |
| | | | ENQDKKERTPYQWLKGTEKWNEFKQRVRLNLNISNKKKENLLFEDE |
| | | | VVNNEFLERELHATSYSSRLALNIFQRLIPVSDEDKYDEHGNEKVKY |
| | | | LYNRNVIAFQGKMTSMLRNLYSLNKYTHSFESDTLDIDNKAFILKEF |
| | | | EINKDNLKMTAYNVNTGLEITSETKVEKNKSGEFKTIKDELLKKELD |
| | | | KKENLIEISDYFKDKVLFDLKIVDFEEIDTVEPEIISILYKMITALKEEV |
| | | | YSKNRENHLHHSLDAFLLTIMNRSMQMKLIKYNQLISVLKNKEIEIFN |
| | | | EEKGEYIDSKEFAEELQKQKIIDIDGNERGIKFNVNGKTHRLNLVKPY |
| | | | ENFLDDIKNKIFDKRENNKLPYHVVKSKVSGALHAETILGESKGEITK |
| | | | RISVFDINYKNLGKIFDKDGSQKEIYETLVKWLEAKSKDYPKLKNGN |
| | | | IIKKVKIVDGNKDKLIKLGNKRYVEMGRTTVKILVLKKEAEEGLKFA |
| | | | SIGRYKYNLLKSEKDVNISIWKDAIHFCKVRYNKLKENGYRLIHELIP |
| | | | GETIELELNKGNISKCLVVGFSGGKIEISSVIGDALDLINDKISTRIKER |
| | | | YYITVSTIKSIKKINKNILGD (SEQ ID NO 32) |
| WP_ | Helicobacter | Cas9 | MIRTLGIDIGIASIGWAVIEGEYTDKGLENKEIVASGVRVFTKAENPK |
| 013022389.1 | mustelae | | NKESLALPRTLARSARRRNARKKGRIQQVKHYLSKALGLDLECFVQ |
| | 12198 | | GEKLATLFQTSKDFLSPWELRERALYRVLDKEELARVILHIAKRRGY |
| | | | DDITYGVEDNDSGKIKKAIAENSKRIKEEQCKTIGEMMYKLYFQKSL |
| | | | NVRNKKESYNRCVGRSELREELKTIFQIQQELKSPWVNEELIYKLLG |
| | | | NPDAQSKQEREGLIFYQRPLKGFGDKIGKCSHIKKGENSPYRACKHA |
| | | | PSAEEFVALTKSINFLKNLTNRHGLCFSQEDMCVYLGKILQEAQKNE |
| | | | KGLTYSKLKLLLDLPSDFEFLGLDYSGKNPEKAVFLSLPSTFKLNKIT |
| | | | QDRKTQDKIANILGANKDWEAILKELESLQLSKEQIQTIKDAKLNFSK |
| | | | HINLSLEALYHLLPLMREGKRYDEGVEILQERGIFSKPQPKNRQLLPP |
| | | | LSELAKEESYFDIPNPVLRRALSEFRKVVNALLEKYGGFHYFHIELTR |
| | | | DVCKAKSARMQLEKINKKNKSENDAASQLLEVLGLPNTYNNRLKC |
| | | | KLWKQQEEYCLYSGEKITIDHLKDQRALQIDHAFPLSRSLDDSQSNK |
| | | | VLCLTSSNQEKSNKTPYEWLGSDEKKWDMYVGRVYSSNFSPSKKRK |
| | | | LTQKNFKERNEEDFLARNLVDTGYIGRVTKEYIKHSLSFLPLPDGKK |
| | | | EHIRIISGSMTSTMRSFWGVQEKNRDHHLHHAQDAIIIACIEPSMIQKY |
| | | | TTYLKDKETHRLKSHQKAQILREGDHKLSLRWPMSNFKDKIQESIQN |
| | | | IIPSHHVSHKVTGELHQETVRTKEFYYQAFGGEEGVKKALKFGKIREI |
| | | | NQGIVDNGAMVRVDIFKSKDKGKFYAVPIYTYDFAIGKLPNKAIVQG |
| | | | KKNGIIKDWLEMDENYEFCFSLFKNDCIKIQTKEMQEAVLAIYKSTN |
| | | | SAKATIELEHLSKYALKNEDEEKMFTDTDKEKNKTMTRESCGIQGLK |
| | | | VFQKVKLSVLGEVLEHKPRNRQNIALKTTPKHV (SEQ ID NO 33) |
| WP_ | Neisseria | Cas9 | MAAFKPNSINYILGLDIGIASVGWAMVEIDEEENPIRLIDLGVRVFER |
| 014574210.1 | meningitidis | | AEVPKTGDSLAMARRLARSVRRLTRRRAHRLLRTRRLLKREGVLQA |
| | 8013 | | ANFDENGLIKSLPNTPWQLRAAALDRKLTPLEWSAVLLHLIKHRGYL |
| | | | SQRKNEGETADKELGALLKGVAGNAHALQTGDFRTPAELALNKFEK |
| | | | ESGHIRNQRSDYSHTFSRKDLQAELILLFEKQKEFGNPHVSGGLKEGI |
| | | | ETLLMTQRPALSGDAVQKMLGHCTFEPAEPKAAKNTYTAERFIWLT |
| | | | KLNNLRILEQGSERPLTDTERATLMDEPYRKSKLTYAQARKLLGLED |
| | | | TAFFKGLRYGKDNAEASTLMEMKAYHAISRALEKEGLKDKKSPLNL |
| | | | SPELQDEIGTAFSLFKTDEDITGRLKDRIQPEILEALLKHISFDKFVQIS |
| | | | LKALRRIVPLMEQGKRYDEACAEIYGDHYGKKNTEEKIYLPPIPADEI |
| | | | RNPVVLRALSQARKVINGVVRRYGSPARIHIETAREVGKSFKDRKEIE |
| | | | KRQEENRKDREKAAAKFREYFPNFVGEPKSKDILKLRLYEQQHGKC |
| | | | LYSGKEINLGRLNEKGYVEIDHALPFSRTWDDSFNNKVLVLGSENQN |
| | | | KGNQTPYEYFNGKDNSREWQEFKARVETSRFPRSKKQRILLQKFDE |
| | | | DGFKERNLNDTRYVNRFLCQFVADRMRLTGKGKKRVFASNGQITNL |
| | | | LRGFWGLRKVRAENDRHHALDAVVVACSTVAMQQKITRFVRYKE |
| | | | MNAFDGKTIDKETGEVLHQKTHFPQPWEFFAQEVMIRVFGKPDGKP |
| | | | EFEEADTLEKLRTLLAEKLSSRPEAVHEYVTPLFVSRAPNRKMSGQG |
| | | | HMETVKSAKRLDEGVSVLRVPLTQLKLKDLEKMVNREREPKLYEAL |
| | | | KARLEAHKDDPAKAFAEPFYKYDKAGNRTQQVKAVRVEQVQKTG |
| | | | VWVRNHNGIADNATMVRVDVFEKGDKYYLVPIYSWQVAKGILPDR |
| | | | AVVQGKDEEDWQLIDDSFNFKFSLHPNDLVEVITKKARMFGYFASC |
| | | | HRGTGNINIRIHDLDHKIGKNGILEGIGVKTALSFQKYQIDELGKEIRP |
| | | | CRLKKRPPVR (SEQ ID NO 34) |
| WP_ | Neisseria | Cas9 | MAAFKPNPINYILGLDIGIASVGWAMVEIDEDENPICLIDLGVRVFER |
| 002246410.1 | meningitidis | | AEVPKTGDSLAMARRLARSVRRLTRRRAHRLLRARRLLKREGVLQA |
| | WUE | | ADFDENGLIKSLPNTPWQLRAAALDRKLTPLEWSAVLLHLIKHRGYL |
| | 2594 | | SQRKNEGETADKELGALLKGVADNAHALQTGDFRTPAELALNKFEK |
| | | | ESGHIRNQRGDYSHTFSRKDLQAELILLFEKQKEFGNPHVSGGLKEGI |
| | | | ETLLMTQRPALSSDAVQKMLGHCTFEPAEPKAAKNTYTAERFIWLT |
| | | | KLNNLRILEQGSERPLTDTERATLMDEPYRKSKLTYAQARKLLGLED |
| | | | TAFFKGLRYGKDNAEASTLMEMKAYHAISRALEKEGLKDKKSPLNL |
| | | | SPELQDEIGTAFSLFKTDEDITGRLKDRIQPEILEALLKHISFDKFVQIS |
| | | | LKALRRIVPLMEQGKRYDEACAEIYGDHYGKKNTEEKIYLPPIPADEI |
| | | | RNPVVLRALSQARKVINGVVRRYGSPARIHIETAREVGKSFKDRKEIE |
| | | | KRQEENRKDREKAAAKFREYFPNFVGEPKSKDILKLRLYEQQHGKC |
| | | | LYSGKEINLGRLNEKGYVEIDHALPFSRTWDDSFNNKVLVLGSENQN |
| | | | KGNQTPYEYFNGKDNSREWQEFKARVETSRFPRSKKQRILLQKFDE |
| | | | DGFKERNLNDTRYVNRFLCQFVADRMRLTGKGKKRVFASNGQITNL |
| | | | LRGFWGLRKVRAENDRHHALDAVVVACSTVAMQQKITRFVRYKE |
| | | | MNAFDGKTIDKETGEVLHQKTHFPQPWEFFAQEVMIRVFGKPDGKP |
| | | | EFEEADTPEKLRTLLAEKLSSRPEAVHEYVTPLFVSRAPNRKMSGQG |
| | | | HMETVKSAKRLDEGVSVLRVPLTQLKLKDLEKMVNREREPKLYEAL |
| | | | KARLEAHKDDPAKAFAEPFYKYDKAGNRTQQVKAVRVEQVQKTG |
| | | | VWVRNHNGIADNATMVRVDVFEKGDKYYLVPIYSWQVAKGILPDR |
| | | | AVVQGKDEEDWQLIDDSFNFKFSLHPNDLVEVITKKARMFGYFASC |
| | | | HRGTGNINIRIHDLDHKIGKNGILEGIGVKTALSFQKYQIDELGKEIRP |
| | | | CRLKKRPPVR (SEQ ID NO 35) |
| WP_ | Campylobacter | Cas9 | MARILAFDIGISSIGWAFSENDELKDCGVRIFTKAENPKTGESLALPRR |
| 015016497 | jejuni | | LARSARKRLARRKARLNHLKHLIANEFKLNYEDYQSFDESLAKAYK |
| | subsp. | | GSLISPYELRFRALNELLSKQDFARVILHIAKRRGYDDIKNNGDEEKS |
| | jejuni | | EILKAIKQNEEKLVNYQSVGEYLYKEYFQKFKENSKEFINVRNKKES |
| | PT14 | | YERCIAQSFLKDELKLIFQKQREFGFSFSKKFEEEVLSVAFYKRALKD |
| | | | FSHLVGNCSFFTDEKRAPKNSPLAFMFVALTRIINLLNNLKNTEGILY |
| | | | TKDDLNTLLNEVLKNGTLTYKQTKKLLGLSDDYEFKGEKGTYFIEFK |
| | | | KYKEFIKALGEHNLSQDNLNEIAKDITLIKDEIKLKKALAKYDLNQN |
| | | | QIDSLSKLEFKDHLNISFKALKLITPLMLEGKKYDEAYNELNLKVAIN |
| | | | EDKKDFLPAFNETYYKDEVTNPVVLRAIKEYRKVLNALLKKYGKVH |
| | | | KINIELAREVGKNHSQRAKIEKEQNENYKAKKDAELECEKLGLKINS |
| | | | KNILKLRLFKEQKEFCAYSGEKIKISDLQDEKMLEIDHIYPYSRSFDDS |
| | | | YMNKVLVFTKQNQEKLNQTPFEAFGNDSAKWQKIEVLAKNLPTKK |
| | | | QKRILDKNYKDKEQKDFKDRNLNDTRYIARLVLNYTKDYLDFLPLS |
| | | | DDENTKLNDTQKGSKVHVEAKSGMLTSALRHTWGFSAKDRNNHLH |
| | | | HAIDAVIIAYANNSIVKAFSDFKKEQESNSAELYAKKISELDYKNKRK |
| | | | FFEPFSGFRQKVLDKIDEIFVSKPERKKPSGALHEETFRKEEEFYQSYG |
| | | | GKEGVLKALELGKIRKVNGKIVKNGDMFRVDIFKHKKTNKFYPVPIY |
| | | | TMDFALKVLPNKAVVQGKDKKSGLIKDWILMDENYEFCFSLYKDSL |
| | | | ILIQTKDMQEPELVYFNAFTSSTVSLIVSKHDNKFETLSKNQKILFKN |
| | | | ANEKEVIAKSIGIQNLKVFEKYIVSALGEVTKAEFRQREDFKK (SEQ |
| | | | ID NO 36) |
| WP_ | Campylobacter | Cas9 | MARILAFDIGISSIGWAFSENDELKDCGVRIFTKAENPKTGESLALPRR |
| 020836910.1 | jejuni | | LARSARKRLARRKARLNHLKHLIANEFKLNYEDYQSFDESLAKAYK |
| | 32488 | | GSLISPYELRFRALNELLSKQDFARVILHIAKRRGYDDIKNSDDKEKG |
| | | | AILKAIKQNEEKLANYQSVGEYLYKEYFQKFKENSKEFTNVRNKKES |
| | | | YERCIAQSFLKDELKLIFKKQREFGFSFSKKFEEEVLSVAFYKRALKD |
| | | | FSHLVGNCSFFTDEKRAPKNSPLAFMFVALTRIINLLNNLKNTEGILY |
| | | | TKDDLNALLNEVLKNGTLTYKQTKKLLGLSDDYEFKGEKGTYFIEF |
| | | | KKYKEFIKALGDHSLSQDDLNEIAKDITLIKDEIKLKKALAKYDLNQ |
| | | | NQIDSLSKLEFKDHLNISFKALKLITPLMLEGKKYDEACNELNLKVAI |
| | | | NEDKKDFLPAFNETYYKDEVTNPVVLRAIKEYRKVLNALLKKYGKV |
| | | | HKINIELAREVGKNYSQRAKIEKEQNENYKAKKDAELECEKLGLKIN |
| | | | SKNILKLRLFKEQKEFCAYSGEKIKISDLQDEKMLEIDHIYPYSRSFDD |
| | | | SYMNKVLVFTKQNQEKLNQTPFEAFGNDSAKWQKIEVLAKNLPTKK |
| | | | QKRILDKNYKDKEQKDFKDRNLNDTRYIARLVLNYTKDYLDFLPLS |
| | | | DDENTKLNDTQKGSKVHVEAKSGMLTSALRHTWGFSAKDRNNHLH |
| | | | HAIDAVIIAYANNSIVKAFSDFKKEQESNSAELYAKKISELDYKNKRK |
| | | | FFEPFSGFRQKVLDKIDEIFVSKPERKKPSGALHEETFRKEEEFYQSYG |
| | | | GKEGVLKALELGKIRKVNGKIVKNGDMFRVDIFKHKKTNKFYAVPI |
| | | | YTMDFALKVLPNKAVARSKKGEIKDWILMDENYEFCFSLYKDSLILI |
| | | | QTKDMQEPEFVYYNAFTSSTVSLIVSKHDNKFETLSKNQKILFKNAN |
| | | | EKEVIAKSIGIQNLKVFEKYIVSALGEVTKAEFRQREGFKK (SEQ ID |
| | | | NO 37) |
| WP_ | Campylobacter | Cas9 | MVRILAFDIGISSIGWAFSENDELKDCGVRIFTKVENPKTGESLALPRR |
| 002858341.1 | multispecies | | LARSARKRLARRKARLNHLKHLIANEFKLNYEDYQSFDESLAKAYK |
| | | | GSLISPYELRFRALNELLSKQDFARVILHIAKRRGYDDIKNSDDKEKG |
| | | | AILKAIKQNEEKLANYQSVGEYLYKEYFQKFKENSKEFTNVRNKKES |
| | | | YERCIAQSFLKDELKLIFKKQREFGFSFSKKFEEEVLSVAFYKRALKD |
| | | | FSHLVGNCSFFTDEKRAPKNSPLAFMFVALTRIINLLNNLKNTEGILY |
| | | | TKDDLNALLNEVLKNGTLTYKQTKKLLGLSDDYEFKGEKGTYFIEF |
| | | | KKYKEFIKALGEHNLSQDDLNEIAKDITLIKDEIKLKKALAKYDLNQ |
| | | | NQIDSLSKLEFKDHLNISFKALKLITPLMLEGKKYDEACNELNLKVAI |
| | | | NEDKKDFLPAFNETYYKDEVTNPVVLRAIKEYRKVLNALLKKYGKV |
| | | | HKINIELAREVGKNHSQRAKIEKEQNENYKAKKDAELECEKLGLKIN |
| | | | SKNILKLRLFKEQKEFCAYSGEKIKISDLQDEKMLEIDHIYPYSRSFDD |
| | | | SYMNKVLVFTKQNQEKLNKTPFEAFGNDSAKWQKIEVLAKNLPTKK |
| | | | QKRILDKNYKDKEQKDFKDRNLNDTRYIARLVLNYTKDYLDFLPLS |
| | | | DDENTKLNDTQKGSKVHVEAKSGMLTSALRHTWGFSTKDRNNHLH |
| | | | HAIDAVIIAYANNSIVKAFSDFKKEQESNSAELYAKKISELDYKNKRK |
| | | | FFEPFSGFRQKVLDKIDEIFVSKPERKKPSGALHEETFRKEEEFYQSYG |
| | | | GKEGVLKALELGKIRKVNGKIVKNGDMFRVDIFKHKKTNKFYAVPI |
| | | | YTMDFALKVLPNKAVARSKKGEIKDWILMDENYEFCFSLYKDSLILI |
| | | | QTKDMQEPEFVYYNAFTSSTVSLIVSKHDNKFETLSKNQKILFKNAN |
| | | | EKEVIAKSIGIQNLKVFEKYIVSALGEVTKAEFRQREDFKK (SEQ ID |
| | | | NO 38) |
| WP_ | Campylobacter | Cas9 | MARILAFDIGISSIGWAFSENDELKDCGVRIFTKAENPKTGESLALPRR |
| 022552435.1 | multispecies | | LARSARKRLARRKARLNHLKHLIANEFKLNYEDYQSFDESLAKAYK |
| | | | GSLISPYELRFRALNELLSKQDFARVILHIAKRRGYDDIKNSDDKEKG |
| | | | AILKAIKQNEEKLANYQSVGEYLYKEYFQKFKENSKEFTNVRNKKES |
| | | | YERCIAQSFLKDELKLIFKKQREFGFSFSKKFEEEVLSVAFYKRALKD |
| | | | FSHLVGNCSFFTDEKRAPKNSPLAFMFVALTRIINLLNNLKNTEGILY |
| | | | TKDDLNALLNEVLKNGTLTYKQTKKLLGLSDDYEFKGEKGTYFIEF |
| | | | KKYKEFIKALGDHSLSQDDLNEIAKDITLIKDEIKLKKALAKYDLNQ |
| | | | NQIDSLSKLEFKDHLNISFKALKLITPLMLEGKKYDEACNELNLKVAI |
| | | | NEDKKDFLPAFNETYYKDEVTNPVVLRAIKEYRKILNALLKKYGKV |
| | | | HKINIELAREVGKNHSQRAKIEKEQNENYKAKKDAEIECEKLGLKIN |
| | | | SKNILKLRLFKEQKEFCAYSGEKIKLSDLQDEKMLEIDHIYPYSRSFD |
| | | | DSYMNKVLVFTKQNQEKLNKTPFEAFGNDSTKWQKIEVLAKNLPEK |
| | | | KQKRILDKNYKDKEQKDFKDRNLNDTRYIARLVLNYTKDYLDFLPL |
| | | | SDDENTKLNDTQKGSKVHVEAKSGMLTSALRHTWGFSAKDRNNHL |
| | | | HHAIDAAIIAYANNSIVKAFSDFKKEQESNSVELYAKKISELDYKNKR |
| | | | KFFEPFSGFRQKVLDKIDEIFVSKPERKKPSGALHEETFRKEEEFYQSY |
| | | | GGKEGVLKALELGKIRKVNGKIVKNGDMFRVDIFKHKKTNKFYAVP |
| | | | IYTMDFALKVLPNKAVARSKKGEIKDWILMDENYEFCFSLYKDSLILI |
| | | | QTKDMQEPELVYFNAFTSSTVSLIVSKHDNKFETLSKNQKILFKNAN |
| | | | EKEVIAKSIGIQNLKVFEKYIVSALGEVTKAEFRQREDFKK (SEQ ID |
| | | | NO 39) |
| Francisella | Francisella | Cas12a/ | MSIYQEFVNKYSLSKTLRFELIPQGKTLENIKARGLILDDEKRAKDYK |
| tu | tularensis | Cpf1 | KAKQIIDKYHQFFIEEILSSVCISEDLLQNYSDVYFKLKKSDDDNLQK |
| | | | DFKSAKDTIKKQISEYIKDSEKFKNLFNQNLIDAKKGQESDLILWLKQ |
| | | | SKDNGIELFKANSDITDIDEALEIIKSFKGWTTYFKGFHENRKNVYSS |
| | | | NDIPTSIIYRIVDDNLPKFLENKAKYESLKDKAPEAINYEQIKKDLAEE |
| | | | LTFDIDYKTSEVNQRVFSLDEVFEIANFNNYLNQSGITKFNTIIGGKFV |
| | | | NGENTKRKGINEYINLYSQQINDKTLKKYKMSVLFKQILSDTESKSFV |
| | | | IDKLEDDSDVVTTMQSFYEQIAAFKTVEEKSIKETLSLLFDDLKAQKL |
| | | | DLSKIYFKNDKSLTDLSQQVFDDYSVIGTAVLEYITQQIAPKNLDNPS |
| | | | KKEQELIAKKTEKAKYLSLETIKLALEEFNKHRDIDKQCRFEEILANF |
| | | | AAIPMIFDEIAQNKDNLAQISIKYQNQGKKDLLQASAEDDVKAIKDL |
| | | | LDQTNNLLHKLKIFHISQSEDKANILDKDEHFYLVFEECYFELANIVP |
| | | | LYNKIRNYITQKPYSDEKFKLNFENSTLANGWDKNKEPDNTAILFIK |
| | | | DDKYYLGVMNKKNNKIFDDKAIKENKGEGYKKIVYKLLPGANKML |
| | | | PKVFFSAKSIKFYNPSEDILRIRNHSTHTKNGSPQKGYEKFEFNIEDCR |
| | | | KFIDFYKQSISKHPEWKDFGFRFSDTQRYNSIDEFYREVENQGYKLTF |
| | | | ENISESYIDSVVNQGKLYLFQIYNKDFSAYSKGRPNLHTLYWKALFD |
| | | | ERNLQDVVYKLNGEAELFYRKQSIPKKITHPAKEAIANKNKDNPKKE |
| | | | SVFEYDLIKDKRFTEDKFFFHCPITINFKSSGANKFNDEINLLLKEKAN |
| | | | DVHILSIDRGERHLAYYTLVDGKGNIIKQDTFNIIGNDRMKTNYHDK |
| | | | LAAIEKDRDSARKDWKKINNIKEMKEGYLSQVVHEIAKLVIEYNAIV |
| | | | VFEDLNFGFKRGRFKVEKQVYQKLEKMLIEKLNYLVFKDNEFDKTG |
| | | | GVLRAYQLTAPFETFKKMGKQTGIIYYVPAGFTSKICPVTGFVNQLY |
| | | | PKYESVSKSQEFFSKFDKICYNLDKGYFEFSFDYKNFGDKAAKGKW |
| | | | TIASFGSRLINFRNSDKNHNWDTREVYPTKELEKLLKDYSIEYGHGE |
| | | | CIKAAICGESDKKFFAKLTSVLNTILQMRNSKTGTELDYLISPVADVN |
| | | | GNFFDSRQAPKNMPQDADANGAYHIGLKGLMLLGRIKNNQEGKKL |
| | | | NLVIKNEEYFEFVQNRNN (SEQ ID NO 40) |
| Prevotella | Prevotella | Cas12a/ | MENYQEFTNLFQLNKTLRFELKPIGKTCELLEEGKIFASGSFLEKDKV |
| dis | disiens | Cpf1 | RADNVSYVKKEIDKKHKIFIEETLSSFSISNDLLKQYFDCYNELKAFK |
| | | | KDCKSDEEEVKKTALRNKCTSIQRAMREAISQAFLKSPQKKLLAIKN |
| | | | LIENVFKADENVQHFSEFTSYFSGFETNRENFYSDEEKSTSIAYRLVH |
| | | | DNLPIFIKNIYIFEKLKEQFDAKTLSEIFENYKLYVAGSSLDEVFSLEYF |
| | | | NNTLTQKGIDNYNAVIGKIVKEDKQEIQGLNEHINLYNQKHKDRRLP |
| | | | FFISLKKQILSDREALSWLPDMFKNDSEVIKALKGFYIEDGFENNVLT |
| | | | PLATLLSSLDKYNLNGIFIRNNEALSSLSQNVYRNFSIDEAIDANAELQ |
| | | | TFNNYELIANALRAKIKKETKQGRKSFEKYEEYIDKKVKAIDSLSIQEI |
| | | | NELVENYVSEFNSNSGNMPRKVEDYFSLMRKGDFGSNDLIENIKTKL |
| | | | SAAEKLLGTKYQETAKDIFKKDENSKLIKELLDATKQFQHFIKPLLGT |
| | | | GEEADRDLVFYGDFLPLYEKFEELTLLYNKVRNRLTQKPYSKDKIRL |
| | | | CFNKPKLMTGWVDSKTEKSDNGTQYGGYLFRKKNEIGEYDYFLGIS |
| | | | SKAQLFRKNEAVIGDYERLDYYQPKANTIYGSAYEGENSYKEDKKR |
| | | | LNKVIIAYIEQIKQTNIKKSIIESISKYPNISDDDKVTPSSLLEKIKKVSID |
| | | | SYNGILSFKSFQSVNKEVIDNLLKTISPLKNKAEFLDLINKDYQIFTEV |
| | | | QAVIDEICKQKTFIYFPISNVELEKEMGDKDKPLCLFQISNKDLSFAKT |
| | | | FSANLRKKRGAENLHTMLFKALMEGNQDNLDLGSGAIFYRAKSLDG |
| | | | NKPTHPANEAIKCRNVANKDKVSLFTYDIYKNRRYMENKFLFHLSIV |
| | | | QNYKAANDSAQLNSSATEYIRKADDLHIIGIDRGERNLLYYSVIDMK |
| | | | GNIVEQDSLNIIRNNDLETDYHDLLDKREKERKANRQNWEAVEGIK |
| | | | DLKKGYLSQAVHQIAQLMLKYNAIIALEDLGQMFVTRGQKIEKAVY |
| | | | QQFEKSLVDKLSYLVDKKRPYNELGGILKAYQLASSITKNNSDKQNG |
| | | | FLFYVPAWNTSKIDPVTGFTDLLRPKAMTIKEAQDFFGAFDNISYND |
| | | | KGYFEFETNYDKFKIRMKSAQTRWTICTFGNRIKRKKDKNYWNYEE |
| | | | VELTEEFKKLFKDSNIDYENCNLKEEIQNKDNRKFFDDLIKLLQLTLQ |
| | | | MRNSDDKGNDYIISPVANAEGQFFDSRNGDKKLPLDADANGAYNIA |
| | | | RKGLWNIRQIKQTKNDKKLNLSISSTEWLDFVREKPYLK (SEQ ID NO |
| | | | 41) |
| Acidamin- | Acidamin- | Cas12a/ | MTQFEGFTNLYQVSKTLRFELIPQGKTLKHIQEQGFIEEDKARNDHY |
| ococcus | ococcus | Cpf1 | KELKPIIDRIYKTYADQCLQLVQLDWENLSAAIDSYRKEKTEETRNA |
| | sp. | | LIEEQATYRNAIHDYFIGRTDNLTDAINKRHAEIYKGLFKAELFNGKV |
| | BV3L6 | | LKQLGTVTTTEHENALLRSFDKFTTYFSGFYENRKNVFSAEDISTAIP |
| | | | HRIVQDNFPKFKENCHIFTRLITAVPSLREHFENVKKAIGIFVSTSIEEV |
| | | | FSFPFYNQLLTQTQIDLYNQLLGGISREAGTEKIKGLNEVLNLAIQKN |
| | | | DETAHIIASLPHRFIPLFKQILSDRNTLSFILEEFKSDEEVIQSFCKYKTL |
| | | | LRNENVLETAEALFNELNSIDLTHIFISHKKLETISSALCDHWDTLRN |
| | | | ALYERRISELTGKITKSAKEKVQRSLKHEDINLQEIISAAGKELSEAFK |
| | | | QKTSEILSHAHAALDQPLPTTLKKQEEKEILKSQLDSLLGLYHLLDWF |
| | | | AVDESNEVDPEFSARLTGIKLEMEPSLSFYNKARNYATKKPYSVEKF |
| | | | KLNFQMPTLASGWDVNKEKNNGAILFVKNGLYYLGIMPKQKGRYK |
| | | | ALSFEPTEKTSEGFDKMYYDYFPDAAKMIPKCSTQLKAVTAHFQTHT |
| | | | TPILLSNNFIEPLEITKEIYDLNNPEKEPKKFQTAYAKKTGDQKGYRE |
| | | | ALCKWIDFTRDFLSKYTKTTSIDLSSLRPSSQYKDLGEYYAELNPLLY |
| | | | HISFQRIAEKEIMDAVETGKLYLFQIYNKDFAKGHHGKPNLHTLYWT |
| | | | GLFSPENLAKTSIKLNGQAELFYRPKSRMKRMAHRLGEKMLNKKLK |
| | | | DQKTPIPDTLYQELYDYVNHRLSHDLSDEARALLPNVITKEVSHEIIK |
| | | | DRRFTSDKFFFHVPITLNYQAANSPSKFNQRVNAYLKEHPETPIIGIDR |
| | | | GERNLIYITVIDSTGKILEQRSLNTIQQFDYQKKLDNREKERVAARQA |
| | | | WSVVGTIKDLKQGYLSQVIHEIVDLMIHYQAVVVLENLNFGFKSKRT |
| | | | GIAEKAVYQQFEKMLIDKLNCLVLKDYPAEKVGGVLNPYQLTDQFT |
| | | | SFAKMGTQSGFLFYVPAPYTSKIDPLTGFVDPFVWKTIKNHESRKHF |
| | | | LEGFDFLHYDVKTGDFILHFKMNRNLSFQRGLPGFMPAWDIVFEKNE |
| | | | TQFDAKGTPFIAGKRIVPVIENHRFTGRYRDLYPANELIALLEEKGIVF |
| | | | RDGSNILPKLLENDDSHAIDTMVALIRSVLQMRNSNAATGEDYINSP |
| | | | VRDLNGVCFDSRFQNPEWPMDADANGAYHIALKGQLLLNHLKESK |
| | | | DLKLQNGISNQDWLAYIQELRN (SEQ ID NO 42) |
| Prevotella | Prevotella | Cas12a/ | MNIKNFTGLYPLSKTLRFELKPIGKTKENIEKNGILTKDEQRAKDYLI |
| alb | albensis | Cpf1 | VKGFIDEYHKQFIKDRLWDFKLPLESEGEKNSLEEYQELYELTKRND |
| | | | AQEADFTEIKDNLRSSITEQLTKSGSAYDRIFKKEFIREDLVNFLEDEK |
| | | | DKNIVKQFEDFTTYFTGFYENRKNMYSSEEKSTAIAYRLIHQNLPKF |
| | | | MDNMRSFAKIANSSVSEHFSDIYESWKEYLNVNSIEEIFQLDYFSETL |
| | | | TQPHIEVYNYIIGKKVLEDGTEIKGINEYVNLYNQQQKDKSKRLPFLV |
| | | | PLYKQILSDREKLSWIAEEFDSDKKMLSAITESYNHLHNVLMGNENE |
| | | | SLRNLLLNIKDYNLEKINITNDLSLTEISQNLFGRYDVFTNGIKNKLRV |
| | | | LTPRKKKETDENFEDRINKIFKTQKSFSIAFLNKLPQPEMEDGKPRNIE |
| | | | DYFITQGAINTKSIQKEDIFAQIENAYEDAQVFLQIKDTDNKLSQNKT |
| | | | AVEKIKTLLDALKELQHFIKPLLGSGEENEKDELFYGSFLAIWDELDT |
| | | | ITPLYNKVRNWLTRKPYSTEKIKLNFDNAQLLGGWDVNKEHDCAGI |
| | | | LLRKNDSYYLGIINKKTNHIFDTDITPSDGECYDKIDYKLLPGANKML |
| | | | PKVFFSKSRIKEFEPSEAIINCYKKGTHKKGKNFNLTDCHRLINFFKTS |
| | | | IEKHEDWSKFGFKFSDTETYEDISGFYREVEQQGYRLTSHPVSASYIH |
| | | | SLVKEGKLYLFQIWNKDFSQFSKGTPNLHTLYWKMLFDKRNLSDVV |
| | | | YKLNGQAEVFYRKSSIEHQNRIIHPAQHPITNKNELNKKHTSTFKYDII |
| | | | KDRRYTVDKFQFHVPITINFKATGQNNINPIVQEVIRQNGITHIIGIDR |
| | | | GERHLLYLSLIDLKGNIIKQMTLNEIINEYKGVTYKTNYHNLLEKREK |
| | | | ERTEARHSWSSIESIKELKDGYMSQVIHKITDMMVKYNAIVVLEDLN |
| | | | GGFMRGRQKVEKQVYQKFEKKLIDKLNYLVDKKLDANEVGGVLNA |
| | | | YQLTNKFESFKKIGKQSGFLFYIPAWNTSKIDPITGFVNLENTRYESIK |
| | | | ETKVFWSKFDIIRYNKEKNWFEFVFDYNTFTTKAEGTRTKWTLCTH |
| | | | GTRIQTFRNPEKNAQWDNKEINLTESFKALFEKYKIDITSNLKESIMQ |
| | | | ETEKKFFQELHNLLHLTLQMRNSVTGTDIDYLISPVADEDGNFYDSRI |
| | | | NGKNFPENADANGAYNIARKGLMLIRQIKQADPQKKFKFETITNKD |
| | | | WLKFAQDKPYLKD (SEQ ID NO 43) |

TABLE 5
Promoters

| | |
|---|---|
| >Promoter1 | CAGCCACTGCCCCAGACTCAGGGCTTTGCCATTGGTCCCCACCTCCTCTGCTCCG |
| | GAGTTTTTCTCCAGCTCCCCACCAAGCCACACAAAGTGACTTCTCGGAAACATTA |
| | GCCGATTCTGCTGAGCAGGAAGGGAGGAAAGGGATGATGGGGGCGGGGGTGAG |
| | ATAAGGGAAGGGCTCTTCTGGCTGCTGGACACACACACACACACACTCAAACAC |
| | ACACACGCCCCACCCAATGGGTGGCCGTGGATGGCAGGTCGTGCAACCCCCTCCT |
| | CCGCCTTCTATTAGCGCATGGTGCAGAGGCTACAGCGTCGCCACCACCGCGCCCC |
| | TAGCTGGGTCCCCGCCCTGCGCCGCCCGCAGGAGTGGAGAGAGGGAGGGAGGGA |
| | GGGAGCAAGGGGTGGGGACCCGGGCGCGCTGGGAGGAGTGGAGGAGGCAAAGC |
| | GGCGCAGCTGCCCTCGGGGAGGCGGGGCTGCTACCTCCACGGGCGCGCCCTGGC |
| | AGGAGGGGCGCAGTCTGCTTGCAGGCGGTCGCCAGCGCTCCAGCGGCGGCTGTC |
| | GGCTTTCCAATTCCGCCAGCTCGGCTGAGGCTGGGCTAGCCTGGGTGCCAGTGGC |
| | TGC (SEQ ID NO 44) |
| >Promoter2 | CTTGAGCGTCACCGCCCCACTCGCGGTTGTGAGCAAAGCCCTACGGAAGAAACC |
| | AATTCCCAGCCTAGACTCTTCAGAGCCCAAGGTCGGGGAGGCGCTGGCCTGGCG |
| | GTGTTGTCTGGCTCCCCAGCCACTGCCCCAGACTCAGGGCTTTGCCATTGGTCCC |
| | CACCTCCTCTGCTCCGGAGTTTTTCTCCAGCTCCCCACCAAGCCACACAAAGTGA |
| | CTTCTCGGAAACATTAGCCGATTCTGCTGAGCAGGAAGGGAGGAAAGGGATGAT |
| | GGGGGCGGGGGTGAGATAAGGGAAGGGCTCTTCTGGCTGCTGGACACACACACA |
| | CACACACTCAAACACACACACGCCCCACCCAATGGGTGGCCGTGGATGGCAGGT |
| | CGTGCAACCCCCTCCTCCGCCTTCTATTAGCGCATGGTGCAGAGGCTACAGCGTC |
| | GCCACCACCGCGCCCCTAGCTGGGTCCCCGCCCTGCGCCGCCCGCAGGAGTGGA |
| | GAGAGGGAGGGAGGGAGGGAGCAAGGGGTGGGGACCCGGGCGCGCTGGGAGG |
| | AGTGGAGGAGGCAAAGCGGCGCAGCTGCCCTCGGGGAGGCGGGGCTGCTACCTC |
| | CACGGGCGCGCCCTGGCAGGAGGGGCGCAGTCTGCTTGCAGGCGGTCGCCAGCG |
| | CTCCAGCGGCGGCTGTCGGCTTTCCAATTCCGCCAGCTCGGCTGAGGCTGGGCTA |
| | GCCTGGGTGCCAGTGGCTGC (SEQ ID NO 45) |
| >Promoter3 | GAGTGGAGAGAGGGAGGGAGGGAGGGAGCAAGGGGTGGGGACCCGGGCGCGCT |
| | GGGAGGAGTGGAGGAGGCAAAGCGGCGCAGCTGCCCTCGGGGAGGCGGGGCTG |
| | CTACCTCCACGGGCGCGCCCTGGCAGGAGGGGCGCAGTCTGCTTGCAGGCGGTC |
| | GCCAGCGCTCCAGCGGCGGCTGTCGGCTTTCCAATTCCGCCAGCTCGGCTGAGGC |
| | TGGGCTAGCCTGGGTGCCAGTGGCTGCTAGCGGCAGGCGTCCCCTGAGCAACAG |
| | GAGCCCAGAGAAAAAGAAGCAGCCCTGAGAGAGCGCCGGGGAAGGAGAGGCCC |
| | GCGCCCTCTCCTGGAGCCAGATTCTGCAGGTGCACTGGGTGGGGATGATCGGCG |
| | GGCTAGGTTGCAAGTAAGTGCCTTTTCTTTTGCTGCTTTCTTTATTTCTTTGTTTCC |
| | ATCCAGGCCTCTTATGTGAGGAGCTGAAGAGGAATTAAAATATACAGGATGAAA |
| | AGGCGGCCGCCATGGTGAGCAAGGGCGAGGAGGATAA (SEQ ID NO 46) |
| >Promoter4 | TCGAATTAACACAAGACAGAAGTCATATAAATCTTAAGAAGGTAGAAAATGTTA |
| | CTTCTAGAAAACTCCACCAAAAGTTGGTTGGATAATTTCAGGTCTTCTTTCTGGA |
| | AAACGAAGAGTCACAAAGTCTATTTTTCTTTCTACTCCCAAGTTCCACTCATTCTC |
| | CAATTAAATGTGACTCATAGGTGACTGAATGTCATGTTTTGGCCACTCTAAGCAA |
| | AGGGATTCCTTGGACTTGGGTACACATATTTTAAGAATATTGTATATATAAGAAT |
| | ATTGTATATATTCAACTGTCCATCTTGCTGCTTCTCTCTTACACCAACTTCATTTTG |
| | TAGCATCAGCTGCATAAGCTCTTGCTTTTCCCCCTATGCAGACATCTATTCATCAG |
| | GCTCTGTGTACACTGGCAACATAGGAATAAACTGGATAAGGTTGCTGTTCCCAGA |
| | AGAATCTCAGAATATTGTATTTATTTAATGGCATCAGGAAAAACCAGTCCACCAA |
| | TTTTAGAAAGAAAAAAGTCTTAAAGCTGCACATTTTTATGAACTTAGACAATTAC |
| | TATATTTTGACTATTATTGACTCTAATCTATTCCAAGAATTATACAGACAGAAAG |
| | AGCTTTGGTAACAGCCTACATATCAATATTTACTGAACCAAGAACTATCACAAAA |
| | CGTCTGTTGAAAATTTCCATGTTTTAATGATGATGATGCTAATGGCAGCTATGATT |
| | TATTAAACACTTTCTATGTGCAGGATGTAAGCTAAGTAATTTGTACACATTATCTT |
| | ATTTAAACTTATTACAAGTCTTATTTTCTTGAGGTAGATGTTATTATCCTATTTTC |
| | ACAGACAGACTTATGCCTTGAAGAATTTGGTAAAGTCACTAAGTAGTCAGTAGCC |
| | AAATCATGTATTCTTAATCCTTATTCAATATTGTCCCCCTATAGAAGAAACCTTGA |
| | GTTTGTAACTTTTCAGTAAGGCTATTTAGCTTGTGTCCTGAAGACACTCTCACCTA |
| | TA (SEQ ID NO 47) |
| >Promoter5 | TCGAATTAACACAAGACAGAAGTCATATAAATCTTAAGAAGGTAGAAAATGTTA |
| | CTTCTAGAAAACTCCACCAAAAGTTGGTTGGATAATTTCAGGTCTTCTTTCTGGA |
| | AAACGAAGAGTCACAAAGTCTATTTTTCTTTCTACTCCCAAGTTCCACTCATTCTC |
| | CAATTAAATGTGACTCATAGGTGACTGAATGTCATGTTTTGGCCACTCTAAGCAA |
| | AGGGATTCCTTGGACTTGGGTACACATATTTTAAGAATATTGTATATATAAGAAT |
| | ATTGTATATATTCAACTGTCCATCTTGCTGCTTCTCTCTTACACCAACTTCATTTTG |
| | TAGCATCAGCTGCATAAGCTCTTGCTTTTCCCCCTATGCAGACATCTATTCATCAG |
| | GCTCTGTGTACACTGGCAACATAGGAATAAACTGGATAAGGTTGCTGTTCCCAGA |
| | AGAATCTCAGAATATTGTATTTATTTAATGGCATCAGGAAAAACCAGTCCACCAA |
| | TTTTAGAAAGAAAAAAGTCTTAAAGCTGCACATTTTTATGAACTTAGACAATTAC |
| | TATATTTTGACTATTATTGACTCTAATCTATTCCAAGAATTATACAGACAGAAAG |
| | AGCTTTGGTAACAGCCTACATATCAATATTTACTGAACCAAGAACTATCACAAAA |
| | CGTCTGTTGAAAATTTCCATGTTTTAATGATGATGATGCTAATGGCAGCTATGATT |
| | TATTAAACACTTTCTATGTGCAGGATGTAAGCTAAGTAATTTGTACACATTATCTT |
| | ATTTAAACTTATTACAAGTCTTATTTTCTTGAGGTAGATGTTATTATCCTATTTTC |
| | ACAGACAGACTTATGCCTTGAAGAATTTGGTAAAGTCACTAAGTAGTCAGTAGCC |
| | AAATCATGTATTCTTAATCCTTATTCAATATTGTCCCCCTATAGAAGAAACCTTGA |
| | GTTTGTAACTTTTCAGTAAGGCTATTTAGCTTGTGTCCTGAAGACACTCTCACCTA |
| | TA (SEQ ID NO 48) |
| >Promoter6 | TCAATTATAACAGACATGTCTTAGTCTACCTTTTATTGCTGTAACTGAATACCTGA |
| | GACTGGTTAAAGTATTCAATAAAGAAATTTATTTCTTAAAGTTCTGGAAGCTGGG |
| | AAGTCTAAGGTGAAGGGTCCACATCTGGTGGGAACTTTCTTGCTGGTGGGCACAC |
| | CAGAGAGTCCCAAGGCAGCTCATGGCATCACATGGCGAGGGGTGTTCACTGAGA |
| | GCCAAACTGGGTTTTATAATAAACCCACTCTTGTGATATCTAACTCACTCCCATG |
| | ATAACCCATTAAGCCCTTACTCTATATTCCATTCATGAAGGCAGAACCTTCATGA |
| | CCTCATCACCTTTTAAAGGCTCTGCCTCTTAATACTGTTACATTGGGAATTAAGTT |
| | CCAACATGAGTTTCAAAAGGGACAAACATTCAAACCACAGCAAAACATTAAAAA |
| | AAAAATTTTCTGATTAAAGAGGGAGCATACATATTTTCAAGTGTTCTTAAGTGGA |
| | AAATAATGGATGTGAATGACTGGGCAAATTTAAGAGTGGCATGAGGAGTAATTT |
| | ATTCTGCTAAATACGTAGCTTTAGGAATTTAAAAAAAAAAGCCATGTTACATTTA |
| | GTGAGACATCTGGAAATACTACATGAGGATGAGCACTTTAAAACACTTCCCGCTT |
| | CATCAGTCAGTTTACCTGTCTCTGATGGTAACAGCAATGAACTTTTTGTGAACAA |
| | TGACTTTTTCTACGCCTGAAATAGATAAATATATAAAGTTGGCTCTCATCTGTTCC |
| | TATTCTTCCCTCGAAAAATTGAATCTCTCAGTCACTAACTTATTCGTAATGAAGA |
| | ACTGCATTCCAGACATTTTTCTTAGACATCGTCTCCTGCTACACAGGATGTGAGG |
| | CAAATGAATATTTTCCATGGAACTAATTCAGAGAGTAAACATATTTATAGATCCA |
| | GATCTAAGGTCATGGAGGAGCTCATTGCTTTGTAACTAAAATGTGTATTGATTGT |
| | ATTGCTGTTC (SEQ ID NO 49) |
| >Promoter7 | CAGTCTCAGTCACTGAAGTTTCCTTCCTTCAATAGCACATCTTACAAGTCAAGAA |
| | TGTAAGGTCCAAAGCCCCAGGACAAATCTCAGAAGCAAAGAAAGCAGCAGGGA |
| | AAAGTAGACCCTGGGATTTGATTTCCTTCCGGTTATCTCTAAGCATCATTTCCATG |
| | ATAGAAGGTGTGGAAGCAAATAATAAAAGTGCCCGTCACTAGTGTTTATCCTGC |
| | AAAGTGGTCTGCCCTTTTGAGAGGCACCCTGCCCTATGGCCATCCTTTGATTTCTT |
| | CCCTTGGTGGAAATTTCCTGTTTCTCTTTGAGAAAGATAATTCAACCCTGTTTCCA |
| | TTGTTCTTCCTCCCAGGCTGCTGTAGGAAAGCGCACGCACACACTCCACACAAAA |
| | TGAATTTTTAAAAAATTTATTTTCACAGTCGCTCCTACCAGCTCTGAAATTCAGAA |
| | CCCATATGACTGATGGCATATTCAGATAATCGGGTCCCAGGTCTGGAAAAGCAG |
| | CCTTTTCCCCACGTTTCTTTCCCCACCTAGGACCTCCTCTGATTCTTCACTGCATCT |
| | TCGAAAGAAAATGTATTATTTGCTTGCCTGGAAGACGCTGCAATTCAATTGATTT |
| | TATATATACATATATATAAAGAAAACAGAAAACATAGCCTAGATACCGGTCTTG |
| | AGCGTCACCGCCCCACTCGCGGTTGTGAGCAAAGCCCTACGGAAGAAACCAATT |
| | CCCAGCCTAGACTCTTCAGAGCCCAAGGTCGGGGAGGCGCTGGCCTGGCGGTGT |
| | TGTCTGGCTCCCCAGCCACTGCCCCAGACTCAGGGCTTTGCCATTGGTCCCCACC |
| | TCCTCTGCTCCGGAGTTTTTCTCCAGCTCCCCACCAAGCCACACAAAGTGACTTCT |
| | CGGAAACATTAGCCGATTCTGCTGAGCAGGAAGGGAGGAAAGGGATGATGGGG |
| | GCGGGGGTGAGATAAGGGAAGGGCTCTTCTGGCTGCTGGACACACACACACACA |
| | CACTCAAACACACACACGCCCCACCCAATGGGTGGCCGTGGATGGCAGGTCGTG |
| | CAACCCCCTCCTCCGCCTTCTATTAGCGCATGGTGCAGAGGCTACAGCGTCGCCA |
| | CCACCGCGCCCCTAGCTGGGTCCCCGCCCTGCGCCGCCCGCAGGAGTGGAGAGA |
| | GGGAGGGAGGGAGGGAGCAAGGGGTGGGGACCCGGGCGCGCTGGGAGGAGTGG |
| | AGGAGGCAAAGCGGCGCAGCTGCCCTCGGGGAGGCGGGGCTGCTACCTCCACGG |
| | GCGCGCCCTGGCAGGAGGGGCGCAGTCTGCTTGCAGGCGGTCGCCAGCGCTCCA |
| | GCGGCGGCTGTCGGCTTTCCAATTCCGCCAGCTCGGCTGAGGCTGGGCTAGCCTG |
| | GGTGCCAGTGGCTGCTAGCGGCAGGCGTCCCCTGAGCAACAGGAGCCCAGAGAA |
| | AAAGAAGCAGCCCTGAGAGA (SEQ ID NO 50) |
| >hSyn | CAGTGCAAGTGGGTTTTAGGACCAGGATGAGGCGGGGTGGGGGTGCCTACCTGA |
| | CGACCGACCCCGACCCACTGGACAAGCACCCAACCCCCATTCCCCAAATTGCGC |
| | ATCCCCTATCAGAGAGGGGGAGGGGAAACAGGATGCGGCGAGGCGCGTGCGCA |
| | CTGCCAGCTTCAGCACCGCGGACAGTGCCTTCGCCCCCGCCTGGCGGCGCGCGCC |
| | ACCGCCGCCTCAGCACTGAAGGCGCGCTGACGTCACTCGCCGGTCCCCCGCAAA |
| | CTCCCCTTCCCGGCCACCTTGGTCGCGTCCGCGCCGCCGCCGGCCCAGCCGGACC |
| | GCACCACGCGAGGCGCGAGATAGGGGGGCACGGGCGCGACCATCTGCGCTGCGG |
| | CGCCGGCGACTCAGCGCTGCCTCAGTCTGCGGTGGGCAGCGGAGGAGTCGTGTC |
| | GTGCCTGAGAGCGCAG (SEQ ID NO 51) |

TABLE 6
Examples of gRNA sequences

| | | SEQ | Human gene |
|---|---|---|---|
| Variant | gRNA target sequences | ID NO | target |
| Campylobacter | GGCTGCTAGCGGCAGGCGTCCC | 52 | SCN9A |
| jejuni dCas9 | GCAGAATCTGGCTCCAGGAGAG | 53 | SCN9A |
| | GGTGGGGATGATCGGCGGGCTA | 54 | SCN9A |
| | TAATTCCTCTTCAGCTCCTCAC | 55 | SCN9A |
| | AGGGCTCTTCTGGCTGCTGGAC | 56 | SCN9A |
| | GGGACGCCTGCCGCTAGCAGCC | 57 | SCN9A |
| | TCCCCACAGAAGCAGCAAAAGA | 58 | SCN9A |
| | TTTCGCCCGTGCTCGCCTCAAC | 59 | SCN9A |
| | GTTTTTCTCCAGCTCCCCACCA | 60 | SCN9A |
| | AAGGGAAGGGCTCTTCTGGCTG | 61 | SCN9A |
| | GTTCCAGAACCGAGATGTGAAA | 62 | SCN9A |
| | TTTTCTCCAGCTCCCCACCA | 63 | SCN9A |
| | CCTCTCTTCTTTCCAGGTGG | 64 | SCN9A |
| | GGGGTCCAAGTCCTCCAGGGGC | 65 | SCN9A |
| Staphylococcus | CCCAGCGCGCCCGGGTCCCC | 66 | SCN9A |
| aureus dCas9 | GCGCGCTGGGAGGAGTGGAG | 67 | SCN9A |
| | GCTAGCCCAGCCTCAGCCGA | 68 | SCN9A |
| | TGCACCTGCAGAATCTGGCT | 69 | SCN9A |
| | ACCCAGTGCACCTGCAGAAT | 70 | SCN9A |
| | CAGGTGCACTGGGTGGGGAT | 71 | SCN9A |
| | GCCTCTTATGTGAGGAGCTG | 72 | SCN9A |
| | CTTCAGCTCCTCACATAAGA | 73 | SCN9A |
| | TATATTTTAATTCCTCTTCA | 74 | SCN9A |
| | CCCGGCATGGTGTCAGAGCC | 75 | SCN9A |
| Streptococcus | GGCGGTCGCCAGCGCTCCAG | 76 | SCN9A |
| pyogenes | GCCACCTGGAAAGAAGAGAG | 77 | SCN9A |
| dCas9 | GGTCGCCAGCGCTCCAGCGG | 78 | SCN9A |
| | GCCAGCAATGGGAGGAAGAA | 79 | SCN9A |
| | GTTCCAGGTGGCGTAATACA | 80 | SCN9A |
| | GGCGGGGCTGCTACCTCCAC | 81 | SCN9A |
| | GGGCGCAGTCTGCTTGCAGG | 82 | SCN9A |
| | GGCGCTCCAGCGGCGGCTGT | 83 | SCN9A |
| | GACCGGGTGGTTCCAGCAAT | 84 | SCN9A |
| | GGGGTGGTTCCAGCAATGGG | 85 | SCN9A |
| Streptococcus | GTTTACTCCGGAGTCACTGG | 86 | SCN10A |
| pyogenes | GCTATCTCCACCAGTGACTC | 87 | SCN10A |
| dCas9 | GACATCACCCAGGGCCAAGG | 88 | SCN10A |
| | GTAGTTTCGAGGGATCCAAT | 89 | SCN10A |
| | GCTCCCAGCAGAACTGATCG | 90 | SCN10A |
| | GTGACTCCGGAGTAAAGCGA | 91 | SCN10A |
| | GGGAGCTCACCATAGAACTT | 92 | SCN10A |
| | GACGGATCTAGATCCTCCAG | 93 | SCN10A |
| | GCCGGGTAAGAGCTACTAGT | 94 | SCN10A |
| | GCCCGGTGTGTGCTGTAGAA | 95 | SCN10A |

TABLE 7
Examples of peptide sequences

| Peptides used to increase targeting | SEQ | | |
|---|---|---|---|
| of SCN9A expressing cells | ID | | |
| (delivery vector modification) | NO | Name | Origin |
| YCQKWMWTCDSERKCCEGMVCRLWCKKKLW | 96 | Protoxin-II | Thrixopelma |
| GPYCQKWMQTCDSERKCCEGMVCRLWCKKKLL | 97 | Protoxin-II | prurient |
| | | variant | |
| | | (JNJ63955) | |
| DCRYMFGDCEKDEDCCKHLGCKRKMKYCAWDFTFT | 98 | μ-TRTX-Pn3a | Pamphobeteus |
| | | | nigricolor |
| YCQKWMWTCDSKRACCEGLRCKLWCRKII | 99 | Jz-Tx-V | Chilobrachys |
| YCQKWMWTCDSKRACCEGLRCKLWCRKEI | 100 | Jz-Tx-V | jingzhao |
| | | variant | |
| YCQKWLWTCDSERKCCEDMVCRLWCKKRL | 101 | GsAFI | Grammostola |
| | | | spatulata |
| ACLGQWDSCDPKASKCCPNYACEWKYPWCRYKLF | 102 | Phlotoxin 1 | Phlogiellus |
| ACLGQWASCDPKASKCCPNYACEWKYPWCRYKLF | 103 | Phlotoxin 1 | genus |
| | | variant | |
| DCLKFGWKCNPRNDKCCSGLKCGSNHNWCKLHL | 104 | Tpla | Thrixopelma |
| | | (Protoxin-III) | pruriens |
| DCLGFMRKCIPDNDKCCRPNLVCSRTHKWCKYVF | 105 | GpTx-1 | Grammostola |
| DCLGFFRKCIPDNDKCCRPNLVCSRLHRWCKYVF | 106 | GpTx-1 | porteri |
| DCLGAMRKCIPDNDKCCRPNLVCSRTHKWCKYVF | 107 | variants | |
| ECLEIFKACNPSNDQCCKSSKLVCSRKTRWCKYQI | 108 | HwTx-IV | Selenocosmia |
| GCLGIFKACNPSNDQCCKSSKLVCSRKTRWCKWQI | 109 | m3-HwTx-IV | huwena |
| | | variant (m3- | |
| | | Huwentoxin-IV) | |
| DCLGMFKSCDPENDKCCKRLVCSRSHRWCKWKL | 110 | CcoTx1 | Ceratogyrus |
| ICLGMFKSCDPENDKCCKRLVCSRSHRWCKWKL | 111 | variants | cornuatus |
| DCGTIWHYCGTDQSECCEGWKCSRQLCKYVIDW | 112 | HpTx1 | Heteropoda |
| | | | venatoria |
| ECRYLFGGCSSTSDCCKHLSCRSDWKYCAWDGTFS | 113 | Hm1a | Heteroscodra |
| | | | maculata |
| YCQKW[Nle]WTCDSKRACC[Pra]GLRCKLWCRKEI | 114 | AM-0422 | Odonthobuthus |
| GVRDAYIADDKNCVYTCASNGYCNTECTKNGAESG | 115 | OD-1 | doriae |
| YCQWIGRYGNACWCIKLPDEVPIRIPGKCR | | | |
| LLIILRRRIRKQAHAHSK | 116 | Penetration | |
| | | enhancing | |
| | | peptide | |
| KETWWETWWTEWSQPKKKRKV | 117 | Penetration | |
| | | enhancing | |
| | | peptide | |
| KLALKLALKALKAALKLA | 118 | Penetration | |
| | | enhancing | |
| | | peptide | |
| LSTAADMQGVVTDGMASGLDKDYLKPDD | 119 | Penetration | |
| | | enhancing | |
| | | peptide | |
| LLKKKCWLRCVMGECCKRESDCTQMWKQCYPG | 120 | Penetration | |
| | | enhancing | |
| | | peptide | |

TABLE 8
Examples of ZFP sequences and DNA target.

| | | SEQ ID | |
|---|---|---|---|
| NAME | TARGET | NO | SEQUENCE |
| ZF1 | gcAGTCTGCTTG | 121 | MDYKDHDGDYKDHDIDYKDDDDKMAPKKKRKVGIHGVPAAMA |
| | CAGGCGGTcgcc | | ERPFQCRICMRNFSTSGHLSRHIRTHTGEKPFACDICGRKFADR |
| | agcg (SEQ ID | | SHLARHTKIHTGSQKPFQCRICMRNFSQSGDLTRHIRTHTGEKP |
| | NO: 145) | | FACDICGRKFAYRWLRNNHTKIHTGSQKPFQCRICMRNFSRSD |
| | | | TLSEHIRTHTGEKPFACDICGRKFARAQHLQQHTKIHLRQKDAA |
| | | | RGSRTLVTFKDVFVDFTREEWKLLDTAQQIVYRNVMLENYKNLV |
| | | | SLGYQLTKPDVILRLEKGEEPWLVDYKDDDDKRS |
| ZF2 | ctCCAGCGGCGG | 122 | MDYKDHDGDYKDHDIDYKDDDDKMAPKKKRKVGIHGVPAAMA |
| | CTGTCGGCtttc | | ERPFQCRICMRNFSDRSHLSRHIRTHTGEKPFACDICGRKFADR |
| | cnnn (SEQ ID | | SALARHTKIHTGSQKPFQCRICMRNFSQSSDLSRHIRTHTGEKP |
| | NO: 146) | | FACDICGRKFARSDDLTRHTKIHTGSQKPFQCRICMRNFSRSDD |
| | | | LTRHIRTHTGEKPFACDICGRKFAQRSTLSSHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |
| ZF3 | taGCCTGGGTGC | 123 | MDYKDHDGDYKDHDIDYKDDDDKMAPKKKRKVGIHGVPAAMA |
| | CAGTGGCTgcta | | ERPFQCRICMRNFSQSSDLSRHIRTHTGEKPFACDICGRKFARS |
| | gcgg (SEQ ID | | DALARHTKIHTGSQKPFQCRICMRNFSQSGDLTRHIRTHTGEKP |
| | NO: 147) | | FACDICGRKFARSWGLQVHTKIHTGSQKPFQCRICMRNFSRSD |
| | | | HLSQHIRTHTGEKPFACDICGRKFADSSTRKKHTKIHLRQKDAA |
| | | | RGSRTLVTFKDVFVDFTREEWKLLDTAQQIVYRNVMLENYKNLV |
| | | | SLGYQLTKPDVILRLEKGEEPWLVDYKDDDDKRS |
| ZF4 | agTGGCTGCTAG | 124 | MDYKDHDGDYKDHDIDYKDDDDKMAPKKKRKVGIHGVPAAMA |
| | CGGCAGGCgtcc | | ERPFQCRICMRNFSDRSHLTRHIRTHTGEKPFACDICGRKFAQS |
| | cctg (SEQ ID | | GDLTRHTKIHTGSQKPFQCRICMRNFSRSDDLTRHIRTHTGEKP |
| | NO: 148) | | FACDICGRKFAQRSTLSSHTKIHTGSQKPFQCRICMRNFSRSDV |
| | | | LSEHIRTHTGEKPFACDICGRKFARNQHRKTHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |
| ZF5 | agGCGTCCCCTG | 125 | MDYKDHDGDYKDHDIDYKDDDDKMAPKKKRKVGIHGVPAAMA |
| | AGCAACAGgagc | | ERPFQCRICMRNFSRSDNLSEHIRTHTGEKPFACDICGRKFAER |
| | ccag (SEQ ID | | ANRNSHTKIHTGSQKPFQCRICMRNFSRSDNLARHIRTHTGEKP |
| | NO: 149) | | FACDICGRKFAWRGDRVKHTKIHTGSQKPFQCRICMRNFSDRS |
| | | | DLSRHIRTHTGEKPFACDICGRKFARRTDLRRHTKIHLRQKDAA |
| | | | RGSRTLVTFKDVFVDFTREEWKLLDTAQQIVYRNVMLENYKNLV |
| | | | SLGYQLTKPDVILRLEKGEEPWLVDYKDDDDKRS |
| ZF6 | ggAAGGAGAGGC | 126 | MDYKDHDGDYKDHDIDYKDDDDKMAPKKKRKVGIHGVPAAMA |
| | CCGCGCCCtctc | | ERPFQCRICMRNFSDRSDLSRHIRTHTGEKPFACDICGRKFARR |
| | ctgg (SEQ ID | | TDLRRHTKIHTGSQKPFQCRICMRNFSDRSDLSRHIRTHTGEKP |
| | NO: 150) | | FACDICGRKFARSHHLKAHTKIHTGSQKPFQCRICMRNFSRSAN |
| | | | LARHIRTHTGEKPFACDICGRKFARSDNLREHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |
| ZF7 | ctGCAGGTGCAC | 127 | MDYKDHDGDYKDHDIDYKDDDDKMAPKKKRKVGIHGVPAAMA |
| | TGGGTGGGgatg | | ERPFQCRICMRNFSRSAHLSRHIRTHTGEKPFACDICGRKFATS |
| | atcg (SEQ ID | | GHLSRHTKIHTGSQKPFQCRICMRNFSRSDALSEHIRTHTGEKP |
| | NO: 151) | | FACDICGRKFAQNATRTKHTKIHTGSQKPFQCRICMRNFSTSGH |
| | | | LSRHIRTHTGEKPFACDICGRKFAQSGDLTRHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |
| ZF8 | gcGCGCCCGTGG | 128 | MDYKDHDGDYKDHDIDYKDDDDKMAPKKKRKVGIHGVPAAMA |
| | AGGTAGCAgccc | | ERPFQCRICMRNFSQSGDLTRHIRTHTGEKPFACDICGRKFAQS |
| | cgcc (SEQ ID | | GARNIHTKIHTGSQKPFQCRICMRNFSRSANLARHIRTHTGEKP |
| | NO: 152) | | FACDICGRKFARSDALARHTKIHTGSQKPFQCRICMRNFSDRSD |
| | | | LSRHIRTHTGEKPFACDICGRKFARRTDLRRHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |
| ZF9 | CCTGCCAGGGCG | 129 | MDYKDHDGDYKDHDIDYKDDDDKMAPKKKRKVGIHGVPAAMA |
| | CGCCCGTGgagg | | ERPFQCRICMRNFSRSDVLSRHIRTHTGEKPFACDICGRKFADS |
| | tagc (SEQ ID | | RDRKNHTKIHTGSQKPFQCRICMRNFSRSADLTRHIRTHTGEKP |
| | NO: 153) | | FACDICGRKFADRSHLARHTKIHTGSQKPFQCRICMRNFSRSDN |
| | | | LSEHIRTHTGEKPFACDICGRKFASKQYLIKHTKIHLRQKDAARG |
| | | | SRTLVTFKDVFVDFTREEWKLLDTAQQIVYRNVMLENYKNLVSL |
| | | | GYQLTKPDVILRLEKGEEPWLVDYKDDDDKRS |
| ZF10 | cgACAGCCGCCG | 130 | MDYKDHDGDYKDHDIDYKDDDDKMAPKKKRKVGIHGVPAAMA |
| | CTGGAGCGctgg | | ERPFQCRICMRNFSRSADLTRHIRTHTGEKPFACDICGRKFAQS |
| | cgac (SEQ ID | | GHLSRHTKIHTGSQKPFQCRICMRNFSQSSDLSRHIRTHTGEKP |
| | NO: 154) | | FACDICGRKFAHRKSLSRHTKIHTGSQKPFQCRICMRNFSDRSD |
| | | | LSRHIRTHTGEKPFACDICGRKFAQSSTRARHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |
| ZF11 | ctCCAGGAGAGG | 131 | MDYKDHDGDYKDHDIDYKDDDDKMAPKKKRKVGIHGVPAAMA |
| | GCGCGGGCctct | | ERPFQCRICMRNFSDRSHLTRHIRTHTGEKPFACDICGRKFARS |
| | cctt (SEQ ID | | DDLTRHTKIHTGSQKPFQCRICMRNFSDRSHLTRHIRTHTGEKP |
| | NO: 155) | | FACDICGRKFARSDNLTRHTKIHTGSQKPFQCRICMRNFSQSGH |
| | | | LARHIRTHTGEKPFACDICGRKFAQKGTLGEHTKIHLRQKDAAR |
| | | | GSRTLVTFKDVFVDFTREEWKLLDTAQQIVYRNVMLENYKNLVS |
| | | | LGYQLTKPDVILRLEKGEEPWLVDYKDDDDKRS |

TABLE 9
Examples of repressor domains

| | SEQ ID | |
|---|---|---|
| Domain | NO | Sequence |
| ZIM3 | 132 | VTFEDVTVNFTQGEWQRLNPEQRNLYRDVMLENYSNLVSVGQGETT |
| | | KPDVILRLEQGKEPWL |
| KOX1 | 133 | VTFKDVFVDFTREEWKLLDTAQQIVYRNVMLENYKNLVSLGYQLTK |
| | | PDVILRLEKGEEPWLV |
| ZNF554 | 134 | VTFEDVSMDFSQEEWELLEPAQKNLYREVMLENYRNVVSLEALKNQ |
| | | CTDVGIKEGPLSPAQT |
| ZNF264 | 135 | VTFDDVAVTFTKEEWGQLDLAQRTLYQEVMLENCGLLVSLGCPVPK |
| | | AELICHLEHGQEPWT |
| ZNF324 | 136 | MAFEDVAVYFSQEEWGLLDTAQRALYRRVMLDNFALVASLGLSTSR |
| | | PRVVIQLERGEEPWV |
| MeCP2 | 137 | VQVKRVLEKSPGKLLVKMPFQASPGGKGEGGGATTSAQVMVIKRPG |
| | | RKRKAEADPQAIPKKRGRKPGSVVAAAAAEAKKKAVKESSIRSVQET |
| | | VLPIKKRKTRETVSIEVKEVVKPLLVSTLGEKSGKGLKTCKSPGRKSK |
| | | ESSPKGRSSSASSPPKKEHHHHHHHAESPKAPMPLLPPPPPPEPQSSED |
| | | PISPPEPQDLSSSICKEEKMPRAGSLESDGCPKEPAKTQPMVAAAATTT |
| | | TTTTTTVAEKYKHRGEGERKDIVSSSMPRPNREEPVDSRTPVTERVS |
| MBD2b | 138 | ARYLGNTVDLSSFDFRTGKMMPSKLQKNKQRLRNDPLNQNKGKPDL |
| | | NTTLPIRQTASIFKQPVTKVTNHPSNKVKSDPQRMNEQPRQLFWEKRL |
| | | QGLSASDVTEQIIKTMELPKGLQGVGPGSNDETLLSAVASALHTSSAPI |
| | | TGQVSAAVEKNPAVWLNTSQPLCKAFIVTDEDIRKQEERVQQVRKIL |
| | | EDALMADILSRAADTEEMDIEMDSGDEAEF |
| SID | 139 | MNIQMLLEAADYLERREREAEHGYASMLP |
| HP1a | 140 | MKEGENNKPREKSEGNKRKSSFSNSADDIKSKKKREQSNDIARGFER |
| | | GLEPEKIIGATDSCGDLMFLMKWKDTDEADLVLAKEANVKCPQIVIA |
| | | FYEERLTWHAYPEDAENKEKESAKSEF |
| SIRT5 | 141 | SSSMADFRKFFAKAKHIVIISGAGVSAESGVPTFRGAGGYWRKWQAQ |
| | | DLATPLAFAHNPSRVWEFYHYRREVGSKEPNAGHRAIAECETRLGKQ |
| | | GRRVWITQNIDELHRKAGTKNLLEIHGSLFKTRCTSCGWAENYKSPIC |
| | | PALSGKGAPEPGTQDASIPVEKLPRCEEAGCGGLLRPHWWFGENLDP |
| | | AILEEVDRELAHCDLCLWGTSSWYPAAFAPQVAARGVPVAEFNTETT |
| | | PATNRFRFHFQGPCGTTLPEALACHENETVS |
| SETD8 | 142 | SCDSTNAAIAKQALKKPIKGKQAPRKKAQGKTQQNRKLTDFYPVRRS |
| | | SRKSKAELQSEERKRIDELIESGKEEGKIDLIDGKGRGVIATKQFSRGD |
| | | FVVEYHGDLIEITDAKKREALYAQDPSTGCYYYFQYLSKTYCVDATR |
| | | ETNRLGRLINHSKCGNCQTKLHDIDGVPHLILIASRDIAAGEELLY |
| | | DYGDRSKASIEAFPWLKHEF |
| HDT1 | 143 | MEFWGIEVKSGKPVTVTPEEGILIHVSQASLGECKNKKGEFVPLHVKV |
| | | GNQNLVLGTLSTENIPQLFCDLVFDKEFELSHTWGKGSVYFVGYKTP |
| | | NIEPQGYSEEEEEEEEEVPAGNAAKAVAKPKAKPAEVKPAVDDEEDE |
| | | SDSDGMDEDDSDGEDSEEEEPTPKKPASSKKRANETTPKAPVSAKKA |
| | | KVAVTPQKTDEKKKGGKAANQSEF |
| SUPRD | 144 | DLELRL |

1. A nucleic acid comprising a sequence encoding a nucleic acid binding domain, a sequence encoding an epigenetic modulator that regulates transcription of one or more target molecules, and a promoter comprising a Nav1.7 promoter, a promoter having a sequence at least about 90% identical to a sequence of Table 5, or a promoter of a gene selected from Tables 1-3.

2. The nucleic acid of claim 1, wherein the one or more target molecules comprises a nucleic acid encoding an ion channel and/or is associated with an ion channel.

3. The nucleic acid of claim 1, wherein the one or more target molecules comprises SCN9A, SON10A, or SCN11A, or a combination thereof.

4. The nucleic acid of claim 1, wherein the one or more target molecules is associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), erythromelalgia, diabetic neuropathy, small fiber neuropathy, rheumatoid arthritis, trigeminal neuralgia, or a combination thereof.

5. The nucleic acid of claim 1, wherein the one or more target molecules comprises one or more genes of Table 1.

6. The nucleic acid of claim 1, wherein the one or more target molecules is associated with pain.

7. (canceled)

8. The nucleic acid of claim 1, wherein the nucleic acid binding domain comprises a zinc finger protein.

9. The nucleic acid of claim 1, wherein the nucleic acid binding domain comprises a nuclease dead Clustered Regularly Interspaced Short Palindromic Repeats associated protein (dCas).

10. The nucleic acid of claim 1, wherein the epigenetic modulator comprises a domain having transcription repression activity (repressor domain).

11. The nucleic acid of claim 10, wherein the repressor domain comprises a ZIM3 repressor domain or a Krueppel-associated box (KRAB) domain (recruitment of histone methyltransferases and deacetylases).

12. The nucleic acid of claim 1, wherein the epigenetic modulator comprises a domain having transcription activation activity (activator domain).

13. The nucleic acid of claim 1, wherein the epigenetic modulator comprises ZIM3, KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, ZNF257, VP64, Rta, P16, P65, p300, TET1 catalytic domain, TDG, Ldb1 self-association domain, SAM activator (VP64, p65, HSF1), VPR (VP64, p65, Rta), CD, or SunTag, or a variant or combination thereof.

14. The nucleic acid of claim 1, wherein the promoter comprises the Nav1.7 promoter.

15. The nucleic acid of claim 1, wherein the promoter comprises the sequence at least about 90% identical to a sequence of Table 5, optionally promoter 1 or promoter 2.

16. (canceled)

17. (canceled)

18. A composition comprising a) a nucleic acid comprising a sequence encoding a nucleic acid binding domain and a sequence encoding an epigenetic modulator that regulates transcription of one or more target molecules, and b) a delivery vehicle for delivering the nucleic acid, wherein the delivery vehicle comprises a targeting peptide comprising JNJ63955, m3-Huwentoxin-IV, Phlotoxin 1 (PhITx1), Protoxin-II (ProTx-II), or Ceratotoxin-1 (CcoTx1), or a variant thereof; or a sequence at least about 90% identical to a peptide of Table 7.

19.-32. (canceled)
